(19) United States
(12) Patent Application Publication
(10) Pub. No.: US 2019/0209704 A1
(43) Pub. Date: Jul. 11, 2019

(54) NOVEL ANTIBODY-DRUG CONJUGATES AND RELATED COMPOUNDS, COMPOSITIONS AND METHODS OF USE

(71) Applicant: IGENICA BIOTHERAPEUTICS, INC.

(72) Inventors: David Y. JACKSON, Belmont, CA (US); Edward HA, Solana Beach, CA (US); Paul SAUER, Scotts Valley, CA (US); Simeon BOWERS, Oakland, CA (US); Maureen Fitch Bruhns, San Mateo, CA (US); Jorge Monteon, Fremont, CA (US); Christopher Behrens, San Francisco, CA (US); Randall L. Halcomb, Foster City, CA (US)

(21) Appl. No.: 15/520,401

(22) PCT Filed: Oct. 19, 2015
(86) PCT No.: PCT/US2015/056260

§ 371 Date: Apr. 19, 2017

Related U.S. Application Data
(60) Provisional application No. 62/066,357, filed on Oct. 20, 2014.
(60) Provisional application No. 62/069,826, filed on Oct. 28, 2014.
(60) Provisional application No. 62/106,211, filed on Jan. 21, 2015.

(51) Int. Cl. A61K 47/68; A61K 38/07; A61P 35/00

(52) U.S. Cl. CPC A61K 47/6817; A61K 38/07; A61P 35/00; A61K 47/6889; A61K 47/6855

(57) ABSTRACT

The present disclosure provides novel linker-cytotoxin conjugates and antibody-drug conjugates, including homogenous antibody-drug conjugates, comprising the novel linker-cytotoxin conjugates.

### CROSS-REFERENCE TO RELATED APPLICATIONS

This application claims the benefit of priority of U.S. Provisional Application No. 62/066,357, filed Oct. 20, 2014, U.S. Provisional Application No. 62/069,826, filed Oct. 28, 2014, and U.S. Provisional Application No. 62/106,211, filed Jan. 21, 2015, the entire contents of which are each incorporated herein by reference in their entireties.

### FIELD

This disclosure relates to novel linker-cytotoxin conjugates and antibody-drug conjugates, including homogenous antibody-drug conjugates, comprising such novel linker-cytotoxin conjugates, and methods of their making and use.

### BACKGROUND

In recent years, antibody-drug conjugates (ADCs) have become a novel strategy in the development of cancer therapeutics. The ability to combine the specificity of an antibody directed to a cell-surface antigen with the cytotoxicity of potent drugs, theoretically should allow for higher efficacy and an improved therapeutic index compared to more traditional approaches. Although there are currently many ADCs in clinical development, and although some promising results have been reported, the available data suggests that developing highly efficacious therapeutics through this modality may be more complex than initially expected.

One of the challenges in the development of efficacious ADCs is the selection and synthesis of a linker-toxin combination suitable for chemical conjugation to an antibody. There remains a need for linker-toxin conjugates, particularly linker-toxins that when conjugated to antibodies are able to generate homogeneous ADCs and site specific ADCs.

### SUMMARY

The present disclosure provides novel linker-cytotoxin conjugates and antibody-drug conjugates, including homogenous antibody-drug conjugates, comprising such novel linker-cytotoxin conjugates.

The present disclosure provides substituted maleimide linkers, for example, monosubstituted and disubstituted maleimide linkers, conjugated to cytotoxins, and antibody-drug conjugates, including homogenous antibody-drug conjugates, comprising such maleimide conjugated linkers.

In certain embodiments, the cytotoxin is an auristatin, such as monomethylauristatin F (MMAF) and monomethylauristatin E (MMAE). In certain embodiments, the cytotoxin is a pyrrolobenzodiazepine (PBD), a calicheamicin, doxorubicin, camptothecin, duocarmycin, DM1, DM4, a maytansinoid, or a tubulysin.

The present disclosure also provides antibody-drug conjugates of the following formula (I):

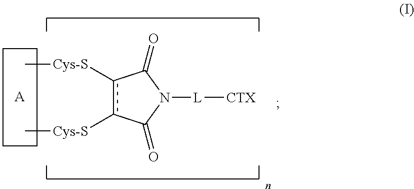

or a pharmaceutically acceptable salt thereof,
wherein:
A is an antibody;
the two depicted cysteine residues are from an opened cysteine-cysteine disulfide bond in A;
L is a cleavable or a noncleavable linker;
CTX is cytotoxin bonded to L by an amide bond, a carbamate bond, a disulfide bond, an ether bond, a thioether bond, or an ester bond;
the [symbol] bond re resents a single or a double bond; and
n is an integer of 1 to 4.

The present disclosure also provides antibody-drug conjugates of the following formula (Ia):

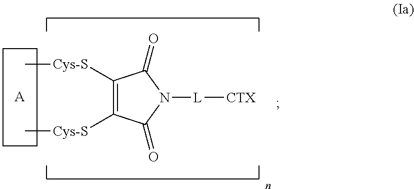

or a pharmaceutically acceptable salt thereof,
wherein:
A is an antibody;
the two depicted cysteine residues are from an opened cysteine-cysteine disulfide bond in A;
L is a cleavable or a noncleavable linker;
CTX is cytotoxin bonded to L by an amide bond, a carbamate bond, a disulfide bond, an ether bond, a thioether bond, or an ester bond; and
n is an integer of 1 to 4.

The present disclosure also provides antibody-drug conjugates of the following formula (Ib):

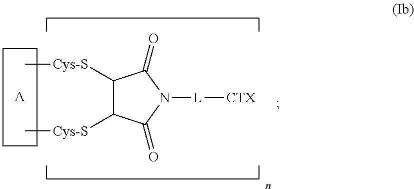

or a pharmaceutically acceptable salt thereof,
wherein:
A is an antibody;
the two depicted cysteine residues are from an opened cysteine-cysteine disulfide bond in A;
L is a cleavable or a noncleavable linker;
CTX is cytotoxin bonded to L by an amide bond, a carbamate bond, a disulfide bond, an ether bond, a thioether bond, or an ester bond; and
n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is an auristatin, a pyrrolobenzodiazepine (PDB), calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond;
and n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is an auristatin bonded to L by an amide bond or a carbamate bond;
and n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is MMAF bonded to L by an amide bond;
and n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is MMAE bonded to L by a carbamate bond;
and n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is a PBD bonded to L by an amide bond or a carbamate bond;
and n is an integer of 1 to 4.

The present disclosure also provides antibody-drug conjugates of formula (I), (Ia) or (Ib), wherein

A is an antibody;
the two depicted cysteine residues are from an opened cysteine-cysteine disulfide bond in A;
L is a cleavable or a noncleavable linker;
CTX is a calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond;
and n is an integer of 1 to 4.

In certain embodiments of the antibody-drug conjugate of formula (I), (Ia) or (Ib), n is an integer of 2. In certain embodiments, n is an integer of 3. In certain embodiments, n is an integer of 4.

In certain embodiments of the antibody-drug conjugate of formula (I), (Ia) or (Ib), where CTX is MMAF, and L is a noncleavable linker.

In certain embodiments of the antibody-drug conjugate of formula (I), (Ia) or (Ib), where CTX is MMAF, and L is —(CH₂)ₘC(O)—, wherein m is an integer of 5 to 11.

In certain embodiments of the antibody-drug conjugate of formula (I), (Ia) or (Ib), where CTX is MMAF, and L is a cleavable linker.

In certain embodiments of the antibody-drug conjugate of formula (I), (Ia) or (Ib), where CTX is MMAF, and L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the antibody-drug conjugate of formula (I), (Ia) or (Ib), where CTX is MMAF, L is —(CH₂)₅C(O)—, and n is 4.

In certain embodiments of the antibody-drug conjugate of formula (Ia), the antibody-drug conjugate is of the following formula:

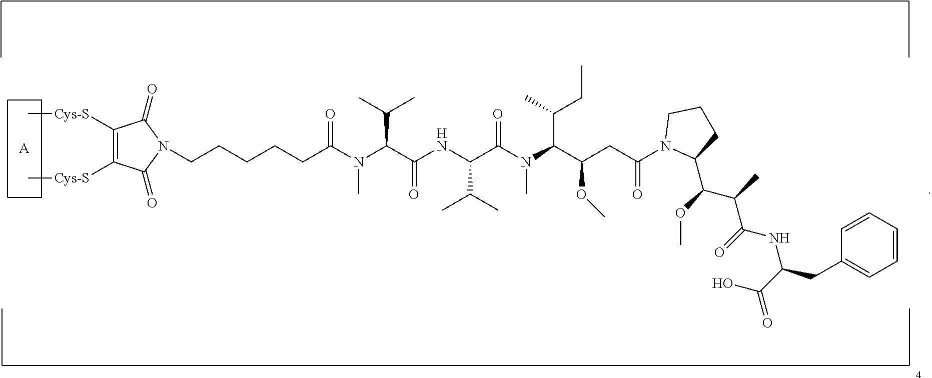

In certain embodiments of the antibody-drug conjugate of formula (Ib), the antibody-drug conjugate is of the following formula:

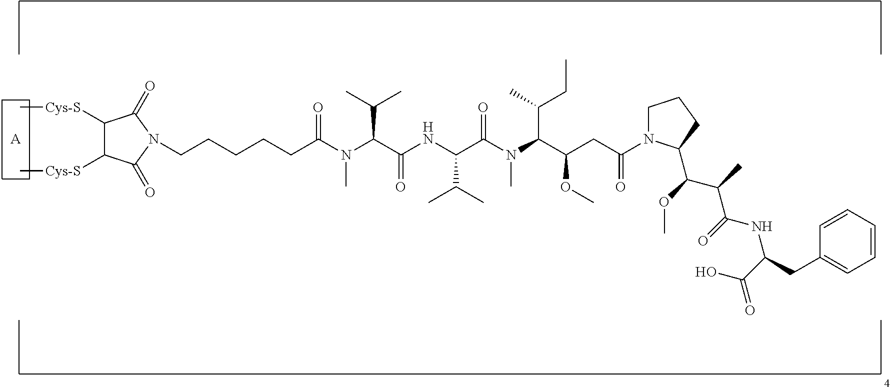

In certain embodiments of the antibody-drug conjugate of formula (I), (Ia) or (Ib), where CTX is MMAE, and L is a cleavable linker.

In certain embodiments of the antibody-drug conjugate of formula (I), (Ia) or (Ib), where CTX is MMAE, and L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the antibody-drug conjugate of formula (I), (Ia) or (Ib), where CTX is MMAE, and L is —(CH₂)₅C(O)-Val-Ala-PAB-O—C(O)—, or —(CH₂)₅C(O)-Val-Cit-PAB-O—C(O)—, an n is 4.

In certain embodiments of the antibody-drug conjugate of formula (Ia), the antibody-drug conjugate is of the following formula:

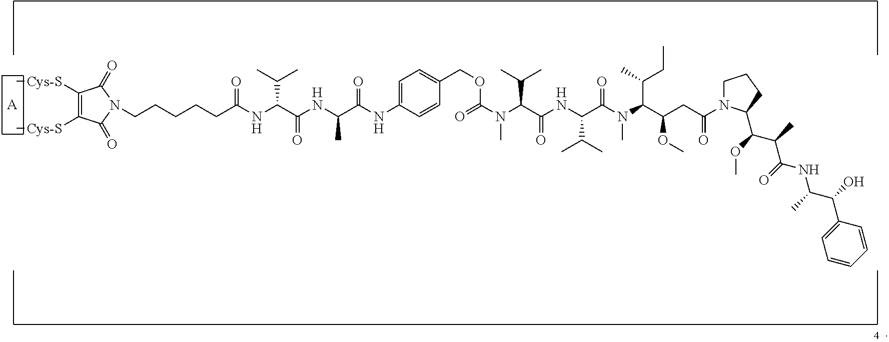

In certain embodiments of the antibody-drug conjugate of formula (Ib), the antibody-drug conjugate is of the following formula:

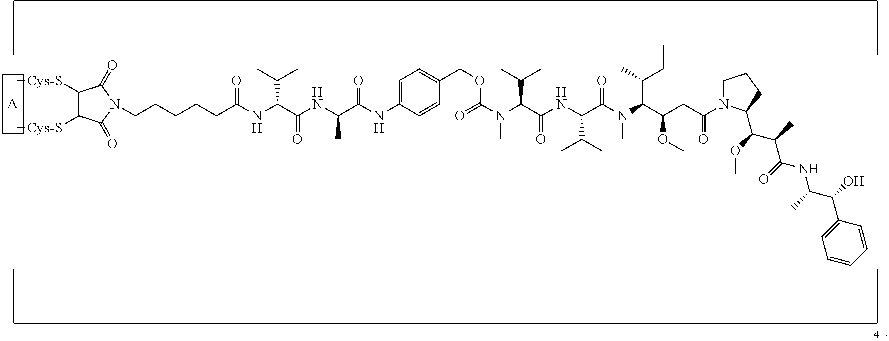

In certain embodiments of the antibody-drug conjugate of formula (I), (Ia) or (Ib), where CTX is a PBD, and L is a cleavable linker.

In certain embodiments of the antibody-drug conjugate of formula (I), (Ia) or (Ib), where CTX is a PBD, L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the antibody-drug conjugate of formula (I), (Ia) or (Ib), where CTX is a PBD, L is —(CH₂)₅C(O)-Val-Ala-PAB-O—C(O)—, or —(CH₂)₅C(O)-Val-Cit-PAB-O—C(O)—, an n is 4.

In certain embodiments of the antibody-drug conjugate of formula (Ia), the antibody-drug conjugate is of one of the following formulas:

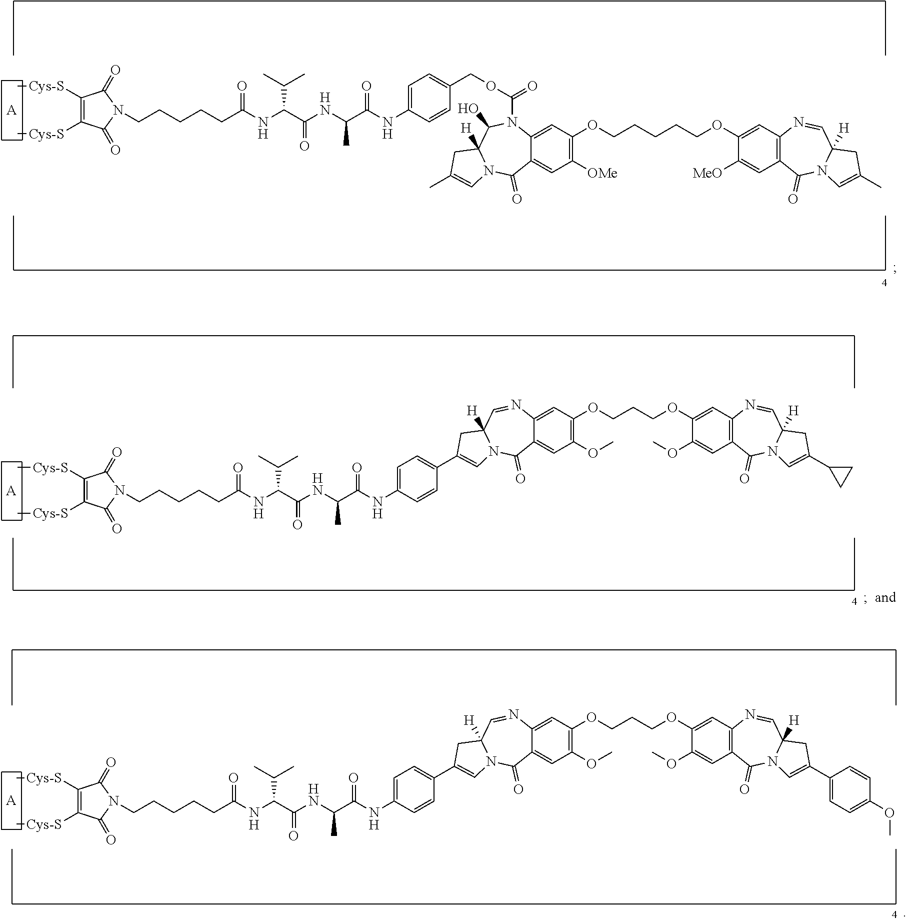

In certain embodiments of the antibody-drug conjugate of formula (Ib), the antibody-drug conjugate is of one of the following formulas:

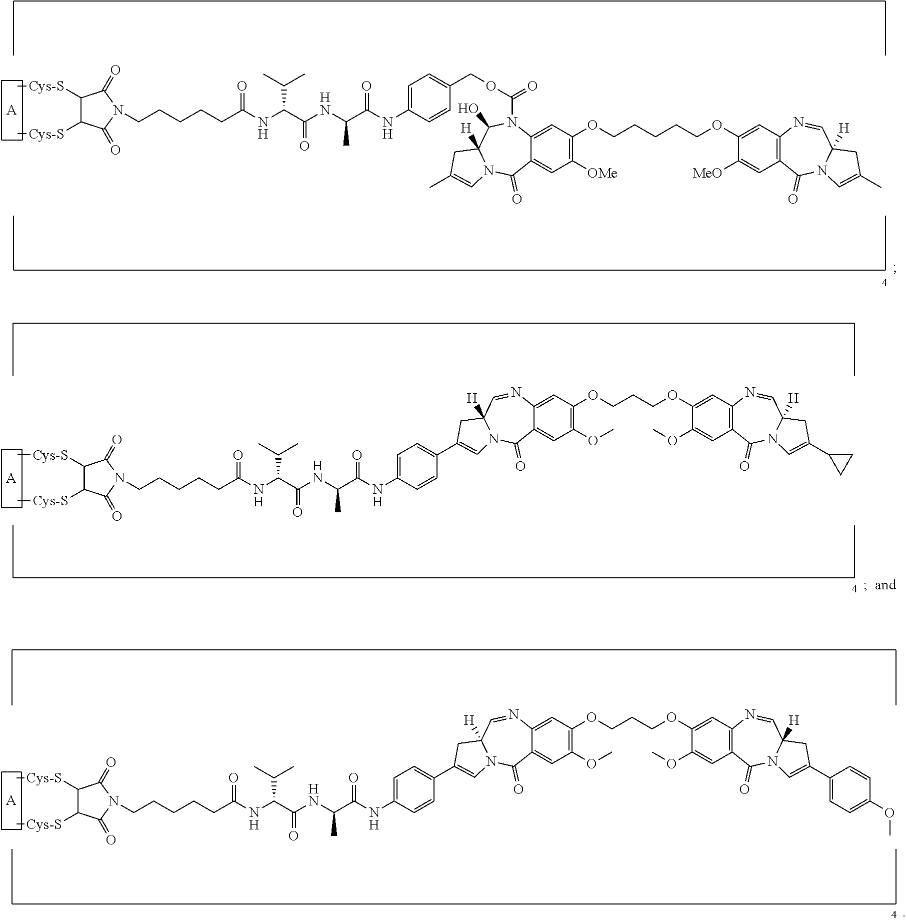

In certain embodiments of the antibody-drug conjugate of formula (I), (Ia) or (Ib), A is a monoclonal antibody.

In certain embodiments of the antibody-drug conjugate of formula (I), (Ia) or (Ib), A is an antibody that is specific to a cancer antigen. In certain embodiments, the cancer antigen is CD33 (Siglec3), CD30 (TNFRSF8), HER2 (ERbB-2), EGFR, CD22 (Siglec2), CD79b, CD22 (Siglec2), GPNMB, CD19 (B4), CD56 (NCAM), CD138 (SDC1), PSMA (FOLH1), CD74 (DHLAG), PSMA (FOLH1), CEACAM5 (CD66e), EGP1 (TROP2), FOLR1, CD37, Muc-16, Endothelial receptor (ETB), STEAP1, CD19, CD70 (TNFSF7), SLC44A4, Nectin-4, AGS-16, Guanylyl cyclase C, Muc-1, CD70 (TNFSF7), Her3 (ErbB-3), mesothelin, NaPi2b, LIV1, SLITRK6, ENPP3, TF, 5T4, BCMA, SCLC, Integrin, CD70 (TNFSF7), CA9 (MN), or CFC1B (Cripto). In certain embodiments, the cancer antigen is HER2, VEGF-A, EGFR, CD20, C10orf54, CD98, or C16orf54.

In certain embodiments of the antibody-drug conjugate of formula (I), (Ia) or (Ib), A is selected from the group consisting of alemtuzumab, anitumumab, bevacizumab, brentuximab, cetuximab, gemtuzumab, glembatumumab, inotuzumab, ipilimumab, lovortumumab, milatuzumab, ofatumumab, rituximab, tositumomab, and trastuzumab.

In certain embodiments of the antibody-drug conjugate of formula (I), (Ia) or (Ib), A is selected from the group consisting of adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab, votumumab, and zalutumumab.

In certain embodiments, of the antibody-drug conjugate of formula (I), (Ia) or (Ib), A is trastuzumab.

In certain embodiments of the antibody-drug conjugate of formula (I), (Ia) or (Ib), n is 4.

In certain embodiments of the antibody-drug conjugate of formula (I), (Ia) or (Ib), A comprises: a VH sequence that comprises SEQ ID NO: 1 and a VL sequence that comprises SEQ ID NO: 2; a VH sequence that comprises SEQ ID NO: 3 and a VL sequence that comprises SEQ ID NO: 4; or a VH sequence that comprises SEQ ID NO: 5 and a VL sequence that comprises SEQ ID NO: 6.

In certain embodiments of the antibody-drug conjugate of formula (I), (Ia) or (Ib), A comprises: a heavy chain sequence that comprises SEQ ID NO: 7 and a light chain sequence that comprises SEQ ID NO: 11; a heavy chain sequence that comprises SEQ ID NO: 8 and a light chain sequence that comprises SEQ ID NO: 11; a heavy chain sequence that comprises SEQ ID NO: 9 and a light chain sequence that comprises SEQ ID NO: 11; or a heavy chain sequence that comprises SEQ ID NO: 10 and a light chain sequence that comprises SEQ ID NO: 11.

In certain embodiments of the antibody-drug conjugate of formula (I), (Ia) or (Ib), A comprises: a heavy chain sequence that comprises SEQ ID NO: 12 and a light chain sequence that comprises SEQ ID NO: 16; a heavy chain sequence that comprises SEQ ID NO: 13 and a light chain sequence that comprises SEQ ID NO: 16; a heavy chain sequence that comprises SEQ ID NO: 14 and a light chain sequence that comprises SEQ ID NO: 16; a heavy chain sequence that comprises SEQ ID NO: 15 and a light chain sequence that comprises SEQ ID NO: 16.

In certain embodiments of the antibody-drug conjugate of formula (I), (Ia) or (Ib), A comprises: a heavy chain sequence that comprises SEQ ID NO: 17 and a light chain sequence that comprises SEQ ID NO: 21; a heavy chain sequence that comprises SEQ ID NO: 18 and a light chain sequence that comprises SEQ ID NO: 21; a heavy chain sequence that comprises SEQ ID NO: 19 and a light chain sequence that comprises SEQ ID NO: 21; or a heavy chain sequence that comprises SEQ ID NO: 20 and a light chain sequence that comprises SEQ ID NO: 21.

In certain embodiments of the antibody-drug conjugate of formula (I), (Ia) or (Ib), A comprises: a heavy chain sequence that comprises SEQ ID NO: 22 and a light chain sequence that comprises SEQ ID NO: 26; a heavy chain sequence that comprises SEQ ID NO: 23 and a light chain sequence that comprises SEQ ID NO: 26; a heavy chain sequence that comprises SEQ ID NO: 24 and a light chain sequence that comprises SEQ ID NO: 26; or a heavy chain sequence that comprises SEQ ID NO: 25 and a light chain sequence that comprises SEQ ID NO: 26.

In certain embodiments of the antibody-drug conjugate of formula (I), (Ia) or (Ib), the opened cysteine-cysteine disulfide bond in A is an interchain disulfide bond. In certain embodiments, where the opened cysteine-cysteine disulfide bond in A is an interchain disulfide bond n is 4 (e.g., two heavy chain-light chain interchain disulfide bonds and two hinge heavy chain-heavy chain interchain disulfide bonds). In certain embodiments, where the opened cysteine-cysteine disulfide bond in A is an interchain disulfide bond n is 3 (e.g., two heavy chain-light chain interchain disulfide bonds and one hinge heavy chain-heavy chain interchain disulfide bond). In certain embodiments, where the opened cysteine-cysteine disulfide bond in A is an interchain disulfide bond n is 2 (e.g., two heavy chain-light chain interchain disulfide bonds).

The present disclosure also provides linker-cytotoxin conjugates of one of the following formulas (IIa), (IIb), and (IIc):

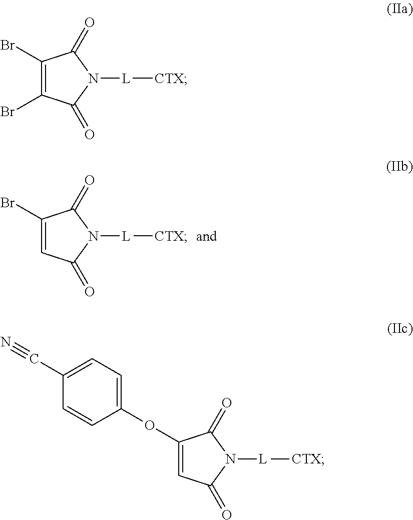

or an enantiomer, diasteriomer, or mixtures thereof;
wherein:
L is a cleavable or noncleavable linker; and
CTX is an auristatin, a pyrrolobenzodiazepine, calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond.

In certain embodiments of the linker-cytotoxin conjugate of formula (IIa), (IIb) or (IIc), L is a cleavable or a noncleavable linker; and CTX is an auristatin bonded to L by an amide bond or a carbamate bond.

In certain embodiments of the linker-cytotoxin conjugate of formula (IIa), (IIb) or (IIc), L is a cleavable or a noncleavable linker; and CTX is monomethylauristatin F (MMAF) bonded to L by an amide bond or a carbamate bond. In certain embodiments, MMAF is bonded to L by an amide bond.

In certain embodiments of the linker-cytotoxin conjugate of formula (IIa), (IIb) or (IIc), L is a cleavable or a noncleavable linker; and CTX is monomethylauristatin E (MMAE) bonded to L by an amide bond or a carbamate bond. In certain embodiments, MMAE is bonded to L by a carbamate bond.

In certain embodiments of the linker-cytotoxin conjugate of formula (IIa), (IIb) or (IIc), where CTX is MMAF, L is a noncleavable linker.

In certain embodiments of the linker-cytotoxin conjugate of formula (IIa), (IIb) or (IIc), where CTX is MMAF, L is —(CH₂)ₘC(O)—, wherein m is an integer of 5 to 11.

In certain embodiments of the linker-cytotoxin conjugate of formula (IIa), (IIb) or (IIc), where CTX is MMAF, L is a cleavable linker.

In certain embodiments of the linker-cytotoxin conjugate of formula (IIa), (IIb) or (IIc), where CTX is MMAF, L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the linker-cytotoxin conjugate of formula (IIa), (IIb) or (IIc), where CTX is MMAF, L is —(CH₂)₅C(O)—.

In certain embodiments of the linker-cytotoxin conjugate of formula (IIa), the linker-cytotoxin conjugate has the following structure:

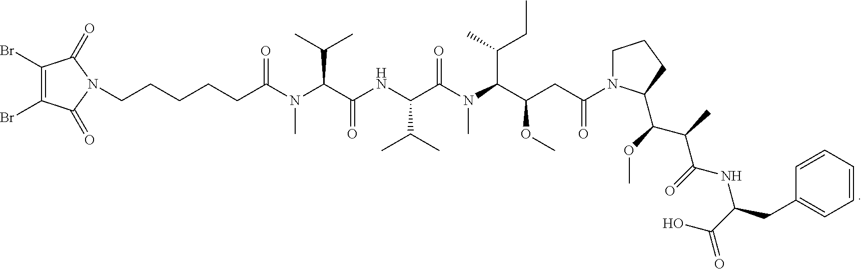 [SMILES: CC[C@@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C(Br)=C(Br)C1=O)C(C)C]

In certain embodiments of the linker-cytotoxin conjugate of formula (IIb), the linker-cytotoxin conjugate has the following structure:

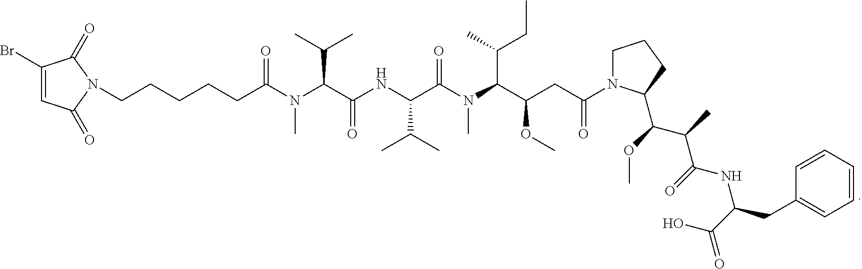 [SMILES: CC[C@@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=C(Br)C1=O)C(C)C]

In certain embodiments of the linker-cytotoxin conjugate of formula (IIc), the linker-cytotoxin conjugate has the following structure:

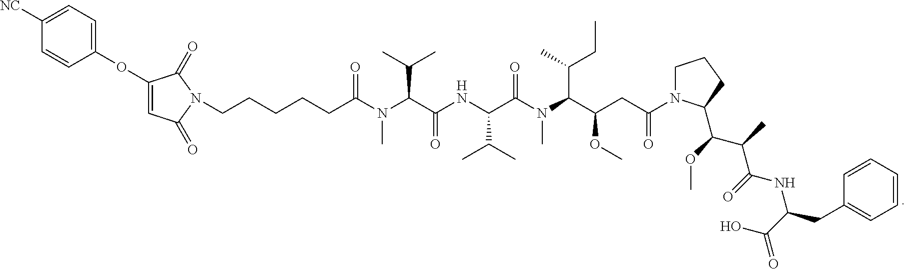 [SMILES: CC[C@@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=C(Oc2ccc(C#N)cc2)C1=O)C(C)C]

In certain embodiments of the linker-cytotoxin conjugate of formula (IIa), (IIb) or (IIc), where CTX is MMAE, L is a cleavable linker.

In certain embodiments of the linker-cytotoxin conjugate of formula (IIa), (IIb) or (IIc), where CTX is MMAE, L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the linker-cytotoxin conjugate of formula (IIa), (IIb) or (IIc), where CTX is MMAE, L is —(CH₂)₅C(O)-Val-Ala-PAB-O—C(O)—, or —(CH₂)₅C(O)-Val-Cit-PAB-O—C(O)—.

In certain embodiments of the linker-cytotoxin conjugate of formula (IIa), the linker-cytotoxin conjugate has the following structure:

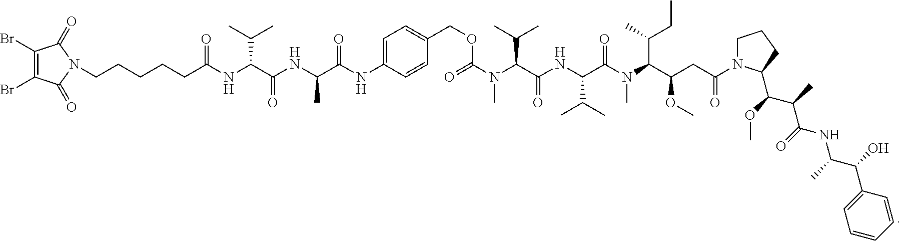 [SMILES: CC[C@@H](C)[C@@H]([C@@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@@H](C)NC(=O)[C@H](NC(=O)CCCCCN2C(=O)C(Br)=C(Br)C2=O)C(C)C)cc1)C(C)C]

In certain embodiments of the linker-cytotoxin conjugate of formula (IIb), the linker-cytotoxin conjugate has the following structure:

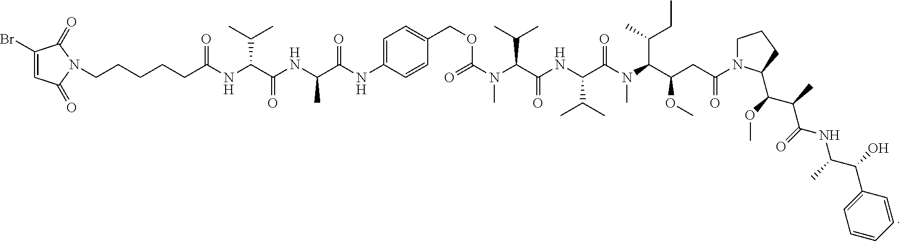 [SMILES: CC[C@@H](C)[C@@H]([C@@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@@H](C)NC(=O)[C@H](NC(=O)CCCCCN2C(=O)C=C(Br)C2=O)C(C)C)cc1)C(C)C]

In certain embodiments of the linker-cytotoxin conjugate of formula (IIc), the linker-cytotoxin conjugate has the following structure:

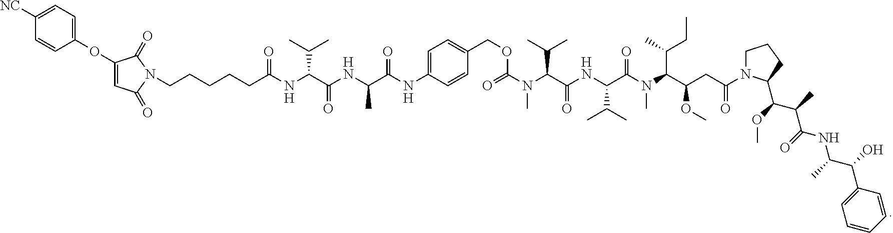 [SMILES: CC[C@@H](C)[C@@H]([C@@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@@H](C)NC(=O)[C@H](NC(=O)CCCCCN2C(=O)C=C(Oc3ccc(C#N)cc3)C2=O)C(C)C)cc1)C(C)C]

In certain embodiments of the linker-cytotoxin conjugate of formula (IIa), (IIb) or (IIc), where CTX is a PBD, L is a cleavable linker.

In certain embodiments of the linker-cytotoxin conjugate of formula (IIa), (IIb) or (IIc), where CTX is a PBD, L is —(CH₂)ₘC(O)-Val-Ala-PAB-O—C(O)—, or —(CH₂)ₘC(O)-Val-Cit-PAB-O—C(O)—, wherein m is an integer of 5 to 11.

In certain embodiments of the linker-cytotoxin conjugate of formula (IIa), (IIb) or (IIc), where CTX is a PBD, L is —(CH₂)₅C(O)-Val-Ala-PAB-O—C(O)—, or —(CH₂)₅C(O)-Val-Cit-PAB-O—C(O)—.

In certain embodiments of the linker-cytotoxin conjugate of formula (IIa), the linker-cytotoxin conjugate has one of the following structures: and

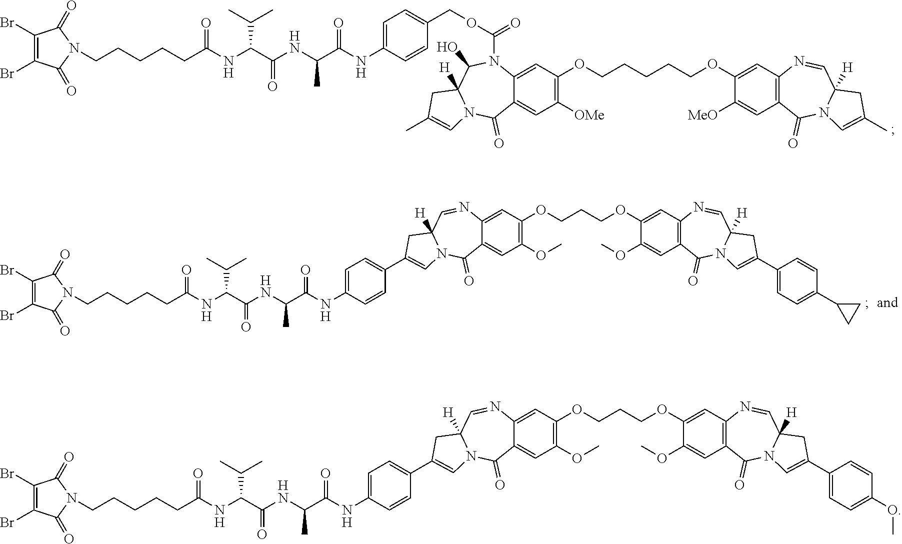

In certain embodiments of the linker-cytotoxin conjugate of formula (IIb), the linker-cytotoxin conjugate has one of the following structures:

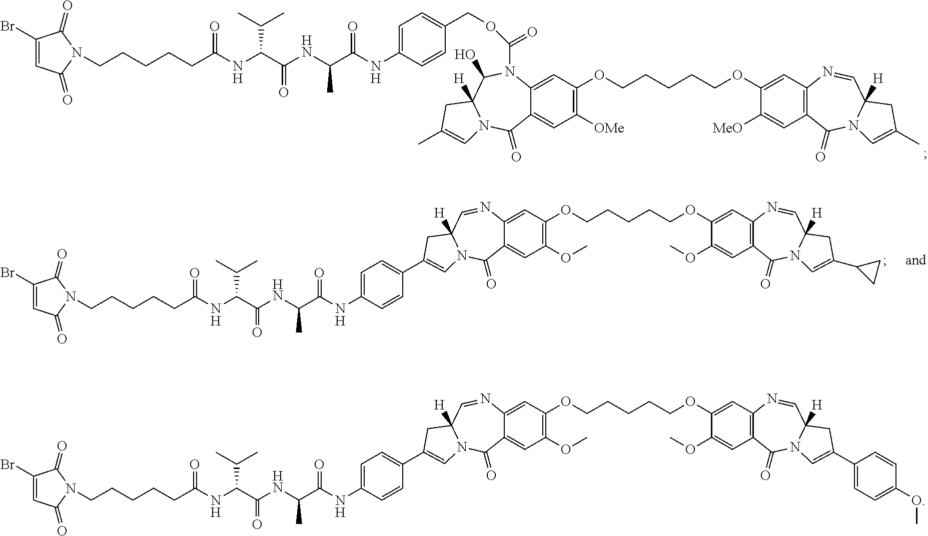 [SMILES: COc1cc2c(cc1OCCCCCOc1cc3c(cc1OC)C(=O)N1C=C(C)C[C@@H]1[C@@H](O)N3C(=O)OCc1ccc(NC(=O)[C@@H](C)NC(=O)[C@H](NC(=O)CCCCCN3C(=O)C=C(Br)C3=O)C(C)C)cc1)N=C[C@@H]1CC(C)=CN1C2=O ; COc1cc2c(cc1OCCCCCOc1cc3c(cc1OC)C(=O)N1C=C(c4ccc(NC(=O)[C@@H](C)NC(=O)[C@H](NC(=O)CCCCCN5C(=O)C=C(Br)C5=O)C(C)C)cc4)C[C@@H]1C=N3)N=C[C@@H]1CC(C3CC3)=CN1C2=O ; COc1ccc(C2=CN3C(=O)c4cc(OC)c(OCCCCCOc5cc6c(cc5OC)C(=O)N5C=C(c7ccc(NC(=O)[C@@H](C)NC(=O)[C@H](NC(=O)CCCCCN8C(=O)C=C(Br)C8=O)C(C)C)cc7)C[C@@H]5C=N6)cc4N=C[C@@H]3C2)cc1]

In certain embodiments of the linker-cytotoxin conjugate of formula (IIc), the linker-cytotoxin conjugate has one of the following structures:

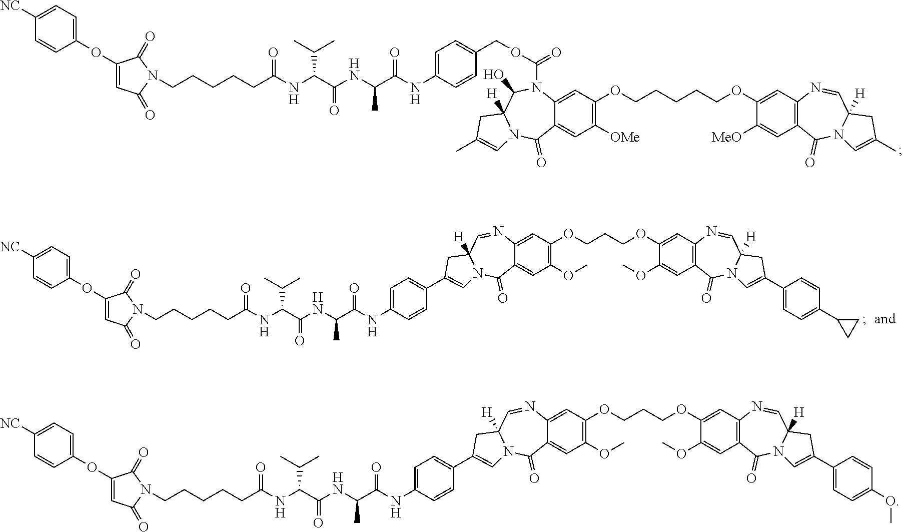

The present disclosure also provides pharmaceutical compositions comprising the antibody-drug conjugates of formula (I), (Ia) or (Ib) or a pharmaceutically acceptable salts thereof, and a pharmaceutically acceptable diluents, carrier or excipient.

The present disclosure also provides methods of treating a cancer by administering to a human suffering therefrom an effective amount of the antibody-drug conjugates of formula (I), (Ia) or (Ib) or pharmaceutical compositions comprising such antibody-drug conjugates.

The present disclosure also provides methods of making antibody-drug conjugates of the following formula (I):

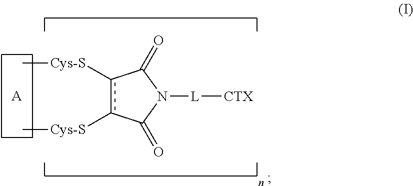

or a pharmaceutically acceptable salt thereof, wherein:
A is an antibody; the two depicted cysteine residues are from an opened cysteine-cysteine disulfide bond in A; L is a cleavable or a noncleavable linker; CTX is a cytotoxin bonded to L by an amide bond, a carbamate bond, a disulfide bond, an ether bond, a thioether bond, or an ester bond; and n is 4.

In certain embodiments of the method of making an antibody-drug conjugate of formula (I), the method comprises the steps of:
a) providing a solution comprising A;
b) contacting the solution of a) with a solution comprising TCEP;
c) contacting the solution of b) with a solution comprising a cytotoxin-linker conjugate.

In certain embodiments of the method of making an antibody-drug conjugate of formula (I), the CTX is an auristatin, a pyrrolobenzodiazepine (PDB), calicheamicin, doxorubicin, camptothecin, duocarmycin, DM1, DM4, a maytansinoid, or a tubulysin.

In certain embodiments of the method of making an antibody-drug conjugate of formula (I), the cytotoxin-linker conjugate is a disubstituted maleimide-cytotoxin linker conjugate, for example, a dibromomaleimido-cytotoxin linker conjugate.

In certain embodiments of the method of making an antibody-drug conjugate of formula (I), the cytotoxin-linker conjugate is a monosubstituted maleimide-cytotoxin linker conjugate, for example, a bromomaleimido-cytotoxin linker conjugate, or a cyanophenolmaleimido-cytotoxin linker conjugate.

In certain embodiments of the method of making an antibody-drug conjugate of formula (I), the dibromomaleimido-cytotoxin linker conjugate is of the following formula (II):

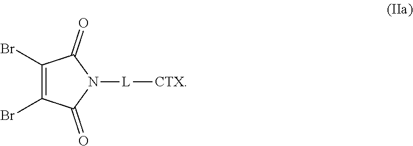

In certain embodiments of the method of making an antibody-drug conjugate of formula (I), the bromomaleimido-cytotoxin linker conjugate is of the following formula (IIb):

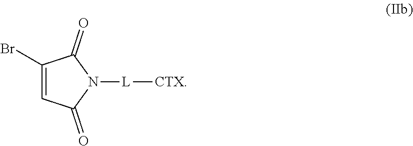

In certain embodiments of the method of making an antibody-drug conjugate of formula (I), the cyanophenolmaleimido-cytotoxin linker conjugate is of the following formula (IIc):

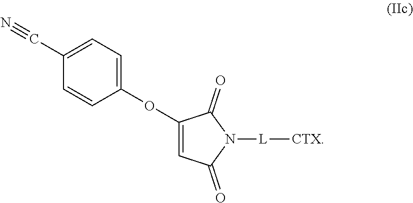

In certain embodiments of the method of making an antibody-drug conjugate of formula (I), (Ia) or (Ib), the solution of step a) comprises 20 mM sodium phosphate, 20 mM Borate, and 5 mM EDTA. In certain embodiments, the pH of the solution of steps a), b) and/or c) is between about 7.0 to about 8.2. In certain embodiments, the pH of the solution of steps a), b) and/or c) is between about 7.4 to about 8.2. In certain embodiments, the pH of the solution of steps a), b) and/or c) is between about 7.0 to about 7.8. In certain embodiments, the pH of the solution of steps a), b) and/or c) is about 7.2. In certain embodiments, the pH of the solution of step b) is 7.2. In certain embodiments, steps a), b) and/or c) are performed at a temperature of about 22° C. to about 37° C. In certain embodiments, steps a), b) and/or c) are performed at a temperature of about 22° C. to about 27° C. In certain embodiments, steps b) and c) are performed at a temperature of about 22° C. to about 27° C. In certain embodiments, the ratio of molar equivalents of TCEP to antibody in step b) is about 4 to about 10. In certain embodiments, the ratio of TCEP to antibody in step b) is about 9.5. In certain embodiments, the ratio of molar equivalents of cytotoxin linker conjugate to antibody in step c) is about 4 to about 10. In certain embodiments, In certain embodiments, the ratio of molar equivalents of cytotoxin linker conjugate to antibody in step c) is about 4.5 to about 6.0. In certain embodiments, In certain embodiments, the ratio of molar equivalents of cytotoxin linker conjugate to antibody in step c) is about 4.5 to about 5.5. In certain embodiments, In certain embodiments, the ratio of molar equivalents of cytotoxin linker conjugate to antibody in step c) is about 5.0 to about 6.0. In certain embodiments, the ratio of molar equivalents of cytotoxin linker conjugate to antibody in step c) is about 5.1 to about 5.8.

The present disclosure also provides methods of making a compound of formula (19):

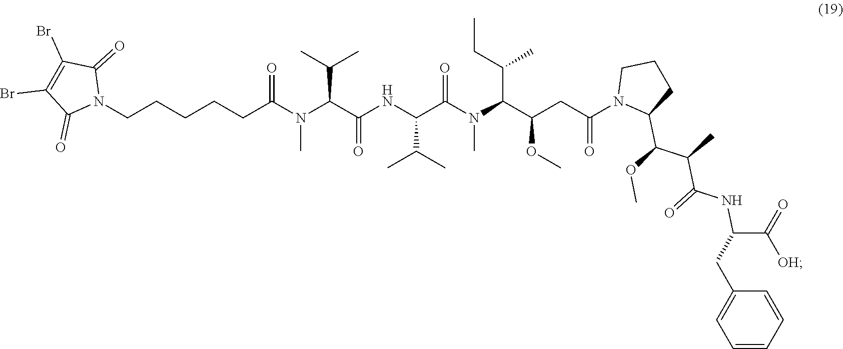 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C(Br)=C(Br)C1=O)C(C)C]

or a salt thereof.

The present disclosure also provides methods of making a compound of formula (25):

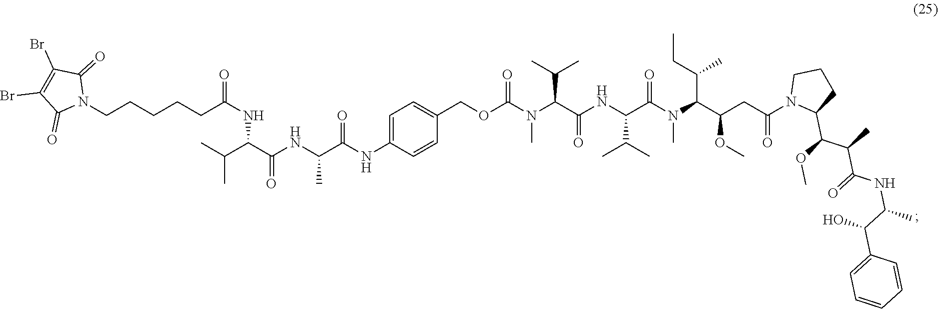 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1)C(C)C]

or a salt thereof.

The present disclosure also provides antibody-drug conjugates of the following formula (III):

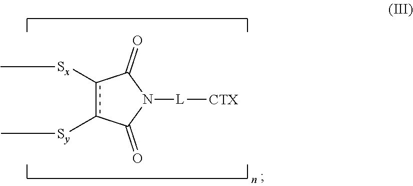

wherein:
L is a cleavable or a noncleavable linker;
CTX is a cytotoxin bonded to L by an amide bond, a carbamate bond, a disulfide bond, an ether bond, a thioether bond, or an ester bond;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody;
the [symbol] bond represents a single or a double bond;
and
n is an integer of 1 to 4.

The present disclosure also provides antibody-drug conjugates of the following formula (IIIa):

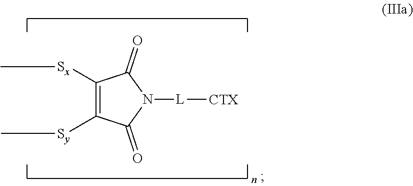

wherein:
L is a cleavable or a noncleavable linker;
CTX is a cytotoxin bonded to L by an amide bond, a carbamate bond, a disulfide bond, an ether bond, a thioether bond, or an ester bond;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody; and
n is an integer of 1 to 4.

The present disclosure also provides antibody-drug conjugates of the following formula (IIIb):

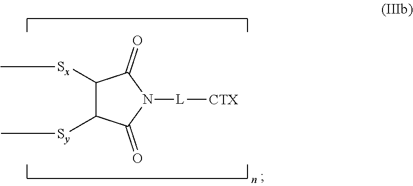

wherein:
L is a cleavable or a noncleavable linker;
CTX is a cytotoxin bonded to L by an amide bond, a carbamate bond, a disulfide bond, an ether bond, a thioether bond, or an ester bond;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody; and
n is an integer of 1 to 4.

In certain embodiments of the antibody-drug conjugate of formula (III), (IIIa) or (IIIb), CTX is an auristatin, pyrrolobenzodiazepine (PDB), calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond.

In certain embodiments of the antibody-drug conjugate of formula (III), (IIIa) or (IIIb), CTX is an auristatin bonded to L by an amide bond or a carbamate bond; wherein the auristatin is MMAF or MMAE.

In certain embodiments of the antibody-drug conjugate of formula (III), (IIIa) or (IIIb), CTX is a PBD bonded to L by an amide bond or a carbamate bond.

In certain embodiments of the antibody-drug conjugate of formula (III), (IIIa) or (IIIb), CTX is a calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond.

In certain embodiments of the antibody-drug conjugate of formula (III), (IIIa) or (IIIb), the multi-chain antibody comprises two heavy chains and two light chains.

In certain embodiments of the antibody-drug conjugate of formula (III), (IIIa) or (IIIb), the first cysteine residue is from a first heavy chain and the second cysteine residue is from a second heavy chain of the multi-chain antibody.

In certain embodiments of the antibody-drug conjugate of formula (III), (IIIa) or (IIIb), the first cysteine residue is from a heavy chain and the second cysteine residue is from a light chain of the multi-chain antibody.

In certain embodiments of the antibody-drug conjugate of formula (III), (IIIa) or (IIIb), the first and second cysteine residues are from the same heavy chain of the multi-chain antibody.

In certain embodiments of the antibody-drug conjugate of formula (III), the antibody-drug conjugate is of the following formula:

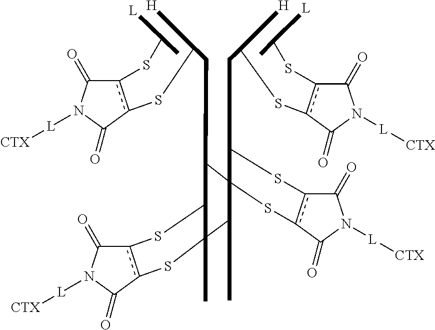

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (III), the antibody-drug conjugate is of the following formula:

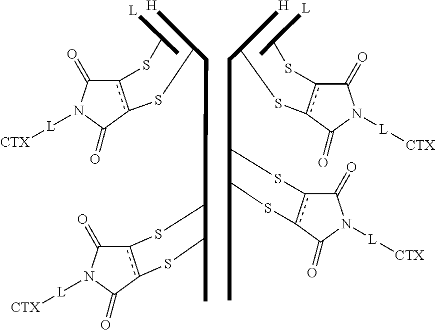

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (III), the antibody-drug conjugate is of the following formula:

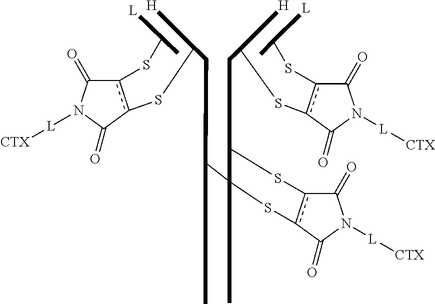

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (III), the antibody-drug conjugate is of the following formula:

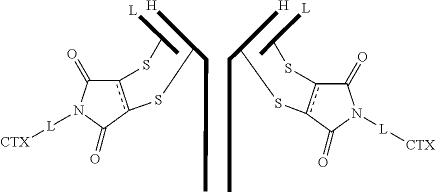

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (IIIa), the antibody-drug conjugate is of the following formula:

where each heavy chain of the multi-chain antibody is denoted by the letter H, and each light chain of the multi-chain antibody is denoted by the letter L.

In certain embodiments of the antibody-drug conjugate of formula (IIa), the antibody-drug conjugate is of the following formula:

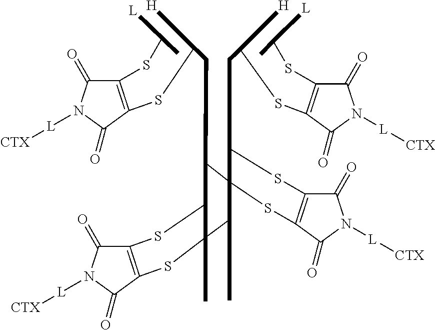

where each heavy chain of the multi-chain antibody is denoted by the letter H, and each light chain of the multi-chain antibody is denoted by the letter L.

In certain embodiments of the antibody-drug conjugate of formula (IIIa), the antibody-drug conjugate is of the following formula:

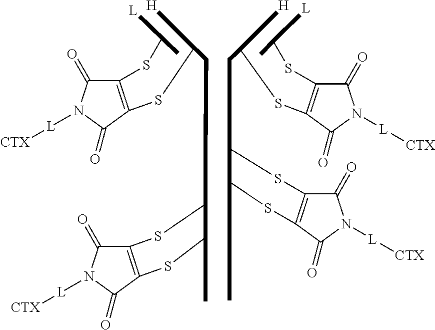

where each heavy chain of the multi-chain antibody is denoted by the letter H, and each light chain ol the multi-chain antibody is denoted by the letter L.

In certain embodiments of the antibody-drug conjugate of formula (IIIa), the antibody-drug conjugate is of the following formula:

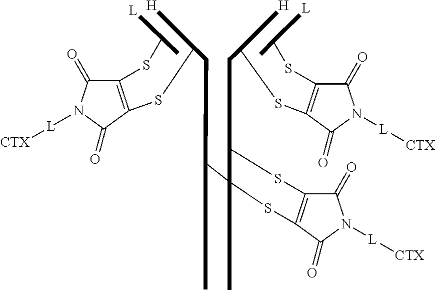

where each heavy chain of the multi-chain antibody is denoted by the letter H, and each light chain of the multi-chain antibody is denoted by the letter L.

In certain embodiments of the antibody-drug conjugate of formula (IIIa), the antibody-drug conjugate is of the following formula:

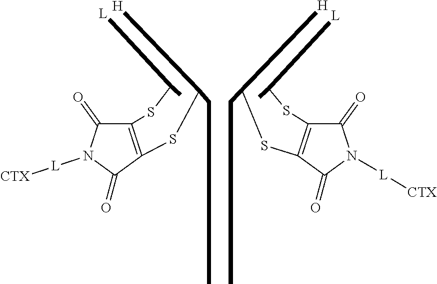

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

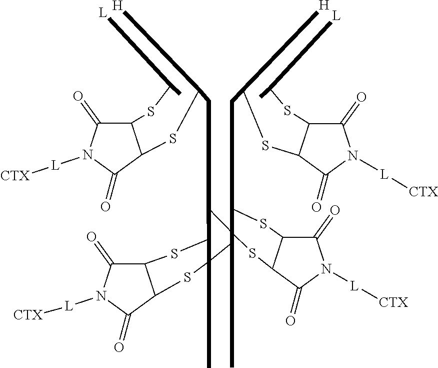

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

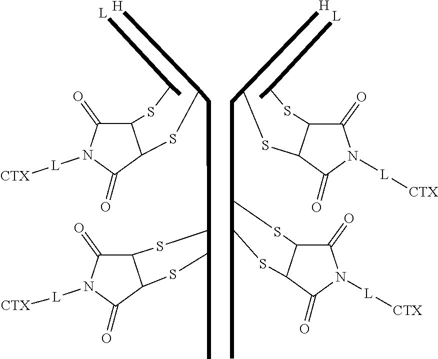

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

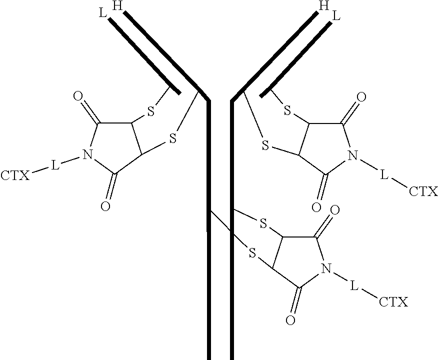

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

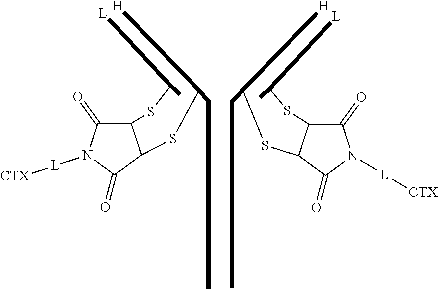

where each heavy chain of the multi-chain antibody is denoted by the letter H, and each light chain of the multi-chain antibody is denoted by the letter L.

In certain embodiments of the antibody-drug conjugate of formula (III), (IIIa) or (IIIb), L is a noncleavable linker.

In certain embodiments of the antibody-drug conjugate of formula (III), (IIIa) or (IIIb), L is —(CH₂)ₘC(O)—, wherein m is an integer of 5 to 11.

In certain embodiments of the antibody-drug conjugate of formula (III), (IIIa) or (IIIb), L is a cleavable linker.

In certain embodiments of the antibody-drug conjugate of formula (III), (IIIa) or (IIIb), L is —(CH₂)ₘC(O)-Val-Ala-PAB-O—C(O)—, or —(CH₂)ₘC(O)-Val-Cit-PAB-O—C(O)—. wherein m is an integer of 5 to 11.

In certain embodiments of the antibody-drug conjugate of formula (III), (IIIa) or (IIIb), the multi-chain antibody is a monoclonal antibody.

In certain embodiments of the antibody-drug conjugate of formula (III), (IIIa) or (IIIb), the multi-chain antibody is an antibody that is specific to a cancer antigen. In certain embodiments, the cancer antigen is HER2, VEGF-A, EGFR, CD20, C10orf54, CD98, or C16orf54.

In certain embodiments of the antibody-drug conjugate of formula (III), (IIIa) or (IIIb), the multi-chain antibody is selected from the group consisting of alemtuzumab, anitumumab, bevacizumab, brentuximab, cetuximab, gemtuzumab, glembatumumab, inotuzumab, ipilimumab, lovortumumab, milatuzumab, ofatumumab, rituximab, tositumomab, and trastuzumab.

In certain embodiments of the antibody-drug conjugate of formula (III), (IIIa) or (IIIb), the multi-chain antibody is selected from the group consisting of adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab, votumumab, and zalutumumab.

In certain embodiments of the antibody-drug conjugate of formula (III), (IIIa) or (IIIb), the multi-chain antibody comprises: a VH sequence that comprises SEQ ID NO: 1 and a VL sequence that comprises SEQ ID NO: 2; a VH sequence that comprises SEQ ID NO: 3 and a VL sequence that comprises SEQ ID NO: 4; or a VH sequence that comprises SEQ ID NO: 5 and a VL sequence that comprises SEQ ID NO: 6.

In certain embodiments of the antibody-drug conjugate of formula (III), (IIIa) or (IIIb), the multi-chain antibody comprises: a heavy chain sequence that comprises SEQ ID NO: 7 and a light chain sequence that comprises SEQ ID NO: 11; a heavy chain sequence that comprises SEQ ID NO: 8 and a light chain sequence that comprises SEQ ID NO: 11; a heavy chain sequence that comprises SEQ ID NO: 9 and a light chain sequence that comprises SEQ ID NO: 11; or a heavy chain sequence that comprises SEQ ID NO: 10 and a light chain sequence that comprises SEQ ID NO: 11.

In certain embodiments of the antibody-drug conjugate of formula (III), (IIIa) or (IIIb), the multi-chain antibody comprises: a heavy chain sequence that comprises SEQ ID NO: 12 and a light chain sequence that comprises SEQ ID NO: 16; a heavy chain sequence that comprises SEQ ID NO: 13 and a light chain sequence that comprises SEQ ID NO: 16; a heavy chain sequence that comprises SEQ ID NO: 14 and a light chain sequence that comprises SEQ ID NO: 16; a heavy chain sequence that comprises SEQ ID NO: 15 and a light chain sequence that comprises SEQ ID NO: 16.

In certain embodiments of the antibody-drug conjugate of formula (III), (IIIa) or (IIIb), the multi-chain antibody comprises: a heavy chain sequence that comprises SEQ ID NO: 17 and a light chain sequence that comprises SEQ ID NO: 21; a heavy chain sequence that comprises SEQ ID NO: 18 and a light chain sequence that comprises SEQ ID NO: 21; a heavy chain sequence that comprises SEQ ID NO: 19 and a light chain sequence that comprises SEQ ID NO: 21; or a heavy chain sequence that comprises SEQ ID NO: 20 and a light chain sequence that comprises SEQ ID NO: 21.

In certain embodiments of the antibody-drug conjugate of formula (III), (IIIa) or (IIIb), the multi-chain antibody comprises: a heavy chain sequence that comprises SEQ ID NO: 22 and a light chain sequence that comprises SEQ ID NO: 26; a heavy chain sequence that comprises SEQ ID NO: 23 and a light chain sequence that comprises SEQ ID NO: 26; a heavy chain sequence that comprises SEQ ID NO: 24 and a light chain sequence that comprises SEQ ID NO: 26; or a heavy chain sequence that comprises SEQ ID NO: 25 and a light chain sequence that comprises SEQ ID NO: 26.

In certain embodiments of the antibody-drug conjugate of formula (III), (IIIa) or (IIIb), n is 4. In certain embodiments, CTX is MMAF, L is —(CH₂)₅C(O)—, and n is 4. In certain embodiments, CTX is MMAE, L is —(CH₂)₅C(O)-Val-Ala-PAB-O—C(O)—, and n is 4.

The present disclosure also provides a composition comprising an antibody-drug conjugate of the following formula:

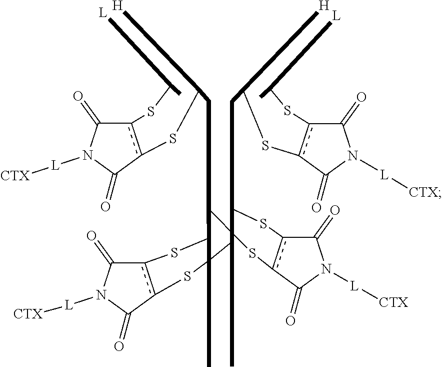

and/or
an antibody-drug conjugate of the following formula:

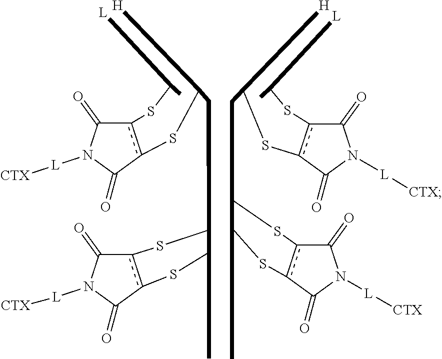

where each heavy chain of the multi-chain antibody is denoted by the letter H, and each light chain of the multi-chain antibody is denoted by the letter L; and the [symbol] bond represents a single or a double bond.

The present disclosure also provides a composition comprising an antibody-drug conjugate of the following formula:

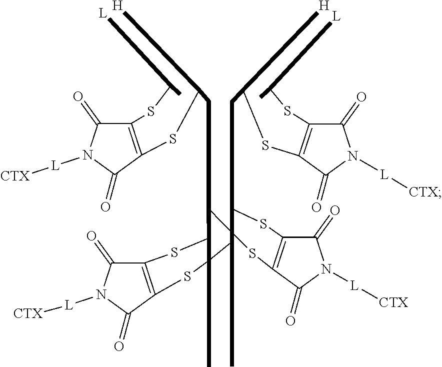

and/or
an antibody-drug conjugate of the following formula:

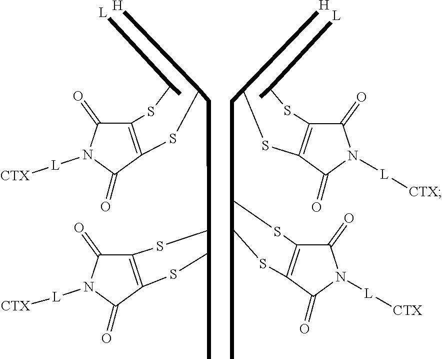

where each heavy chain of the multi-chain antibody is denoted by the letter H, and each light chain of the multi-chain antibody is denoted by the letter L.

The present disclosure also provides a composition comprising an antibody-drug conjugate of the following formula:

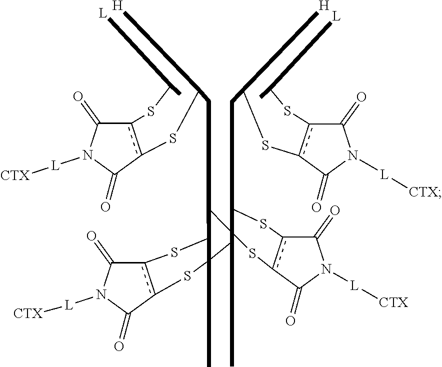

and/or
an antibody-drug conjugate of the following formula:

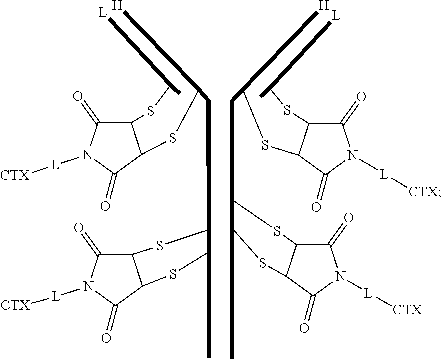

where each heavy chain of the multi-chain antibody is denoted by the letter H, and each light chain of the multi-chain antibody is denoted by the letter L.

The present disclosure also provides an antibody-drug conjugate comprising an antibody comprising: a VH sequence that comprises SEQ ID NO: 1 and a VL sequence that comprises SEQ ID NO: 2; a VH sequence that comprises SEQ ID NO: 3 and a VL sequence that comprises SEQ ID NO: 4; or a VH sequence that comprises SEQ ID NO: 5 and a VL sequence that comprises SEQ ID NO: 6.

The present disclosure also provides an antibody-drug conjugate comprising an antibody comprising: a heavy chain sequence that comprises SEQ ID NO: 7 and a light chain sequence that comprises SEQ ID NO: 11; a heavy chain sequence that comprises SEQ ID NO: 8 and a light chain sequence that comprises SEQ ID NO: 11; a heavy chain sequence that comprises SEQ ID NO: 9 and a light chain sequence that comprises SEQ ID NO: 11; or a heavy chain sequence that comprises SEQ ID NO: 10 and a light chain sequence that comprises SEQ ID NO: 11.

The present disclosure also provides an antibody-drug conjugate comprising an antibody comprising: a heavy chain sequence that comprises SEQ ID NO: 12 and a light chain sequence that comprises SEQ ID NO: 16; a heavy chain sequence that comprises SEQ ID NO: 13 and a light chain sequence that comprises SEQ ID NO: 16; a heavy chain sequence that comprises SEQ ID NO: 14 and a light chain sequence that comprises SEQ ID NO: 16; a heavy chain sequence that comprises SEQ ID NO: 15 and a light chain sequence that comprises SEQ ID NO: 16.

The present disclosure also provides an antibody-drug conjugate comprising an antibody comprising: a heavy chain sequence that comprises SEQ ID NO: 17 and a light chain sequence that comprises SEQ ID NO: 21; a heavy chain sequence that comprises SEQ ID NO: 18 and a light chain sequence that comprises SEQ ID NO: 21; a heavy chain sequence that comprises SEQ ID NO: 19 and a light chain sequence that comprises SEQ ID NO: 21; or a heavy chain sequence that comprises SEQ ID NO: 20 and a light chain sequence that comprises SEQ ID NO: 21.

The present disclosure also provides an antibody-drug conjugate comprising an antibody comprising: a heavy chain sequence that comprises SEQ ID NO: 22 and a light chain sequence that comprises SEQ ID NO: 26; a heavy chain sequence that comprises SEQ ID NO: 23 and a light chain sequence that comprises SEQ ID NO: 26; a heavy chain sequence that comprises SEQ ID NO: 24 and a light chain sequence that comprises SEQ ID NO: 26; or a heavy chain sequence that comprises SEQ ID NO: 25 and a light chain sequence that comprises SEQ ID NO: 26.

The present disclosure also provides antibodies comprising any of the sequences disclosed herein.

In certain embodiments, the antibody comprises a VH sequence that comprises SEQ ID NO: 1 and a VL sequence that comprises SEQ ID NO: 2. In certain embodiments, the antibody comprises a VH sequence that comprises SEQ ID NO: 3 and a VL sequence that comprises SEQ ID NO: 4. In certain embodiments, the antibody comprises a VH sequence that comprises SEQ ID NO: 5 and a VL sequence that comprises SEQ ID NO: 6.

In certain embodiments, the antibody comprises a heavy chain sequence which comprises SEQ ID NO: 7 and a light chain sequence which comprises SEQ ID NO: 11. In certain embodiments, the antibody comprises a heavy chain sequence which comprises SEQ ID NO: 8 and a light chain sequence which comprises SEQ ID NO: 11. In certain embodiments, the antibody comprises a heavy chain sequence which comprises SEQ ID NO: 9 and a light chain sequence which comprises SEQ ID NO: 11. In certain embodiments, the antibody comprises a heavy chain sequence which comprises SEQ ID NO: 10 and a light chain sequence which comprises SEQ ID NO: 11. In certain embodiments, the antibody comprises a heavy chain sequence which comprises SEQ ID NO: 12 and a light chain sequence which comprises SEQ ID NO: 16. In certain embodiments, the antibody comprises a heavy chain sequence which comprises SEQ ID NO: 13 and a light chain sequence which comprises SEQ ID NO: 16. In certain embodiments, the antibody comprises a heavy chain sequence which comprises SEQ ID NO: 14 and a light chain sequence which comprises SEQ ID NO: 16. In certain embodiments, the antibody comprises a heavy chain sequence which comprises SEQ ID NO: 15 and a light chain sequence which comprises SEQ ID NO: 16. In certain embodiments, the antibody comprises a heavy chain sequence which comprises SEQ ID NO: 17 and a light chain sequence which comprises SEQ ID NO: 21. In certain embodiments, the antibody comprises a heavy chain sequence which comprises SEQ ID NO: 18 and a light chain sequence which comprises SEQ ID NO: 21. In certain embodiments, the antibody comprises a heavy chain sequence which comprises SEQ ID NO: 19 and a light chain sequence which comprises SEQ ID NO: 21. In certain embodiments, the antibody comprises a heavy chain sequence which comprises SEQ ID NO: 20 and a light chain sequence which comprises SEQ ID NO: 21. In certain embodiments, the antibody comprises a heavy chain sequence which comprises SEQ ID NO: 22 and a light chain sequence which comprises SEQ ID NO: 26. In certain embodiments, the antibody comprises a heavy chain sequence which comprises SEQ ID NO: 23 and a light chain sequence which comprises SEQ ID NO: 26. In certain embodiments, the antibody comprises a heavy chain sequence which comprises SEQ ID NO: 24 and a light chain sequence which comprises SEQ ID NO: 26. In certain embodiments, the antibody comprises a heavy chain sequence which comprises SEQ ID NO: 25 and a light chain sequence which comprises SEQ ID NO: 26.

The present disclosure also provides antibody-drug conjugates comprising any of the antibodies disclosed herein.

### DETAILED DESCRIPTION

### Brief Description of the Drawings

FIG. 1: Human IgG Sub-types

FIG. 2: Representative Size Exclusion Chromatography (“SEC”) chromatograms of (A) trastuzumab-DBM(C6)-MMAF, (B) IGN523-DBM(C6)-MMAF, and (C) IGN786-DBM(C6)-MMAF

FIG. 3: Representative Hydrophobic Interaction Chromatography (“HIC”) chromatograms of (A) IGN523-DBM(C6)-MMAF, (B) trastuzumab-DBM(C6)-MMAF, and (C) IGN786-DBM(C6)-MMAF

FIG. 4: Native Mass Spectrometry (“MS”) analysis of trastuzumab-DBM(C6)-MMAF demonstrates >95% homogeneity and DAR=4 drugs/antibody

FIG. 5: Representative SEC chromatograms of (A) trastuzumab-CPM(C6)-MMAF, (B) IGN523-CPM(C6)-MMAF, and (C) IGN786-CPM(C6)-MMAF

FIG. 6: Representative HIC chromatograms of (A) IGN523-CPM(C6)-MMAF, (B) trastuzumab-CPM(C6)-MMAF, and (C) IGN786-CPM(C6)-MMAF

FIG. 7: Native MS analysis of IGN523-CPM(C6)-MMAF demonstrates DAR=4 drugs/antibody

FIG. 8: Native MS analysis of trastuzumab-CPM(C6)-MMAF demonstrates DAR=4 drugs/antibody

FIG. 9: Native MS analysis of IGN786-CPM(C6)-MMAF demonstrates DAR=4 drugs/antibody

FIG. 10: HIC chromatograms of IGN523-DBM(C6)-MMAF

FIG. 11: Pareto Plot of linker-cytotoxin conjuation to antibody for IGN523-DBM(C6)-MMAF

FIG. 12: DoE model contour plots of linker-cytototoxin versus TCEP for IGN523-DBM(C6)-MMAF

FIG. 13: DoE model contour plots of Conjugation Temperature versus pH for IGN523-DBM(C6)-MMAF at (A) 6, (B) 7 and (C) 8 molar equivalents TCEP

FIG. 14: HIC chromatograms of (A) IGN523-DBM(C6)-MMAF, and (B) trastuzumab-DBM(C6)-MMAF

FIG. 15: DoE model contour plots of linker-cytototoxin versus TCEP shows overlapping optimal subregion or “sweet spot” for (A) IGN523-DBM(C6)-MMAF, and (B) trastuzumab-DBM(C6)-MMAF

FIG. 16: HIC chromatograms confirm DoE model prediction for (A) IGN523-DBM(C6)-MMAF, (B) trastuzumab-DBM(C6)-MMAF, and (C) IGN786-DBM(C6)-MMAF

FIG. 17: HIC chromatograms versus MS confirm DoE model prediction for (A) IGN523-DBM(C6)-MMAF, (B) trastuzumab-DBM(C6)-MMAF and, (C) IGN786-DBM(C6)-MMAF

FIG. 18: Native MS analysis of IGN523-DBM(C6)-MMAF demonstrates DAR=4 drugs/antibody

FIG. 19: Native MS analysis of trastuzumab-DBM(C6)-MMAF demonstrates DAR=4 drugs/antibody

FIG. 20: Native MS analysis of IGN786-DBM(C6)-MMAF demonstrates DAR=4 drugs/antibody

FIG. 21: HIC chromatograms showing scale-up for (A) 0.2 mL (1.0 g), (B) 5.0 mL (25 mg), and (C) 200 mL (1.0 g) of trastuzumab-DBM(C6)-MMAF

FIG. 22: Fidelity of “snap” coupling reaction versus DAR homogeneity of the ADC

FIG. 23: HIC chromatograms comparing DBM(C6)-MMAF ADCs ((A) trastuzumab-DBM(C6)-MMAF and (B) IGN18-DBM(C6)-MMAF) with (C) trastuzumab-M(C6)-MMAF and (D) IGN18-M(C6)-MMAF

FIG. 24: LC/MS comparing DBM(C6)-MMAF ADCs ((A) trastuzumab-DBM(C6)-MMAF and (B) IGN18-DBM(C6)-MMAF) with (C) trastuzumab-M(C6)-MMAF and (D) IGN18-M(C6)-MMAF

FIG. 25: Size exclusion chromatograms comparing DBM(C6)-MMAF ADCs ((A) trastuzumab-DBM(C6)-MMAF and (B) IGN18-DBM(C6)-MMAF) with (C) trastuzumab-M(C6)-MMAF and (D) IGN18-M(C6)-MMAF

FIG. 26: HIC chromatograms showing homogenous DBM(C6)-MMAF ADCs from four different antibodies: (B) trastuzumab-DBM(C6)-MMAF, (C) bevacizumab-DBM(C6)-MMAF, (D) rituximab-DBM(C6)-MMAF, and (E) cetuximab-DBM(C6)-MMAF; comparison to (A) trastuzumab-M(C6)-MMAF

FIG. 27: HIC chromatograms showing homogenous DBM(C6)-MMAF ADCs from fourteen (14) different antibodies: (A) trastuzumab-DBM(C6)-MMAF, (B) bevacizumab-DBM(C6)-MMAF, (C) rituximab-DBM(C6)-MMAF, (D) cetuximab-DBM(C6)-MMAF; (E) ADCs 1-5, and (F) ADCs 6-10

FIG. 28: IC₅₀ measurements for DBM(C6)-MMAF ADCs: (A) SKOV3; (B) H446 (X+); and (C) SKBR3 (Her2 positive)

FIG. 29: Affinity and specificity of DBM(C6)-MMAF ADCs for antigen transfected sarcoma cells in vitro: (A) CD98 transfected F279 sarcomas; and (B) ErB2 transfected F244 sarcomas

FIG. 30: Rat PK of trastuzumab DBM(C6)-MMAF ADCs

FIG. 31: Ovarian cancer (SKOV-3) xenograft model of DBM(C6)-MMAF ADCs

FIG. 32: IC₅₀ measurements for DBM(C6)-MMAF and CPM(C6)-MMAF ADCs: (A) SKOV3 (Her2⁺ & CD98⁺); (B) H446 (CD98⁺); and (C) RAMOS (CD98+)

FIG. 33: Rat PK of trastuzumab DBM(C6)-MMAF and CPM(C6)-MMAF ADCs

FIG. 34: Xenograft models for DBM(C6)-MMAF and CPM(C6)-MMAF ADCs: (A) Ovarian cancer (SKOV-3) xenograft model, (B) Acute myeloid leukemia (OCI-AML3 cells) xenograft model (C) Acute myeloid leukemia (THP-1 cells) xenograft model

FIG. 35: Hinge sequences of human IgG1, IgG2, IgG3 and IgG4 antibodies

FIG. 36: HIC chromatograms and MS showing homogenous ADCs with DAR=2 or 3 made by coupling DBM(C6)-MMAF to hinge cysteine mutants of trastuzumab: (A) HIC of trastuzumab(C226A)-DBM(C6)-MMAF; (B) MS of trastuzumab(C226A)-DBM(C6)-MMAF; (C) HIC of trastuzumab(C226AC229A)-DBM(C6)-MMAF; and (D) MS of trastuzumab(C226AC229A)-DBM(C6)-MMAF

FIG. 37: MS showing homogenous ADCs with DAR=2, 3 or 4 made by coupling DBM(C6)-Val-Ala-PAB-MMAE to wild-type trastuzumab, and hinge cysteine mutants of trastuzumab: (A) trastuzumab(C226AC229A)-DBM(C6)-Val-Ala-PAB-MMAE; (B) trastuzumab(C226A)-DBM(C6)-Val-Ala-PAB-MMAE; and (C) trastuzumab-DBM(C6)-Val-Ala-PAB-MMAE

FIG. 38: Representative SEC chromatograms of (A) trastuzumab (C226AC229A)-CPM(C6)-Val-Ala-PBD, (B) IGN523(C226AC229A)-CPM(C6)-Val-Ala-PBD, and (C) IGN786(C226AC229A)-CPM(C6)-Val-Ala-PBD

FIG. 39: Representative reversed phase HPLC chromatogram for IGN786(C226AC229A)-CPM(C6)-Val-Ala-PBD

FIG. 40: Native MS analysis of (A) trastuzumab(C226AC229A)-CPM(C6)-Val-Ala-PBD, (B) IGN523(C226AC229A)-CPM(C6)-Val-Ala-PBD, and (C) IGN786(C226AC229A)-CPM(C6)-Val-Ala-PBD

FIG. 41: In vitro cytotoxicity of trastuzumab(C226AC229A)-CPM(C6)-Val-Ala-PBD, IGN523(C226AC229A)-CPM(C6)-Val-Ala-PBD, and IGN786(C226AC229A)-CPM(C6)-Val-Ala-PBD on MOLM13 cells

### DEFINITIONS

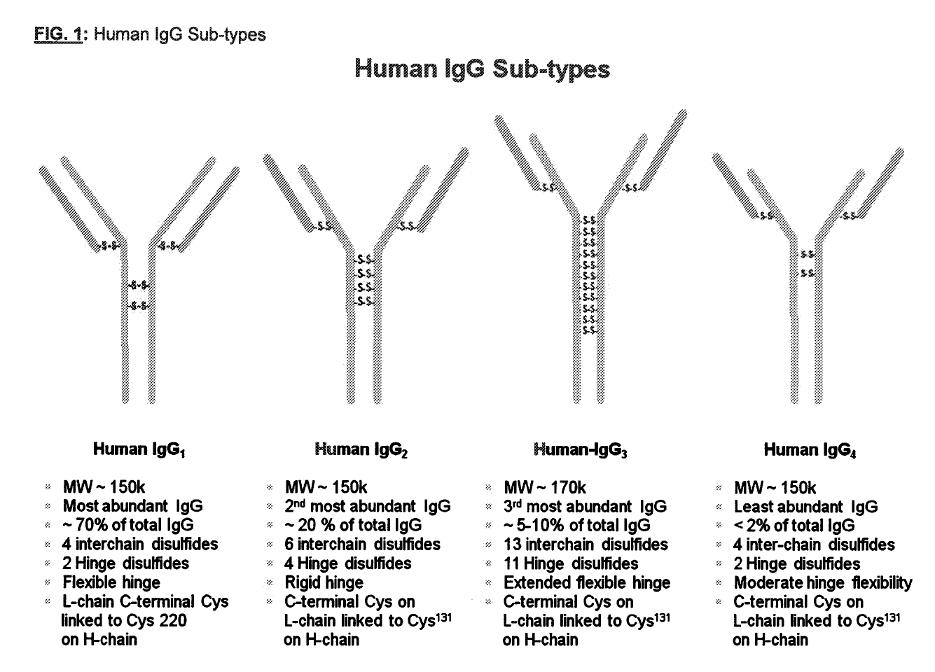

An “antibody,” also known as an immunoglobulin, is a large (e.g., Y-shaped) protein that binds to an antigen. Antibodies are used by the immune system to identify and neutralize foreign objects such as bacteria and viruses. The antibody recognizes a unique part of the antigen, because each tip of the “Y” of the antibody contains a site that is specific to a site on an antigen, allowing these two structures to bind with precision. An antibody (e.g., a multi-chain antibody) may consist of four polypeptide chains, two heavy chains and two light chains connected by interchain cysteine disulfide bonds. For example, antibodies (e.g., multi-chain antibodies) include human IgG1 and human IgG4 which have four interchain disulfide bonds (e.g., two heavy chain-light chain interchain disulfide bonds and two hinge heavy chain-heavy chain interchain disulfide bonds), human IgG2 which has six interchain disulfide bonds (e.g., four heavy chain-light chain interchain disulfide bonds and two hinge heavy chain-heavy chain interchain disulfide bonds), and human IgG3 which has thirteen interchain disulfide bonds (e.g., eleven heavy chain-light chain interchain disulfide bonds and two hinge heavy chain-heavy chain interchain disulfide bonds) (see, e.g., FIG. 1).

(e.g., two heavy chain-light chain interchain disulfide bonds and two hinge heavy chain-heavy chain interchain disulfide bonds). In certain embodiments, where the opened cysteine-cysteine disulfide bond in A is an interchain disulfide bond n is 3 (e.g., two heavy chain-light chain interchain disulfide bonds and one hinge heavy chain-heavy chain interchain disulfide bond). In certain embodiments, where the opened cysteine-cysteine disulfide bond in A is an interchain disulfide bond n is 2 (e.g., two heavy chain-light chain interchain disulfide bonds).

A “monoclonal antibody” is a monospecific antibody where all the antibody molecules are identical because they are made by identical immune cells that are all clones of a unique parent cell. Initially, monoclonal antibodies are typically prepared by fusing myeloma cells with the spleen cells from a mouse (or B-cells from a rabbit) that has been immunized with the desired antigen, then purifying the resulting hybridomas by such techniques as affinity purification. Recombinant monoclonal antibodies are prepared in viruses or yeast cells rather than in mice, through technologies referred to as repertoire cloning or phage display/yeast display, the cloning of immunoglobulin gene segments to create libraries of antibodies with slightly different amino acid sequences from which antibodies with desired specificities may be obtained. The resulting antibodies may be prepared on a large scale by fermentation. “Chimeric” or “humanized” antibodies are antibodies containing a combination of the original (usually mouse) and human DNA sequences used in the recombinant process, such as those in which mouse DNA encoding the binding portion of a monoclonal antibody is merged with human antibody-producing DNA to yield a partially-mouse, partially-human monoclonal antibody. Full-humanized antibodies are produced using transgenic mice (engineered to produce human antibodies) or phage display libraries. Antibodies (Abs) and “immunoglobulins” (Igs) are glycoproteins having similar structural characteristics. While antibodies exhibit binding specificity to a specific antigen, immunoglobulins include both antibodies and other antibody-like molecules which generally lack antigen specificity. Polypeptides of antibody-like molecules are produced at low levels by the lymph system and at increased levels by myelomas. The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity). An antibody can be chimeric, human, humanized and/or affinity matured. Antibodies of particular interest are those that are specific to cancer antigens, are non-immunogenic, have low toxicity, and are readily internalized by cancer cells; and suitable antibodies include alemtuzumab, bevacizumab, brentuximab, cetuximab, gemtuzumab, ipilimumab, ofatumumab, panitumumab, rituximab, tositumomab, inotuzumab, glembatumumab, lovortuzumab and trastuzumab. Additional antibodies include adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab, votumumab, and zalutumumab. Additional antibodies include anti-HER2 antibodies, anti-CD98 antibodies, and anti-C16orf54 antibodies.

The heavy chain variable region (VH) and light chain variable region (VL) sequences of an exemplary anti-HER2 antibody trastuzumab (e.g. Herceptin®) are shown in Table A.

TABLE A
VH
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
(SEQ ID NO: 1)
VL
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK
(SEQ ID NO: 2)

The heavy chain variable region (VH) and light chain variable region (VL) sequences of an exemplary anti-CD98 antibody, designated herein as IGN523, are shown in Table B.

TABLE B
VH
MEWSWVFLFFLSVTTGVHSQVQLVQSGAEVKKPGSSVKVSCKASGNAFTN
YLIEWVRQAPGQGLEWMGVINPGSGITNYNEKFKGKATITADKSTSTAYM
ELSSLRSEDTAVYYCSGSANWFAYWGQGTLVTVSS
(SEQ ID NO: 3)
VL
MSVPTQVLGLLLLWLTDARCDIVMTQSPDSLAVSLGERATINCKSSQSLL
YSSNQKNYLAWYQQKPGQPPKLLIYWASTRDSGVPDRFTGSGSGTDFTLT
ISSLQAEDVAVYYCQRYYGYPWTFGGGTKVEIK
(SEQ ID NO: 4)
(each with a signal sequence)

Heavy and light chain leader sequences are shown underlined. Exemplary complementarity-determining regions (CDRs), are shown in bold.

The heavy chain variable region (VH) and light chain variable region (VL) sequences of an exemplary anti-C16orf54 antibody, designated herein as IGN786, are shown in Table C.

TABLE C
VH
QVQLQESGPGLVKPSDTLSLTCAVSGYSITSDYAWNWIRQPPGKGLEWMG
YISYSGSIRYNPSLKSRITISRDTSKNQFSLKLSSVTAVDTAVYYCAREK
YDNYYYAMDYWGQGTLVTVSS
(SEQ ID NO: 5)
VL
DIVMTQSPDSLAVSLGERVTLNCKSSQNLLYSTNQKNYLAWYQQKPGQPP
KLLIYWASTRESGVPDRFSGSGSGTDFTLTISSVQAEDLAVYYCQQYYSY
RTFGQGTKLEIK
(SEQ ID NO: 6)

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, ADC function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody,” as used herein, refers to an antibody obtained from a population of substantially homogeneous antibodies, e.g., the individual antibodies comprising the population are identical except for possible mutations, e.g., naturally occurring mutations, that may be present in minor amounts. The modifier term “monoclonal” indicates the character of the antibody as not being a mixture of discrete antibodies. In certain aspects, such a monoclonal antibody may include an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In addition to their specificity, monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. (See, Kohler et al., Nature, 256: 495 (1975); Harlow et al., Antibodies: A Laboratory Manual, (Cold Spring Harbor Laboratory Press, 2.sup.nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), and technologies for producing human or human-like antibodies in animals that have parts or all of the human immunoglobulin loci or genes encoding human immunoglobulin sequences (see, WO98/24893; WO96/34096; WO96/33735 and WO91/10741). The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). “Humanized” forms of non-human (e.g., murine) antibodies are chimeric antibodies that contain minimal sequence derived from non-human immunoglobulin. In one aspect, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In another aspect, framework region (FR) residues of the human immunoglobulin are replaced by corresponding non-human residues. In general, a humanized antibody will comprise substantially all of at least one, and typically two, variable domains, in which all or substantially all of the hypervariable loops correspond to those of a non-human immunoglobulin, and all or substantially all the FRs are those of a human immunoglobulin sequence. The humanized antibody may comprise at least a portion of an immunoglobulin constant region (Fc), typically that of a human immunoglobulin. See Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

“Framework” or “FR” residues are those variable domain residues other than the hypervariable region residues. “Fc receptor” or “FcR” is a receptor that binds to the Fc region of an antibody. In certain embodiments, an FcR is a native human FcR. In one aspect, an FcR is one which binds an IgG antibody (a gamma receptor) and includes receptors of the FcγRI, FcγRII and FcγRIII subclasses. (See Daeron, Annu. Rev. Immunol. 15:203-234 (1997)).

The term “thiol,” as used herein, refers to the radical —SH. The term “substituted thiol,” as used herein, refers to a radical such as —SR wherein R is any optionally substituted chemical group described herein. In certain embodiments, “substituted thiol” refers to a radical —SR where R is an alkyl, cycloalkyl, aryl or heteroaryl group as defined herein that may be optionally substituted as defined herein. Representative examples of substituted thiol include, but are not limited to, thiophenyl, thionaphthyl, thiopyridyl, thioisoquinolinyl, as depicted below:

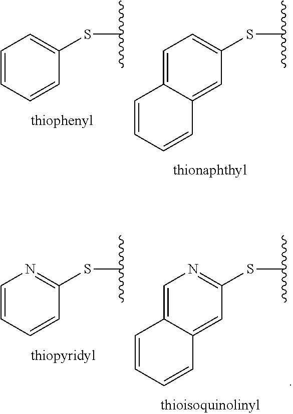

The term “sulfonate,” as used herein, refers to the radical —OS(O₂)H. “Substituted sulfonate” refers to a radical such as —OS(O₂)R wherein R is an alkyl, cycloalkyl, aryl or heteroaryl group as defined herein that may be optionally substituted as defined herein. In certain embodiments, R is selected from lower alkyl, alkyl, aryl and heteroaryl. Representative examples of substituted sulfonate include, but are not limited to, tosylate, mesylate and triflate, as depicted below:

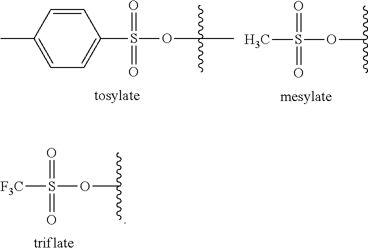

The terms “phenyloxy” or “phenol,” as used herein, refers to the radical —O-phenyl. “Substituted phenyloxy” or “substituted phenol” refers to the radical —O-phenyl wherein the phenyl ring is substituted with 1 to 5 substituents selected from the group consisting of halo, cyano, nitro, CF₃—, CF₃O—, CH₃O—, —CO₂H, —C(O)CH₃, —NH₂, —OH, —SH, —NHCH₃, —N(CH₃)₂, —SMe and C₁₋₃ alkyl.

The term “carboxyl protecting group,” as used herein, refers to a protecting group that serves to protect a carboxylic acid functional group. The term includes, without limitation, a methyl ester, a tert-butyl ester, a benzyl ester, an S-tert-butyl ester, 2-alkyl-1,3-oxazoline, and the like.

The term “amide bond,” as used herein, refers to a bond comprising an optionally substituted amide group. For example, the amide bond may comprise the following structure:

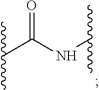 [SMILES: *NC(*)=O]

where the squiggly lines indicate attachment points to the rest of the molecule.

The term “carbamate bond,” as used herein, refers to a bond comprising an optionally substituted carbamate group. For example, the carbamate bond may comprise the following structure:

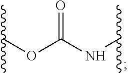 [SMILES: *NC(=O)O*]

where the squiggly lines indicate attachment points to the rest of the molecule.

A “cytotoxin” (CTX) is a molecule that, when released within a cancer cell, is toxic to that cell.

A “linker” (noted as L) is a molecule with two reactive termini, one for conjugation to an antibody or to another linker and the other for conjugation to a cytotoxin. The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo or iodo or an R-sulfanyl group or sulfonyl group, or an amine-reactive group such as a carboxyl group or as defined herein; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. In one embodiment, when the term “linker” is used in describing the linker in conjugated form, one or both of the reactive termini will be absent (such as the leaving group of the thiol-reactive group) or incomplete (such as the being only the carbonyl of the carboxylic acid) because of the formation of the bonds between the linker and/or the cytotoxin.

The term “cleavable linker,” as used herein, refers to a linker that is hydrolyzed in vivo, for example, that is hydrolyzed in vivo by an enzymatic process.

The term “noncleavable linker” or “stable linker,” as used herein, refers to a linker that is not hydrolyzed in vivo, for example, that is resistant to cleavage by an enzymatic process in vivo.

The term “leaving group,” as used herein, refers to any group that leaves in the course of a chemical reaction involving the group as described herein and includes but is not limited to halogen, sulfonates (brosylate, mesylate, tosylate triflate etc. . . . ), p-nitrobenzoate, phosphonate, and p-cyanophenol groups, for example.

The term “electrophilic leaving group,” as used herein, refers to a leaving group that accepts an electron pair to make a covalent bond. In general, electrophiles are susceptible to attack by complementary nucleophiles, including the reduced thiols from the disulfide bond of an antibody.

The term “electrophilic leaving group that reacts selectively with thiols,” as used herein, refers to electrophilic leaving group that reacts selectively with thiols, over other nucleophiles. In certain embodiments, an electrophilic leaving group that reacts selectively with thiols reacts selectively with the reduced thiols from the disulfide bond of an antibody.

An “antibody-drug conjugate” (ADC) is an antibody that is conjugated to one or more cytotoxins, through one or more linkers. The antibody is typically a monoclonal antibody specific to a therapeutic target such as a cancer antigen.

A “cytotoxic agent” or “cytotoxin” is a molecule that has a cytotoxic effect on cells (e.g., when released within a cancer cell, is toxic to that cell).

The term “MMAF” generally refers to monomethylauristatin F, for which a chemical name is (S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid.

The term “MMAE” generally refers to refers to monomethylauristatin E, for which a chemical name is (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide.

The term “pyrrolobenzodiazepine” or “pyrrolobenzodiazepines” generally refers to a family of pyrrolo[2,1-c][1,4]benzodiazepine (PBD) dimers which are synthetic sequence-selective interstrand DNA minor-groove cross-linking agents developed from anthramycins. Examples of pyrrolobenzodiazepines include, but are not limited to, abbeymycin, chicamycin, DC-81, mazethramycin, neothramycins A and B, porothramycin, prothracarcin, sibanomicin (DC-102), sibiromycin and tomamycin. Exemplary pyrrolobenzodiazepines include those disclosed in U.S. Pat. Nos. 7,049,311, 7,741,319, 8,697,688 (see, e.g., (26) in Example 5), and 8,765,740; International Publication Nos. WO 2011/130598 A1, WO 2012/112708 A1, WO 2013/055987 A1, WO 2013/165940 A1; and Jeffrey et al., Bioconjugate Chem. 2013, 24, 1256-1263, and Sutherland et al., Blood 2013, 122(8), 1455-1463; the content of each of which is incorporated by reference in its entirety.

The terms “cell proliferative disorder” and “proliferative disorder” refer to disorders that are associated with some degree of abnormal cell proliferation. In one aspect, the cell-proliferative disorder is cancer.

“Tumor,” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive. The terms “cancer” and “cancerous” refer to the physiological condition in mammals that is typically characterized by unregulated cell growth. Examples of cancer include, but are not limited to, carcinoma, lymphoma, blastoma, sarcoma and leukemia or lymphoid malignancies.

A “therapeutically effective amount” means that amount of an ADC or composition disclosed herein which, when administered to a human suffering from a cancer, is sufficient to effect treatment for the cancer. “Treating” or “treatment” of the cancer includes one or more of:

(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

As used herein, the term “pharmaceutically acceptable salt” refers to those salts of the ADCs formed by the process of the present application which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66:1-19 (1977). The salts can be prepared in situ during the final isolation and purification of the ADC compounds, or separately by reacting the free base function or group of a compound with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid, etc., or with organic acids such as acetic acid, maleic acid, tartaric acid, citric acid, succinic acid or malonic acid. Other pharmaceutically acceptable salts include, but are not limited to, adipate, alginate, ascorbate, benzenesulfonate, benzoate, bisulfate, citrate, digluconate, dodecylsulfate, ethanesulfonate, formate, fumarate, gluconate, 2-hydroxy-ethanesulfonate, lactate, laurate, malate, maleate, malonate, methanesulfonate, oleate, oxalate, palmitate, phosphate, propionate, stearate, succinate, sulfate, tartrate, p-toluenesulfonate, valerate salts, and the like. Representative alkali or alkaline earth metal salts include sodium, lithium, potassium, calcium, or magnesium salts, and the like. Further pharmaceutically acceptable salts include, nontoxic ammonium, quaternary ammonium, and amine cations formed using counterions such as halide, hydroxide, carboxylate, sulfate, phosphate, nitrate, alkyl groups having from 1 to 6 carbon atoms (e.g., C₁₋₆ alkyl), sulfonate and aryl sulfonate.

Cancers of interest for treatment include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer including, for example, HER2-positive breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, acute myeloid leukemia (AML), chronic lymphocytic leukemia (CML), multiple myeloma and B-cell lymphoma, brain cancer, head and neck cancers and associated metastases.

### Abbreviations/Acronyms

ADC: antibody-drug conjugate; BOC: tert-butyloxycarbonyl; BRM: bromomaleimide; Cbz: benzyl carbamate; CPM: cyanophenolmaleimide; DAR: Drug-to-antibody ratio; dbm or DBM: dibromomaleimide; DIPC: 1,3-diisopropylcarbodiimide; DIPEA: diisopropylethylamine; DMA: dimethyacetamide; DMF: N,N-dimethylformamide; DPBS: Dulbecco's phosphate-buffered saline; DTNB: 5,5′-dithiobis-(2-nitrobenzoic acid); DTPA: diethylenetriaminepentaacetic acid; DTT: dithiothreitol; EEDQ: ethoxycarbonyl-ethoxy-dihydroquinoline; Fmoc or FMOC: 9-fluorenylmethoxycarbonyl chloride; HATU: O-(7-azabenzotriazol-1-yl)-N,N,N′,N′-tetramethyluronium hexafluorophosphate; HIC: hydrophobic interaction chromatography; HPLC: High Performance Liquid Chromatography; MC or mc: maleimido caproyl, maleimidocaproyl, 6-(2,5-dioxopyrrolyl)hexanoyl; MMAE: monomethylauristatin E; MMAF: monomethylauristatin F; NMM: N-methylmorpholine; PAB: para amino benzyl; PBD: pyrrolobenzodiazepine; PBS: phosphate-buffered saline; PEG: poly(ethyleneglycol); p-TOS: p-toluenesulfonamide; TBTU: 2 (1H benzotriazol-1 yl)-1,1,3,3 tetramethyluronium tetrafluoroborate; TCEP: tris(2-carboxyethyl)phosphine; TGI: tumor growth inhibition; TEA: triethanolamine; THF: tetrahydrofuran; VA: Valine-Alanine; VAP: Valine-Alanine-para amino benzyl; VA(PAB): Valine-Alanine-para amino benzyl; VC: Valine-Citrulline; VCP: Valine-Citrulline-para amino benzyl; VC(PAB): Valine-Citrulline-para amino benzyl.

### Preparation of the Linkers

The linkers disclosed herein may be cleavable under normal physiological and/or intracellular conditions, or may remain stable (e.g., uncleaved or non-cleavable) under those same conditions.

For example, cleavable linkers may remain stable during systemic circulation but may be cleaved under certain intracellular conditions, such as in an acidic environment. For example, where an ADC is processed in a lysosome of a cell, the linker may be cleaved by the acidic environment and/or the enzymes in the lysosome, releasing the cytotoxin from the antibody. Examples of cleavable linkers are linkers which contain dipeptide moieties, where the peptide bond connecting the two peptides has the potential to be selectively cleaved by lysosomal proteases (e.g., cathepsin-B). Valine-alanine (“Val-Ala” or “VA”) and valine-citruline (“Val-Cit” or “VC”) are dipeptide moieties commonly used in cleavable linkers.

Noncleavable linkers may remain stable, both during systemic circulation and under certain intracellular conditions, such as in an acidic environment. Examples of stable linkers are linkers which do not contain dipeptide moieties, for example, alkyl and/or PEG linkers.

The following schemes a, b, c, d, e, and f illustrate general synthetic schemes for stable linkers (e.g., uncleaved or non-cleavable) as disclosed herein, which may be synthesized by the methods disclosed herein:

### Illustrative General Synthetic Schemes for Stable Linkers as Disclosed Herein:

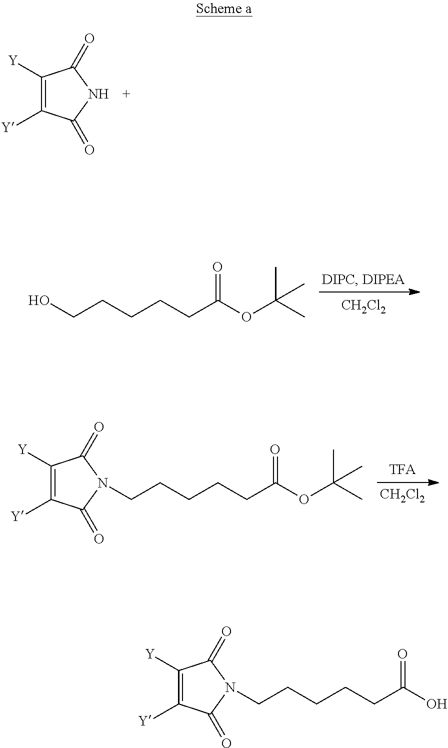

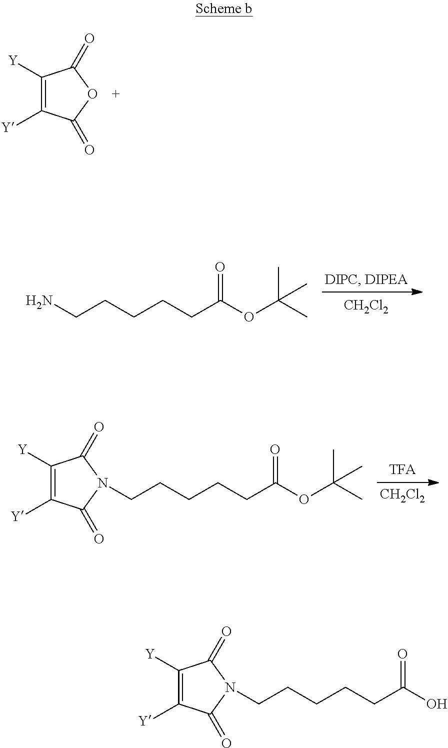

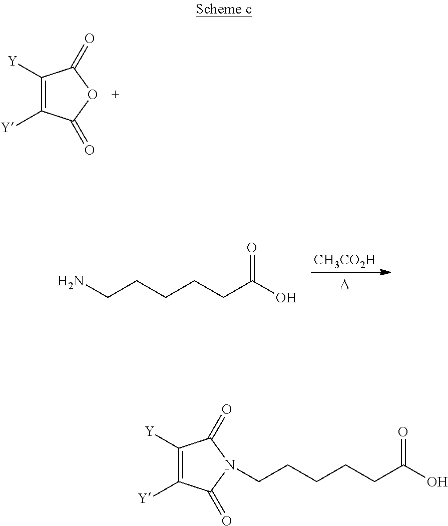

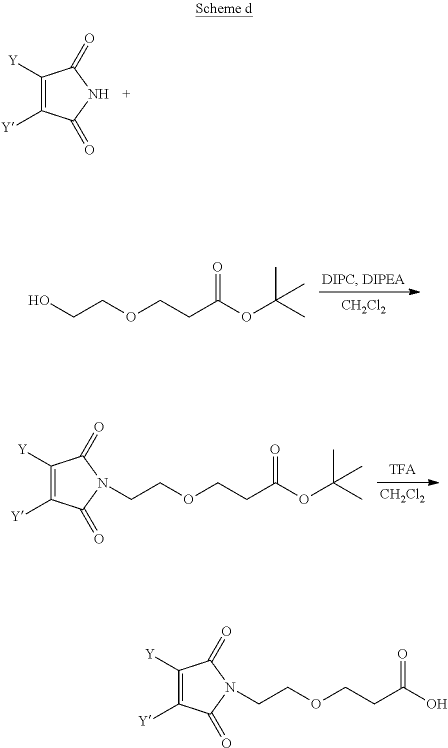

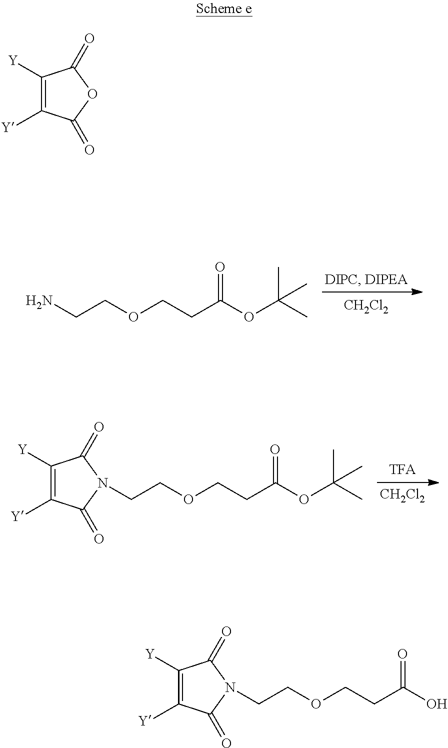

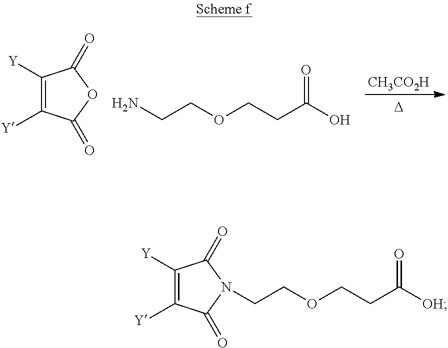

wherein Y and Y′ are as defined herein.

The above schemes are merely illustrative, and not meant to be limiting.

The following schemes g, h, i, and j illustrate general synthetic schemes for cleavable linkers as disclosed herein, which may be synthesized by the methods disclosed herein:

### Illustrative General Synthetic Schemes for Cleavable Linkers as Disclosed Herein:

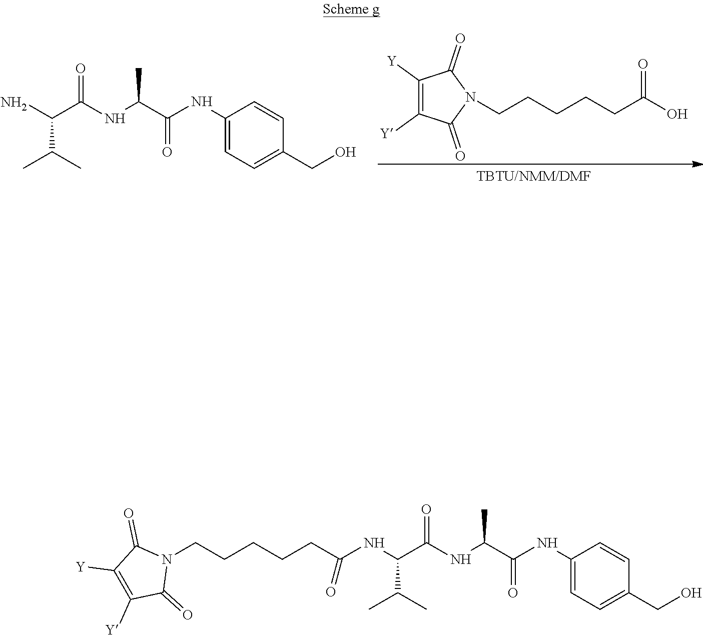

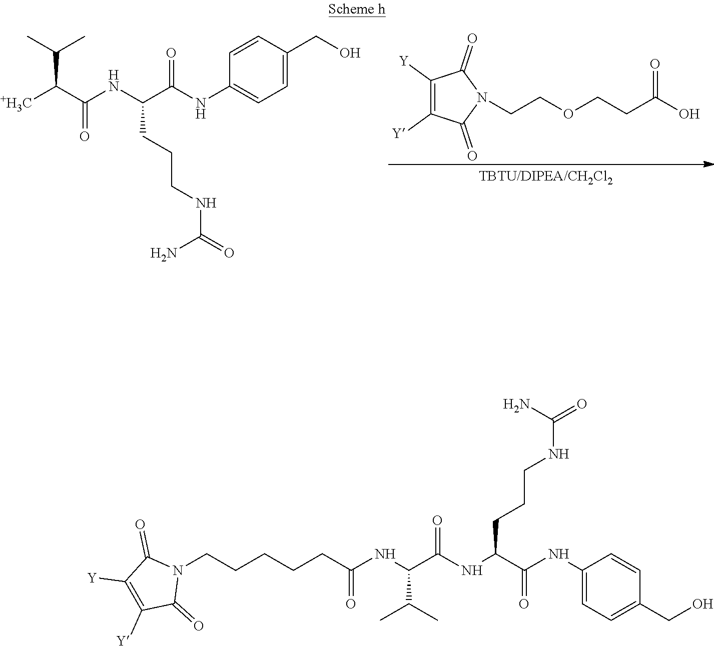

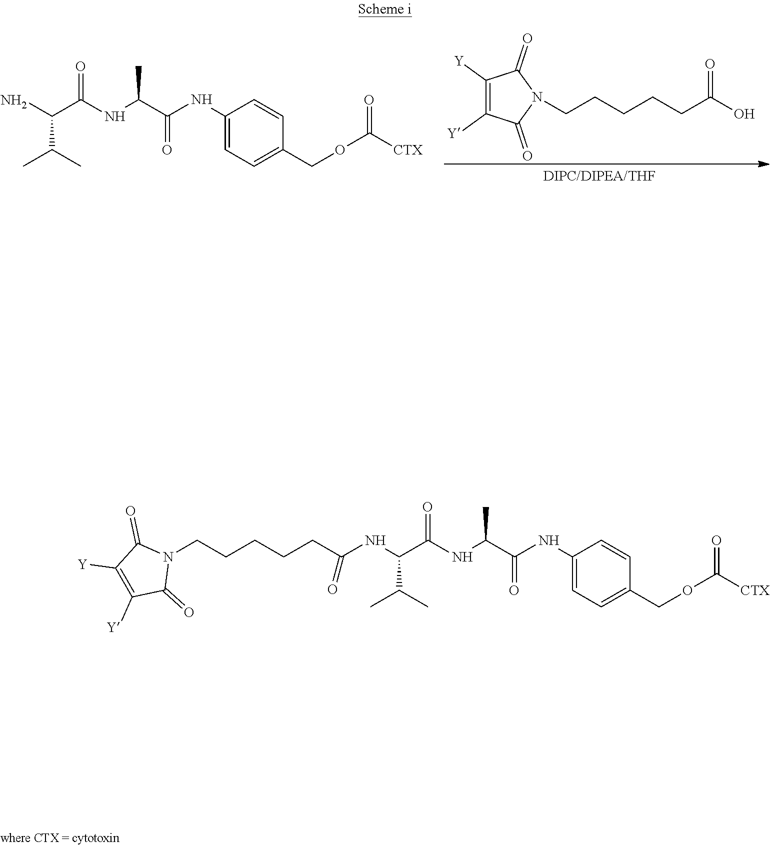

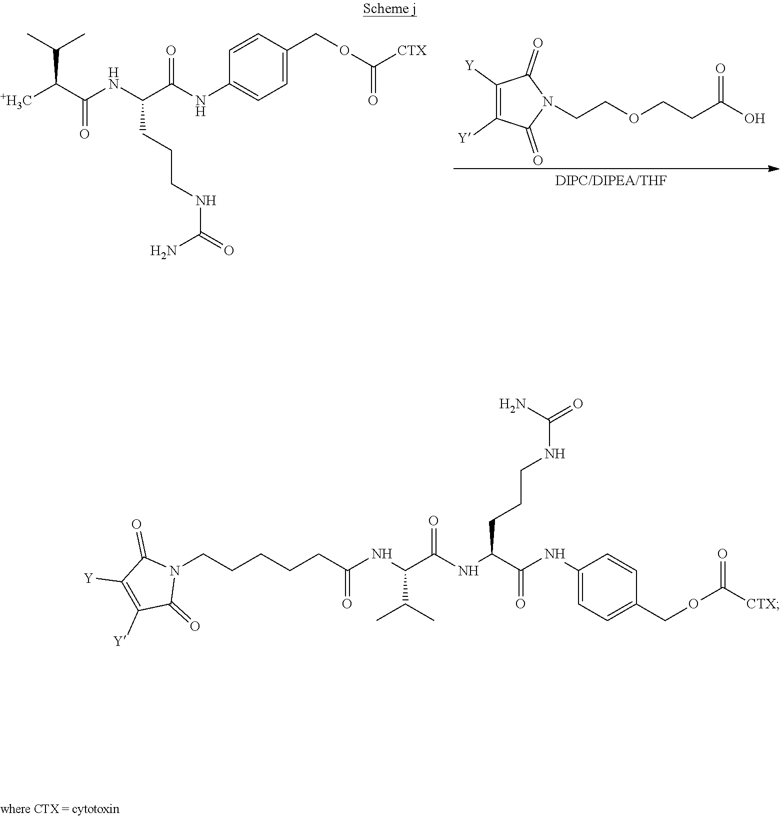

and wherein Y and Y′ are as defined herein.

The above schemes are merely illustrative, and not meant to be limiting.

### Preparation of Linker-Cytotoxin Conjugates

Linker-Cytotoxin conjugates may be prepared by methods analogous to those of Doronina et al., Bioconjugate Chem. 2006, 17, 114-124, and similar documents. The linker, 1 equivalent, and HATU, 1 equivalent, are dissolved in anhydrous DMF, followed by the addition of DIPEA, 2 equivalents. The resulting solution is added to the cytotoxin, 0.5 equivalents, dissolved in DMF, and the reaction stirred at ambient temperature for 3 hr. The linker-cytotoxin conjugate is purified by reverse phase HPLC on a C-18 column.

The following schemes illustrate general synthetic schemes of linker-cytotoxin conjugates as disclosed herein, which may be synthesized by the methods disclosed herein:

### Illustrative General Synthetic Schemes for Linker-Cytotoxin Conjugates as Disclosed Herein (e.g., Stable Linkers):

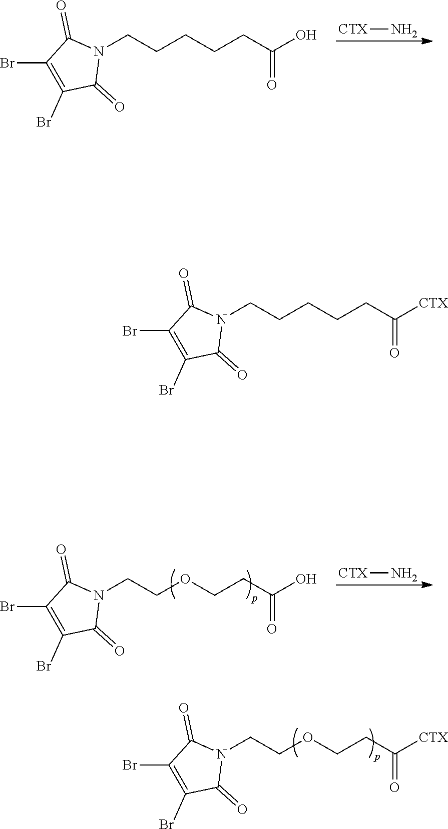

### Additional Illustrative General Synthetic Schemes for Linker-Cytotoxin Conjugates as Disclosed Herein (e.g., Stable Linkers):

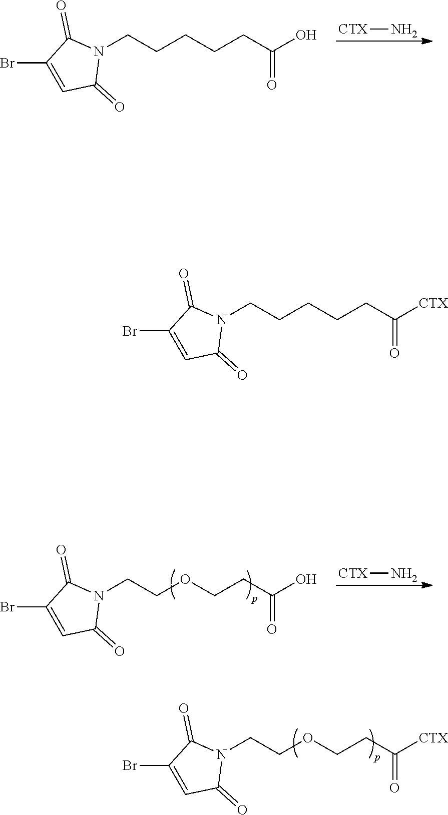

### Additional Illustrative General Synthetic Schemes for Linker-Cytotoxin Conjugates as Disclosed Herein (e.g., Stable Linkers):

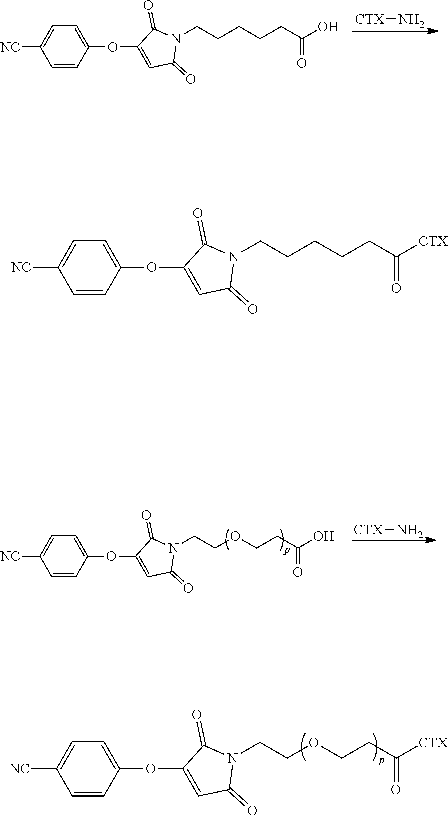

### Illustrative General Synthetic Schemes for Linker-Cytotoxin Conjugates as Disclosed Herein (e.g., Cleavable Linkers):

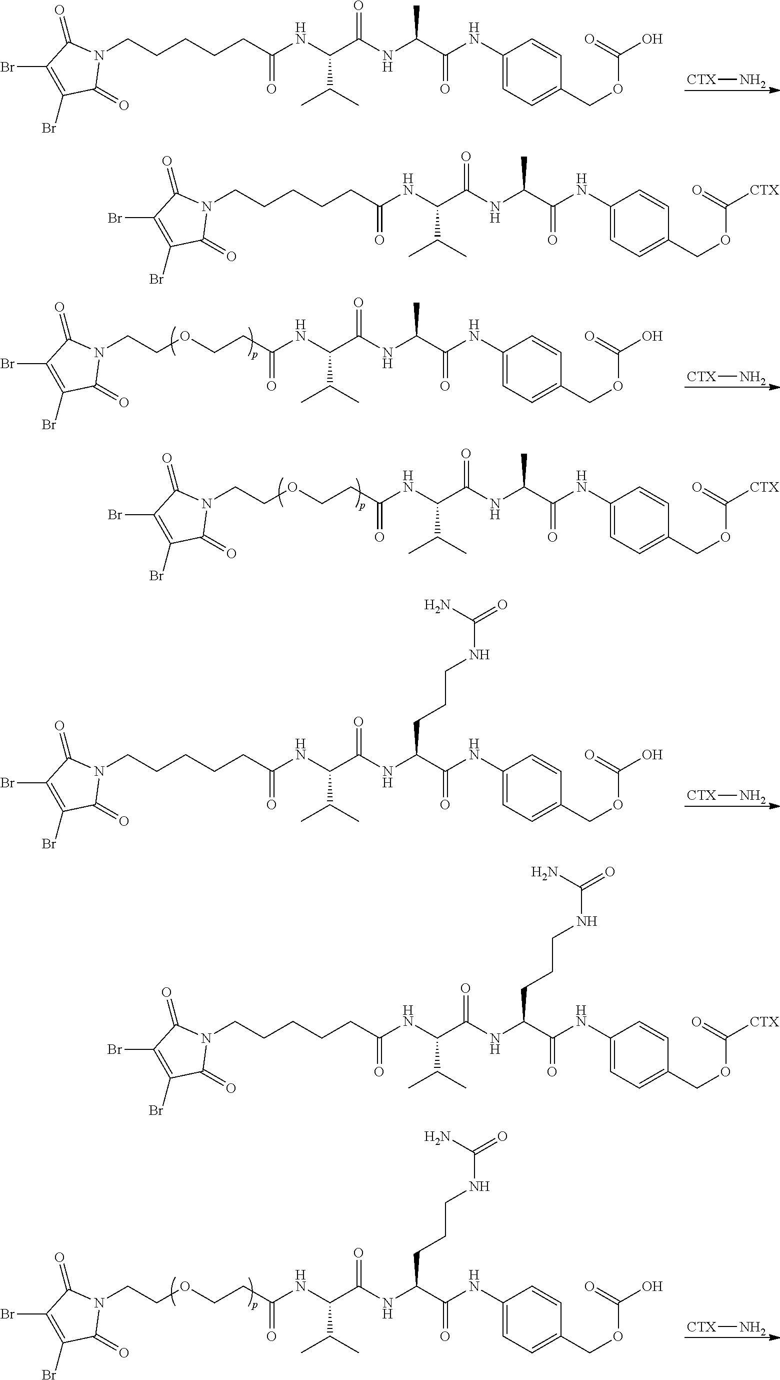

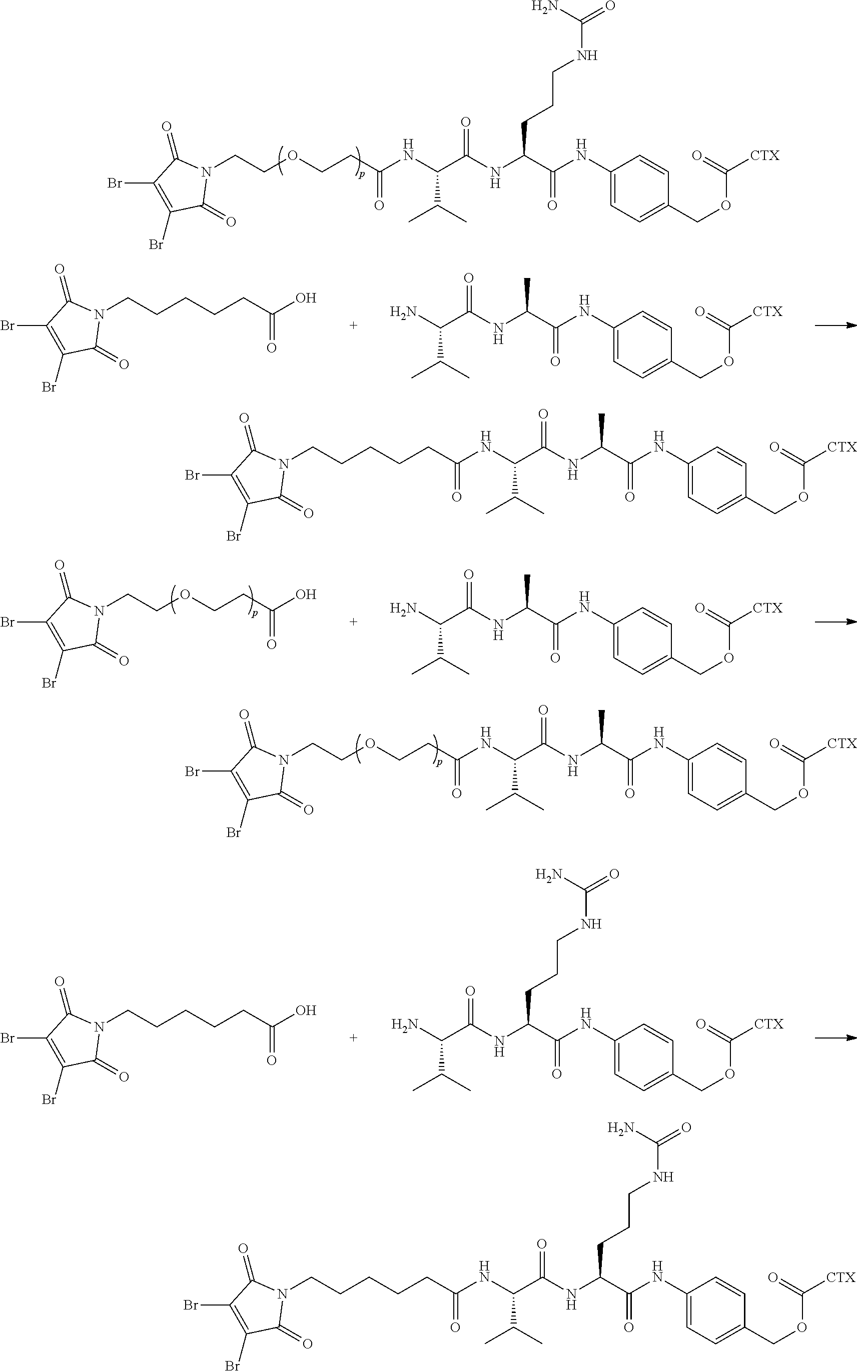

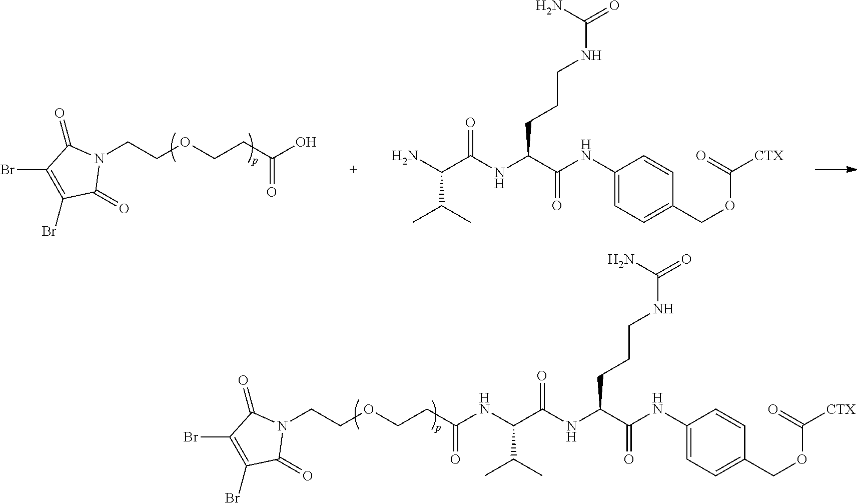

### Additional Illustrative General Schemes for Linker-Cytotoxin Conjugates as Disclosed Herein (e.g., Cleavable Linkers):

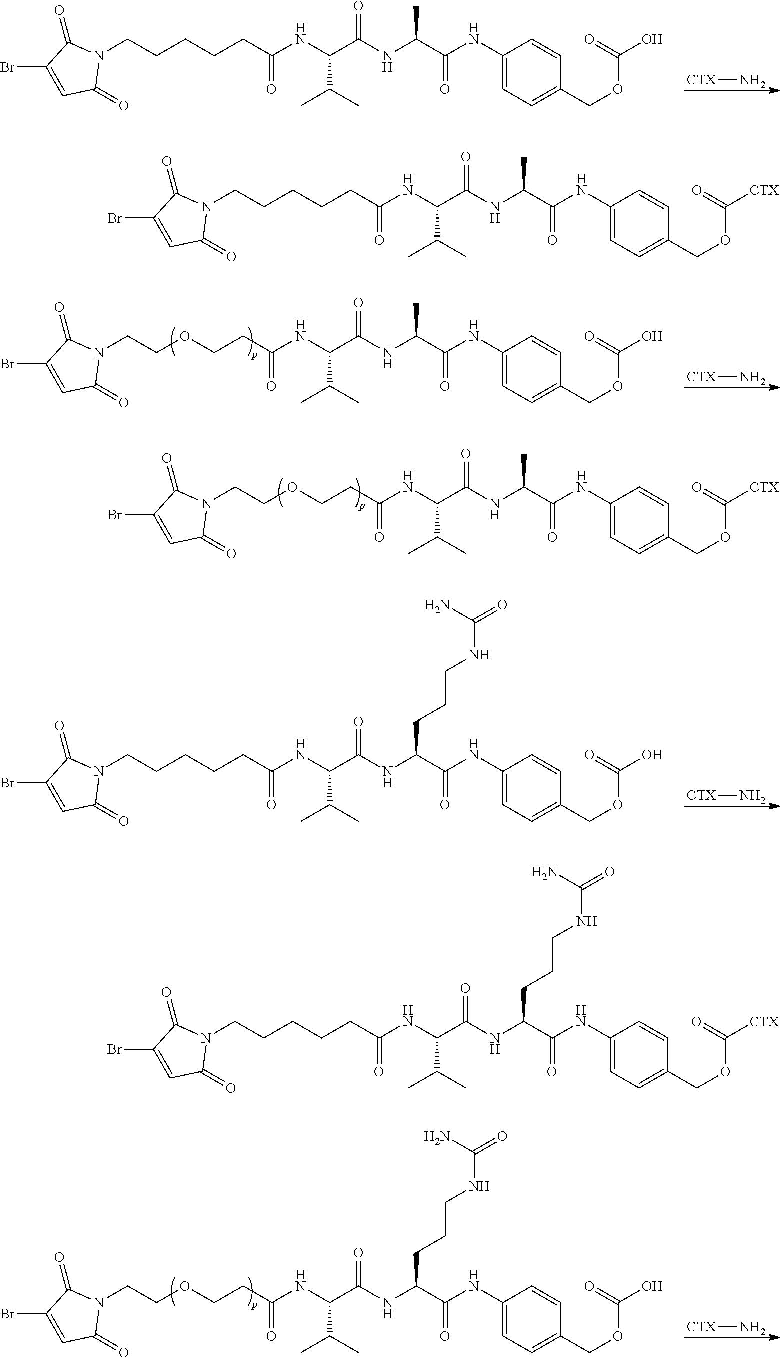

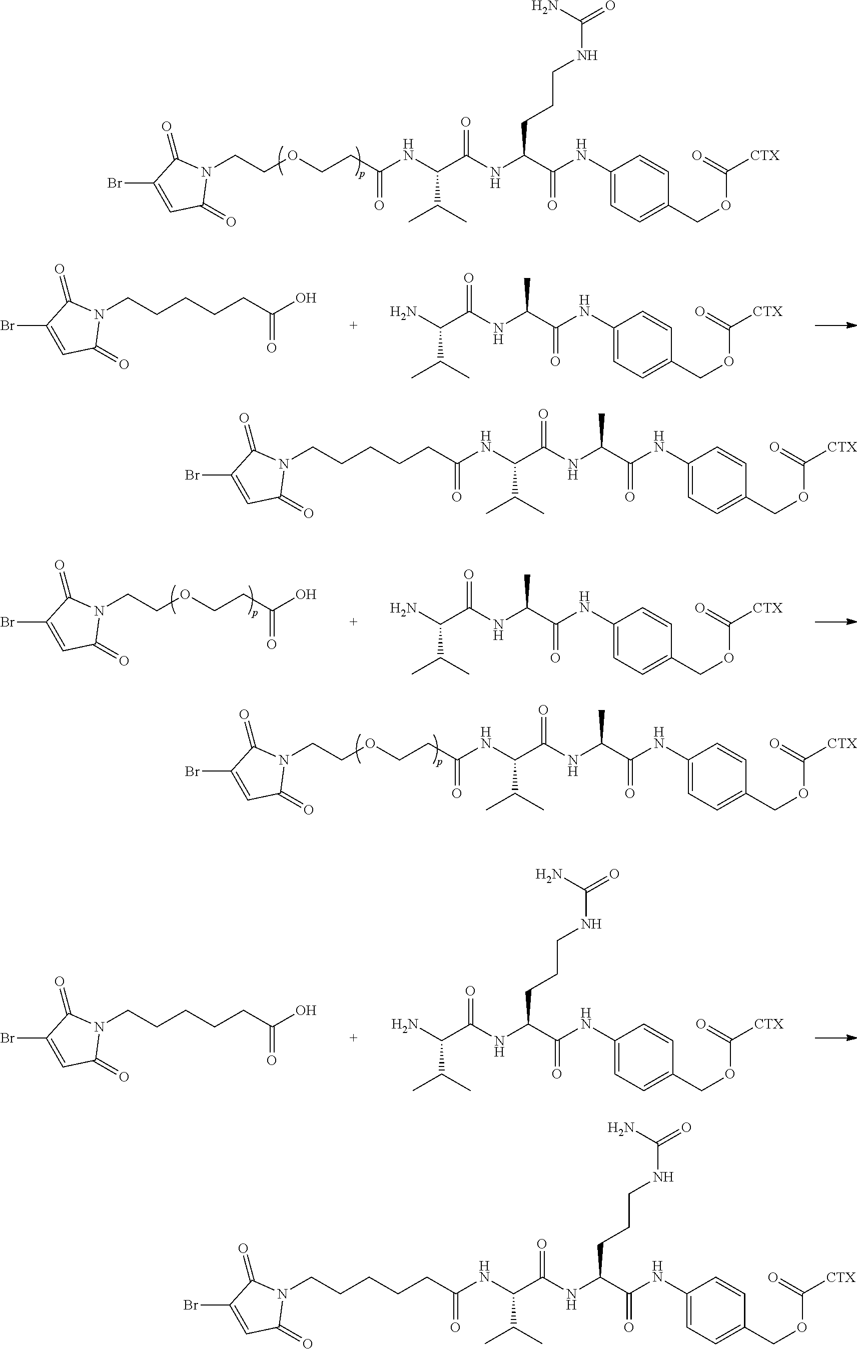

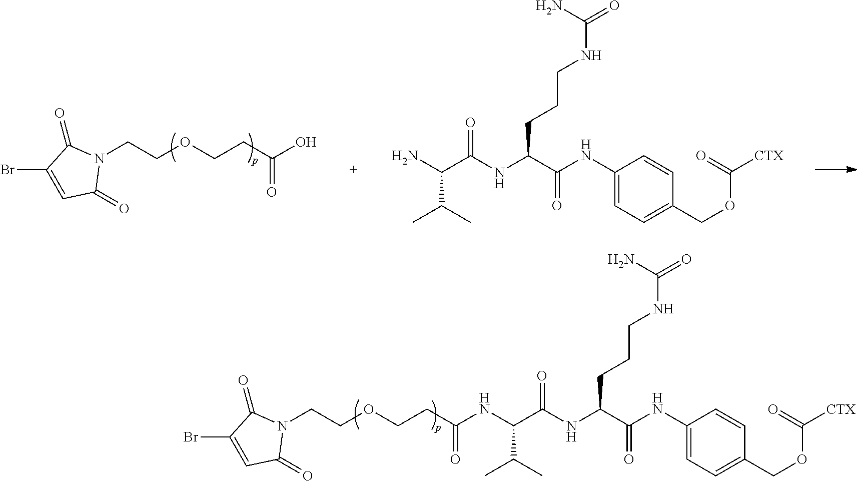

### Additional Illustrative General Synthetic Schemes for Linker-Cytotoxin Conjugates as Disclosed Herein (e.g., Cleavable Linkers):

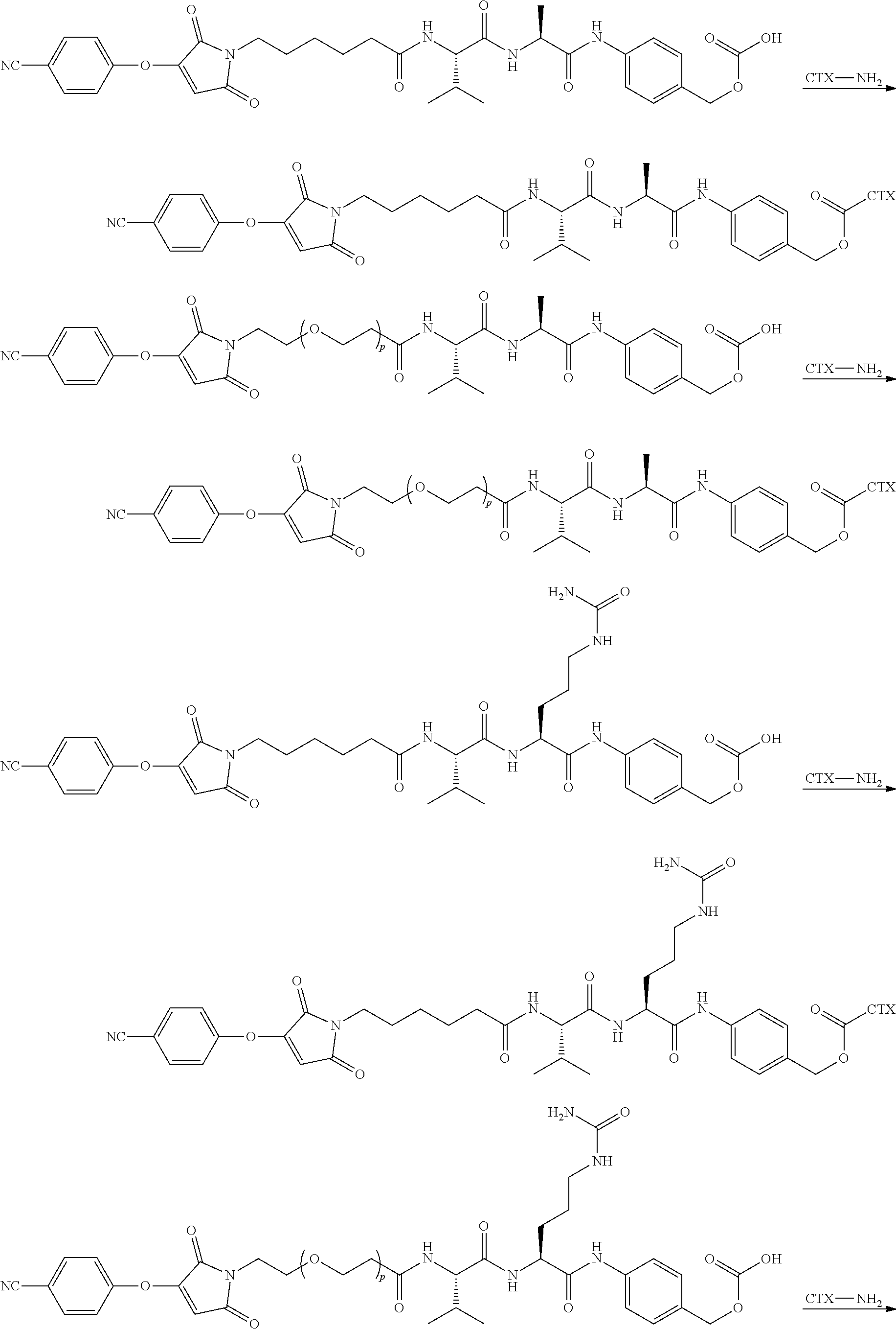

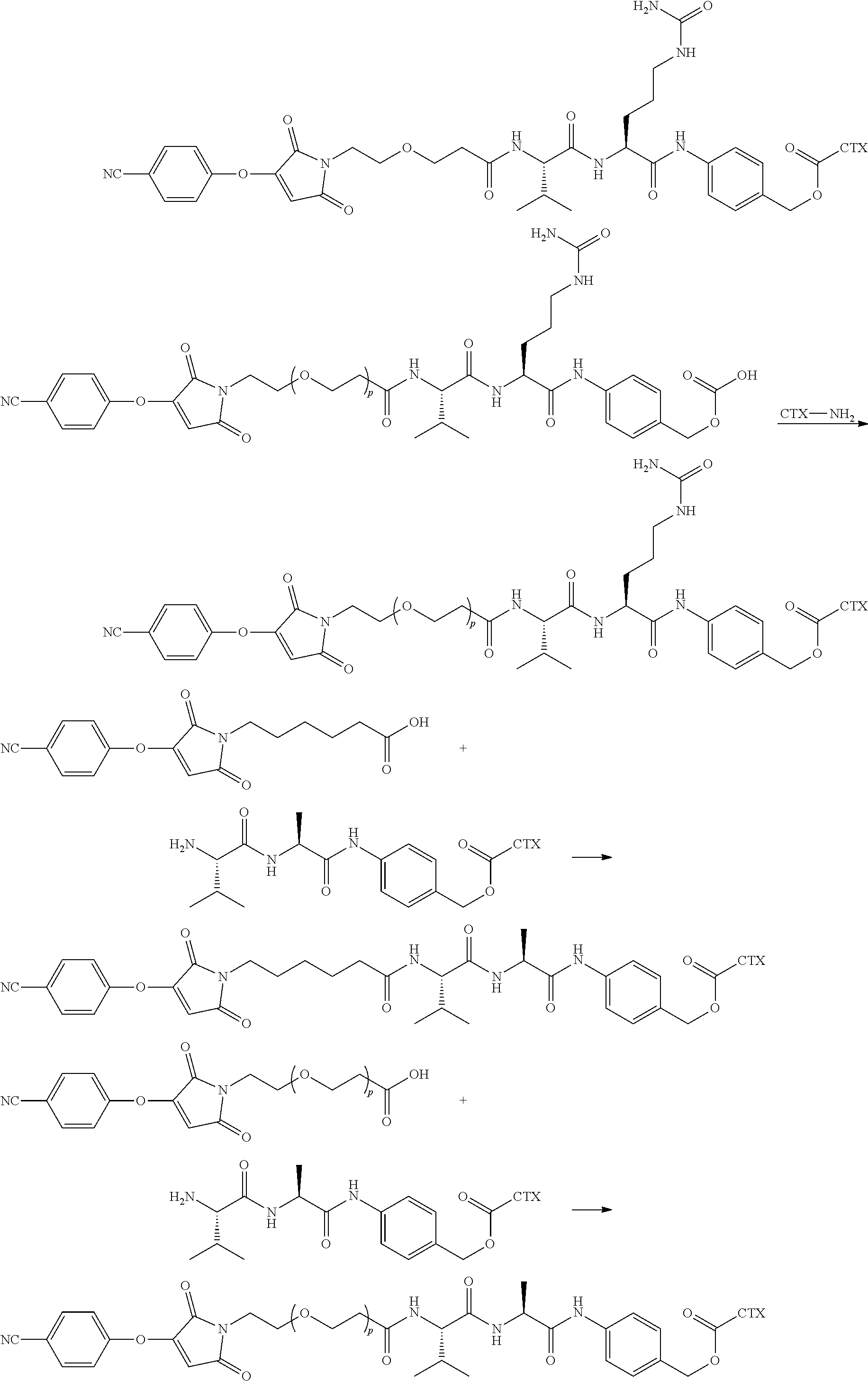

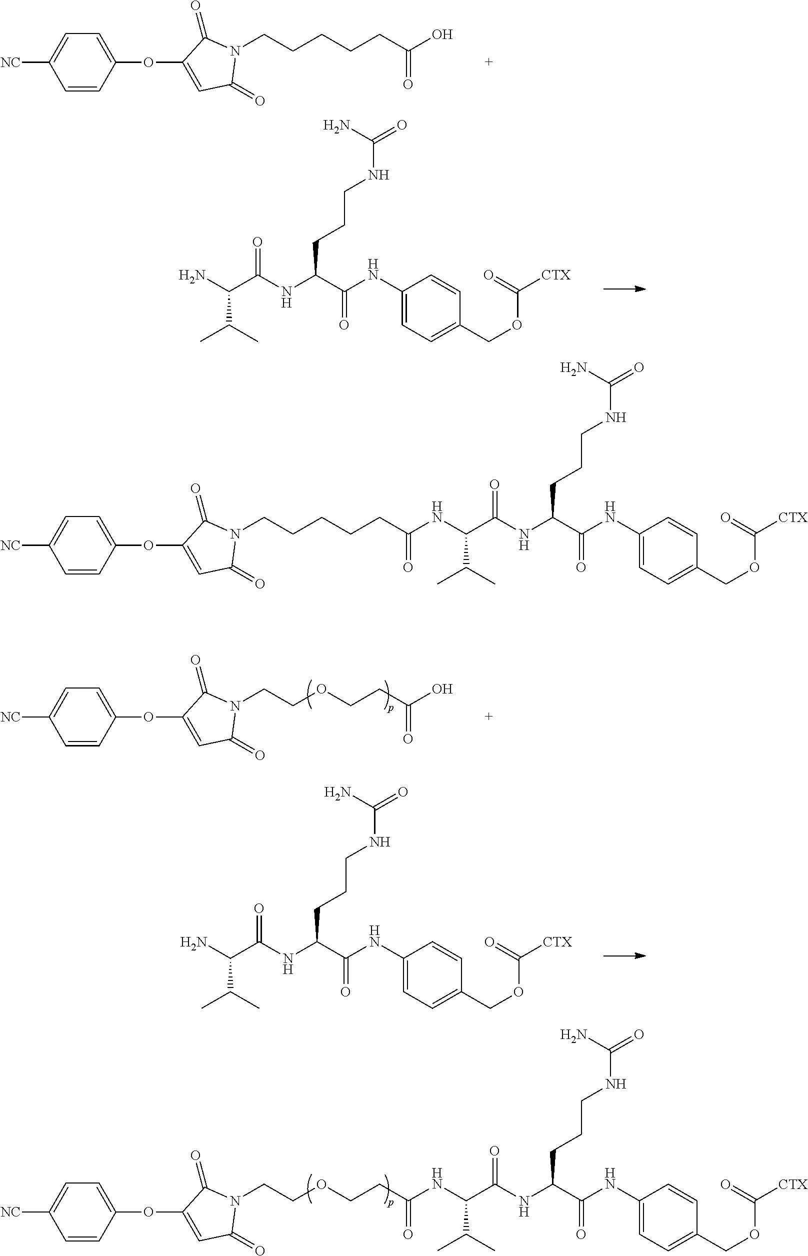

The following schemes illustrate an additional embodiment of linker-cytotoxin conjugates as disclosed herein, which may be synthesized by the methods disclosed herein:

Illustrative General Synthetic Schemes for Linker-Cytotoxin Conjugates a Disclosed Herein e.g. Cleavable Linkers:

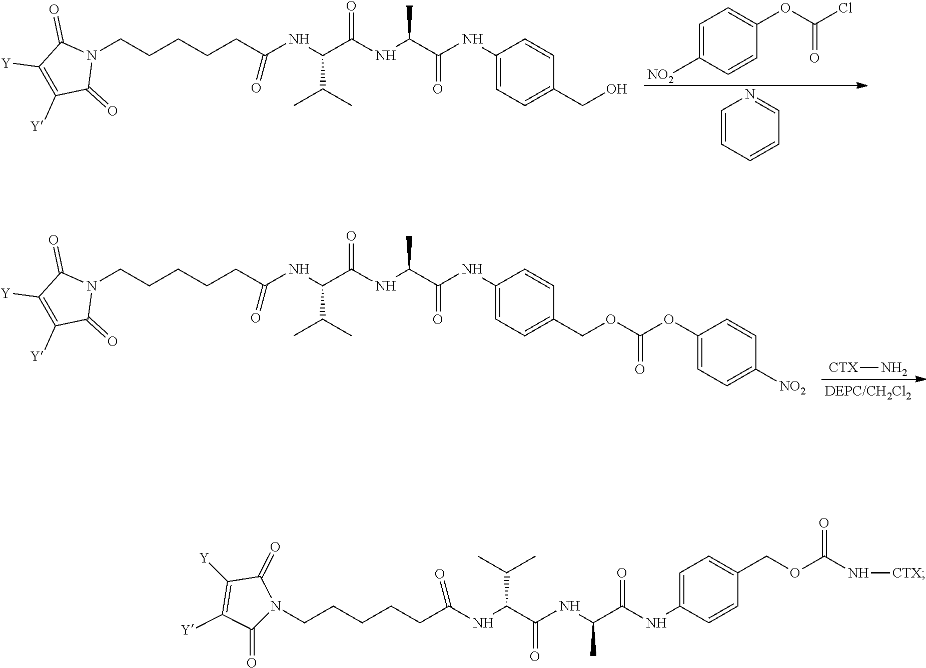

wherein Y and Y′ are as defined herein.

The above schemes are merely illustrative, and are not meant to be limiting. The linker-cytotoxin conjugates may be synthesized using any possible combination of linker and cytotoxin disclosed herein.

Exemplary linker-cytotoxin conjugates (stable or cleavable linkers), where CTX may be any cytotoxin disclosed herein, and which may be synthesized by methods disclosed herein, are provided below:

Examples of Linker-Cytotoxin Conjugates

| Stable | Cleavable |
|---|---|
| 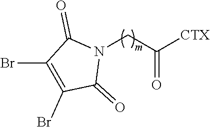 | 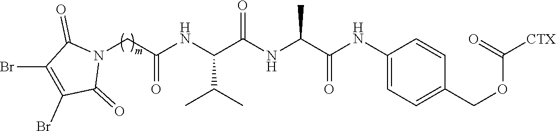 |
| 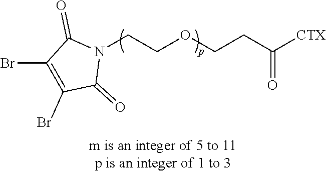 | 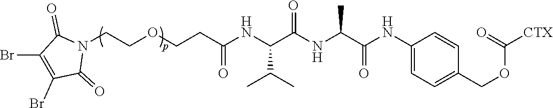 |
| | 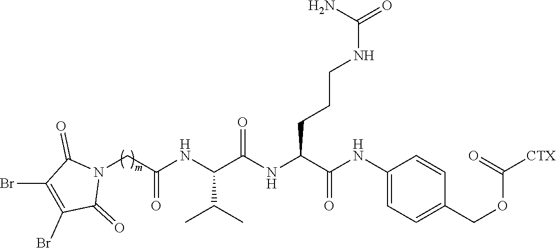 |
| | 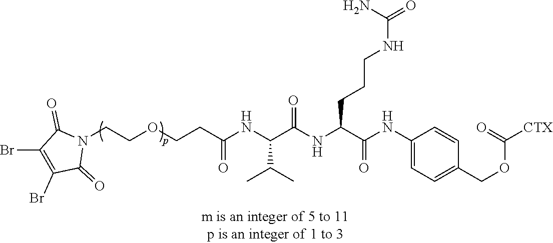 |

Additional Examples of Linker-Cytotoxin Conjugates

| Stable | Cleavable |
|---|---|
| 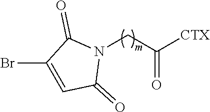 | 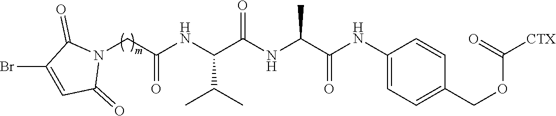 |
| 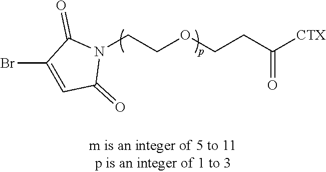 | 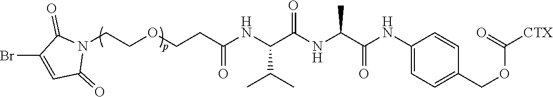 |
| | 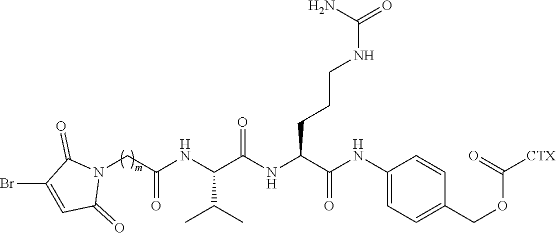 |
| | 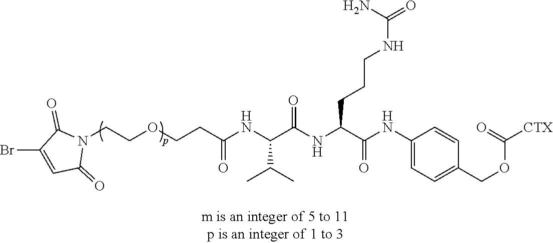 |

Additional Examples of Linker-Cytotoxin Conjugates

| Stable | Cleavable |
|---|---|
| 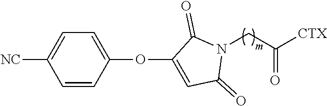 | 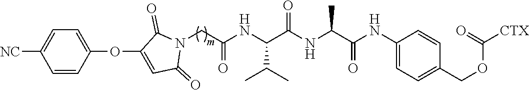 |
| 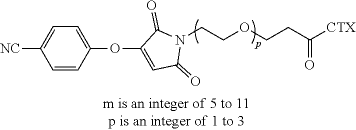 | 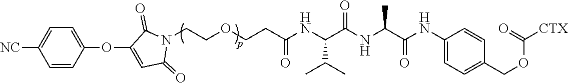 |
| | 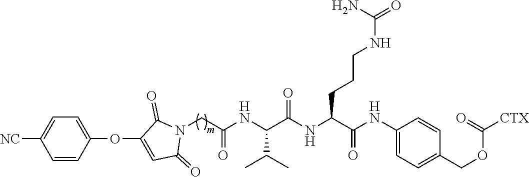 |
| | 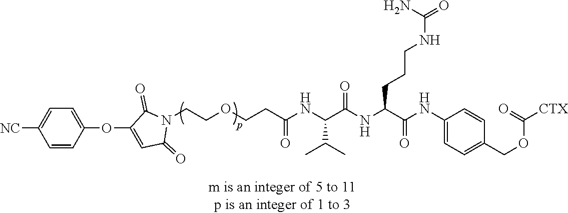 |

### Aspects of the Disclosure

### Antibody-Drug Conjugates (ADCs):

In one aspect, provided herein is antibody-drug conjugate of the following formula (I):

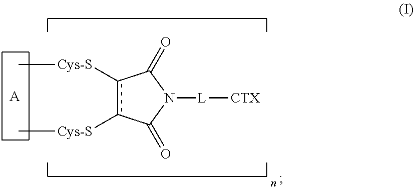

or pharmaceutically acceptable salt thereof,
wherein:
A is an antibody;
the two depicted cysteine residues are from an opened cysteine-cysteine disulfide bond in A;
L is a cleavable or a noncleavable linker;
CTX is a cytotoxin bonded to L by an amide bond, a carbamate bond, a disulfide bond, an ether bond, a thioether bond, or an ester bond;
the [symbol] bond represents a single or a double bond; and
n is an integer of 1 to 4.

In certain embodiments, provided herein is antibody-drug conjugate of the following formula (Ia):

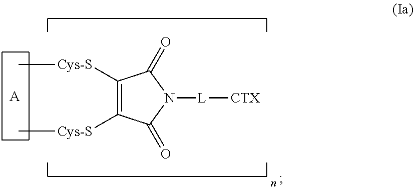

or pharmaceutically acceptable salt thereof,
wherein:
A is an antibody;
the two depicted cysteine residues are from an opened cysteine-cysteine disulfide bond in A;
L is a cleavable or a noncleavable linker;
CTX is a cytotoxin bonded to L by an amide bond, a carbamate bond, a disulfide bond, an ether bond, a thioether bond, or an ester bond; and
n is an integer of 1 to 4.

In certain embodiments, provided herein is antibody-drug conjugate of the following formula (Ib):

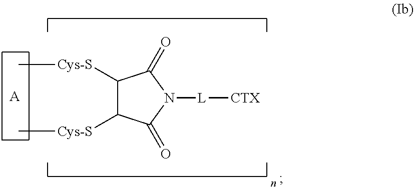

or pharmaceutically acceptable salt thereof,
wherein:
A is an antibody;
the two depicted cysteine residues are from an opened cysteine-cysteine disulfide bond in A;
L is a cleavable or a noncleavable linker;
CTX is a cytotoxin bonded to L by an amide bond, a carbamate bond, a disulfide bond, an ether bond, a thioether bond, or an ester bond; and
n is an integer of 1 to 4.

In certain embodiments of the antibody-drug conjugate of formula (I), (Ia) or (Ib), n is an integer of 2 (e.g., two heavy chain-light chain interchain disulfide bonds). In certain embodiments, n is an integer of 3 (e.g., two heavy chain-light chain interchain disulfide bonds and one hinge heavy chain-heavy chain interchain disulfide bond). In certain embodiments, n is an integer of 4 (e.g., two heavy chain-light chain interchain disulfide bonds and two hinge heavy chain-heavy chain interchain disulfide bonds).

In certain embodiments of the antibody-drug conjugate of formula (I), (Ia) or (Ib), L is a noncleavable linker.

In certain embodiments of the antibody-drug conjugate of formula (I), (Ia) or (Ib), L is:

—(CH₂)ₘC(O)—,

—(CH₂CH₂O)ₚ(CH₂CH₂)C(O)—, or

—(CH₂CH₂)(OCH₂CH₂)ₚC(O)—;

wherein m is an integer of 5 to 11, and p is an integer of 1 to 3.

In certain embodiments of the antibody-drug conjugate of formula (I), (Ia) or (Ib), L is a cleavable linker.

In certain embodiments of the antibody-drug conjugate of formula (I), (Ia) or (Ib), L is:

—(CH₂)ₘC(O)-Val-Ala-PAB-C(O)—,

—(CH₂)ₘC(O)-Val-Cit-PAB-C(O)—,

—(CH₂CH₂O)ₚ(CH₂CH₂)C(O)-Val-Ala-PAB-C(O)—,

—(CH₂CH₂O)ₚ(CH₂CH₂)C(O)-Val-Cit-PAB-C(O)—,

—(CH₂CH₂)(OCH₂CH₂)ₚC(O)-Val-Ala-PAB-C(O)—, or

—(CH₂CH₂)(OCH₂CH₂)ₚC(O)-Val-Cit-PAB-C(O)—;

wherein m is an integer of 5 to 11, and p is an integer of 1 to 3; and

wherein PAB has the following structure:

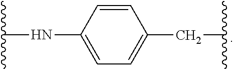 [SMILES: *Cc1ccc(N*)cc1]

In certain embodiments of the antibody-drug conjugate of formula (I), (Ia) or (Ib), A is an antibody that is specific to a cancer antigen. In certain embodiments, A is selected from the group consisting of alemtuzumab, anitumumab, bevacizumab, brentuximab, cetuximab, gemtuzumab, glembatumumab, inotuzumab, ipilimumab, lovortumumab, milatuzumab, ofatumumab, rituximab, tositumomab, and trastuzumab.

In certain embodiments of the antibody-drug conjugate of formula (I), (Ia) or (Ib), A comprises: a VH sequence that comprises SEQ ID NO: 1 and a VL sequence that comprises SEQ ID NO: 2; a VH sequence that comprises SEQ ID NO: 3 and a VL sequence that comprises SEQ ID NO: 4; or a VH sequence that comprises SEQ ID NO: 5 and a VL sequence that comprises SEQ ID NO: 6.

In certain embodiments of the antibody-drug conjugate of formula (I), (Ia) or (Ib), A comprises: a heavy chain sequence that comprises SEQ ID NO: 7 and a light chain sequence that comprises SEQ ID NO: 11; a heavy chain sequence that comprises SEQ ID NO: 8 and a light chain sequence that comprises SEQ ID NO: 11; a heavy chain sequence that comprises SEQ ID NO: 9 and a light chain sequence that comprises SEQ ID NO: 11; or a heavy chain sequence that comprises SEQ ID NO: 10 and a light chain sequence that comprises SEQ ID NO: 11.

In certain embodiments of the antibody-drug conjugate of formula (I), (Ia) or (Ib), A comprises: a heavy chain sequence that comprises SEQ ID NO: 12 and a light chain sequence that comprises SEQ ID NO: 16; a heavy chain sequence that comprises SEQ ID NO: 13 and a light chain sequence that comprises SEQ ID NO: 16; a heavy chain sequence that comprises SEQ ID NO: 14 and a light chain sequence that comprises SEQ ID NO: 16; a heavy chain sequence that comprises SEQ ID NO: 15 and a light chain sequence that comprises SEQ ID NO: 16.

In certain embodiments of the antibody-drug conjugate of formula (I), (Ia) or (Ib), A comprises: a heavy chain sequence that comprises SEQ ID NO: 17 and a light chain sequence that comprises SEQ ID NO: 21; a heavy chain sequence that comprises SEQ ID NO: 18 and a light chain sequence that comprises SEQ ID NO: 21; a heavy chain sequence that comprises SEQ ID NO: 19 and a light chain sequence that comprises SEQ ID NO: 21; or a heavy chain sequence that comprises SEQ ID NO: 20 and a light chain sequence that comprises SEQ ID NO: 21.

In certain embodiments of the antibody-drug conjugate of formula (I), (Ia) or (Ib), A comprises: a heavy chain sequence that comprises SEQ ID NO: 22 and a light chain sequence that comprises SEQ ID NO: 26; a heavy chain sequence that comprises SEQ ID NO: 23 and a light chain sequence that comprises SEQ ID NO: 26; a heavy chain sequence that comprises SEQ ID NO: 24 and a light chain sequence that comprises SEQ ID NO: 26; or a heavy chain sequence that comprises SEQ ID NO: 25 and a light chain sequence that comprises SEQ ID NO: 26.

In certain embodiments of the antibody-drug conjugate of formula (I), (Ia) or (Ib), CTX is an auristatin. In certain embodiments the CTX is monomethylauristatin F (MMAF). In certain embodiments the CTX is monomethylauristatin E (MMAE). In certain embodiments the CTX is a pyrrolobenzodiazepine (PBD). In certain embodiments the CTX is a calicheamicin, doxorubicin, camptothecin, duocarmycin, DM1, DM4, a maytansinoid, or a tubulysin.

In another aspect, provided herein is an antibody-drug conjugate of the following formula (III):

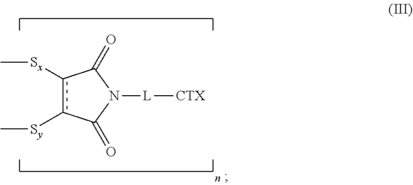

wherein:
L is a cleavable or a noncleavable linker;
CTX is an auristatin bonded to L by an amide bond or a carbamate bond; wherein the auristatin is MMAF or MMAE;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody;
the [symbol] bond represents a single or a double bond; and
n is an integer of 1 to 4.

In certain embodiments, provided herein is an antibody-drug conjugate of the following formula (IIIa):

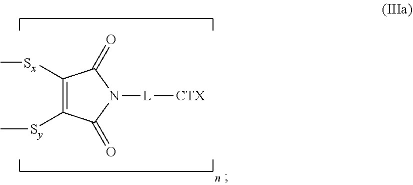

wherein:
L is a cleavable or a noncleavable linker;
CTX is an auristatin bonded to L by an amide bond or a carbamate bond; wherein the auristatin is MMAF or MMAE;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody; and
n is an integer of 1 to 4.

In certain embodiments, provided herein is an antibody-drug conjugate of the following formula (IIIb):

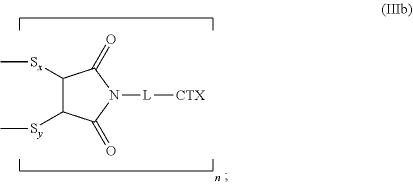

wherein:
L is a cleavable or a noncleavable linker;
CTX is an auristatin bonded to L by an amide bond or a carbamate bond; wherein the auristatin is MMAF or MMAE;
Sₓ is a sulfur atom from a first cysteine residue, and S_{y} is a sulfur atom from a second cysteine residue, wherein the first cysteine residue and the second cysteine residue are from different chains and/or from the same chain of a multi-chain antibody; and
n is an integer of 1 to 4.

In certain embodiments of the antibody-drug conjugate of formula (III), (IIIa) or (IIIb), n is an integer of 1. In certain embodiments, n is an integer of 2. In certain embodiments, n is an integer of 3. In certain embodiments, n is an integer of 4.

In certain embodiments of the antibody-drug conjugate of formula (III), (IIIa) or (IIIb), CTX is an auristatin bonded to L by an amide bond or a carbamate bond; wherein the auristatin is MMAF or MMAE.

In certain embodiments of the antibody-drug conjugate of formula (III), (IIIa) or (IIIb), CTX is a PBD bonded to L by an amide bond or a carbamate bond.

In certain embodiments of the antibody-drug conjugate of formula (III), (IIIa) or (IIIb), the multi-chain antibody comprises two heavy chains and two light chains.

In certain embodiments of the antibody-drug conjugate of formula (III), (IIIa) or (IIIb), the first cysteine residue is from a first heavy chain and the second cysteine residue is from a second heavy chain of the multi-chain antibody.

In certain embodiments of the antibody-drug conjugate of formula (III), (IIIa) or (IIIb), the first cysteine residue is from a heavy chain and the second cysteine residue is from a light chain of the multi-chain antibody.

In certain embodiments of the antibody-drug conjugate of formula (III), (IIIa) or (IIIb), the first and second cysteine residues are from the same heavy chain of the multi-chain antibody.

In certain embodiments of the antibody-drug conjugate of formula (III), the antibody-drug conjugate is of the following formula:

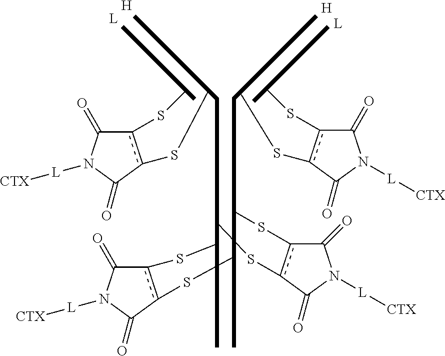

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (III), the antibody-drug conjugate is of the following formula:

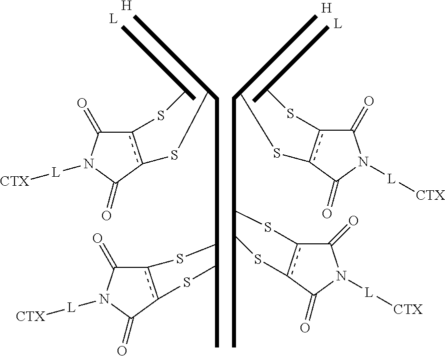

where each heavy chain of the multi-chain antibody is denoted by the letter H, and each light chain of the multi-chain antibody is denoted by the letter L; and the [symbol] bond represents a single or a double bond.

In certain embodiments of the antibody-drug conjugate of formula (IIIa), the antibody-drug conjugate is of the following formula:

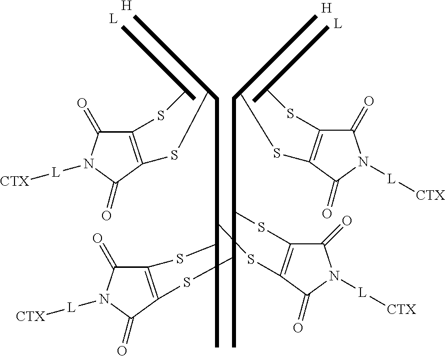

where each heavy chain of the multi-chain antibody is denoted by the letter H, and each light chain of the multi-chain antibody is denoted by the letter L.

In certain embodiments of the antibody-drug conjugate of formula (IIIa), the antibody-drug conjugate is of the following formula:

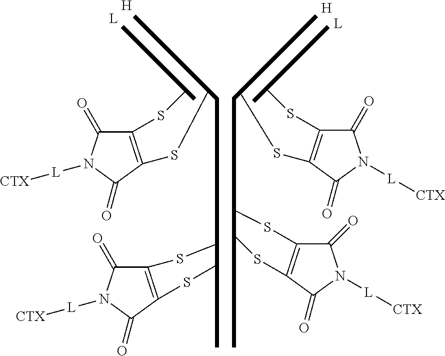

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

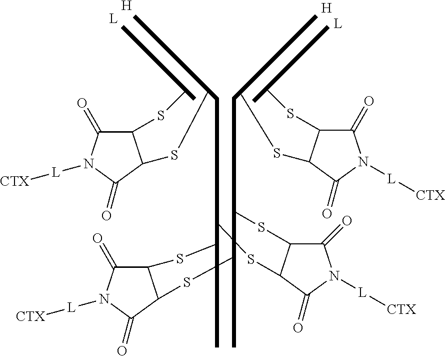

where each heavy chain of the multi-chain antibody is denoted by the letter H, and each light chain of the multi-chain antibody is denoted by the letter L.

In certain embodiments of the antibody-drug conjugate of formula (IIIb), the antibody-drug conjugate is of the following formula:

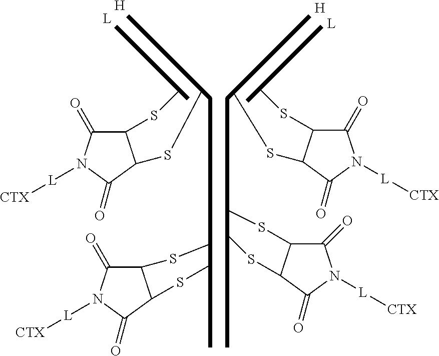

where each heavy chain of the multi-chain antibody is denoted by the letter H, and each light chain of the multi-chain antibody is denoted by the letter L.

In certain embodiments of the antibody-drug conjugate of formula (III), (IIIa) or (IIIb), the multi-chain antibody comprises mutations in one or more cysteines in the hinge regions of two heavy chains. In certain embodiments, the one or more cysteine residues are mutated to structurally related amino acids. In certain embodiments, the one or more cysteine residues are mutated to alanines.

In certain embodiments of the antibody-drug conjugate of formula (III), wherein the multi-chain antibody comprises mutations in one or more cysteines in the hinge regions of two heavy chains, the antibody-drug conjugate is of the following formula:

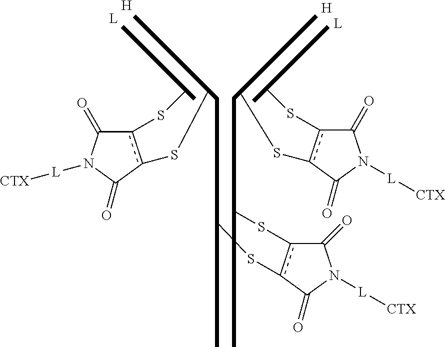

where each heavy chain of the multi-chain antibody is denoted by the letter H, and each light chain of the multi-chain antibody is denoted by the letter L; and the [symbol] bond represents a single or a double bond. For the embodiments of the antibody-drug conjugate of formula (III) depicted above, the ADC has a DAR=3 (three drugs per antibody). As described herein, such ADCs may be prepared (e.g., a described in Example 13) by mutating one or more of the hinge cysteine residues of a human IgG1 (e.g., 1 hinge cysteine), IgG2 (e.g., 3 hinge cysteines), IgG3 (e.g., 10 hinge cysteines), or IgG4 (e.g., 1 hinge cysteine).

In certain embodiments of the antibody-drug conjugate of formula (III), wherein the multi-chain antibody comprises mutations in one or more cysteines in the hinge regions of two heavy chains, the antibody-drug conjugate is of the following formula:

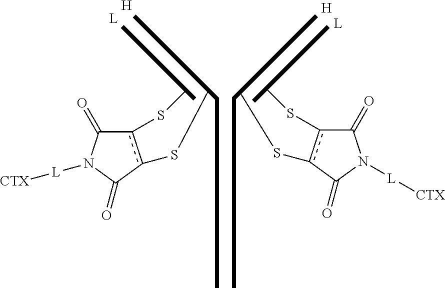

where each heavy chain of the multi-chain antibody is denoted by the letter H, and each light chain of the multi-chain antibody is denoted by the letter L; and the [symbol] bond represents a single or a double bond. For the embodiments of the antibody-drug conjugate of formula (III) depicted above, the ADC has a DAR=2 (two drugs per antibody). As described herein, such ADCs may be prepared (e.g., a described in Example 13) by mutating one or more of the hinge cysteine residues of a human IgG1 (e.g., 2 hinge cysteines), IgG2 (e.g., 4 hinge cysteines), IgG3 (e.g., 11 hinge cysteines), or IgG4 (e.g., 2 hinge cysteines). In certain embodiments of the antibody-drug conjugate of formula (III), L is a noncleavable linker.

In certain embodiments of the antibody-drug conjugate of formula (IIIa), wherein the multi-chain antibody comprises mutations in one or more cysteines in the hinge regions of two heavy chains, the antibody-drug conjugate is of the following formula:

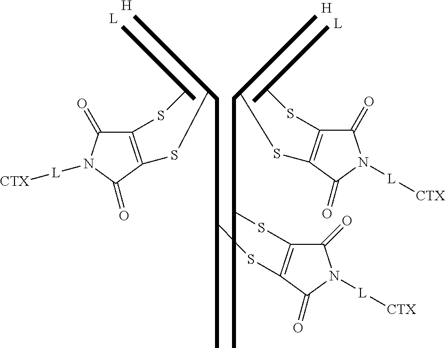

where each heavy chain of the multi-chain antibody is denoted by the letter H, and each light chain of the multi-chain antibody is denoted by the letter L. For the embodiments of the antibody-drug conjugate of formula (IIIa) depicted above, the ADC has a DAR=3 (three drugs per antibody). As described herein, such ADCs may be prepared (e.g., a described in Example 13) by mutating one or more of the hinge cysteine residues of a human IgG1 (e.g., 1 hinge cysteine), IgG2 (e.g., 3 hinge cysteines), IgG3 (e.g., 10 hinge cysteines), or IgG4 (e.g., 1 hinge cysteine).

In certain embodiments of the antibody-drug conjugate of formula (IIIa), wherein the multi-chain antibody comprises mutations in one or more cysteines in the hinge regions of two heavy chains, the antibody-drug conjugate is of the following formula:

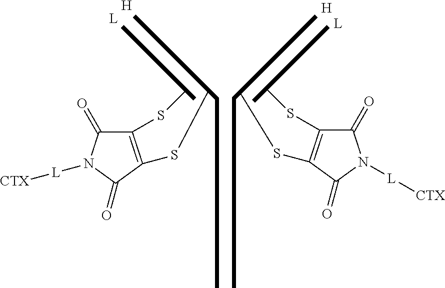

where each heavy chain of the multi-chain antibody is denoted by the letter H, and each light chain of the multi-chain antibody is denoted by the letter L. For the embodiments of the antibody-drug conjugate of formula (IIIa) depicted above, the ADC has a DAR=2 (two drugs per antibody). As described herein, such ADCs may be prepared (e.g., a described in Example 13) by mutating one or more of the hinge cysteine residues of a human IgG1 (e.g., 2 hinge cysteines), IgG2 (e.g., 4 hinge cysteines), IgG3 (e.g., 11 hinge cysteines), or IgG4 (e.g., 2 hinge cysteines). In certain embodiments of the antibody-drug conjugate of formula (IIIa), L is a noncleavable linker.

In certain embodiments of the antibody-drug conjugate of formula (IIIb), wherein the multi-chain antibody comprises mutations in one or more cysteines in the hinge regions of two heavy chains, the antibody-drug conjugate is of the following formula:

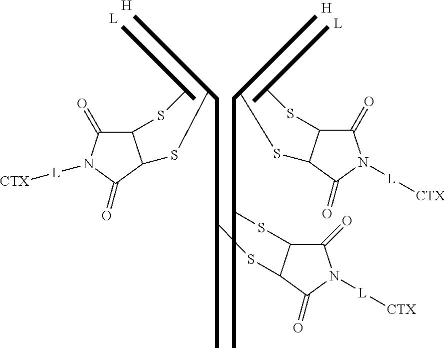

where each heavy chain of the multi-chain antibody is denoted by the letter H, and each light chain of the multi-chain antibody is denoted by the letter L. For the embodiments of the antibody-drug conjugate of formula (IIIb) depicted above, the ADC has a DAR=3 (three drugs per antibody). As described herein, such ADCs may be prepared (e.g., a described in Example 13) by mutating one or more of the hinge cysteine residues of a human IgG1 (e.g., 1 hinge cysteine), IgG2 (e.g., 3 hinge cysteines), IgG3 (e.g., 10 hinge cysteines), or IgG4 (e.g., 1 hinge cysteine).

In certain embodiments of the antibody-drug conjugate of formula (IIIb), wherein the multi-chain antibody comprises mutations in one or more cysteines in the hinge regions of two heavy chains, the antibody-drug conjugate is of the following formula:

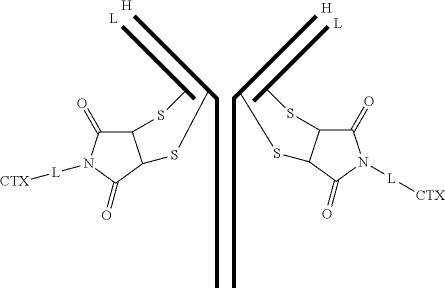

where each heavy chain of the multi-chain antibody is denoted by the letter H, and each light chain of the multi-chain antibody is denoted by the letter L. For the embodiments of the antibody-drug conjugate of formula (IIIb) depicted above, the ADC has a DAR=2 (two drugs per antibody). As described herein, such ADCs may be prepared (e.g., a described in Example 13) by mutating one or more of the hinge cysteine residues of a human IgG1 (e.g., 2 hinge cysteines), IgG2 (e.g., 4 hinge cysteines), IgG3 (e.g., 11 hinge cysteines), or IgG4 (e.g., 2 hinge cysteines). In certain embodiments of the antibody-drug conjugate of formula (IIIb), L is a noncleavable linker.

In certain embodiments of the antibody-drug conjugate of formula (III), (IIIa) or (IIIb), L is:

—(CH₂)ₘC(O)—,

—(CH₂CH₂O)ₚ(CH₂CH₂)C(O)—, or

—(CH₂CH₂)(OCH₂CH₂)ₚC(O)—;

wherein m is an integer of 5 to 11, and p is an integer of 1 to 3.

In certain embodiments of the antibody-drug conjugate of formula (III), (IIIa) or (IIIb), L is a cleavable linker.

In certain embodiments of the antibody-drug conjugate of formula (III), (IIIa) or (IIIb), L is:

—(CH₂)ₘC(O)-Val-Ala-PAB-C(O)—,

—(CH₂)ₘC(O)-Val-Cit-PAB-C(O)—,

—(CH₂CH₂O)ₚ(CH₂CH₂)C(O)-Val-Ala-PAB-C(O)—,

—(CH₂CH₂O)ₚ(CH₂CH₂)C(O)-Val-Cit-PAB-C(O)—,

—(CH₂CH₂)(OCH₂CH₂)ₚC(O)-Val-Ala-PAB-C(O)—, or

—(CH₂CH₂)(OCH₂CH₂)ₚC(O)-Val-Cit-PAB-C(O)—;

wherein m is an integer of 5 to 11, and p is an integer of 1 to 3; and

wherein PAB has the following structure:

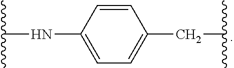 [SMILES: *Cc1ccc(N*)cc1]

In certain embodiments of the antibody-drug conjugate of formula (III), (IIIa) or (IIIb), the multi-chain antibody is an antibody that is specific to a cancer antigen. In certain embodiments, the multi-chain antibody is selected from the group consisting of alemtuzumab, anitumumab, bevacizumab, brentuximab, cetuximab, gemtuzumab, glembatumumab, inotuzumab, ipilimumab, lovortumumab, milatuzumab, ofatumumab, rituximab, tositumomab, and trastuzumab.

In certain embodiments of the antibody-drug conjugate of formula (III), (IIIa) or (IIIb), CTX is monomethylauristatin F (MMAF). In certain embodiments the CTX is monomethylauristatin E (MMAE). In certain embodiments the CTX is a pyrrolobenzodiazepine (PBD). In certain embodiments the CTX is a pyrrolobenzodiazepine (PBD). In certain embodiments the CTX is a calicheamicin, doxorubicin, camptothecin, duocarmycin, DM1, DM4, a maytansinoid, or a tubulysin.

In certain embodiments of the antibody-drug conjugate of formula (III), (IIIa) or (IIIb), n is 4. In certain embodiments, CTX is MMAF, L is —(CH₂)₅C(O)— and n is 4. In certain embodiments, CTX is MMAE, L is —(CH₂)₅C(O)-Val-Ala-PAB-C(O)— and n is 4.

In certain embodiments of the antibody-drug conjugate of formula (III), (IIIa) or (IIIb), the multi-chain antibody comprises: a VH sequence that comprises SEQ ID NO: 1 and a VL sequence that comprises SEQ ID NO: 2; a VH sequence that comprises SEQ ID NO: 3 and a VL sequence that comprises SEQ ID NO: 4; or a VH sequence that comprises SEQ ID NO: 5 and a VL sequence that comprises SEQ ID NO: 6.

In certain embodiments of the antibody-drug conjugate of formula (III), (IIIa) or (IIIb), the multi-chain antibody comprises: a heavy chain sequence that comprises SEQ ID NO: 7 and a light chain sequence that comprises SEQ ID NO: 11; a heavy chain sequence that comprises SEQ ID NO: 8 and a light chain sequence that comprises SEQ ID NO: 11; a heavy chain sequence that comprises SEQ ID NO: 9 and a light chain sequence that comprises SEQ ID NO: 11; or a heavy chain sequence that comprises SEQ ID NO: 10 and a light chain sequence that comprises SEQ ID NO: 11.

In certain embodiments of the antibody-drug conjugate of formula (III), (IIIa) or (IIIb), the multi-chain antibody comprises: a heavy chain sequence that comprises SEQ ID NO: 12 and a light chain sequence that comprises SEQ ID NO: 16; a heavy chain sequence that comprises SEQ ID NO: 13 and a light chain sequence that comprises SEQ ID NO: 16; a heavy chain sequence that comprises SEQ ID NO: 14 and a light chain sequence that comprises SEQ ID NO: 16; a heavy chain sequence that comprises SEQ ID NO: 15 and a light chain sequence that comprises SEQ ID NO: 16.

In certain embodiments of the antibody-drug conjugate of formula (III), (IIIa) or (IIIb), the multi-chain antibody comprises: a heavy chain sequence that comprises SEQ ID NO: 17 and a light chain sequence that comprises SEQ ID NO: 21; a heavy chain sequence that comprises SEQ ID NO: 18 and a light chain sequence that comprises SEQ ID NO: 21; a heavy chain sequence that comprises SEQ ID NO: 19 and a light chain sequence that comprises SEQ ID NO: 21; or a heavy chain sequence that comprises SEQ ID NO: 20 and a light chain sequence that comprises SEQ ID NO: 21.

In certain embodiments of the antibody-drug conjugate of formula (III), (IIIa) or (IIIb), the multi-chain antibody comprises: a heavy chain sequence that comprises SEQ ID NO: 22 and a light chain sequence that comprises SEQ ID NO: 26; a heavy chain sequence that comprises SEQ ID NO: 23 and a light chain sequence that comprises SEQ ID NO: 26; a heavy chain sequence that comprises SEQ ID NO: 24 and a light chain sequence that comprises SEQ ID NO: 26; or a heavy chain sequence that comprises SEQ ID NO: 25 and a light chain sequence that comprises SEQ ID NO: 26.

In another aspect, provided herein is a composition comprising an antibody-drug conjugate of the following formula:

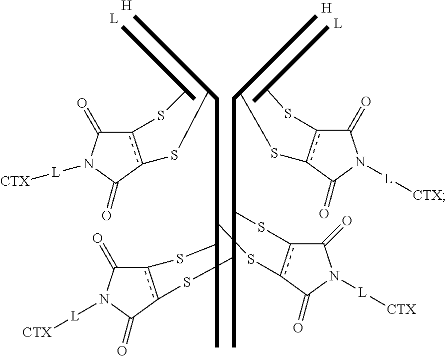

and/or
an antibody-drug conjugate of the following formula:

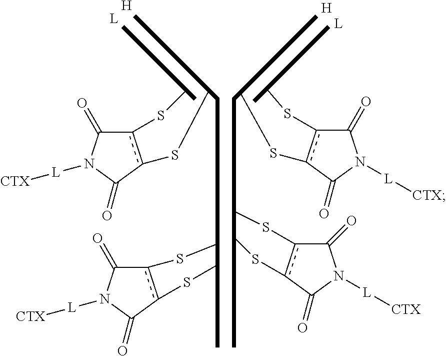

where each heavy chain of the multi-chain antibody is denoted by the letter H, and each light chain of the multi-chain antibody is denoted by the letter L; and the [symbol] bond represents a single or a double bond.

In certain embodiments, provided herein is a composition comprising an antibody-drug conjugate of the following formula:

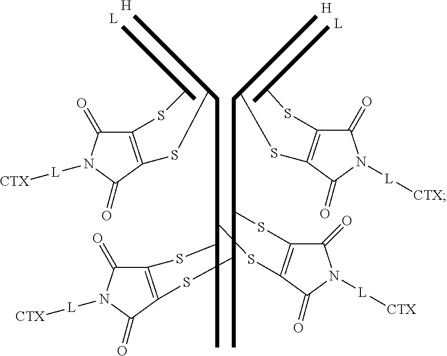

and/or
an antibody-drug conjugate of the following formula:

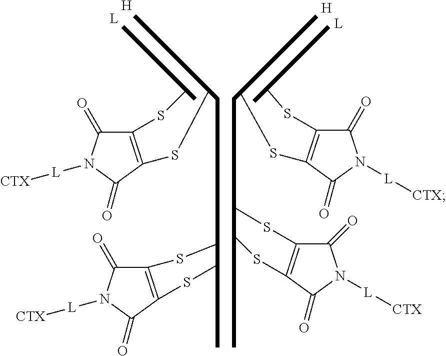

where each heavy chain of the multi-chain antibody is denoted by the letter H, and each light chain of the multi-chain antibody is denoted by the letter L.

In certain embodiments, provided herein is a composition comprising an antibody-drug conjugate of the following formula:

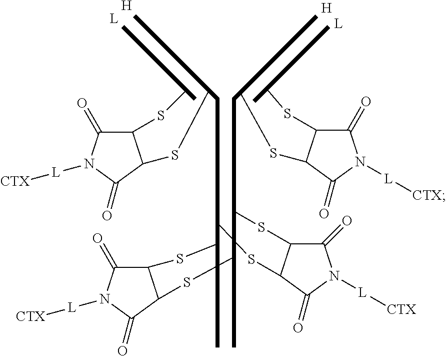

and/or
an antibody-drug conjugate of the following formula:

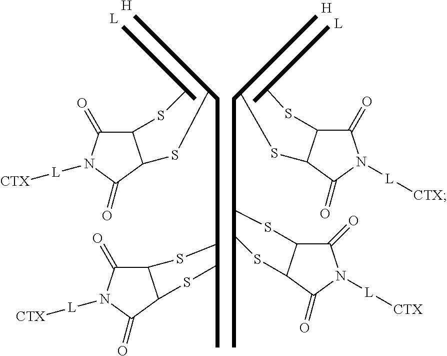

where each heavy chain of the multi-chain antibody is denoted by the letter H, and each light chain of the multi-chain antibody is denoted by the letter L.

### Linker-Cytotoxin Conjugates:

In another aspect, provided herein is a linker-cytotoxin conjugate of the following formula (II):

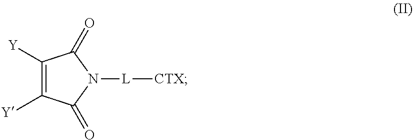

or an enantiomer, diasteriomer, or mixtures thereof;
wherein:
each Y and Y′ is independently hydrogen or an electrophilic leaving group that reacts selectively with thiols, provided if one of Y and Y′ is hydrogen, the other is the electrophilic leaving group;
CTX is a cytotoxin bonded to L by an amide bond or a carbamate bond; and
L is a cleavable or a noncleavable linker.

In certain embodiments of the linker-cytotoxin conjugate of formula (II), each Y and Y′ is an electrophilic leaving group that reacts selectively with thiol.

In certain embodiments of the linker-cytotoxin conjugate of formula (II), one of Y and Y′ is an electrophilic leaving group that reacts selectively with thiol, and the other of Y and Y′ is hydrogen.

In certain embodiments of the linker-cytotoxin conjugate of formula (II), each Y and Y′ is independently selected from the group consisting of a halo, a substituted thiol, and a substituted sulfonate. In certain embodiments, each Y and Y′ is independently selected from the group consisting of a halo, a substituted thiol, a substituted sulfonate, and a substituted phenol. In certain embodiments, each Y and Y′ is independently selected from the group consisting of chloro, bromo, fluoro, and iodo. In certain embodiments, each Y and Y′ is bromo.

In certain embodiments of the linker-cytotoxin conjugate of formula (II), one of Y and Y′ is selected from the group consisting of a halo, a substituted thiol, a substituted sulfonate, and a substituted phenol, and the other of Y and Y′ is hydrogen. In certain embodiments, one of Y and Y′ is selected from the group consisting of chloro, bromo, fluoro, and iodo, and the other of Y and Y′ is hydrogen. In certain embodiments, one of Y and Y′ is bromo, and the other of Y and Y′ is hydrogen. In certain embodiments, one of Y and Y′ is a substituted phenol, and the other of Y and Y′ is hydrogen. In certain embodiments, one of Y and Y′ is cyanophenol, and the other of Y and Y′ is hydrogen. In certain embodiments, one of Y and Y′ is p-cyanophenol, and the other of Y and Y′ is hydrogen.

In certain embodiments of the linker-cytotoxin conjugate of formula (II), the linker-cytotoxin conjugate has one of the following formulas (IIa), (IIb), and (IIc):

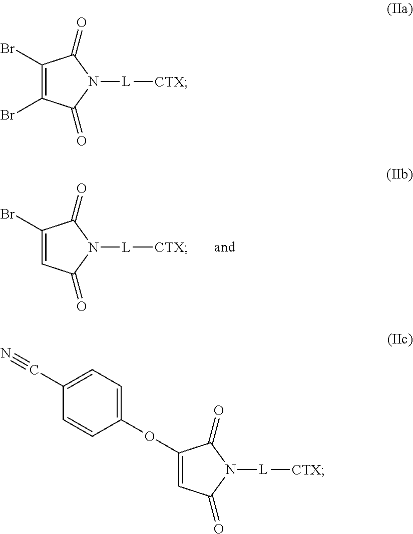

or an enantiomer, diasteriomer, or mixtures thereof;
wherein:
L is a cleavable or noncleavable linker; and
CTX is cytotoxin bonded to L by an amide bond or a carbamate bond.

In certain embodiments of the linker-cytotoxin conjugate of formula (II), L is a noncleavable linker.

In certain embodiments of the linker-cytotoxin conjugate of formula (II), L is:

—(CH₂)ₘC(O)—,

—(CH₂CH₂O)ₚ(CH₂CH₂)C(O)—, or

—(CH₂CH₂)(OCH₂CH₂)ₚC(O)—;

wherein m is an integer of 5 to 11, and p is an integer of 1 to 3.

In certain embodiments of the linker-cytotoxin conjugate of formula (II), L is a cleavable linker.

In certain embodiments of the linker-cytotoxin conjugate of formula (II), L is:

—(CH₂)ₘC(O)-Val-Ala-PAB-C(O)—,

—(CH₂)ₘC(O)-Val-Cit-PAB-C(O)—,

—(CH₂CH₂O)ₚ(CH₂CH₂)C(O)-Val-Ala-PAB-C(O)—,

—(CH₂CH₂O)ₚ(CH₂CH₂)C(O)-Val-Cit-PAB-C(O)—,

—(CH₂CH₂)(OCH₂CH₂)ₚC(O)-Val-Ala-PAB-C(O)—, or

—(CH₂CH₂)(OCH₂CH₂)ₚC(O)-Val-Cit-PAB-C(O)—;

wherein m is an integer of 5 to 11, and p is an integer of 1 to 3; and

wherein PAB has the following structure:

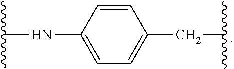 [SMILES: *Cc1ccc(N*)cc1]

In certain embodiments of the linker-cytotoxin conjugate of formula (II), the CTX is an auristatin. In certain embodiments the CTX is MMAF. In certain embodiments the CTX is MMAE. In certain embodiments the CTX is a PBD. In certain embodiments the CTX is a calicheamicin, doxorubicin, camptothecin, duocarmycin, DM1, DM4, a maytansinoid, or a tubulysin.

### Linkers:

In another aspect, provided herein is a linker of the following formula:

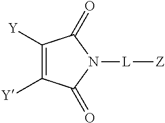

or an enantiomer, diasteriomer, or mixtures thereof;
wherein:
each Y and Y′ is independently hydrogen or an electrophilic leaving group that reacts selectively with thiols, provided if one of Y and Y′ is hydrogen, the other is the electrophilic leaving group;
Z is —CO₂H, —NH₂, —OH, —NH—R^{3a}, or —CO₂R^{3b}; and
L is a cleavable or a noncleavable linker.

In certain embodiments of the linker, each Y and Y′ is an electrophilic leaving group that reacts selectively with thiol.

In certain embodiments of the linker, one of Y and Y′ is an electrophilic leaving group that reacts selectively with thiol, and the other of Y and Y′ is hydrogen.

In certain embodiments of the linker, each Y and Y′ is independently selected from the group consisting of a halo, a substituted thiol, and a substituted sulfonate. In certain embodiments, each Y and Y′ is independently selected from the group consisting of a halo, a substituted thiol, a substituted sulfonate, and a substituted phenol. In certain embodiments, each Y and Y is independently selected from the group consisting of chloro, bromo, fluoro, and iodo. In certain embodiments, each Y and Y′ is bromo.

In certain embodiments of the linker, one of Y and Y′ is selected from the group consisting of a halo, a substituted thiol, a substituted sulfonate, and a substituted phenol, and the other of Y and Y′ is hydrogen. In certain embodiments, one of Y and Y′ is selected from the group consisting of chloro, bromo, fluoro, and iodo, and the other of Y and Y′ is hydrogen. In certain embodiments, one of Y and Y′ is bromo, and the other of Y and Y′ is hydrogen. In certain embodiments, one of Y and Y′ is a substituted phenol, and the other of Y and Y′ is hydrogen. In certain embodiments, one of Y and Y′ is cyanophenol, and the other of Y and Y′ is hydrogen. In certain embodiments, one of Y and Y′ is p-cyanophenol, and the other of Y and Y′ is hydrogen.

In certain embodiments of the linker, Z is —CO₂H, —NH₂, —OH, —NH—R^{3a}, or —CO₂R^{3b}; wherein R^{3a} is an amino protecting group, and R^{3b} is a carboxyl protecting group, as disclosed, for example, in Greene, T. W.; Wuts, P. G. M., 1991, Protective Groups In Organic Synthesis, 3rd ed.; John Wiley & Sons: New York, and similar documents. Those of ordinary skill in the art will be able to select appropriate amino or carboxyl protecting groups.

In certain embodiments of the linker, Z is —CO₂H or —CO₂R^{3b}, and R^{3b} is a carboxyl protecting group.

In certain embodiments of the linker, R^{3a} is selected from the group consisting of 9-fluorenylmethyloxycarbamate (FMOC), tert-butyloxycarbonyl (BOC), benzyl carbamate (Cbz), acetamide, trifluroacetamide, phthalimide, benzylamine, nitrobenzene, triphenylmethylamine, benzylideneamine, and p-toluenesulfonamide (p-TOS).

In certain embodiments of the linker, R^{3b} is selected from the group consisting of a methyl ester, a tert-butyl ester, a benzyl ester, an S-tert-butyl ester, and 2-alkyl-1,3-oxazoline.

In certain embodiments of the linker, L is a noncleavable linker.

In certain embodiments of the linker, L is:

—(CH₂)ₘC(O)—,

—(CH₂CH₂O)ₚ(CH₂CH₂)C(O)—, or

—(CH₂CH₂)(OCH₂CH₂)ₚC(O)—;

wherein m is an integer of 5 to 11, and p is an integer of 1 to 3.

In certain embodiments of the linker, L is a cleavable linker.

In certain embodiments of the linker, L is:

—(CH₂)ₘC(O)-Val-Ala-PAB-C(O)—,

—(CH₂)ₘC(O)-Val-Cit-PAB-C(O)—,

—(CH₂CH₂O)ₚ(CH₂CH₂)C(O)-Val-Ala-PAB-C(O)—,

—(CH₂CH₂O)ₚ(CH₂CH₂)C(O)-Val-Cit-PAB-C(O)—,

—(CH₂CH₂)(OCH₂CH₂)ₚC(O)-Val-Ala-PAB-C(O)—, or

—(CH₂CH₂)(OCH₂CH₂)ₚC(O)-Val-Cit-PAB-C(O)—;

wherein m is an integer of 5 to 11, and p is an integer of 1 to 3; and

wherein PAB has the following structure:

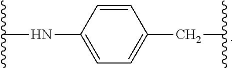 [SMILES: *Cc1ccc(N*)cc1]

### Antibodies:

In certain embodiments, disclosed herein are antibodies (e.g., a multi-chain antibodies) or antibody fragments (e.g., multi-chain antibody fragments) for use in the ADCs disclosed herein.

In certain embodiments, A is an antibody or an antibody fragment. In certain embodiments, A is a monoclonal antibody or monoclonal antibody fragment.

In certain embodiments, the antibody (e.g., multi-chain antibody) is a monoclonal antibody or a humanized antibody. In certain embodiments, the antibody is specific to a cancer antigen. In certain embodiments, the cancer antigen is the cancer antigen is CD33 (Siglec3), CD30 (TNFRSF8), HER2 (ERbB-2), CD22 (Siglec2), CD79b, CD22 (Siglec2), GPNMB, CD19 (B4), CD56 (NCAM), CD138 (SDC1), PSMA (FOLH1), CD74 (DHLAG), PSMA (FOLH1), CEACAM5 (CD66e), EGP1 (TROP2), FOLR1, CD37, Muc-16, Endothelial receptor (ETB), STEAP1, CD19, CD70 (TNFSF7), SLC44A4, Nectin-4, AGS-16, Guanylyl cyclase C, Muc-1, CD70 (TNFSF7), Her3 (ErbB-3), mesothelin, CD70 (TNFSF7), CA9 (MN), or CFC1B (Cripto). In certain embodiments, the cancer antigen is HER2, VEGF-A, EGFR, CD20, C10orf54, CD98, or C16orf54.

In another embodiment, the antibody employed in the ADCs of the present application is selected from the group consisting of alemtuzumab, bevacizumab, cetuximab, ipilimumab, ofatumumab, anitumumab, rituximab, tositumomab, inotuzumab, glembatumumab, lovortuzumab, milatuzumab and trastuzumab. In another embodiment, the antibody employed in the ADCs of the present application is selected from the group consisting of adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab, votumumab, and zalutumumab.

In certain embodiments, the antibody comprises a VH sequence that comprises SEQ ID NO: 1 and a VL sequence that comprises SEQ ID NO: 2. In certain embodiments, the antibody comprises a VH sequence that comprises SEQ ID NO: 3 and a VL sequence that comprises SEQ ID NO: 4. In certain embodiments, the antibody comprises a VH sequence that comprises SEQ ID NO: 5 and a VL sequence that comprises SEQ ID NO: 6.

In certain embodiments, the antibody comprises a heavy chain sequence which comprises SEQ ID NO: 7 and a light chain sequence which comprises SEQ ID NO: 11. In certain embodiments, the antibody comprises a heavy chain sequence which comprises SEQ ID NO: 8 and a light chain sequence which comprises SEQ ID NO: 11. In certain embodiments, the antibody comprises a heavy chain sequence which comprises SEQ ID NO: 9 and a light chain sequence which comprises SEQ ID NO: 11. In certain embodiments, the antibody comprises a heavy chain sequence which comprises SEQ ID NO: 10 and a light chain sequence which comprises SEQ ID NO: 11. In certain embodiments, the antibody comprises a heavy chain sequence which comprises SEQ ID NO: 12 and a light chain sequence which comprises SEQ ID NO: 16. In certain embodiments, the antibody comprises a heavy chain sequence which comprises SEQ ID NO: 13 and a light chain sequence which comprises SEQ ID NO: 16. In certain embodiments, the antibody comprises a heavy chain sequence which comprises SEQ ID NO: 14 and a light chain sequence which comprises SEQ ID NO: 16. In certain embodiments, the antibody comprises a heavy chain sequence which comprises SEQ ID NO: 15 and a light chain sequence which comprises SEQ ID NO: 16. In certain embodiments, the antibody comprises a heavy chain sequence which comprises SEQ ID NO: 17 and a light chain sequence which comprises SEQ ID NO: 21. In certain embodiments, the antibody comprises a heavy chain sequence which comprises SEQ ID NO: 18 and a light chain sequence which comprises SEQ ID NO: 21. In certain embodiments, the antibody comprises a heavy chain sequence which comprises SEQ ID NO: 19 and a light chain sequence which comprises SEQ ID NO: 21. In certain embodiments, the antibody comprises a heavy chain sequence which comprises SEQ ID NO: 20 and a light chain sequence which comprises SEQ ID NO: 21. In certain embodiments, the antibody comprises a heavy chain sequence which comprises SEQ ID NO: 22 and a light chain sequence which comprises SEQ ID NO: 26. In certain embodiments, the antibody comprises a heavy chain sequence which comprises SEQ ID NO: 23 and a light chain sequence which comprises SEQ ID NO: 26. In certain embodiments, the antibody comprises a heavy chain sequence which comprises SEQ ID NO: 24 and a light chain sequence which comprises SEQ ID NO: 26. In certain embodiments, the antibody comprises a heavy chain sequence which comprises SEQ ID NO: 25 and a light chain sequence which comprises SEQ ID NO: 26.

### Cytotoxins:

In certain embodiments, the cytototoxin is an auristatin, for example, monomethylauristatin F (MMAF) or monomethylauristatin E (MMAE) (see, e.g., U.S. Pat. Nos. 6,884,869; 7,498,298; 7,659,241; 7,994,135; 8,703,714; 7,964,567).

In certain embodiments, the cytotoxin is MMAF.

The structure for MMAF is provided below:

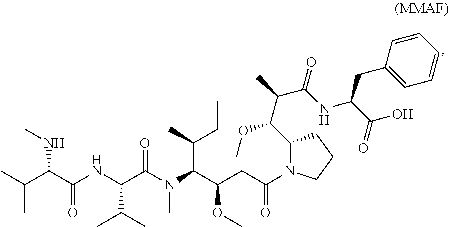 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

for which the chemical name is “(S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid.”

In certain embodiments, the cytotoxin is MMAE.

The structure for MMAE is provided below:

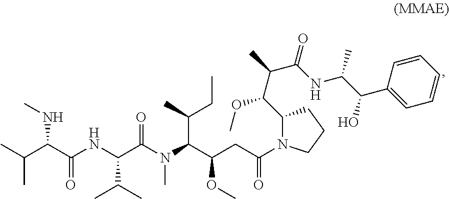 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

for which the chemical name is “(S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide.”

A person of ordinary skill in the art will understand that MMAF is also described in the art as as MeVal-Val-Dil-Dap-Phe, where “Dil” is dolaisoleuine, and “Dap” is dolaproine.

A person of ordinary skill in the art will understand that MMAE is also described in the art as MeVal-Val-Dil-Dap-Norephedrine, where “Dil” is dolaisoleuine, and “Dap” is dolaproine.

In certain embodiments, the cytotoxin is a pyrrolobenzodiazepine (see, e.g., U.S. Pat. Nos. 7,049,311; 7,741,319; 8,697,688; 8,765,740; WO 2011/130598 A1; WO 2012/112708 A1; WO 2013/055987 A1; WO 2013/165940 A1; see also, e.g., Jeffrey et al., Bioconjugate Chem. 2013, 24, 1256-1263, Sutherland et al., Blood 2013, 122(8), 1455-1463).

In certain embodiments, the pyrrolobenzodiazepine has the following structure:

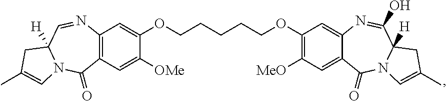

for which the chemical name is “(S)-11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-1H-benzo[e]pyrrolo[1,2-a][1,4]diazepin-5(11aH)-one.”

In certain embodiments, the pyrrolobenzodiazepine has the following structure:

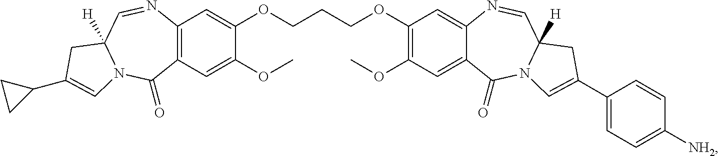 [SMILES: COc1cc2c(cc1OCCCOc1cc3c(cc1OC)C(=O)N1C=C(C4CC4)C[C@H]1C=N3)N=C[C@@H]1CC(c3ccc(N)cc3)=CN1C2=O]

for which the chemical name is “(S)-2-(4-aminophenyl)-8-(3-(((S)-2-cyclopropyl-7-methoxy-5-oxo-5,11a-dihydro-1H-benzo[e]pyrrolo[1,2-a][1,4]diazepin-8-yl)oxy)propoxy)-7-methoxy-1H-benzo[e]pyrrolo[1,2-a][1,4]diazepin-5(11aH)-one” (see, e.g., compound (26) in Example 5 of U.S. Pat. No. 8,697,688).

In certain embodiments, the pyrrolobenzodiazepine has the following structure:

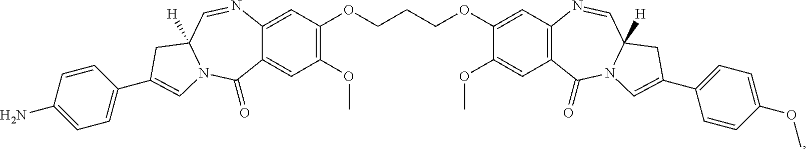 [SMILES: COc1ccc(C2=CN3C(=O)c4cc(OC)c(OCCCOc5cc6c(cc5OC)C(=O)N5C=C(c7ccc(N)cc7)C[C@H]5C=N6)cc4N=C[C@@H]3C2)cc1]

for which the chemical name is (S)-2-(4-aminophenyl)-7-methoxy-8-(3-(((S)-7-methoxy-2-(4-methoxyphenyl)-5-oxo-5,11a-dihydro-1H-benzo[e]pyrrolo[1,2-a][1,4]diazepin-8-yl)oxy)propoxy)-1H-benzo[e]pyrrolo[1,2-a][1,4]diazepin-5(11aH)-one.

In certain embodiments, the cytotoxin is one of any pyrrolobenzodiazepines disclosed in Jeffrey et al., Bioconjugate Chem. 2013, 24, 1256-1263, Sutherland et al., Blood 2013, 122(8), 1455-1463.

In certain embodiments, the cytotoxin is calicheamicin, doxorubicin, camptothecin, duocarmycin, DM1, DM4, a maytansinoid, or a tubulysin.

### Methods of Making:

In another aspect, provided herein is a method of making an antibody-drug conjugate of the following formula (I):

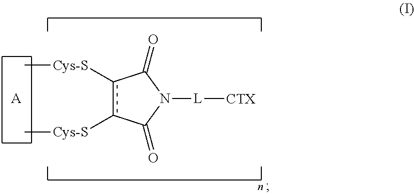

or a pharmaceutically acceptable salt thereof, wherein:
A is an antibody; the two depicted cysteine residues are from an opened cysteine-cysteine disulfide bond in A; L is a cleavable or a noncleavable linker; CTX is a cytotoxin bonded to L by an amide bond or a carbamate bond; the [symbol] bond represents a single or a double bond; and n is 4;
   wherein the method comprises the steps of:
a) providing a solution comprising A;
b) contacting the solution of a) with a solution comprising TCEP;
c) contacting the solution of b) with a solution comprising a cytotoxin linker conjugate.

In certain embodiments of the method of making an antibody-drug conjugate of formula (I), the cytotoxin-linker conjugate is a disubstituted maleimide-cytotoxin linker conjugate, for example, a dibromomaleimido-cytotoxin linker conjugate.

In certain embodiments of the method of making an antibody-drug conjugate of formula (I), the cytotoxin-linker conjugate is a monosubstituted maleimide-cytotoxin linker conjugate, for example, a bromomaleimido-cytotoxin linker conjugate, or a cyanophenolmaleimido-cytotoxin linker conjugate.

In certain embodiments, provided herein is a method of making an antibody-drug conjugate of the following formula (Ia):

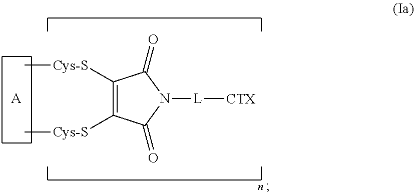

or a pharmaceutically acceptable salt thereof, wherein:
A is an antibody; the two depicted cysteine residues are from an opened cysteine-cysteine disulfide bond in A; L is a cleavable or a noncleavable linker; CTX is a cytotoxin bonded to L by an amide bond or a carbamate bond; and n is 4;
   wherein the method comprises the steps of:
a) providing a solution comprising A;
b) contacting the solution of a) with a solution comprising TCEP;
c) contacting the solution of b) with a solution comprising a cytotoxin linker conjugate.

In certain embodiments of the method of making an antibody-drug conjugate of formula (Ia), the cytotoxin-linker conjugate is a disubstituted maleimide-cytotoxin linker conjugate, for example, a dibromomaleimido-cytotoxin linker conjugate.

In certain embodiments, provided herein is a method of making an antibody-drug conjugate of the following formula (Ib):

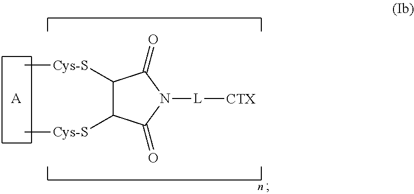

or a pharmaceutically acceptable salt thereof, wherein:
A is an antibody; the two depicted cysteine residues are from an opened cysteine-cysteine disulfide bond in A; L is a cleavable or a noncleavable linker; CTX is a cytotoxin bonded to L by an amide bond or a carbamate bond; and n is 4;
   wherein the method comprises the steps of:
a) providing a solution comprising A;
b) contacting the solution of a) with a solution comprising TCEP;
c) contacting the solution of b) with a solution comprising a cytotoxin linker conjugate.

In certain embodiments of the method of making an antibody-drug conjugate of formula (Ib), the cytotoxin-linker conjugate is a monosubstituted maleimide-cytotoxin linker conjugate, for example, a bromomaleimido-cytotoxin linker conjugate, or a cyanophenolmaleimido-cytotoxin linker conjugate.

In certain embodiments of the method of making an antibody-drug conjugate of formula (I), (Ia) or (Ib), L is a noncleavable linker.

In certain embodiments of the method of making an antibody-drug conjugate of formula (I), (Ia) or (Ib), L is:

—(CH₂)ₘC(O)—,

—(CH₂CH₂O)ₚ(CH₂CH₂)C(O)—, or

—(CH₂CH₂)(OCH₂CH₂)ₚC(O)—;

wherein m is an integer of 5 to 11, and p is an integer of 1 to 3.

In certain embodiments of the method of making an antibody-drug conjugate of formula (I), (Ia) or (Ib), L is a cleavable linker.

In certain embodiments of the method of making an antibody-drug conjugate of formula (I), (Ia) or (Ib), L is:

—(CH₂)ₘC(O)-Val-Ala-PAB-C(O)—,

—(CH₂)ₘC(O)-Val-Cit-PAB-C(O)—,

—(CH₂CH₂O)ₚ(CH₂CH₂)C(O)-Val-Ala-PAB-C(O)—,

—(CH₂CH₂O)ₚ(CH₂CH₂)C(O)-Val-Cit-PAB-C(O)—,

—(CH₂CH₂)(OCH₂CH₂)ₚC(O)-Val-Ala-PAB-C(O)—, or

—(CH₂CH₂)(OCH₂CH₂)ₚC(O)-Val-Cit-PAB-C(O)—;

wherein m is an integer of 5 to 11, and p is an integer of 1 to 3; and

wherein PAB has the following structure:

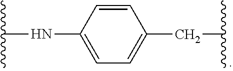 [SMILES: *Cc1ccc(N*)cc1]

In certain embodiments of the method of making an antibody-drug conjugate of formula (I), (Ia) or (Ib), A is an antibody that is specific to a cancer antigen. In certain embodiments, A is selected from the group consisting of alemtuzumab, anitumumab, bevacizumab, brentuximab, cetuximab, gemtuzumab, glembatumumab, inotuzumab, ipilimumab, lovortumumab, milatuzumab, ofatumumab, rituximab, tositumomab, and trastuzumab.

In certain embodiments of the method of making an antibody-drug conjugate of formula (I), (Ia) or (Ib), CTX is an auristatin. In certain embodiments the CTX is monomethylauristatin F (MMAF). In certain embodiments the CTX is monomethylauristatin E (MMAE). In certain embodiments of the method of making an antibody-drug conjugate of formula (I), (Ia) or (Ib), CTX is a pyrrolobenzodiazepine (PBD).

In certain embodiments of the method of making an antibody-drug conjugate of formula (Ia), the cytotoxin linker conjugate is of the following formula (IIa):

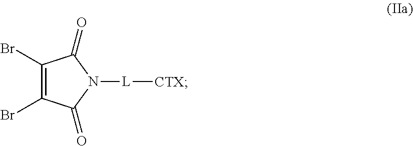

wherein CTX is monomethylauristatin F bonded to L by an amide bond.

In certain embodiments of the method of making an antibody-drug conjugate of formula (Ib), the cytotoxin linker conjugate is of the following formula (IIb):

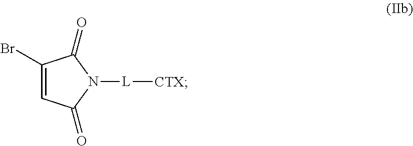

wherein CTX is monomethylauristatin F bonded to L by an amide bond.

In certain embodiments of the method of making an antibody-drug conjugate of formula (Ib), the cytotoxin linker conjugate is of the following formula (IIc):

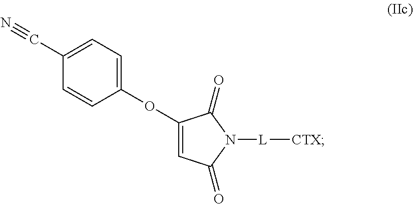

wherein CTX is monomethylauristatin F bonded to L by an amide bond. In certain embodiments of the method of making an antibody-drug conjugate of formula (I), (Ia) or (Ib), the solution of step a) comprises 20 mM sodium phosphate, 20 mM Borate, and 5 mM EDTA. In certain embodiments, the pH of the solution of steps a), b) and/or c) is between about 7.0 to about 8.2. In certain embodiments, the pH of the solution of steps a), b) and/or c) is between about 7.4 to about 8.2. In certain embodiments, the pH of the solution of steps a), b) and/or c) is between about 7.0 to about 7.8. In certain embodiments, the pH of the solution of steps a), b) and/or c) is about 7.2. In certain embodiments, the pH of the solution of step b) is 7.2. In certain embodiments, steps a), b) and/or c) are performed at a temperature of about 22° C. to about 37° C. In certain embodiments, steps a), b) and/or c) are performed at a temperature of about 22° C. to about 27° C. In certain embodiments, steps b) and c) are performed at a temperature of about 22° C. to about 27° C. In certain embodiments, the ratio of molar equivalents of TCEP to antibody in step b) is about 4 to about 10. In certain embodiments, the ratio of TCEP to antibody in step b) is about 9.5. In certain embodiments, the ratio of molar equivalents of cytotoxin linker conjugate to antibody in step c) is about 4 to about 10. In certain embodiments, In certain embodiments, the ratio of molar equivalents of cytotoxin linker conjugate to antibody in step c) is about 4.5 to about 6.0. In certain embodiments, In certain embodiments, the ratio of molar equivalents of cytotoxin linker conjugate to antibody in step c) is about 4.5 to about 5.5. In certain embodiments, In certain embodiments, the ratio of molar equivalents of cytotoxin linker conjugate to antibody in step c) is about 5.0 to about 6.0. In certain embodiments, the ratio of molar equivalents of cytotoxin linker conjugate to antibody in step c) is about 5.1 to about 5.8.

In another aspect, provided herein is a method of making a compound of formula (22):

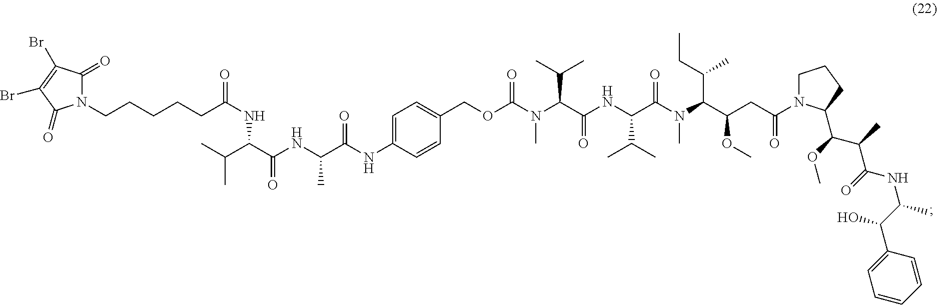 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1)C(C)C]

or salt thereof.

In certain embodiments, the method comprises reacting a compound of formula (21),

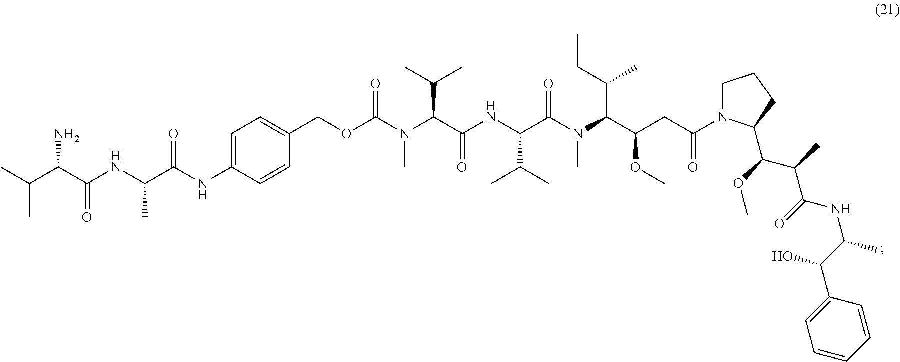 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](N)C(C)C)cc1)C(C)C]

or salt thereof, with a compound of formula (3);

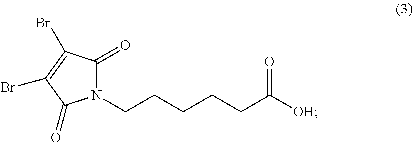 [SMILES: O=C(O)CCCCCN1C(=O)C(Br)=C(Br)C1=O]

in the presence of N,N′-Diisopropylcarbodiimide (DIPC) and
N,N-Diisopropylethylamine (DIPEA) in tetrahydrofuran (THF).

In certain embodiments, the compound of formula (21), or salt thereof, is prepared by reacting a compound of formula (20);

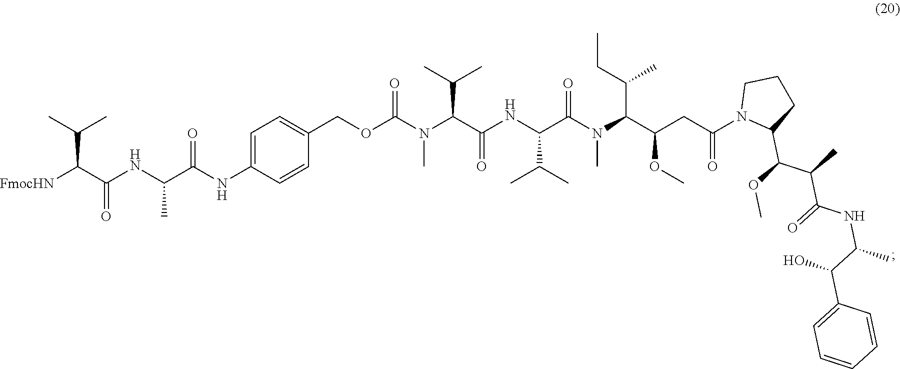

or salt thereof, with piperidine in dimethylformamide (DMF).

In certain embodiments, the compound of formula (20), or salt thereof, is prepared by reacting a compound of formula (19);

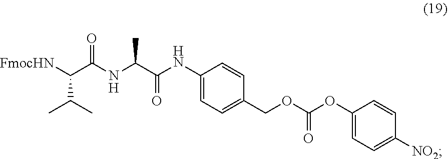

or salt thereof, with monomethylauristatin E, or salt thereof, in the presence of 1-hydroxy-7-aza-benzotriazole (HOAt) and DIPEA in DMF.

In certain embodiments, the compound of formula (19), or salt thereof, is prepared by reacting a compound of formula (18):

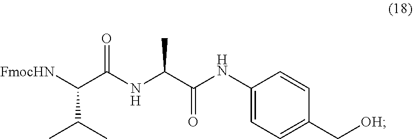

or salt thereof, with bis(4-nitrophenyl) carbonate and DIPEA in DMF.

A person of ordinary skill in the art will understand that using the following compound:

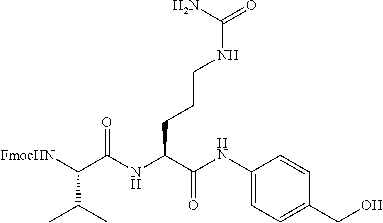

in place of the compound of formula (18) in the method of making disclosed above will give the following compound:

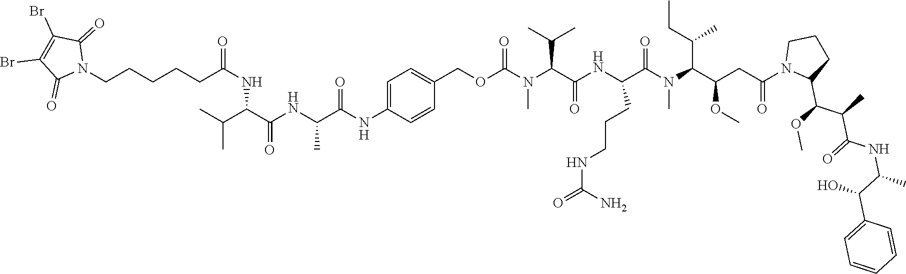

in place of the compound of formula (22).

### Assays:

The ADCs disclosed herein may be assayed for binding affinity to and specificity for the desired antigen by any of the methods conventionally used for the assay of antibodies; and they may be assayed for efficacy as anticancer agents by any of the methods conventionally used for the assay of cytostatic/cytotoxic agents, such as assays for potency against cell cultures, xenograft assays, and the like. A person of ordinary skill in the art will have no difficulty, considering that skill and the literature available, in determining suitable assay techniques; from the results of those assays, in determining suitable doses to test in humans as anticancer agents, and, from the results of those tests, in determining suitable doses to use to treat cancers in humans.

### Formulation and Administration:

The ADCs disclosed herein will typically be formulated as solutions for intravenous administration, or as lyophilized concentrates for reconstitution to prepare intravenous solutions (to be reconstituted, e.g., with normal saline, 5% dextrose, or similar isotonic solutions). They will typically be administered by intravenous injection or infusion. A person of ordinary skill in the art of pharmaceutical formulation, especially the formulation of anticancer antibodies, will have no difficulty, considering that skill and the literature available, in developing suitable formulations.

### EXAMPLES

### Example 1: Synthesis of Linkers

### Example 1A

Linkers, such as 6-(3,4-dibromo-2,5-dioxo-2,5-dihydro-1H-pyrrol-1-yl)hexanoic acid (“DBM(C6)”), may be synthesized as follows.

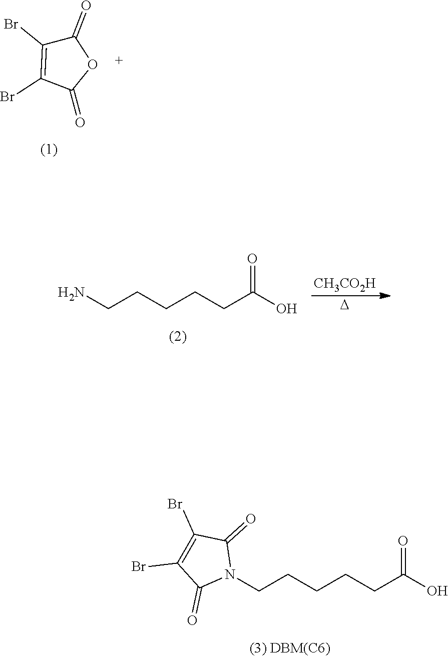

Procedure:

6-aminohexanoic acid (1) (0.512 mg, 3.91 mmol) was added to a solution of dibromomaleic anhydride (2) (1 g, 3.91 mmol) in acetic acid (20 mL) and the solution was stirred at room temperature for 10 minutes until all the solids dissolved. The reaction mixture was then heated to 100° C. for 18 h, after which time LC/MS indicated the reaction was complete. The solution was concentrated under vacuum and purified by silica gel chromatography on a 24 g silica gel column. The column was eluted with a gradient of 0-40% ethyl acetate in dichloromethane at 25 mL/min over 30 minutes. Elution of product was monitored at 254 nm and analyzed by LC/MS. Concentration of the pure fractions containing the desired “DBM-(C6)” linker, 6-(3,4-dibromo-2,5-dioxo-2,5-dihydro-1H-pyrrol-1-yl)hexanoic acid (3), yielded 1.15 g, (3.12 mmol) of pure linker in 80% yield.

LC/MS: RT=3.172 min (5-95% acetonitrile in water) over 5 min at 0.8 mL/min, m/z neg.− observed 391.9 389.9, 393.9 [M+Na]. ₁H NMR (400 MHz, CDCl₃) δ3.62 (t, J=7.2 Hz, 2H), 2.36 (t, J=7.6 Hz, 2H), 1.68-1.62 (m, 4H) 1.41-1.30 (m, 2H).

Similar synthesis using 7-aminoheptanoic acid, 8-aminoctanoic acid, 9-aminononanoic acid, 10-aminodecanoic acid, 11-aminoundecanoic acid, or 12-aminododecanoic acid in place of 6-aminohexanoic acid (1) give 7-(3,4-dibromo-2,5-dioxo-2,5-dihydro-1H-pyrrol-1-yl)heptanoic acid (“DBM(C7)”), 8-(3,4-dibromo-2,5-dioxo-2,5-dihydro-1H-pyrrol-1-yl)octanoic acid (“DBM(C8)”), 9-(3,4-dibromo-2,5-dioxo-2,5-dihydro-1H-pyrrol-1-yl)nonanoic acid (“DBM(C9)”), 10-(3,4-dibromo-2,5-dioxo-2,5-dihydro-1H-pyrrol-1-yl)decanoic acid (“DBM(C10)”), 11-(3,4-dibromo-2,5-dioxo-2,5-dihydro-1H-pyrrol-1-yl)undecanoic acid (“DBM(C11)”), and 12-(3,4-dibromo-2,5-dioxo-2,5-dihydro-1H-pyrrol-1-yl)dodecanoic acid (“DBM(C12)”), respectively, which are depicted below:

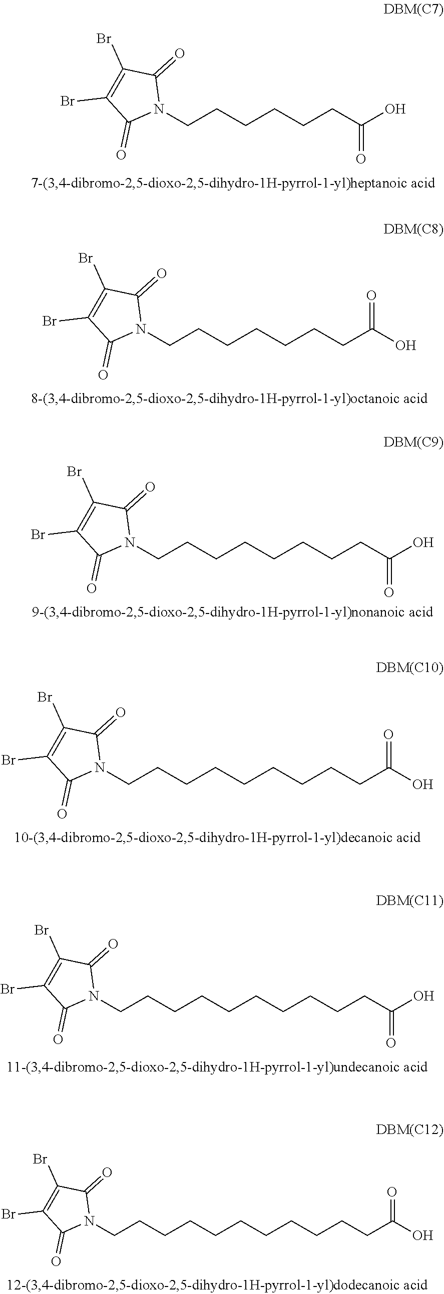 [SMILES: O=C(O)CCCCCCN1C(=O)C(Br)=C(Br)C1=O ; O=C(O)CCCCCCCN1C(=O)C(Br)=C(Br)C1=O ; O=C(O)CCCCCCCCN1C(=O)C(Br)=C(Br)C1=O ; O=C(O)CCCCCCCCCN1C(=O)C(Br)=C(Br)C1=O ; O=C(O)CCCCCCCCCCN1C(=O)C(Br)=C(Br)C1=O ; O=C(O)CCCCCCCCCCCN1C(=O)C(Br)=C(Br)C1=O]

### Example 1B

Linkers, such as 6-(3-(4-cyanophenoxy)-2,5-dioxo-2,5-dihydro-1H-pyrrol-1-yl)hexanoic acid (“CPM(C6)”), may be synthesized as follows.

### Step 1: Synthesis of Monobromo Maleimide (BRM) Intermediate

### Step 1

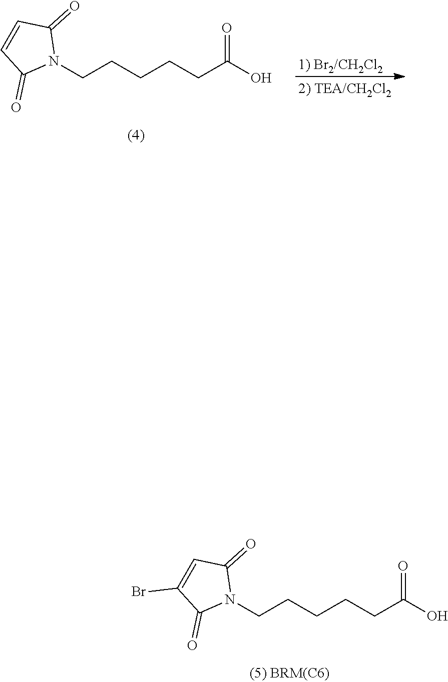

Procedure:

Bromine (5.0 ml, 97.0 mmol) was added to 6-(2,5-dioxo-2,5-dihydro-1H-pyrrol-1-yl) hexanoic acid (4) (13.1 g, 62.0 mmol) in methylene chloride (200 ml) and the mixture was stirred for 18 hours at 20° C. The solution was cooled to 4° C. and triethylamine (20 ml, 143 mmol) was slowly added drop wise via an addition funnel. The reaction was stirred for 1 hour at 4° C. 200 mL of 1N aqueous hydrogen chloride was added. The layers were separated and the aqueous layer extracted twice with 100 mL of ethyl acetate. The combined organic extracts were dried over anhydrous sodium sulfate, filtered, and the filtrate was concentrated via rotary evaporation. The crude residue, 6-(3-bromo-2,5-dioxo-2,5-dihydro-1H-pyrrol-1-yl)hexanoic acid (5), was purified by flash chromatography on silica gel (220 g) with methylene chloride:ethyl acetate as the eluent using a gradient of 0 to 50% ethyl acetate over 25 min. Fractions containing the desired product by LC/MS analysis were combined. Evaporation of purified fractions afforded 15 g (83% yield) of the desired BRM intermediate, 6-(3,4-dibromo-2,5-dioxo-2,5-dihydro-1H-pyrrol-1-yl) hexanoic acid (5) (“BRM(C6)”) as a light yellow solid used without further purification.

### Step 2. Synthesis of CPM Linker

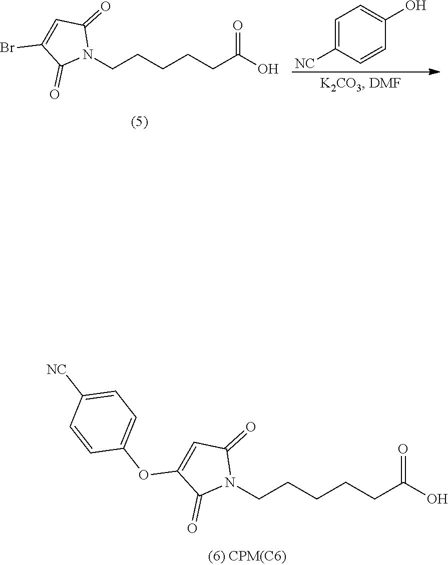 [SMILES: O=C(O)CCCCCN1C(=O)C=C(Br)C1=O ; N#Cc1ccc(O)cc1 ; N#Cc1ccc(OC2=CC(=O)N(CCCCCC(=O)O)C2=O)cc1]

Procedure:

7.6 g 4-cyanophenol was dissolved in 25 mL dimethylformamide, then 13.2 g potassium carbonate was added and the suspension was stirred for 15 min. 3.7 g of purified bromomaleimido hexanoic acid (5) from step 1 was then added and the reaction was stirred at R.T. for 5h. 4 N aqueous hydrogen chloride was added until pH was <2. The product was extracted with ethyl acetate (3×200 mL). The combined organic extracts were washed with brine (3×25 mL) then dried over anhydrous sodium sulfate, filtered, and the filtrate was concentrated under reduced pressure at 37° C. The crude residue was dissolved in 50 mL methylene chloride and flash chromatographed on silica (220 g) with methylene chloride:ethyl acetate as the eluent (0-100% EtOAc over 25 min) to afford 2.2 g (52% yield) 6-(3-(4-cyanophenoxy)-2,5-dioxo-2,5-dihydro-1H-pyrrol-1-yl)hexanoic acid (6) as a white solid. ¹H NMR (400 MHz, CDCl₃) 7.75 (d, 2H), 7.30 (d, 2H), 5.45 (s, 1H), 3.55 (t, 2H), 2.35 (t, 2H), 1.60-1.70 (m, 4H), 1.3-1.4 (m, 2H).

### Example 2: Alternative Synthesis of Linkers

Linkers, such as 6-(3,4-dibromo-2,5-dioxo-2,5-dihydro-1H-pyrrol-1-yl)hexanoic acid (“DBM(C6)”), may be alternatively synthesized as follows.

### Step 1: Synthesis of Monobromo Maleimide (BRM) Intermediate

### Step 1

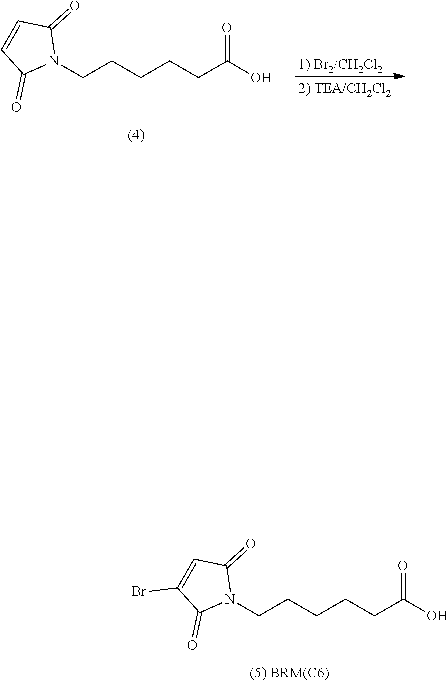

Procedure:

Bromine (5.0 ml, 97.0 mmol) was added to 6-(2,5-dioxo-2,5-dihydro-1H-pyrrol-1-yl) hexanoic acid (4) (13.1 g, 62.0 mmol) in methylene chloride (200 ml) and the mixture was stirred for 18 hours at 20° C. The solution was cooled to 4° C. and triethylamine (20 ml, 143 mmol) was slowly added drop wise via an addition funnel. The reaction was stirred for 1 hour at 4° C. 200 mL of 1 N aqueous hydrogen chloride was added. The layers were separated and the aqueous layer extracted twice with 100 mL of ethyl acetate. The combined organic extracts were dried over anhydrous sodium sulfate, filtered, and the filtrate was concentrated via rotary evaporation. The crude residue, 6-(3-bromo-2,5-dioxo-2,5-dihydro-1H-pyrrol-1-yl)hexanoic acid (5), was purified by flash chromatography on silica gel (220 g) with methylene chloride:ethyl acetate as the eluent using a gradient of 0 to 50% ethyl acetate over 25 min. Fractions containing the desired product by LC/MS analysis were combined. Evaporation of purified fractions afforded 15 g (83% yield) of the desired BRM intermediate, 6-(3,4-dibromo-2,5-dioxo-2,5-dihydro-1H-pyrrol-1-yl) hexanoic acid (5) (“BRM(C6)”) as a light yellow solid used without further purification.

### Step 2. Synthesis of DBM Linker

### Step 2

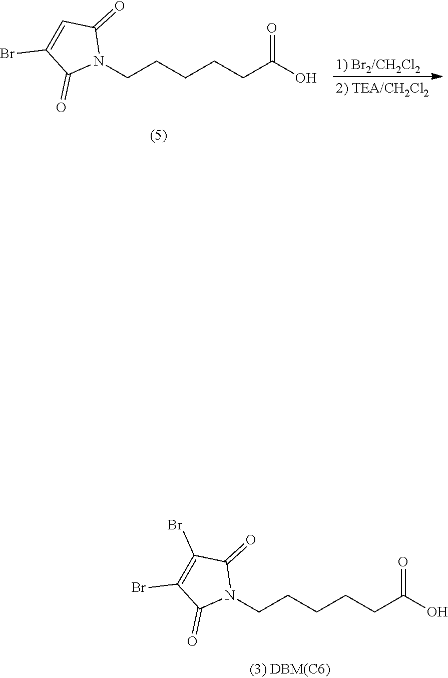

Procedure:

15 g of purified bromomaleimido hexanoic acid (5) from step 1 was dissolved in methylene chloride (200 ml) and bromine (15.0 ml, 291 mmol) was added. The reaction was stirred for 72 h at R.T. and then cooled to 4° C. Triethylamine (80 ml, 574 mmol) was added dropwise via an addition funnel. The mixture was stirred for 1 hour at 4° C. and 2 N aqueous hydrogen chloride was added until pH was <2. The DCM layer was separated and the aqueous layer was extracted with ethyl acetate (2×200 mL). The combined organic extracts were dried over anhydrous sodium sulfate, filtered, and the filtrate was concentrated under reduced pressure at 37° C. The crude residue was flash chromatographed on silica (220 g) with methylene chloride:ethyl acetate as the eluent (0-50% EtOAc over 25 min) to afford 13.1 g (68% yield) 6-(3,4-dibromo-2,5-dioxo-2,5-dihydro-1H-pyrrol-1-yl) hexanoic acid (3) as a white solid.

### Example 3: Synthesis of Additional Linkers

### Example 3A

Synthesis of additional linkers, such as 3-(2-(2-(2-(3,4-dibromo-2,5-dioxo-2,5-dihydro-1H-pyrrol-1-yl) ethoxy) ethoxy) ethoxy) propanoic acid (“DBM(PEG3)”), may be prepared as follows.

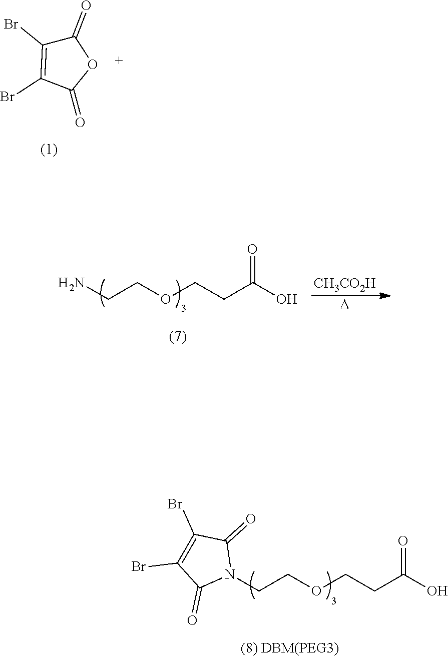

Procedure:

1.03 g (3.9 mmol) of 3-(2-(2-(2-aminoethoxy)ethoxy) ethoxy) propanoic acid (7) was added to a solution of dibromomaleic anhydride (1) (1 g, 3.91 mmol) in acetic acid (20 mL) and the solution was stirred at room temperature for 10 minutes until all the solids dissolved. The reaction mixture was then heated to 100° C. for 18 h, after which time LC/MS indicated the reaction was complete. The solution was concentrated under vacuum and purified by silica gel chromatography on a 24 g silica gel column. The column was eluted with a gradient of 0-50% ethyl acetate in dichloromethane at 25 mL/min over 30 minutes. Elution of product was monitored at 254 nm and and analyzed by LC/MS. Concentration of the pure fractions containing the desired DBM-(PEG3) linker yielded 1.3 g, (3.12 mmol) of pure DBM(PEG3) linker, 3-(2-(2-(2-(3,4-dibromo-2,5-dioxo-2,5-dihydro-1H-pyrrol-1-yl) ethoxy) ethoxy) ethoxy) propanoic acid (8), in 60% yield. MS observed M/Z=504.1 MH+.

Similar synthesis using 3-(2-aminoethoxy)propanoic acid or 3-(2-(2-aminoethoxy)ethoxy)propanoic acid in place of 3-(2-(2-(2-aminoethoxy)ethoxy) ethoxy) propanoic acid (7) give 3-(2-(3,4-dibromo-2,5-dioxo-2,5-dihydro-1H-pyrrol-1-yl)ethoxy)propanoic acid (“DBM(PEG1)”), and 3-(2-(2-(3,4-dibromo-2,5-dioxo-2,5-dihydro-1H-pyrrol-1-yl)ethoxy)ethoxy)propanoic acid (“DBM(PEG2)”), respectively, which are depicted below:

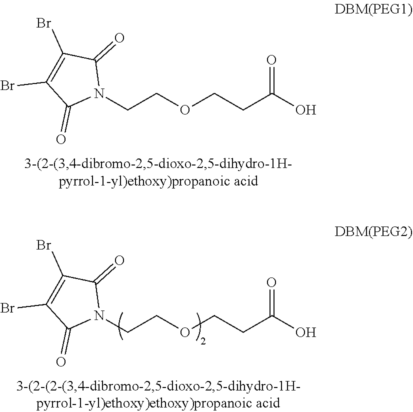

### Example 3B

Linkers, such as 1-(3-(4-cyanophenoxy)-2,5-dioxo-2,5-dihydro-1H-pyrrol-1-yl)-3-oxo-7,10,13,16,19,22,25,28-octaoxa-4-azahentriacontan-31-oic acid (“CPM(C3)PEG8”), may be synthesized as follows.

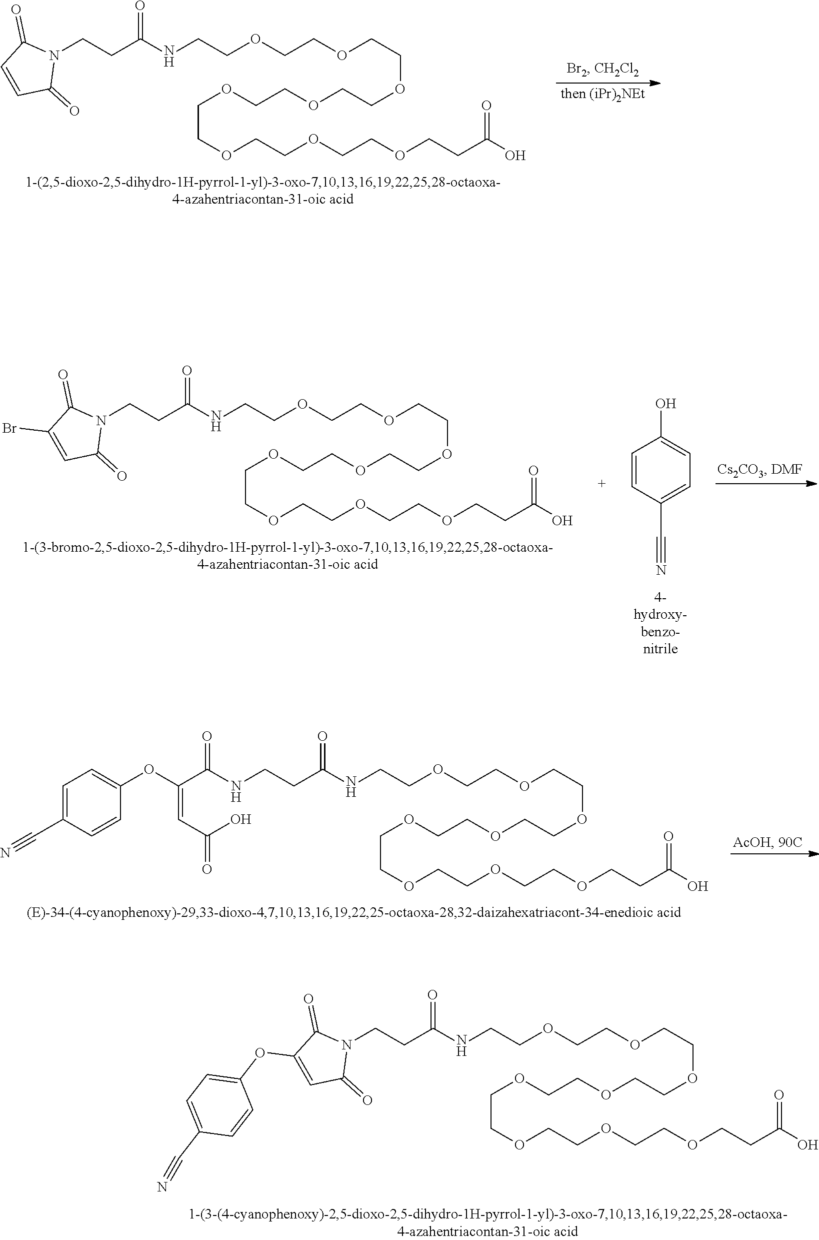 [SMILES: O=C(O)CCOCCOCCOCCOCCOCCOCCOCCOCCNC(=O)CCN1C(=O)C=CC1=O ; O=C(O)CCOCCOCCOCCOCCOCCOCCOCCOCCNC(=O)CCN1C(=O)C=C(Br)C1=O ; N#Cc1ccc(O)cc1 ; N#Cc1ccc(O/C(=C/C(=O)O)C(=O)NCCC(=O)NCCOCCOCCOCCOCCOCCOCCOCCOCCC(=O)O)cc1 ; N#Cc1ccc(OC2=CC(=O)N(CCC(=O)NCCOCCOCCOCCOCCOCCOCCOCCOCCC(=O)O)C2=O)cc1]

### Step 1: Synthesis of 1-(3-bromo-2,5-dioxo-2,5-dihydro-1H-pyrrol-1-yl)-3-oxo-7,10,13,16,19,22,25,28-octaoxa-4-azahentriacontan-31-oic Acid Intermediate

Procedure:

Bromine (0.20 ml, 3.88 mmol) was added to a solution of 1-(2,5-dioxo-2,5-dihydro-1H-pyrrol-1-yl)-3-oxo-7,10,13,16,19,22,25,28-octaoxa-4-azahentriacontan-31-oic acid (1000 mg, 1.69 mmol) in methylene chloride (17 ml). After stirring for 14 h, the solution was cooled to −10 C in an ice/brine bath and diisopropylethylamine (1.5 ml, 8.61 mmol) was slowly added dropwise. After stirring for an additional 24 h, during which time the solution warmed to ambient temperature, the solution was concentrated under reduced pressure to afford crude 1-(3-bromo-2,5-dioxo-2,5-dihydro-1H-pyrrol-1-yl)-3-oxo-7,10,13,16,19,22,25,28-octaoxa-4-azahentriacontan-31-oic acid. UPLC/MS 1.18 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flow rate was 0.8 mL/min.), m/z 671.6 and 673.6 [M+H]⁺.

### Step 2: Synthesis of (E)-34-(4-cyanophenoxy)-29,33-dioxo-4,7,10,13,16,19,22,25-octaoxa-28,32-diazahexatriacont-34-enedioic Acid Intermediate

Procedure:

The residue was diluted with dimethylformamide (10 ml) followed by the simultaneous addition of cesium carbonate (13.0 g, 39.9 mmol) and 4-hydroxybenzonitrile (3.6 g, 30.3 mmol) was added. After stirring for 2 h, the heterogeneous mixture was poured over 2 M aqueous hydrogen chloride (80 ml) at 0 C. The solution was directly purified by reverse phase HPLC to afford (E)-34-(4-cyanophenoxy)-29,33-dioxo-4,7,10,13,16,19,22,25-octaoxa-28,32-diazahexatriacont-34-enedioic acid.

### Step 2: Synthesis of 1-(3-(4-cyanophenoxy)-2,5-dioxo-2,5-dihydro-1H-pyrrol-1-yl)-3-oxo-7,10,13,16,19,22,25,28-octaoxa-4-azahentriacontan-31-oic acid (“CPM(C3)PEG8”)

Procedure:

(E)-34-(4-cyanophenoxy)-29,33-dioxo-4,7,10,13,16,19,22,25-octaoxa-28,32-diazahexatriacont-34-enedioic acid in acetic acid (2 ml) was placed into a preheated oil bath at 90 C for 1 h. The solution was cooled to ambient temperature, diluted with water, and purified by reverse phase HPLC to afford 140 mg (11% yield over 4 steps) of 1-(3-(4-cyanophenoxy)-2,5-dioxo-2,5-dihydro-1H-pyrrol-1-yl)-3-oxo-7,10,13,16,19,22,25,28-octaoxa-4-azahentriacontan-31-oic acid as a brown oil. ¹H NMR (400 MHz, DMSO-d₆) δ ppm 12.15 (broad s, 2H), 8.01 (d, J=8.0 Hz, 2H), 7.57 (d, J=8.0 Hz, 2H), 5.91 (s, 1H), 3.62 (m, 4H), 3.51 (broad s, 30H), 3.17 (q, J=8.0 Hz, 2H), 2.44 (t, J=8.0 Hz, 2H), 2.37 (t, J=8.0 Hz, 2H). UPLC/MS 1.26 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flow rate was 0.8 mL/min.), m/z 710.7 [M+H]+.

### Example 4: Synthesis of Cleavable Linkers

### Example 4A

Cleavable linkers, including DBM cleavable linkers, may be synthesized as follows.

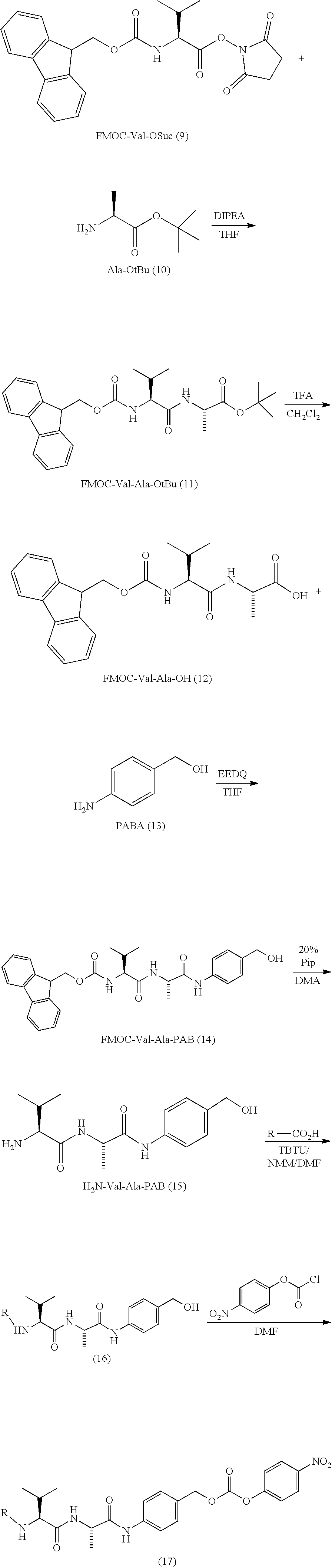

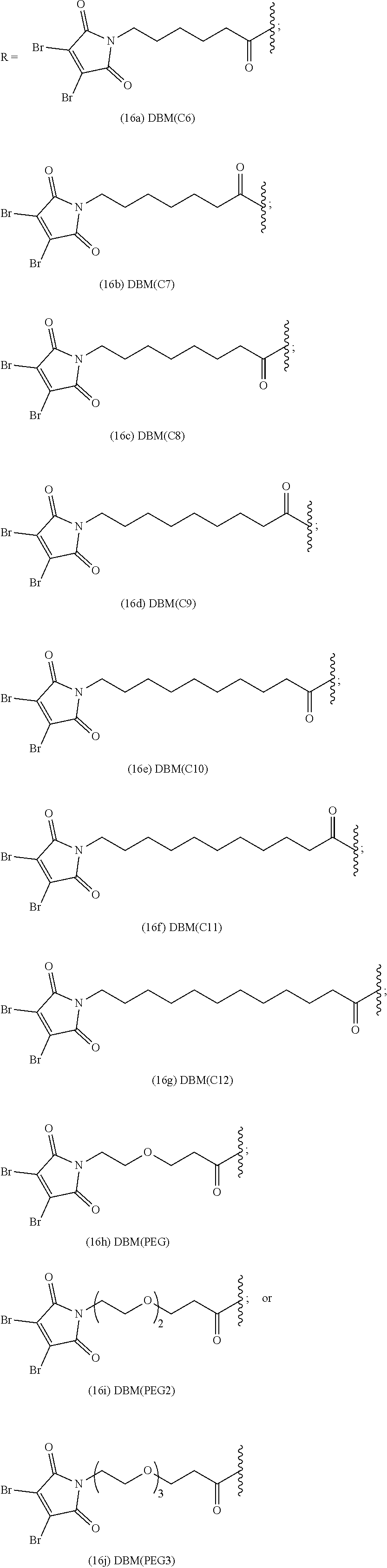

(S)-2,5-dioxopyrrolidin-1-yl 2-((((9H-fluoren-9-yl)methoxy)carbonyl)amino)-3-methylbutanoate (9) was reacted with (S)-tert-butyl 2-aminopropanoate (10) in the presence of 2 equivalents of DIPEA in THF to yield (S)-tert-butyl 2-((S)-2-((((9H-fluoren-9-yl)methoxy)carbonyl)amino)-3-methylbutanamido)propanoate (11). To fully deprotect (11) to the free acid, (S)-2-((S)-2-((((9H-fluoren-9-yl)methoxy)carbonyl)amino)-3-methylbutanamido)propanoic acid (12), the lyophilized material was treated with 5% TFA in CH₂Cl₂. The free carboxylic acid of the purified product (12) was then coupled to (4-aminophenyl)methanol (13), in the presence of 2 equivalents of EEDQ in THF to yield (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (14). The product (14) was treated with 20% piperidine in DMA to yield (S)-2-amino-N—((S)-1-((4-(hydroxymethyl)phenyl)amino)-1-oxopropan-2-yl)-3-methylbutanamide (15). Coupling of the product (15) with linker, R—CO₂H, was performed by activation with 1 equivalent of TBTU in the presence of 2 equivalents of NMM in DMF for 72 hours at room temperature to produce compound (16). Compound (16) was then reacted with 4-nitrophenyl carbonochloridate to produce compound (17).

Similar syntheses using Citrulline-OtBu (“Cit-OtBu”) in place of Ala-OtBu (10) give the corresponding DBM Cleavable Linkers comprising a -Val-Cit- (“VC”) dipeptide in place of an -Val-Ala- (“VA”) dipeptide.

### Example 4B

Cleavable linkers, including CPM cleavable linkers, may be synthesized as follows.

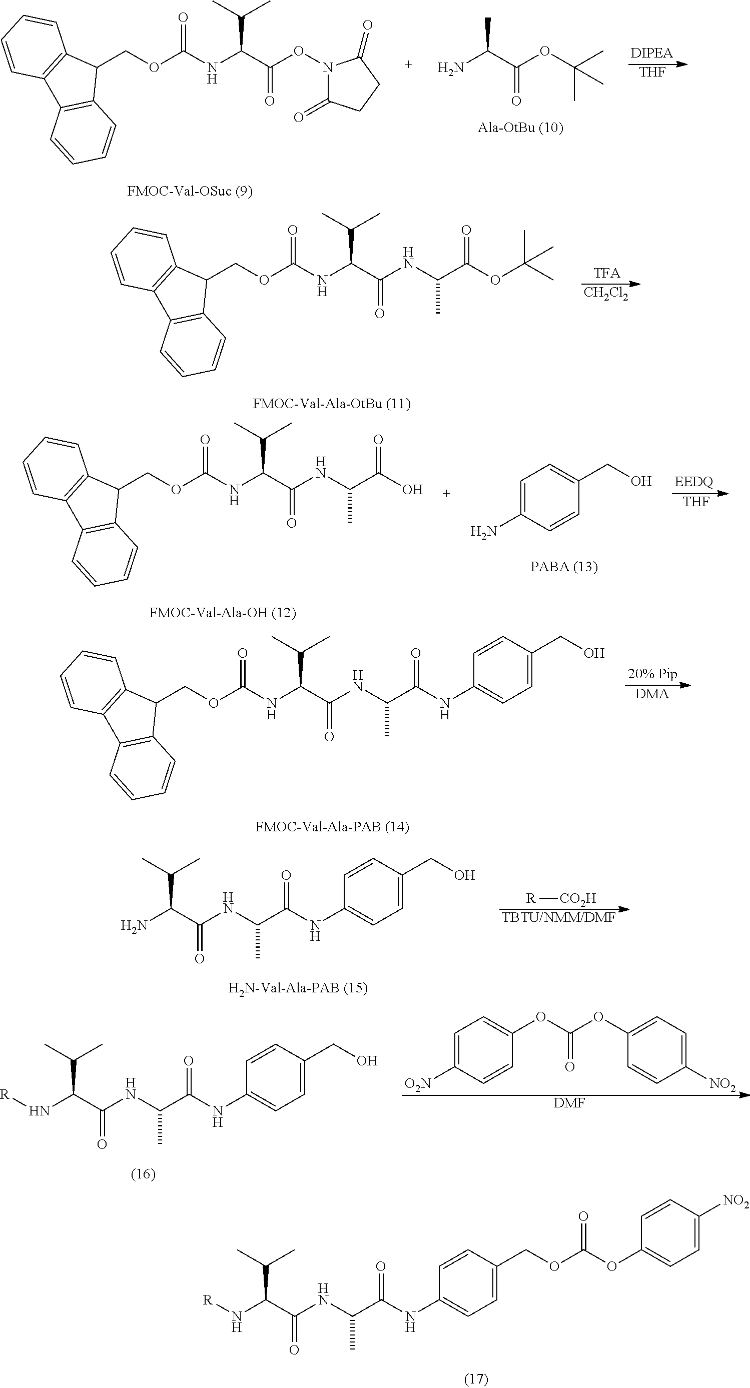

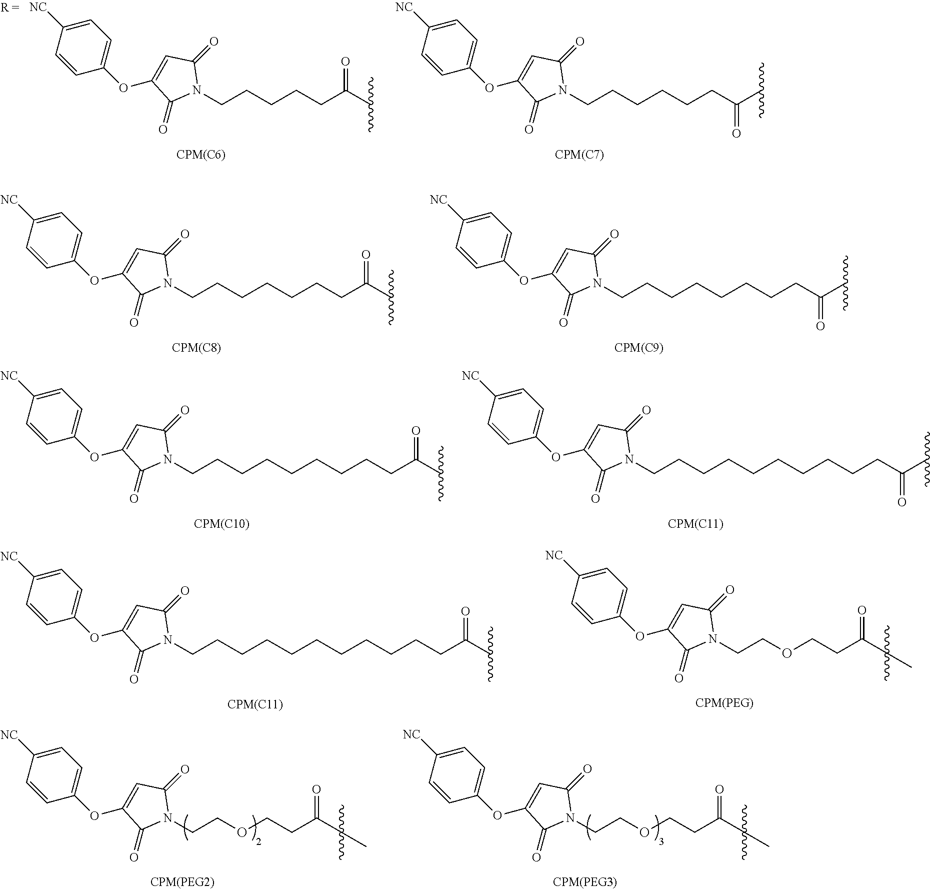

(S)-2,5-dioxopyrrolidin-1-yl 2-((((9H-fluoren-9-yl)methoxy)carbonyl)amino)-3-methylbutanoate (9) was reacted with (S)-tert-butyl 2-aminopropanoate (10) in the presence of 2 equivalents of DIPEA in THF to yield (S)-tert-butyl 2-((S)-2-((((9H-fluoren-9-yl)methoxy)carbonyl)amino)-3-methylbutanamido)propanoate (11). To fully deprotect (11) to the free acid, (S)-2-((S)-2-((((9H-fluoren-9-yl)methoxy)carbonyl)amino)-3-methylbutanamido)propanoic acid (12), the lyophilized material was treated with 5% TFA in CH₂Cl₂. The free carboxylic acid of the purified product (12) was then coupled to (4-aminophenyl)methanol (13), in the presence of 2 equivalents of EEDQ in THF to yield (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (14). The product (14) was treated with 20% piperidine in DMA to yield (S)-2-amino-N—((S)-1-((4-(hydroxymethyl)phenyl)amino)-1-oxopropan-2-yl)-3-methylbutanamide (15). Coupling of the product (15) with linker, R—CO₂H, was performed by activation with 1 equivalent of TBTU in the presence of 2 equivalents of NMM in DMF for 72 hours at room temperature to produce compound (16). Compound (16) was then reacted with bis(4-nitrophenyl) carbonate to produce compound (17).

Similar syntheses using Citrulline-OtBu (“Cit-OtBu”) in place of Ala-OtBu (10) give the corresponding DBM Cleavable Linkers comprising a -Val-Cit- (“VC”) dipeptide in place of an -Val-Ala- (“VA”) dipeptide.

### Synthesis of (6-(3-(4-cyanophenoxy)-2,5-dioxo-2,5-dihydro-1H-pyrrol-1-yl)hexanoyl)-L-valyl-L-alanine (“CPM(C6)-Val-Ala”)

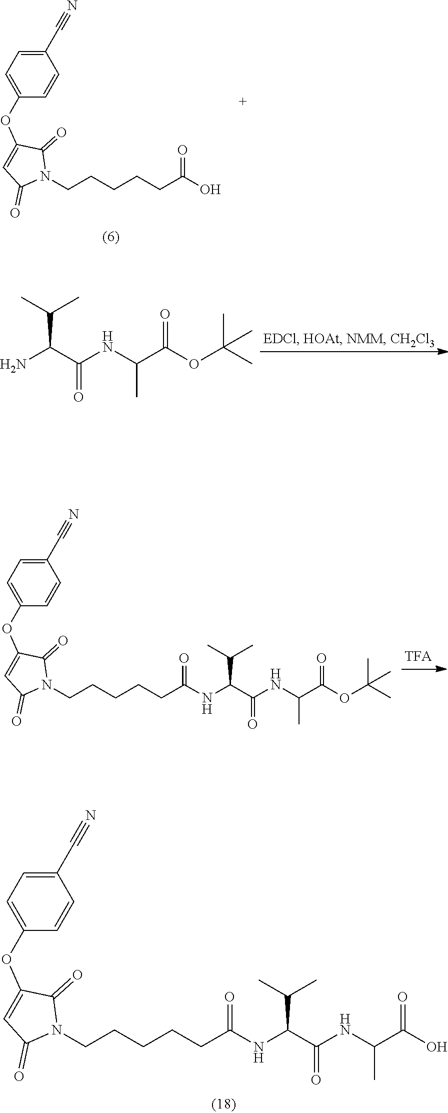 [SMILES: N#Cc1ccc(OC2=CC(=O)N(CCCCCC(=O)O)C2=O)cc1 ; CC(NC(=O)[C@@H](N)C(C)C)C(=O)OC(C)(C)C ; CC(NC(=O)[C@@H](NC(=O)CCCCCN1C(=O)C=C(Oc2ccc(C#N)cc2)C1=O)C(C)C)C(=O)OC(C)(C)C ; CC(NC(=O)[C@@H](NC(=O)CCCCCN1C(=O)C=C(Oc2ccc(C#N)cc2)C1=O)C(C)C)C(=O)O]

### Step 1

6-(3-(4-cyanophenoxy)-2,5-dioxo-2,5-dihydro-1H-pyrrol-1-yl)hexanoic acid (6) (350 mg, 1.07 mmol), tert-butyl L-alaninate hydrochloride (274 mg, 976 umol), 3-(((ethylimino)methylene)amino)-N,N-dimethylpropan-1-amine hydrochloride (330 mg, 1.72 mmol), 3H-[1,2,3]triazolo[4,5-b]pyridin-3-ol (17 mg, 12.4 umol), and N-methylmorpholine (0.20 ml, 1.82 mmol) in methylene chloride (20 ml) was stirred for 18 h. The solution was directly flash chromatographed on silica gel (80 g) with methylene chloride:ethyl acetate as the eluent 100:0 for 5 min then 100:0 to 50:50 over 25 min to afford 501 mg (93% yield) of tert-butyl (6-(3-(4-cyanophenoxy)-2,5-dioxo-2,5-dihydro-1H-pyrrol-1-yl)hexanoyl)-L-valyl-L-alaninate as a white solid. ¹H NMR (400 MHz, DMSO-d₆) δ ppm 8.23 (d, J=8.0 Hz, 1H), 8.00 (d, J=8.0 Hz, 2H), 7.75 (d, J=8.0 Hz, 1H), 7.58 (d, J=8.0 Hz, 2H), 5.86 (s, 1H), 4.21 (dd, J=8.0 Hz and 8.0 Hz, 1H), 4.10 (pent., J=8.0 Hz, 1H), 3.40 (t, J=8.0 Hz, 2H), 2.06-2.18 (broad m, 2H), 1.94 (m, 1H), 1.48 (m, 4H), 1.38 (s, 9H), 1.23 (d, J=8.0 Hz, 3H), 1.20 (m, 2H), 0.87 (d, J=8.0 Hz, 3H), 0.83 (d, J=8.0 Hz, 3H). UPLC/MS 1.88 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flow rate was 0.8 mL/min.), m/z 577.6 [M+Na]+.

### Step 2

Trifluoroacetic acid (5 ml) was added to a solution of tert-butyl (6-(3-(4-cyanophenoxy)-2,5-dioxo-2,5-dihydro-1H-pyrrol-1-yl)hexanoyl)-L-valyl-L-alaninate (501 mg, 887 umol) in methylene chloride (5 ml). After stirring for 2 h, the solution was concentrated under reduced pressure. The residue was diluted with 1:1 acetonitrile:water and lyophilized to yield 466 mg (100% yield) of (6-(3-(4-cyanophenoxy)-2,5-dioxo-2,5-dihydro-1H-pyrrol-1-yl)hexanoyl)-L-valyl-L-alanine (18) as a white solid. ¹H NMR (400 MHz, DMSO-d₆) δ ppm 8.23 (d, J=8.0 Hz, 1H), 8.00 (d, J=8.0 Hz, 2H), 7.75 (d, J=8.0 Hz, 1H), 7.58 (d, J=8.0 Hz, 2H), 5.86 (s, 1H), 4.21 (dd, J=8.0 Hz and 8.0 Hz, 1H), 4.10 (pent., J=8.0 Hz, 1H), 3.40 (t, J=8.0 Hz, 2H), 2.06-2.18 (broad m, 2H), 1.94 (m, 1H), 1.48 (m, 4H), 1.23 (d, J=8.0 Hz, 3H), 1.20 (m, 2H), 0.87 (d, J=8.0 Hz, 3H), 0.83 (d, J=8.0 Hz, 3H). UPLC/MS 1.34 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flow rate was 0.8 mL/min.), m/z 521.6 [M+Na]⁺.

### Example 5: Synthesis of Linker-Cytotoxin Conjugates

### Example 5A

Linker-cytotoxin conjugates, including DBM linker-cytotoxin conjugates, may be synthesized a follows.

### Synthesis of (S)-2-((2R,3R)-3-((S)-1-((3R,4S,5R)-4-((S)-2-((S)-2-(6-(3,4-dibromo-2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (“DBM(C6)-MMAF”)

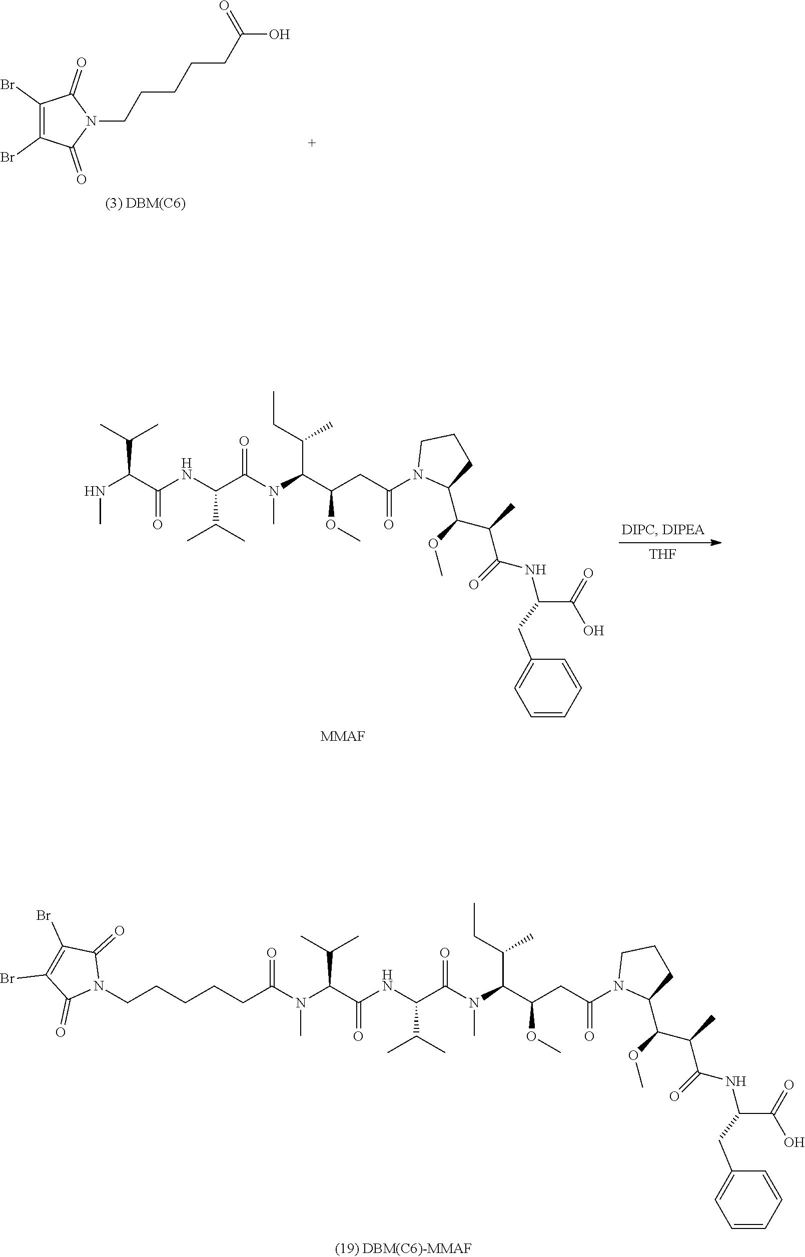

Procedure:

DIPC (34 mg, 0.271 mmol) and DIPEA (35 mg, 0.271 mmol) were added to a solution of 6-(3,4-dibromo-2,5-dioxo-2,5-dihydro-1H-pyrrol-1-yl)hexanoic acid (3) (250 mg, 0.677 mmol) in DCM (3 mL) and the resulting solution was stirred for 1 h at room temperature. (S)-2-((2R,3R)-3-((S)-1-((3R,4S,5S)-4-((S)—N,3-Dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid hydrochloride (MMAF.HCl) (208 mg, 0.271 mmol) was added in 50 mg portions over a 4 hr period and the resulting solution was stirred for a further 16 h. The DCM was removed under vacuum and the residue was purified by preparative HPLC. Lyophilization of the appropriate fractions gave (S)-2-((2R,3R)-3-((S)-1-((3R,4S,5S)-4-((S)-2-((S)-2-(6-(3,4-dibromo-2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (19) (170 mg, 0.156 mmol, 58%). ¹H NMR (500 MHz, CDCl₃) 7.15-7.26 (m, 5H), 4.60-4.92 (m, 4H), 3.70-4.20 (m, 4H), 3.59-3.63 (m, 2H), 3.39-3.42 (m, 1H), 3.26-3.35 (m, 6H), 2.93-3.09 (m, 6H), 2.20-2.60 (m, 6H), 1.70-2.15 (m, 4H), 1.61-1.69 (m, 8H), 1.25-1.37 (m, 3H), 1.15 (dd, J=18.5, 7.5 Hz, 2H), 0.81-1.05 (m, 20H). LC/MS 4.297 min (5-95% acetonitrile in water over 5 min), m/z 1083.3 [M+H].

Similar synthesis using MMAE in place of MMAF gives 6-(3,4-dibromo-2,5-dioxo-2,5-dihydro-1H-pyrrol-1-yl)-N—((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)-N-methylhexanamide (“DBM(C6)-MMAE”), depicted below:

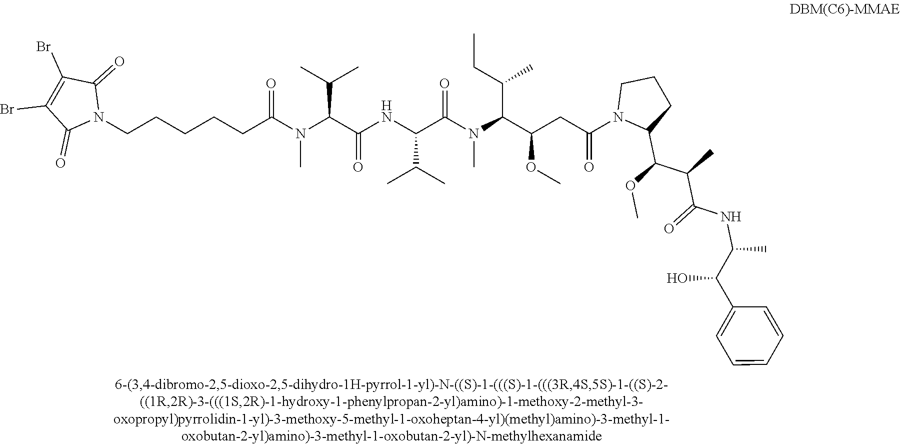

Similarly, by replacing 6-(3,4-dibromo-2,5-dioxo-2,5-dihydro-1H-pyrrol-1-yl)hexanoic acid (3) (DBM(C6)) with any one of the DBM-linkers synthesized according to Examples 1 and 2 (e.g., DBM(C7), DBM(C8), DBM(C9), DBM(C10), DBM(C11), or DBM(C12)) and Example 3 (e.g., DBM(PEG1), DBM(PEG2), or DBM(PEG3)), DBM-linker-MMAF and/or DBM-linker-MMAE conjugates may be made comprising an alkyl or alkylether linker of varying length.

### Example 5B

### Synthesis of (S)-2-((2R,3R)-3-((S)-1-((3R,4S,5S)-4-((S)-2-((S)-2-(6-(3-(4-cyanophenoxy)-2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (“CPM(C6)-MMAF”)

Procedure:

The target compound was synthesized using standard solid phase peptide synthesis protocols using Fmoc protected amino acids. Briefly, 1 gram of Fmoc-phenylalanine-2-chlorotrityl resin (0.6 mmol/gram) was suspended in 20 ml of DMA:dichloromethane (1:1) and purged with argon for 5 minutes. The solvent was then removed under vacuum and 10 mL of 20% piperidine in DMA was added. The suspension was purged with argon for 30 min at 20° C. The solvent was removed via vacuum filtration and the resin washed 3× with 10 mL DMA followed by 3× with dichloromethane. In a separate 20 mL glass vial, Fmoc-Dap (0.82 g, 2 mmol) was dissolved in 10 ml of DMA:dichloromethane and 0.76 g (2 mmol) of HATU was added followed by 0.4 mL (4 eq.) of N-methyl morpholine (NMM). The mixture was shaken gently until the solids had completely dissolved and then added to the deprotected Phe-2-chlorotrityl resin. The resin was gently purged with argon for 2 h at 20° C. and the solvent was removed by vacuum filration. The resin was then washed with DCM (3×20 mL) and DMA (3×20 mL). Fmoc deprotection was achieved by addition of 20 mL of 20% piperidine in DMA and the resin purged with argon for 30 min. Solvent was removed under vacuum and the resin washed with DMA (3×20 mL) and DCM (3×20 mL) to remove residual piperidine. Fmoc-Dil (0.76 g, 2 mmol) was activated with HATU as described above, and coupled to the deprotected Phe resin for 2 hr. The resin was filtered and washed with DMA (3×) and dichloromethane (3×) as described previously. The coupling steps and deprotection were repeated with Fmoc-Val and Fmoc-N-methyl valine and the resin was washed as described above. A small aliquot of resin was removed and treated with 10% acetic acid in DCM to confirm the presence of Fmoc-MMAF. The Fmoc group was deprotected and the final coupling step was performed via addition of 2 eq. of CPM-linker, 2 eq. of HATU and 5 eq. DIPEA in 20 mL of DMF to the resin. The reaction mixture was purged gently with nitrogen for 2 h at 20° C. The resin was washed as described above to remove unreacted reagents and a final wash with 2×50 mL of methanol was performed. The final product was cleaved from the resin via addition of a solution of 20 mL of 10% acetic acid and 10% trifluoroethanol in dichloromethane. The mixture was purged with nitrogen for 30 min. and the mixture was filtered through a course glass funnel. The solvent was evaporated to afford crude product. The crude material was purified via preparative reverse phase HPLC performed on a 50×250 mm C18 column with a flow rate of 20 mL per minute. The product was eluted via a gradient of 30-90% acetonitrile in water over 60 minutes. Pure fractions were combined and lyophilized to afford CPM(C6)-MMAF as a white solid. m/z 1064.5 [M+Na].

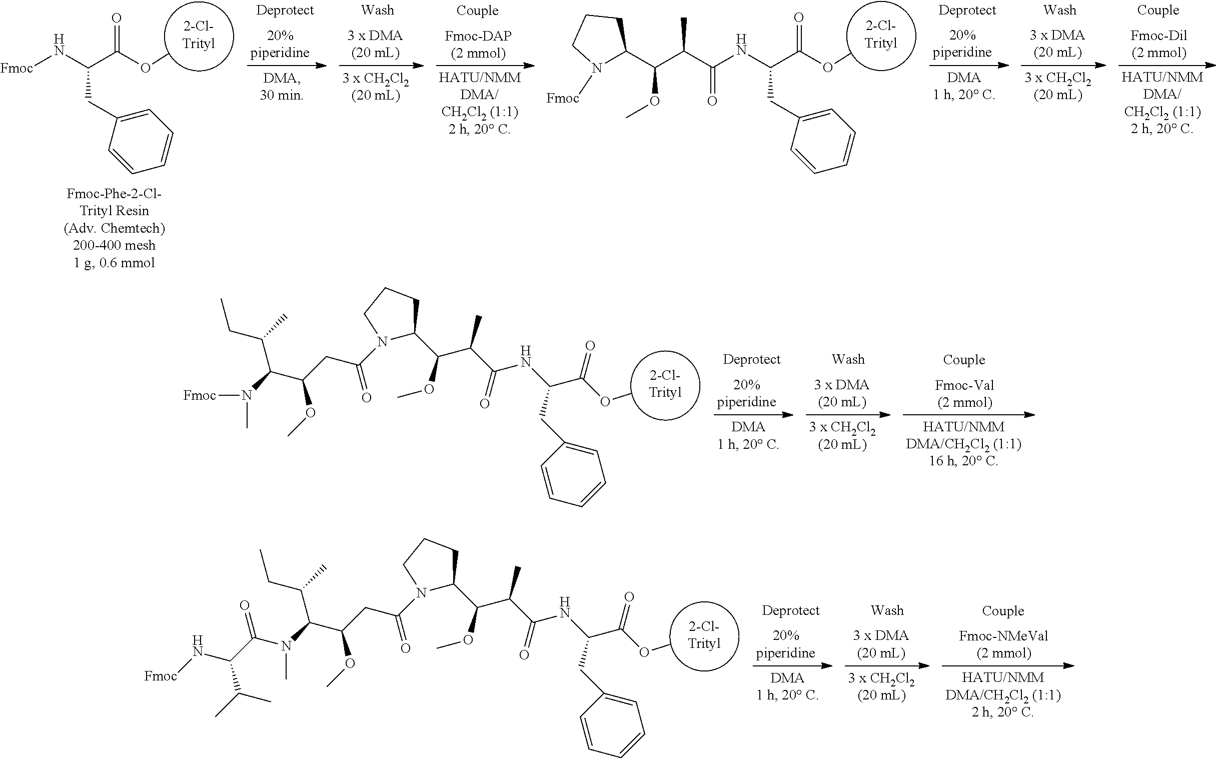

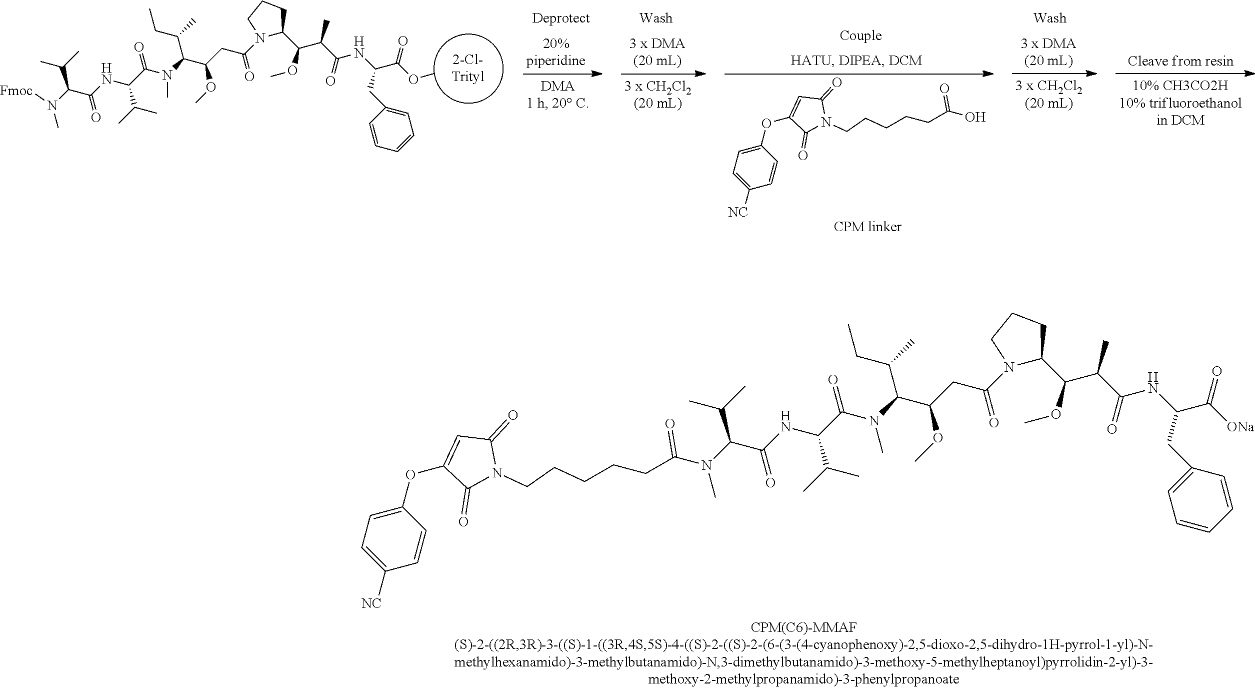

Similarly, by replacing the CPM linker depicted in the scheme above with the corresponding BRM linker, the following linker-cytotoxin conjugate may be synthesized:

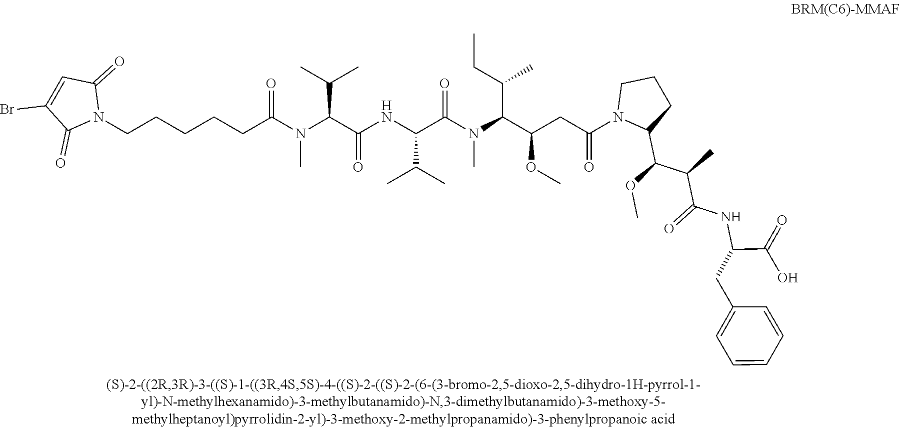

### Example 5C

Linker-cytotoxin conjugates, including CPM linker-cytotoxin conjugates, may be synthesized a follows.

### Synthesis of ((S)-2-((2R,3R)-3-((S)-1-((3R,4S,5S)-4-((S)-2-((S)-2-(6-(3-(4-cyanophenoxy)-2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (“CPM(C6)-MMAF”)

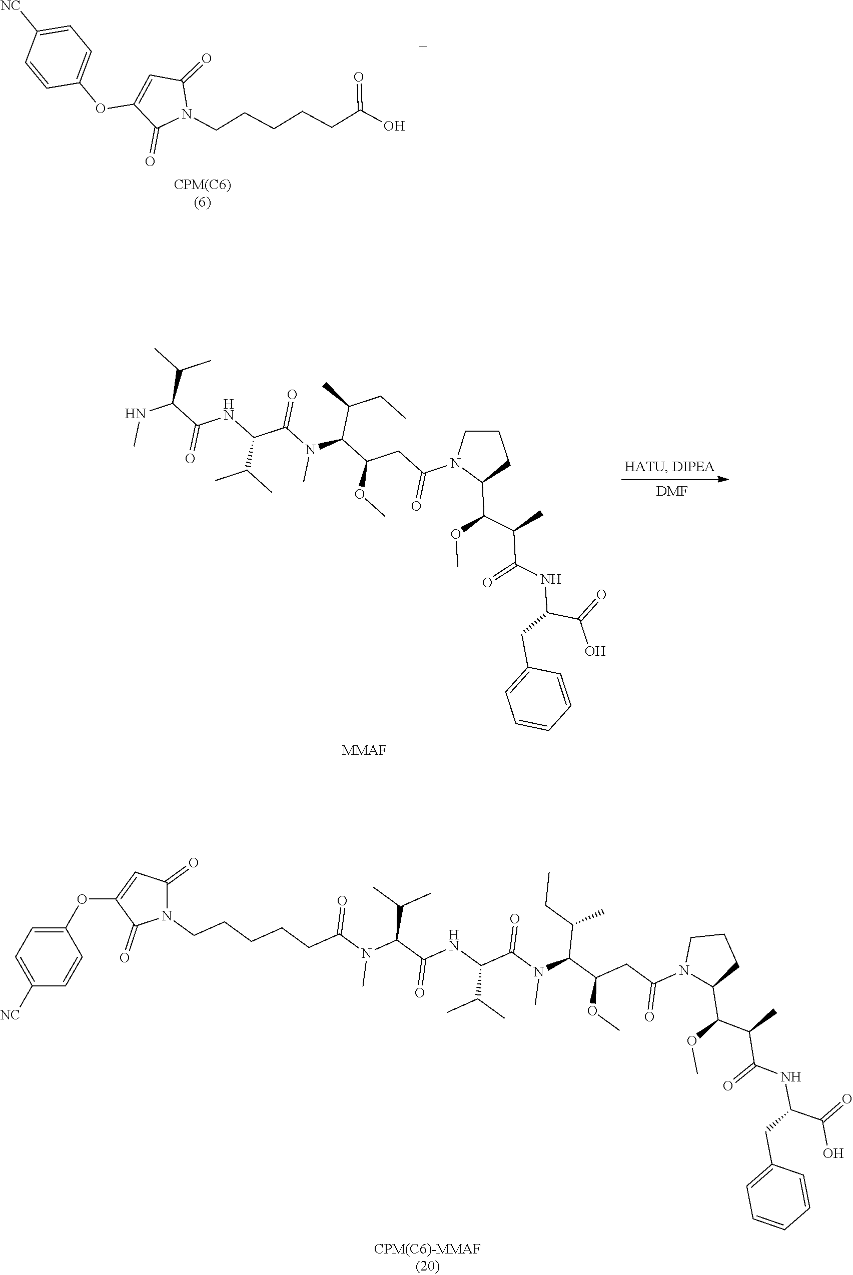

Procedure:

6-(3-(4-cyanophenoxy)-2,5-dioxo-2,5-dihydro-1H-pyrrol-1-yl)hexanoic acid (6) (10.2 mg) and HATU (11.8 mg) were dissolved in 0.25 mL dimethylformamide. DIPEA (12 mg) was added and after stirring solution for 1 min (S)-2-((2R,3R)-3-((S)-1-((3R,4S,5S)-4-((S)—N,3-Dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid hydrochloride (MMAF.HCl) (26.2 mg) was added. After 15 min of stirring at room temperature the product was purified by preparative HPLC. Lyophilization of the appropriate fractions gave ((S)-2-((2R,3R)-3-((S)-1-((3R,4S,5S)-4-((S)-2-((S)-2-(6-(3-(4-cyanophenoxy)-2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (20 mg, 63%) (20). ¹H NMR (400 MHz, CD₃OD) 7.8 (m, 2H), 7.5 (d, 2H) 7.15-7.26 (m, 5H), 5.6 (s, 1H), 4.50-4.92 (m, 4H), 3.60-4.20 (m, 4H), 3.45-3.55 (m, 2H), 3.39-3.42 (m, 1H), 3.26-3.35 (m, 6H), 2.85-3.09 (m, 6H), 2.20-2.50 (m, 6H), 1.70-2.15 (m, 4H), 1.50-1.69 (m, 8H), 1.25-1.37 (m, 3H), 1.15 (dd, 2H), 0.81-1.05 (m, 20H). LC/MS 1.88 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flowrate was 0.8 mL/min.), m/z 1042.65 [M+H]⁺.

Similarly, by replacing 6-(3-(4-cyanophenoxy)-2,5-dioxo-2,5-dihydro-1H-pyrrol-1-yl)hexanoic acid (CPM(C6)), with any one of the CPM-linkers synthesized according to Examples 1-3, CPM-linker-MMAF conjugates may be made comprising an alkyl or alkylether linker of varying length (e.g., CPM(C7), CPM(C8), CPM(C9), CPM(C10), CPM(C11), CPM(C12), CPM(PEG1), CPM(PEG2), or CPM(PEG3)).

### Example 6: Synthesis of Additional Linker-Cytotoxin Conjugates

### Example 6A

Additional linker-cytotoxin conjugates, including conjugates with cleavable linkers, may be synthesized a follows.

### Synthesis of 4-((R)-2-((R)-2-(6-(3,4-dibromo-2,5-dioxo-2,5-dihydro-1H-pyrrol-1-yl)hexanamido)-3-methylbutanamido)propanamido)benzyl ((S)-1-(((S)-1-(((3R,4S,5R)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (“DBM(C6)-Val-Ala-PAB-MMAE”)

### Step 1. Synthesis of (9H-Fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (“Fmoc-VAP-PNC”)

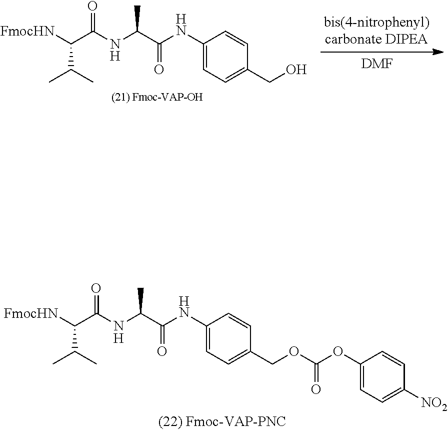

Procedure:

(9H-Fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (21) (Fmoc-VAP-OH) (200 mg, 0.387 mmol) was dissolved in DMF (2 mL) and bis(4-nitrophenyl) carbonate (141 mg, 0.465 mmol) and DIPEA (200 mg, 1.55 mmol) were added. The resulting solution was stirred for 4 h at room temperature. The reaction was concentrated under vacuum and purified by silica gel chromatography (DCM/EtOAc 0-100%). Concentration of the appropriate fractions gave (9H-fluoren-9-yl)methyl((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (Fmoc-VAP-PNC) (22) (242 mg, 0.355 mmol, 92%). LC/MS 4.480 min (5-95% acetonitrile in water over 5 min), m/z 703.3 [M+Na].

### Step 2. Synthesis of 4-((S)-2-((S)-2-((((9H-Fluoren-9-yl)methoxy)carbonyl)amino)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (“Fmoc-VAP-MMAE”)

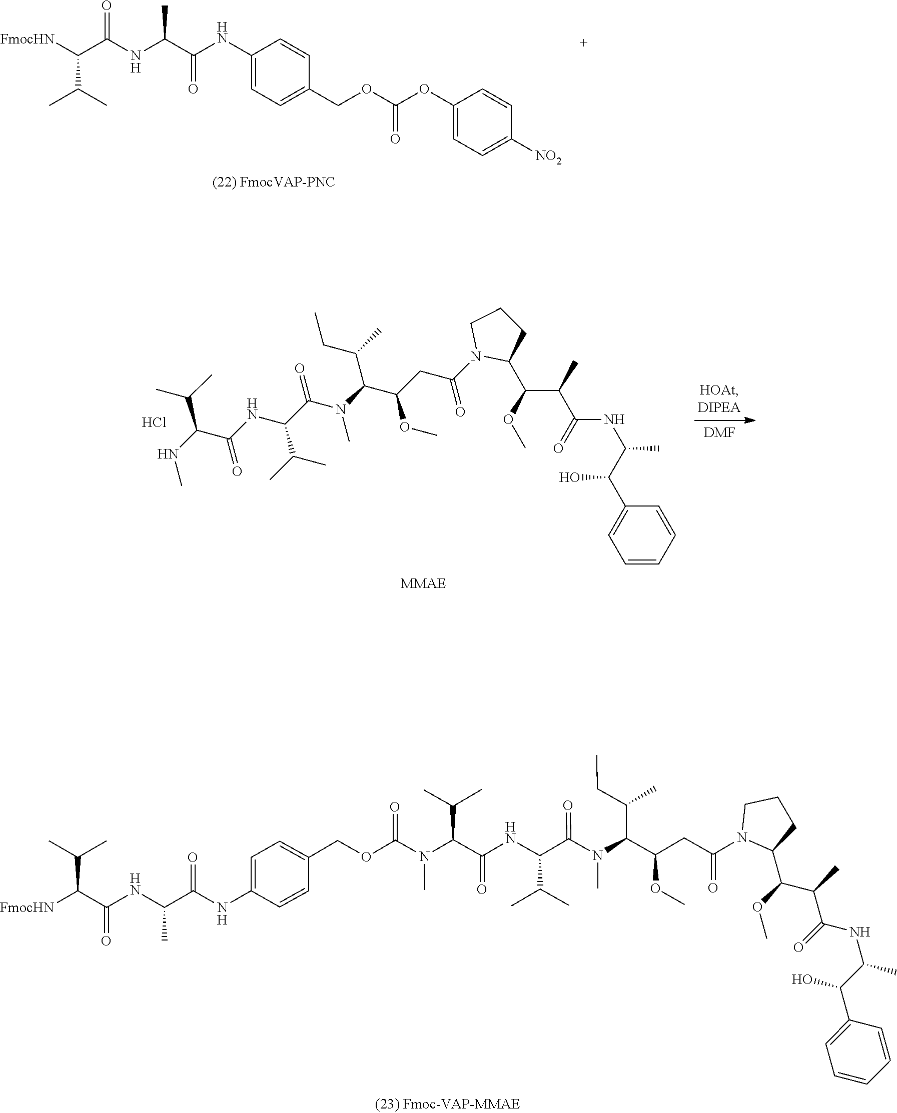

Procedure:

(9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (22) (Fmoc-VAP-PNC) (20 mg, 0.02938 mmol) was dissolved in DMF (0.5 mL) and MMAE.HCl (17 mg, 0.02351 mmol), HOAt (2 mg, 0.01469 mmol) and DIPEA (8 mg, 0.0587 mmol) were added. The resulting solution was stirred for 18 h at room temperature. The DMF was removed under vacuum and the residue was purified by silica gel chromatography (eluent methylene chloride/methanol 0-20%). Concentration of the appropriate fractions gave 4-((S)-2-((S)-2-((((9H-fluoren-9-yl)methoxy)carbonyl)amino)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (23) (Fmoc-VAP-MMAE) (32 mg, 0.025 mmol, 86%). LC/MS 4.649 min (5-95% acetonitrile in water over 5 min), m/z 1259.6 [M+H].

### Step 3. Synthesis of ((S)-1-(((S)-1-(((3R,4S,5S)-1-((S)-2-((1R,2R)-3-(((1S,2R)-1-Hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (“VAP-MMAE”)

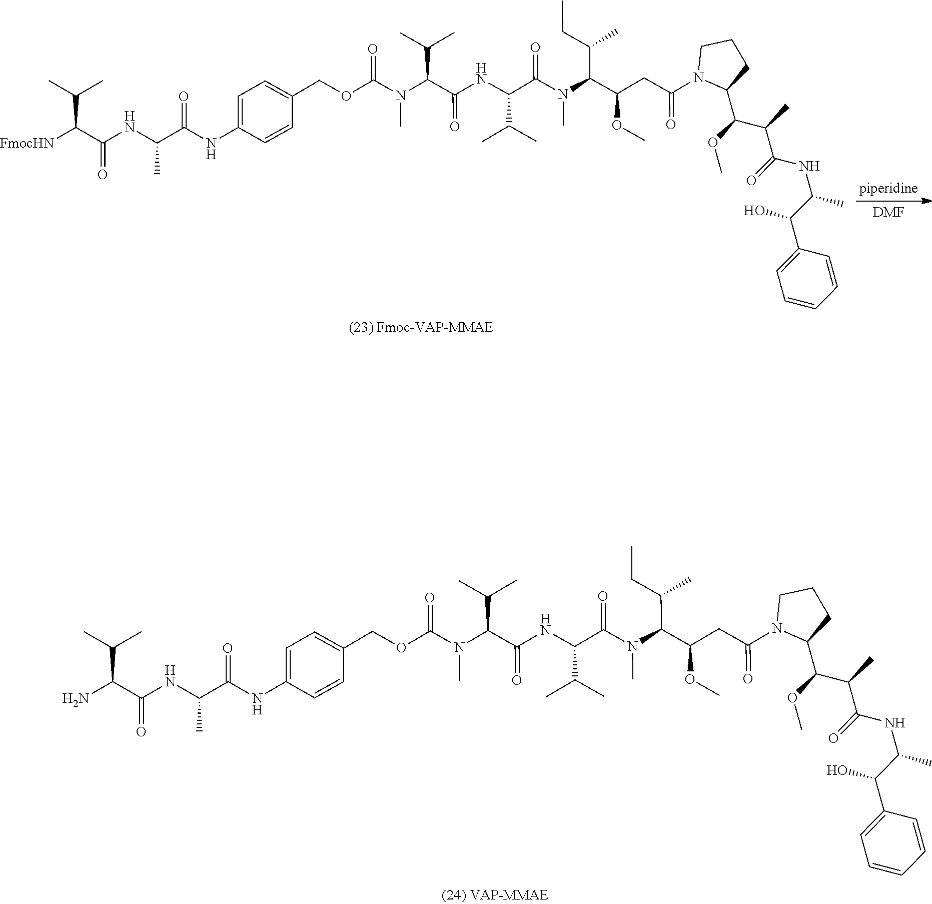

Procedure:

4-((S)-2-((S)-2-((((9H-Fluoren-9-yl)methoxy)carbonyl)amino)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (23) (Fmoc-VAP-MMAE) (42 mg, 0.0334 mmol) was dissolved in DMF (0.5 mL) and piperidine (0.1 mL, of a 20% solution in DMF) was added. The resulting solution was stirred for 1 h at room temperature. The DMF was removed under a stream of air and the residue was purified by preparative HPLC. Lyophilization of the appropriate fractions gave 4-((S)-2-((S)-2-amino-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (24) (VAP-MMAE) (29 mg, 0.02796 mmol, 84%). LC/MS 3.295 min (5-95% acetonitrile in water over 5 min), m/z 1037.6 [M+H].

### Step 4. Synthesis of 4-((S)-2-((S)-2-(6-(3,4-Dibromo-2,5-dioxo-2,5-dihydro-1H-pyrrol-1-yl)hexanamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (“DBM(C6)-VAP-MMAE”)

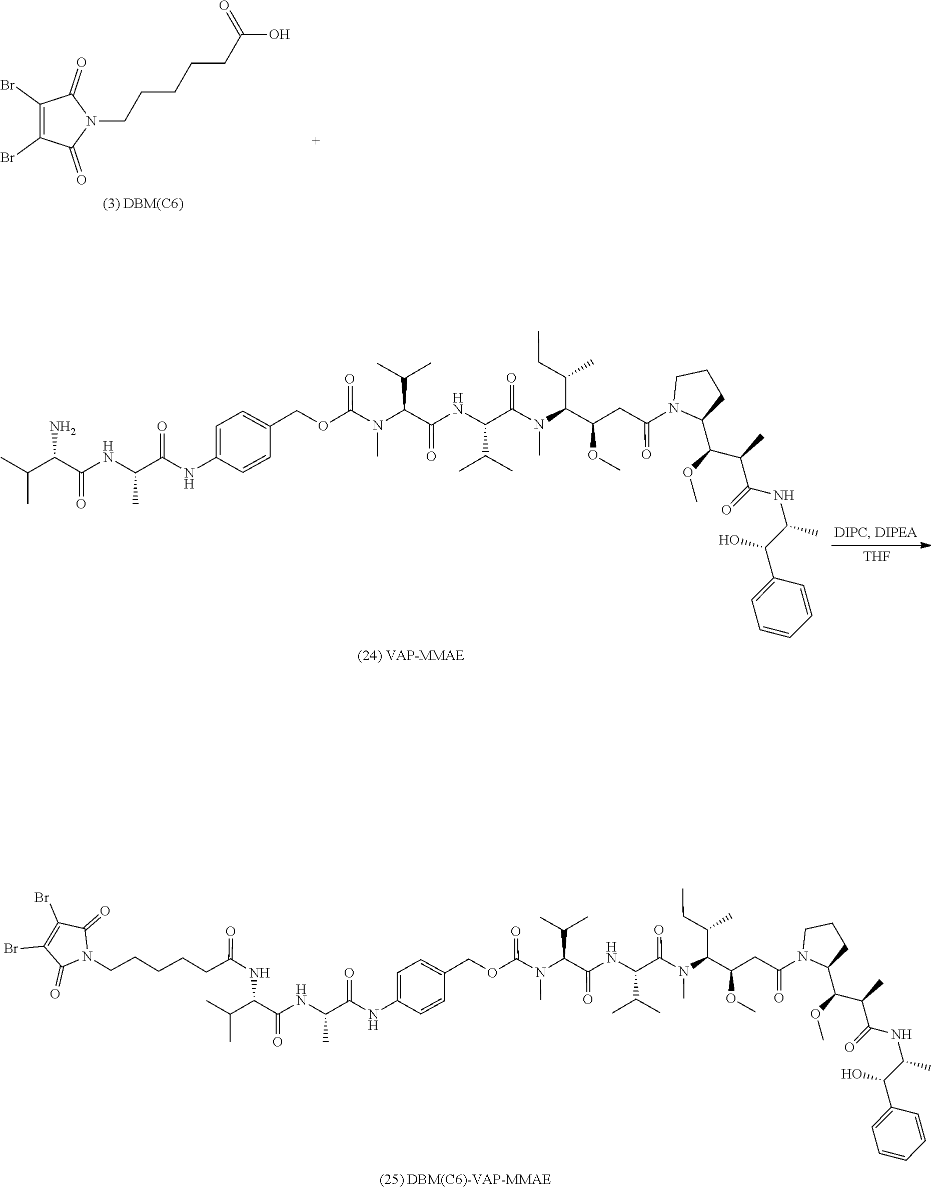 [SMILES: O=C(O)CCCCCN1C(=O)C(Br)=C(Br)C1=O ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](N)C(C)C)cc1)C(C)C ; "THF" ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1)C(C)C]

Procedure:

6-(3,4-Dibromo-2,5-dioxo-2,5-dihydro-1H-pyrrol-1-yl)hexanoic acid (3) (DBM(C6)) (20 mg, 0.0542 mmol) was dissolved in THF (0.5 mL) and DIPC (3.4 mg, 0.0271 mmol) and DIPEA (7 mg, 0.0542 mmol) were added. The resulting solution was stirred for 1 h and LCMS indicated a mixture of unreacted acid, symmetrical anhydride and isourea. 4-((S)-2-((S)-2-Amino-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (VAP-MMAE) (24) (28 mg, 0.0271 mmol) was added and the resulting solution was stirred for a further 6 h. The THF was removed under vacuum and the residue was purified by preparative HPLC. Lyophilization of the appropriate fractions gave 4-((S)-2-((S)-2-(6-(3,4-dibromo-2,5-dioxo-2,5-dihydro-1H-pyrrol-1-yl)hexanamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (25) (DBM(C6)-VAP-MMAE) (35 mg, 0.0252 mmol, 47%). LC/MS 4.306 min (5-95% acetonitrile in water over 5 min), m/z 1388.5 [M+H]. ¹H NMR (400 MHz, CDCl₃) δ7.60-7.48 (m, 2H), 7.41-7.28 (m, 7H), 5.25-4.81 (m, 3H), 4.72-4.59 (m, 3H), 4.38-4.30 (m, 2H), 4.20-4.11 (m, 2H), 3.93-4.08 (m, 1H), 3.72-3.90 (m, 1H), 3.72-3.60 (m, 2H), 3.40 (s, 3H), 3.89-3.29 (m, 3H), 3.01 (s, 3H), 2.90 (s, 3H), 2.81-2.60 (m, 4H), 2.50-2.31 (m, 3H), 2.30-2.18 (m, 3H), 2.15-2.10 (m, 3H), 1.89-1.55 (m, 8H), 1.49-1.40 (m, 3H), 1.38-1.20 (m, 7H), 1.10-0.63 (m, 25H).

Similar synthesis using (9H-fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl)carbamate (Fmoc-VCP-OH) in place of (9H-Fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (21) (Fmoc-VAP-OH) in step 1, gives 4-((S)-2-((S)-2-(6-(3,4-dibromo-2,5-dioxo-2,5-dihydro-1H-pyrrol-1-yl)hexanamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-1-oxo-5-ureidopentan-2-yl)amino)-3-methyl-1-oxobutan-2-yl)(methyl)carbamate (“DBM(C6)-VCP-MMAE”), depicted below:

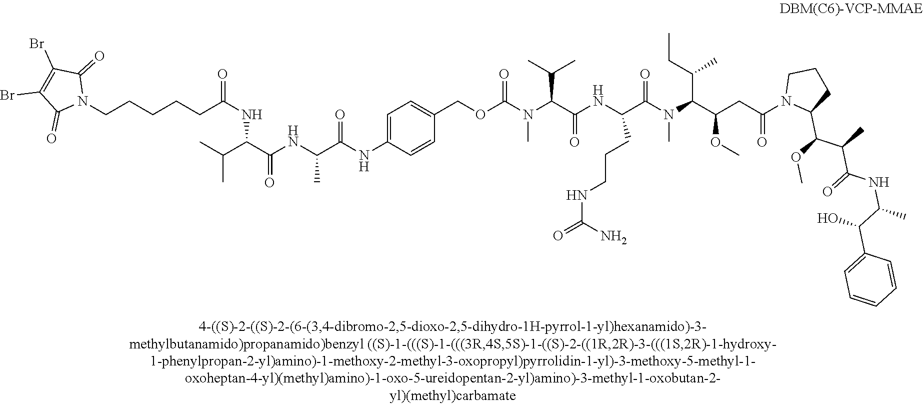 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@H](CCCNC(N)=O)NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1]

Similar synthesis using MMAF in place of MMAE in step 2 gives (S)-2-((2R,3R)-3-((S)-1-((5S,8S,11S,12R)-11-((S)-sec-butyl)-1-(4-((S)-2-((S)-2-(6-(3,4-dibromo-2,5-dioxo-2,5-dihydro-1H-pyrrol-1-yl)hexanamido)-3-methylbutanamido)propanamido)phenyl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-3-phenylpropanoic acid (“DBM(C6)-VAP-MMAF”), depicted below:

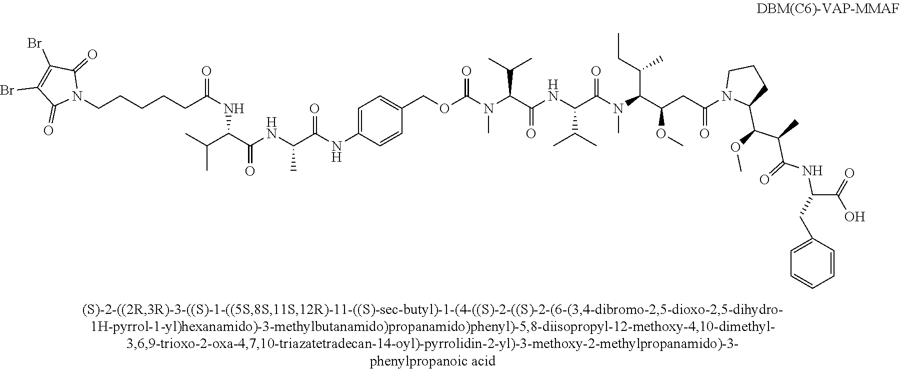 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1)C(C)C]

Similar synthesis using (9H-fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl)carbamate (Fmoc-VCP-OH) in place of (9H-Fluoren-9-yl)methyl ((S)-1-(((S)-1-((4-(hydroxymethyl)phenyl)amino)-1-oxopropan-2-yl)amino)-3-methyl-1-oxobutan-2-yl)carbamate (21) (Fmoc-VAP-OH) in step 1, and MMAF in place of MMAE in step 2, gives (S)-2-((2R,3R)-3-((S)-1-((5S,8S,11S,12R)-11-((S)-sec-butyl)-1-(4-((S)-2-((S)-2-(6-(3,4-dibromo-2,5-dioxo-2,5-dihydro-1H-pyrrol-1-yl)hexanamido)-3-methylbutanamido)propanamido)phenyl)-5-isopropyl-2-methoxy-4,10-dimethyl-3,6,9-trioxo-8-(3-ureidopropyl)-2-oxa-4,7,10-triazatetradecan-14-oyl)pyrrolidin-2-yl)-3-methoxy-2-methylpropanamido)-3-phenylpropanoic acid (“DBM(C6)-VCP-MMAF”), depicted below:

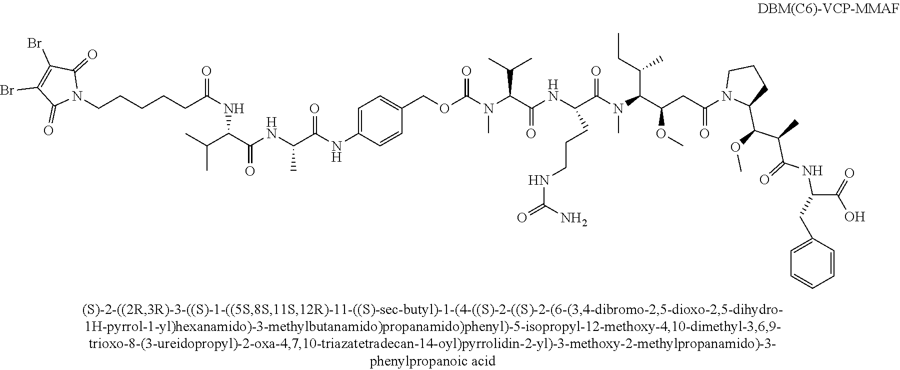 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@H](CCCNC(N)=O)NC(=O)[C@H](C(C)C)N(C)C(=O)OCc1ccc(NC(=O)[C@H](C)NC(=O)[C@@H](NC(=O)CCCCCN2C(=O)C(Br)=C(Br)C2=O)C(C)C)cc1]

Similarly, by replacing 6-(3,4-dibromo-2,5-dioxo-2,5-dihydro-1H-pyrrol-1-yl)hexanoic acid (3) (DBM(C6)) in step 4 with any one of the DBM-linkers synthesized according to Examples 1 and 2 (e.g., DBM(C7), DBM(C8), DBM(C9), DBM(C10), DBM(C11), or DBM(C12)) and Example 3 (e.g., DBM(PEG1), DBM(PEG2), or DBM(PEG3)), DBM-linker-MMAF and/or DBM-linker-MMAE conjugates may be made comprising an alkyl or alkylether linker of varying length.

Alternatively, by replacing 6-(3,4-Dibromo-2,5-dioxo-2,5-dihydro-1H-pyrrol-1-yl)hexanoic acid (3) (DBM(C6)) in step 4 with 6-(2,5-dioxo-2,5-dihydro-1H-pyrrol-1-yl)hexanoic acid, mc-VAP-MMAE, mc-VCP-MMAE, mc-VAP-MMAF, and mc-VCP-MMAE may be made, and converted to their dibrominated forms (DBM(C6)-VAP-MMAE, DBM(C6)-VCP-MMAE, DBM(C6)-VAP-MMAF, and DBM(C6)-VCP-MMAE respectively) through treatment with Br₂ in the presence of TEA.

### Example 6B

### Synthesis of (S)-2-((2R,3R)-3-((S)-1-((5S,8S,11S,12R)-11-((S)-sec-butyl)-1-(4-((S)-2-((S)-2-(6-(3-(4-cyanophenoxy)-2,5-dioxo-2,5-dihydro-1H-pyrrol-1-yl)hexanamido)-3-methylbutanamido)propanamido)phenyl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-3-phenylpropanoic acid (“CPM(C6)-Val-Ala-PAB-MMAF”)

Procedure:

The target compound was synthesized using standard solid phase peptide synthesis protocols using Fmoc protected amino acids. Briefly, 1 gram of Fmoc-phenylalanine-2-chlorotrityl resin (0.6 mmol/gram) was suspended in 20 ml of DMA:dichloromethane (1:1) and purged with argon for 5 minutes. The solvent was then removed under vacuum and 10 mL of 20% piperidine in DMA was added. The suspension was purged with argon for 30 min at 20° C. The solvent was removed via vacuum filtration and the resin washed 3× with 10 mL DMA followed by 3× with dichloromethane. In a separate 20 mL glass vial, Fmoc-Dap (0.82 g, 2 mmol) was dissolved in 10 ml of DMA:dichloromethane and 0.76 g (2 mmol) of HATU was added followed by 0.4 mL (4 eq.) of N-methyl morpholine (NMM). The mixture was shaken gently until the solids had completely dissolved and then added to the deprotected Phe-2-chlorotrityl resin. The resin was gently purged with argon for 2 h at 20° C. and the solvent was removed by vacuum filration. The resin was then washed with DCM (3×20 mL) and DMA (3×20 mL). Fmoc deprotection was achieved by addition of 20 mL of 20% piperidine in DMA and the resin purged with argon for 30 min. Solvent was removed under vacuum and the resin washed with DMA (3×20 mL) and DCM (3×20 mL) to remove residual piperidine. Fmoc-Dil (0.76 g, 2 mmol) was activated with HATU as described above, and coupled to the deprotected Phe resin for 2 hr. The resin was filtered and washed with DMA (3×) and dichloromethane (3×) as described previously. The coupling steps and deprotection were repeated with Fmoc-Val and Fmoc-N-methyl valine and the resin was washed as described above. A small aliquot of resin was removed and treated with 10% acetic acid in DCM to confirm the presence of Fmoc-MMAF. The Fmoc group was deprotected and the final coupling step was performed via addition of 2 eq. of 4-((S)-2-((S)-2-(6-(3-(4-cyanophenoxy)-2,5-dioxo-2,5-dihydro-1H-pyrrol-1-yl)hexanamido)-3-methylbutanamido)propanamido)benzyl hydrogen carbonate (CPM-VAP-NPC) in 10 mL of DMF to the resin. 10 mg of HOAT was then added and the reaction mixture was purged gently with nitrogen for 12 h at 20° C. The resin was washed as described above to remove unreacted reagents and a final wash with 2×50 mL of methanol was performed. The final product was cleaved from the resin via addition of a solution of 20 mL of 10% acetic acid and 10% trifluoroethanol in dichloromethane. The mixture was purged with nitrogen for 30 min. and the mixture was filtered through a course glass funnel. The solvent was evaporated to afford crude product. The crude material was purified via preparative reverse phase HPLC performed on a 50×250 mm C18 column with a flow rate of 20 mL per minute. The product was eluted via a gradient of 30-90% acetonitrile in water over 60 minutes. Pure fractions were combined and lyophilized to afford CPM(C6)-Val-Ala-PAB-MMAF as a white solid. M/z 1385 [M+Na].

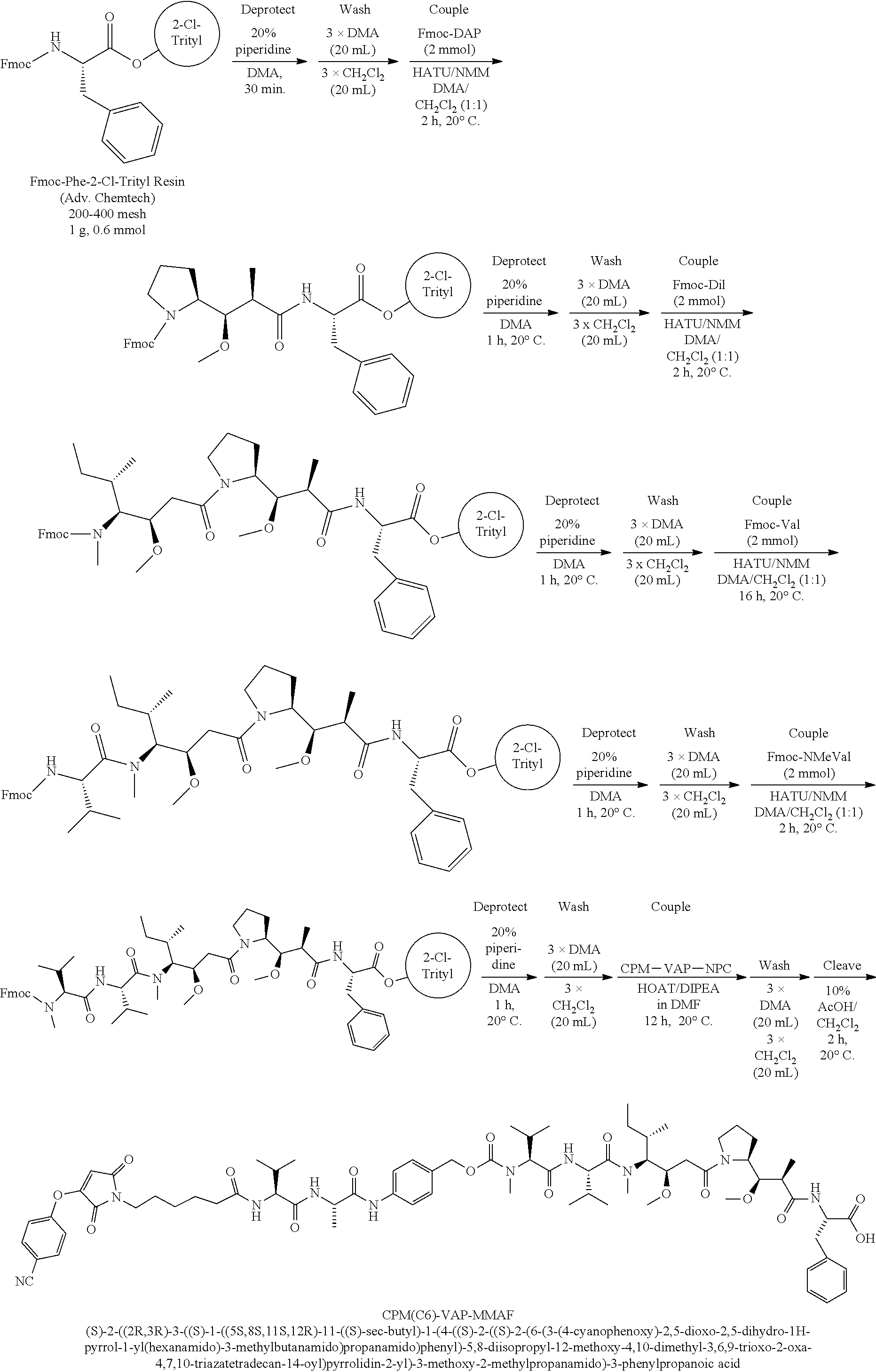

Using similar solid phase peptide synthesis protocols and Fmoc protected amino acids to replace MMAF with MMAE, the following linker-cytotoxin conjugate may be synthesized:

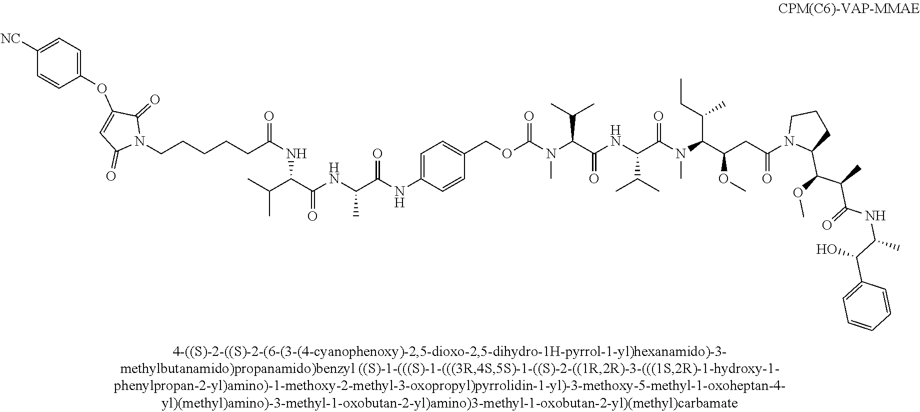 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C=C(Oc3ccc(C#N)cc3)C2=O)C(C)C)cc1)C(C)C]

### Example 6C

Additional linker-cytotoxin conjugates, including conjugates with cleavable linkers, may be synthesized a follows.

### Synthesis of 4-((S)-2-((S)-2-(6-(3-(4-cyanophenoxy)-2,5-dioxo-2,5-dihydro-1H-pyrrol-1-yl)hexanamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (“CPM(C6)-Val-Ala-PAB-MMAE”)

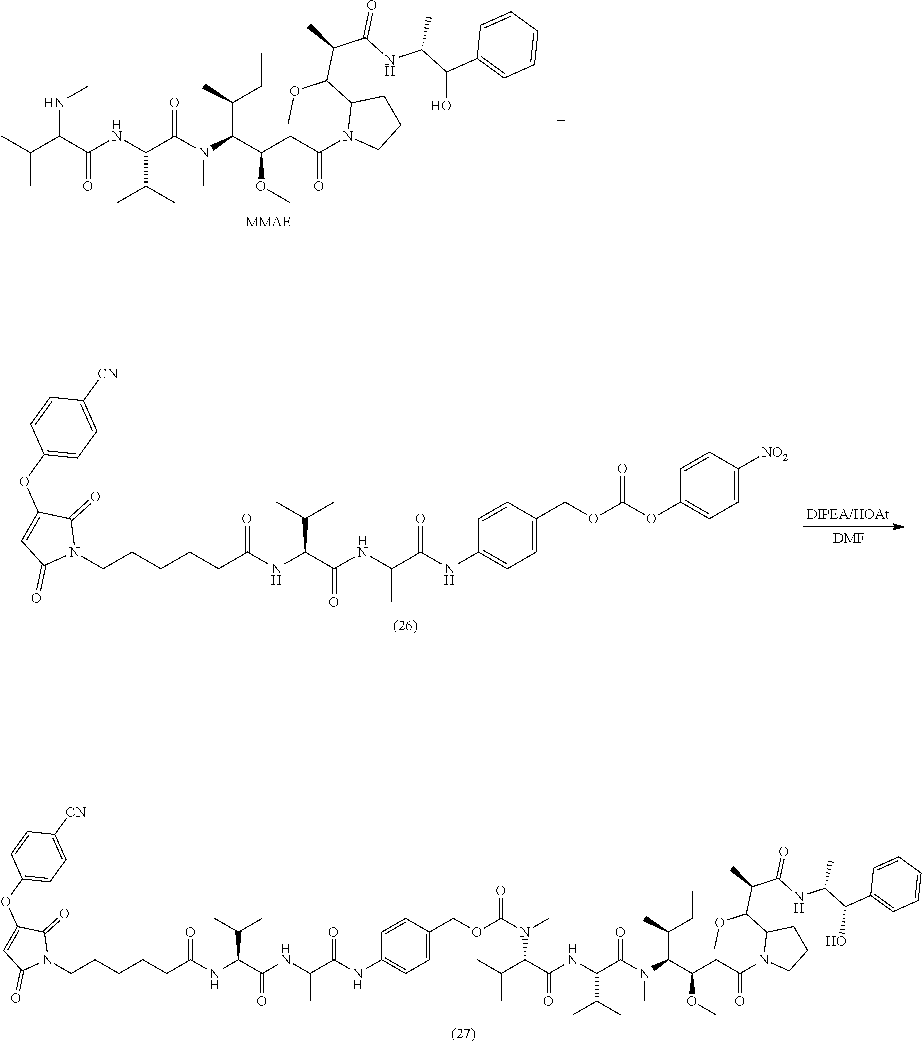

Procedure:

CPM-Val-Ala-PAB-NPC (26) 28 mg, (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (MMAE, 20 mg), DIPEA (6.2 mg), and HoAt (0.5 mg) were dissolved in 0.2 mL dimethylformamide. After 6 h at room temperature the reaction was purified via preparative HPLC and the appropriate fractions were lyophilized. A second purification via silica gel chromatography was required to give the purified product 4-((S)-2-((S)-2-(6-(3-(4-cyanophenoxy)-2,5-dioxo-2,5-dihydro-1H-pyrrol-1-yl)hexanamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (27) (CPM(C6)-Val-Ala-PAB-MMAE) (5 mg, 15%). LC/MS 2.01 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flowrate was 0.8 mL/min.), m/z 1369.86 [M+Na]⁺.

### Synthesis of 6-(3-(4-cyanophenoxy)-2,5-dioxo-2,5-dihydro-1H-pyrrol-1-yl)-N—((S)-1-(((S)-1-((4-((S)-7-methoxy-8-(3-(((S)-7-methoxy-2-(4-methoxyphenyl)-5-oxo-5,11a-dihydro-1H-benzo[e]pyrrolo[1,2-a][1,4]diazepin-8-yl)oxy)propoxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)hexanamide (“CPM(C6)-Val-Ala-PBD”)

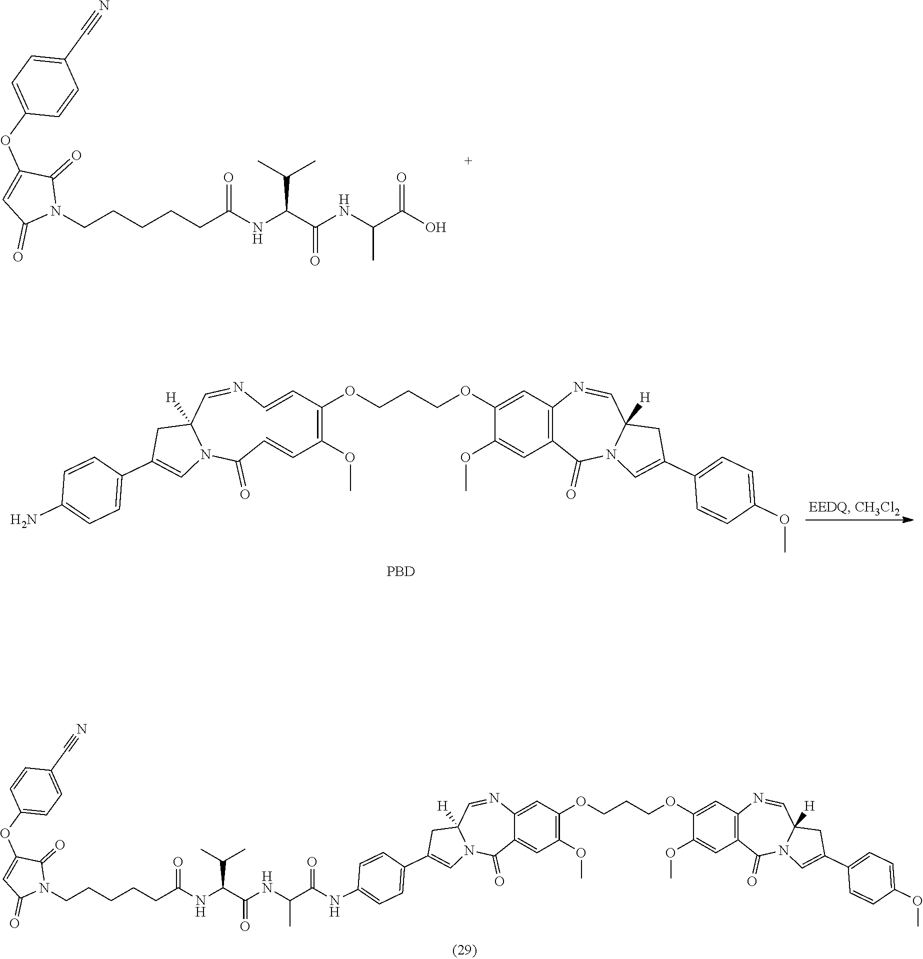 [SMILES: CC(NC(=O)[C@@H](NC(=O)CCCCCN1C(=O)C=C(Oc2ccc(C#N)cc2)C1=O)C(C)C)C(=O)O ; COC1=C(OCCCOc2cc3c(cc2OC)C(=O)N2C=C(c4ccc(OC)cc4)C[C@H]2C=N3)/C=C/N=C\[C@@H]2CC(c3ccc(N)cc3)=CN2C(=O)\C=C\1 ; "PBD" ; COc1ccc(C2=CN3C(=O)c4cc(OC)c(OCCCOc5cc6c(cc5OC)C(=O)N5C=C(c7ccc(NC(=O)C(C)NC(=O)[C@@H](NC(=O)CCCCCN8C(=O)C=C(Oc9ccc(C#N)cc9)C8=O)C(C)C)cc7)C[C@H]5C=N6)cc4N=C[C@@H]3C2)cc1]

Procedure:

6-(3-(4-cyanophenoxy)-2,5-dioxo-2,5-dihydro-1H-pyrrol-1-yl)hexanoyl)-L-valyl-L-alanine (60 mg, 120 umol) and ethyl 2-ethoxyquinoline-1(2H)-carboxylate (48 mg, 194 umol) in methylene chloride (2 ml) was stirred at 0 C for 1 h. (S)-2-(4-aminophenyl)-7-methoxy-8-(3-(((S)-7-methoxy-2-(4-methoxyphenyl)-5-oxo-5,11a-dihydro-1H-benzo[e]pyrrolo[1,2-a][1,4]diazepin-8-yl)oxy)propoxy)-1,11a-dihydro-5H-benzo[e]pyrrolo[1,2-a][1,4]diazepin-5-one (15 mg, 20.7 umol) in methylene chloride (1 ml) was added and the ice bath was removed. After stirring for 3 h, the solution was directly flash chromatographed on silica gel (40 g) with methylene chloride:methanol as the eluent 100:0 for 5 min then 100:0 to 80:20 over 20 min to afford 7 mg (28% yield) of 6-(3-(4-cyanophenoxy)-2,5-dioxo-2,5-dihydro-1H-pyrrol-1-yl)-N—((S)-1-(((S)-1-((4-((S)-7-methoxy-8-(3-(((S)-7-methoxy-2-(4-methoxyphenyl)-5-oxo-5,11a-dihydro-1H-benzo[e]pyrrolo[1,2-a][1,4]diazepin-8-yl)oxy)propoxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)hexanamide (29) (“CPM(C6)-Val-Ala-PBD”). UPLC/MS 1.65 min (5-95% acetonitrile/water+0.1% formic acid over 2 min, hold at 95% for 0.5 min, then 95-5% over 0.1 min, and hold at 5% for 0.4 min. Column used was Waters BEH C18 1.7 μm, 2.1×50 mm, flow rate was 0.8 mL/min.), m/z 1207.0 [M+H]⁺.

### Example 7: Antibody Disulfide Reduction and Linker-Cytotoxin Conjugation to Antibody

This example provides an exemplary protocol for reduction of the disulfides of the antibodies disclosed herein, and conjugation of the reduced antibodies to the linker-cytotoxin conjugates disclosed herein.

Protocol:

Step 1: Antibody Disulfide Reduction

A) Dilute antibody to 15 mg/mL (0.1 mM IgG) in PBS, pH 7.4.

B) Prepare a fresh 20 mM (5.7 mg/mL) stock solution of TCEP in H₂O.

C) Add 25 μL of TCEP stock solution from B to 1 mL of antibody from A (0.5 mM final concentration TCEP).

D) Incubate at 37° C. for 2 hours (check for free thiols using DTNB test).

E) Aliquot the reduced antibody into 4 tubes (250 μL each).

Step 1: Linker-Cytotoxin Conjugation to Antibody

A) Prepare 10 mM stock solution of linker-cytotoxin conjugate in DMSO (DMA, DMF or CH₃CN are also acceptable).

B) Add 5 equivalents of 12.5 μL stock solution from A to each tube of reduced antibody (0.5 mM final concentration linker-cytotoxin conjugate stock solution).

C) Incubate overnight at 4° C. for 4 hours at room temperature; check for free thiols using DTNB test.

D) Run analytical HIC to determine DAR and homogeneity.

### Example 8: Reduction and Purification of Antibodies for Conjugation to Linker-Cytotoxin Conjugate

This example provides an exemplary protocol for reduction and purification of an exemplary antibody, trastuzumab, for conjugation to the linker-cytotoxin conjugates disclosed herein.

Protocol:

Purge all buffers and DMSO stock solutions with Argon for 1 h prior to use.

1) Aliquot 1 mL of trastuzumab from 10 mg/mL stock into a 2 mL eppendorf tube.

2) Dilute with 1 mL 100 mM Borate (pH 8.4) to afford a 10 mg/mL stock solution (67 μM).

3) Prepare a 50 mM stock solution of TCEP in water.

4) Add 20 mL of TCEP to 2 mL of trastuzumab and incubate at 37° C. for 3 hours.

5) Aliquot into 4×0.5 mL eppendorf tubes and place 3 tubes in storage at −20° C.

6) Purify one 0.5 mL aliquot (approx. 5 mg) via SEC on Biorad using degassed PBS.

7) Collect monomeric antibody peak in a sealed tube (approx. 4 mL total volume) at 4° C.

8) Aliquot into 4 equal 1 mL eppendorf tubes (1 mg/mL).

9) Add 6 equivalents of the linker-cytotoxin conjugate from 2 mM stock solutions in DMSO to each tube.

10) Incubate at 4° C. for 48 hours.

11) Analyze by HIC, SDS-PAGE and LC/MS, and compare against control.

### Example 9: Synthesis of ADCs

### Example 9A

This example provides a general protocol for synthesis of ADCs, including DBM(C6)-MMAF ADCs, from any antibody, such as ADCs designated as follows: (A) trastuzumab-DBM(C6)-MMAF, (B) IGN523-DBM(C6)-MMAF, and (C) IGN786-DBM(C6)-MMAF.

Procedure:

All buffers and stock solutions are purged with argon prior to use to remove residual oxygen. Buffers and samples are tightly sealed throughout the duration of the conjugation. At least 1 mL of fresh linker stock solutions @ 2 mM is prepared in DMSO. 60 mg of purified antibody is buffer exchanged into 50 mM Borate pH 8 or PBS pH 7.4, and diluted to a final concentration of 2 mg/mL or 33 μM (30 mL total vol.). 6 molar equivalents of freshly prepared TCEP in water is added. The mixture is incubated at 37° C. for 2.5 h in a sealed tube, and then cooled to 4° C. on ice. To the cooled mixture is added 5 molar equivalents of DBM(C6)-MMAF from a 2 mM DMSO stock solution to give a final linker concentration of 0.2 mM. The resulting ADC product is incubated at 4° C. for 1.0 h. The crude ADC is buffer exchanged into PBS pH 7.4 to remove excess TCEP and DBM-MMAF. The ADC is diluted to a final concentration of 2 mg/mL in PBS pH 7.4, and stored at −20° C.

Following the above procedure, the following ADCs were made:

(A) trastuzumab-DBM(C6)-MMAF,

### (B) IGN523-DBM(C6)-MMAF, and

### (C) IGN786-DBM(C6)-MMAF.

ADC Analysis:

All DBM-MMAF ADCs were characterized for purity (% monomer), drugs/antibody, homogeneity, antigen binding, potency and selectivity for antigen expressing cells in vitro, efficacy in murine xenograft models and pharmacokinetics in rat.

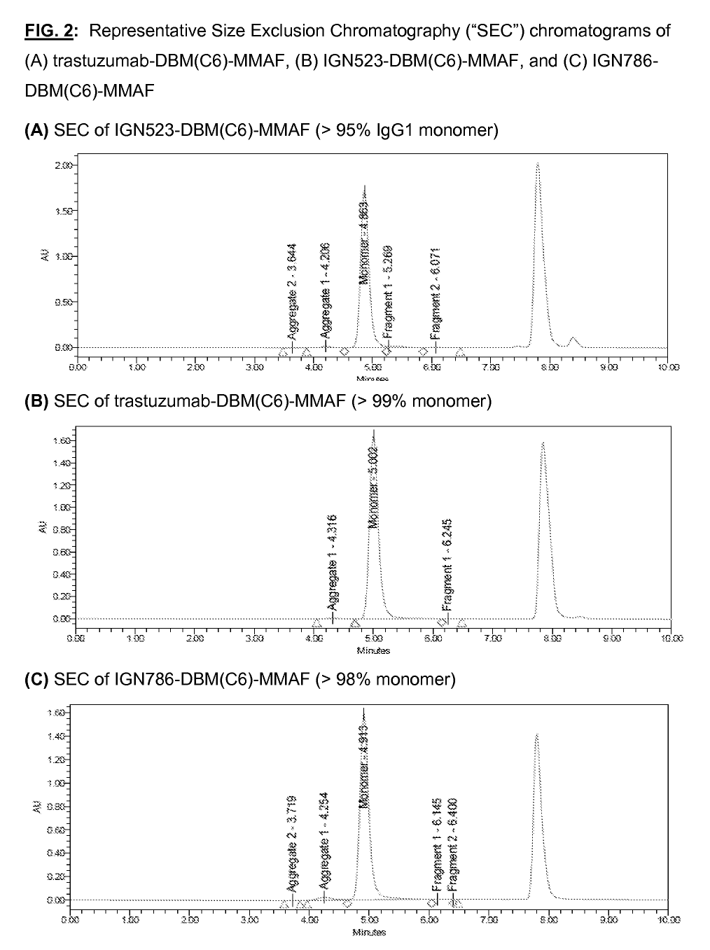

FIG. 2 shows representative Size Exclusion Chromatography (“SEC”) chromatograms of (A) trastuzumab-DBM(C6)-MMAF, (B) IGN523-DBM(C6)-MMAF, and (C) IGN786-DBM(C6)-MMAF, demonstrating >95%, >99%, and >98% monomer, respectively.

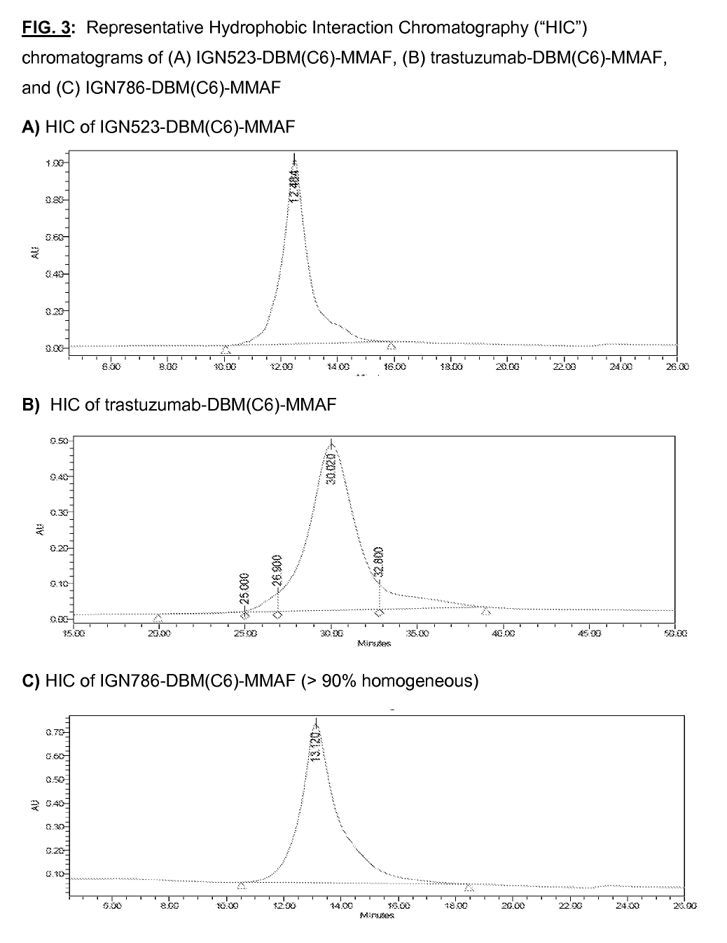

FIG. 3 shows representative Hydrophobic Interaction Chromatography (“HIC”) chromatograms of (A) IGN523-DBM(C6)-MMAF, (B) trastuzumab-DBM(C6)-MMAF, and (C) IGN786-DBM(C6)-MMAF, demonstrating the homogeneity of these ADCs.

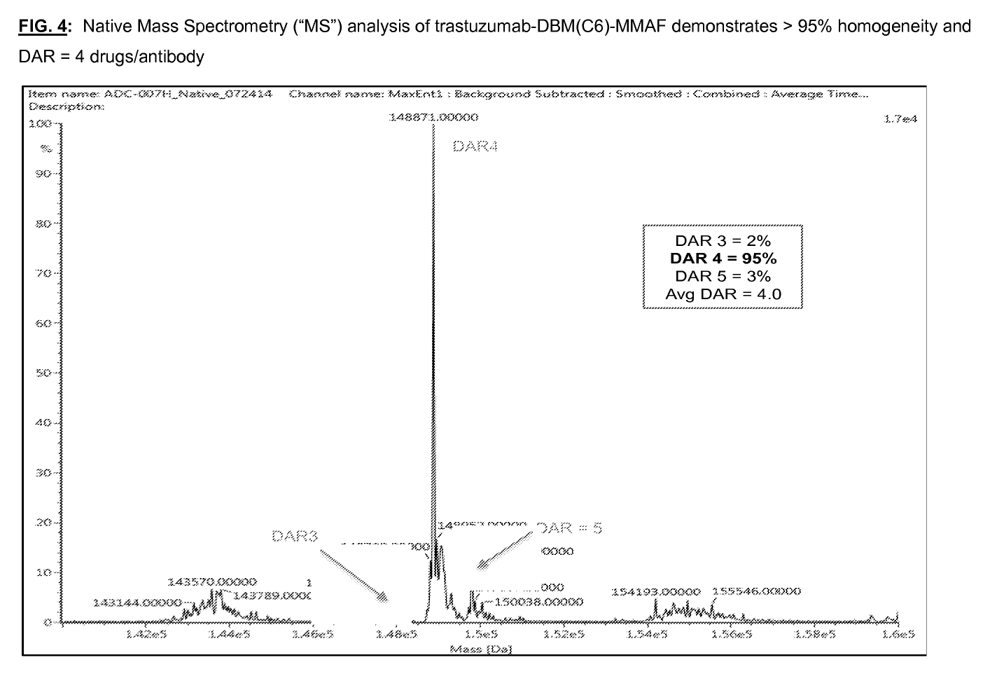

FIG. 4 shows native Mass Spectrometry (“MS”) analysis of trastuzumab-DBM(C6)-MMAF, demonstrating >95% homogeneity and DAR=4 drugs/antibody is obtained.

### Example 9B

This example provides a general protocol for synthesis of ADCs, including CPM(C6)-MMAF ADCs, from any antibody, such as ADCs designated as follows: (A) trastuzumab-CPM(C6)-MMAF, (B) IGN523-CPM(C6)-MMAF, and (C) IGN786-CPM(C6)-MMAF.

Procedure:

Antibody was prepared at 5-10 mg/mL in PBS+5 mM EDTA, pH 7.4. Eight equivalents (relative to antibody concentration) of TCEP from a freshly prepared solution were added to the antibody. The antibody was then incubated at 37° C. for 2 h. The antibody was then allowed to cool to room temperature, meanwhile 5 equivalents (relative to antibody concentration) of linker toxin in volume of DMSO equal to 1/9 the volume of antibody solution was prepared. After addition of the linker-toxin to antibody, the final concentration of DMSO was 10%. After 30 min reaction at room temperature the conjugate was purified by gel filtration or tangential flow filtration.

Following the above procedure, the following ADCs were made:

(A) trastuzumab-CPM(C6)-MMAF,

(B) IGN523-CPM(C6)-MMAF, and

(C) IGN786-CPM(C6)-MMAF.

ADC Analysis:

All CPM-MMAF ADCs were characterized for purity (% monomer), drugs/antibody, homogeneity, antigen binding, potency and selectivity for antigen expressing cells in vitro, efficacy in murine xenograft models and pharmacokinetics in rat.

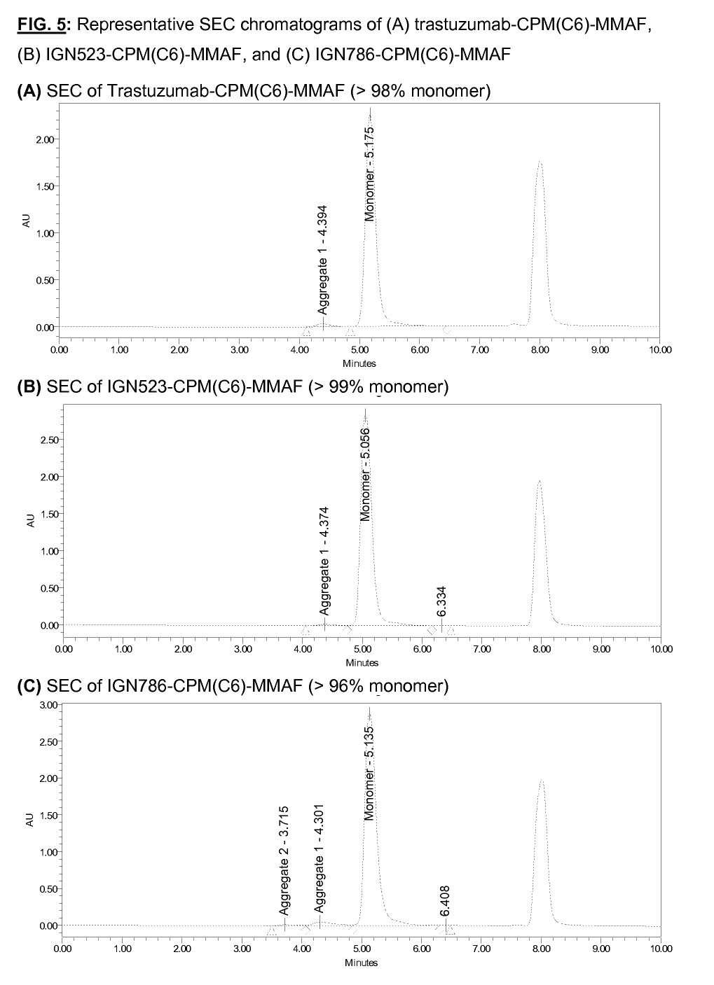

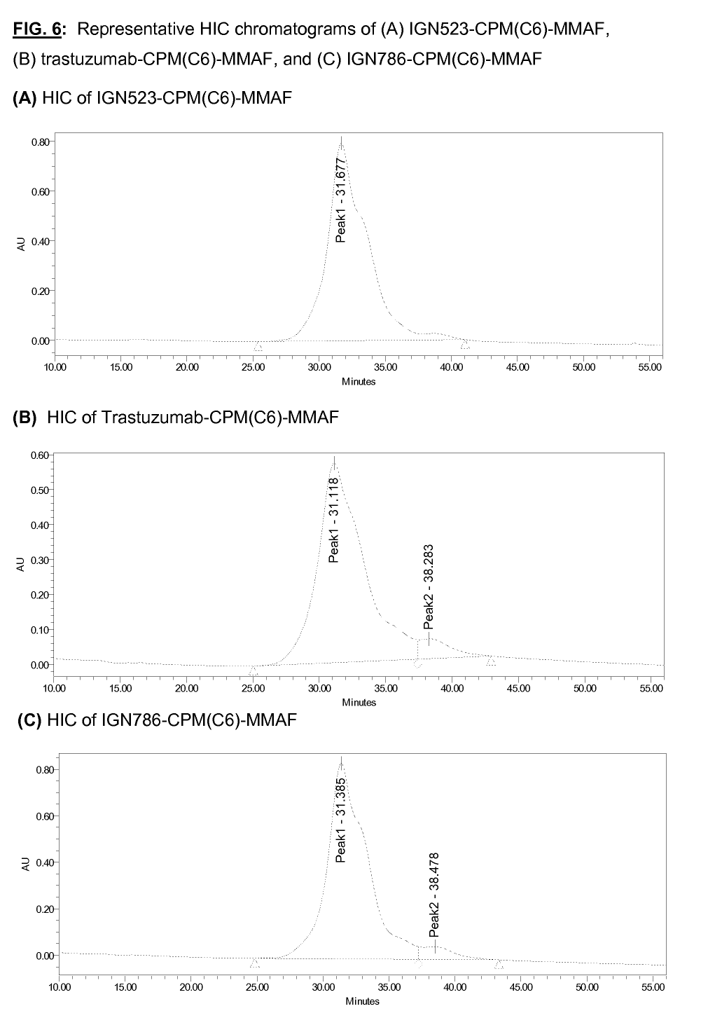

FIG. 5 shows representative SEC chromatograms of (A) trastuzumab-CPM(C6)-MMAF, (B) IGN523-CPM(C6)-MMAF, and (C) IGN786-CPM(C6)-MMAF, demonstrating >95%, >99%, and >98% monomer, respectively.

FIG. 6 shows representative HIC chromatograms of (A) IGN523-CPM(C6)-MMAF, (B) trastuzumab-CPM(C6)-MMAF, and (C) IGN786-CPM(C6)-MMAF, demonstrating the homogeneity of these ADCs.

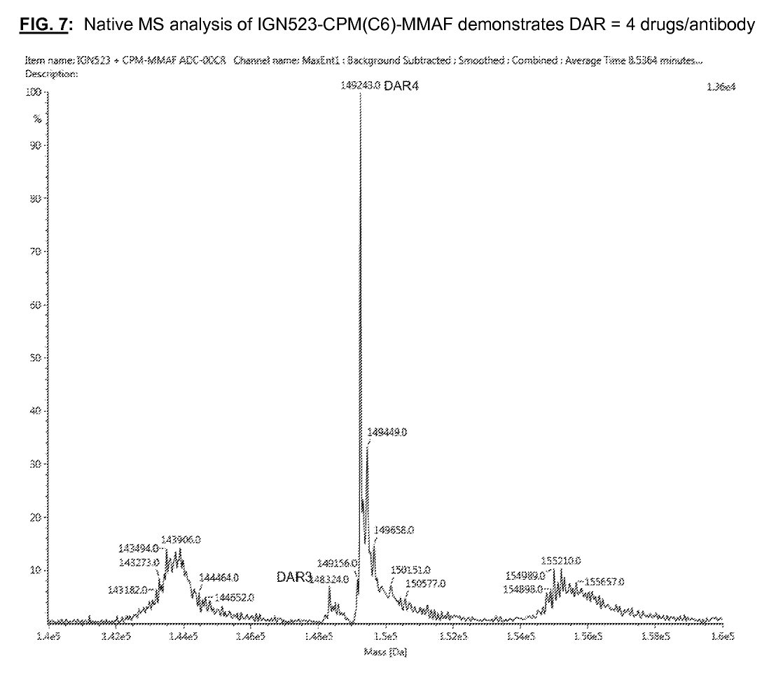

FIG. 7 shows native MS analysis of trastuzumab-CPM(C6)-MMAF.

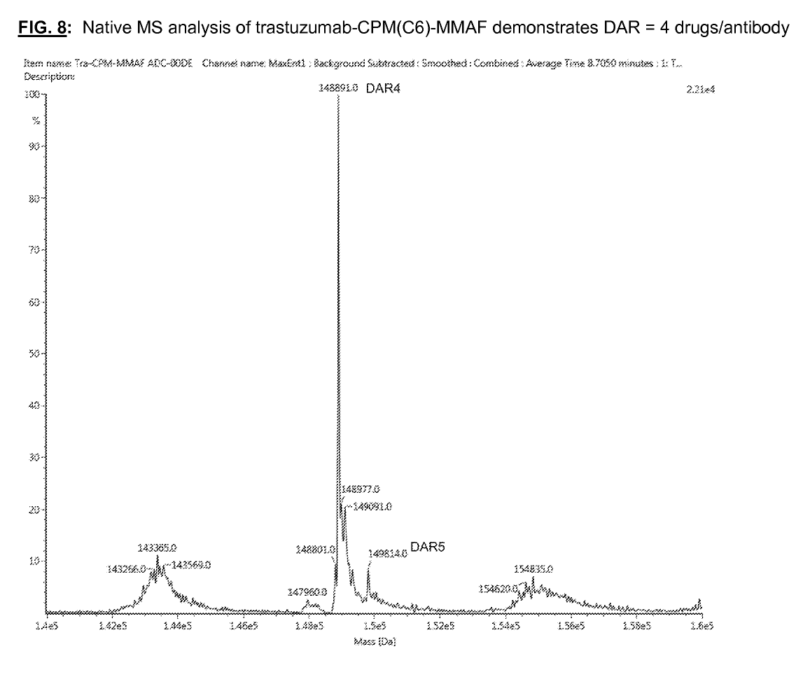

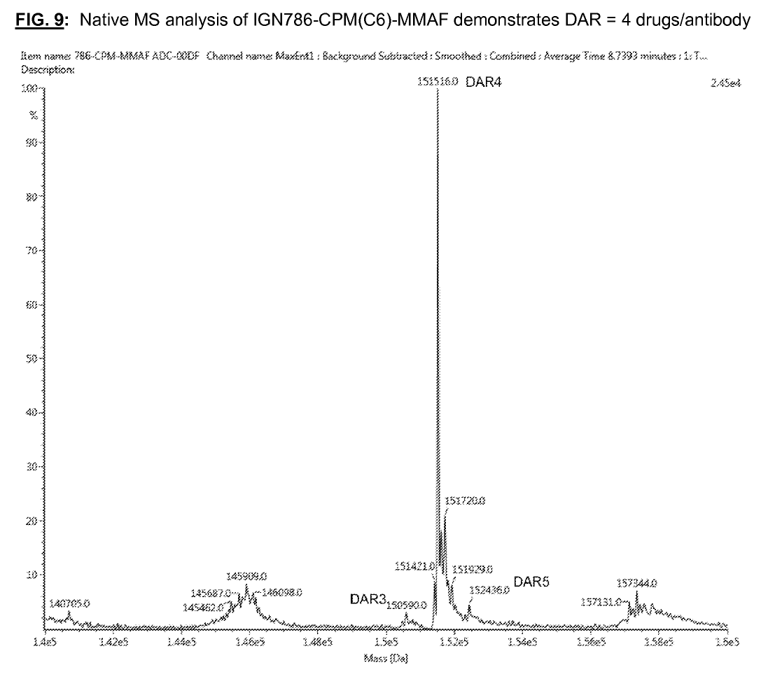

FIG. 8 shows shows native MS analysis of IGN523-CPM(C6)-MMAF. FIG. 9 shows native MS analysis of IGN786-CPM(C6)-MMAF. All figures demonstrate >85% homogeneity and DAR=4 drugs/antibody.

### Example 10: Methods for Making ADCs

This example provides methods for making ADCs. Seven continuous process parameters for such methods were selected and evaluated over a broad range, as shown in Table 1 below. For the evaluation and statistical analysis, JMP®, Version 10.0.0, SAS Institute Inc., Cary, N.C., 1989-2007 was used.

The seven continuous process parameters fell within two groups: (a) reduction parameters ((1) Reduction Temperature, (2) Reduction Time, (3) Reduction pH, and (4) TCEP molar equivalents); and (b) conjugation parameters ((5) Conjugation Temperature, (6) Conjugation Time, and (7) Linker-Cytotoxin molar equivalents).

TABLE 1
Seven Continuous Process Parameters Selected and Evaluated

| | Value/Range | | Value Type |
|---|---|---|---|
| Reduction Parameters | | | |
| Antibody Concentration | 5.0 | mg/mL | Fixed |
| Temperature | 20-37° | C. | Continuous |
| Time | 1-4 | hours | Continuous |
| pH | 7.4-8.2 | | Continuous |
| TCEP | 4-10 molar | | Continuous |
| | equivalents | | |
| Conjugation Parameters | | | |
| Temperature | 20-37° | C. | Continuous |
| Time | 0.5-2 | hours | Continuous |

| | | |
|---|---|---|
| Linker-Cytotoxin | 4-10 molar | Continuous |
| | equivalents | |

A full factorial design was used for the seven process parameters, which resulted in 64 separate experiments, which are described in Table 2. For these experiments, an antibody was reacted with a reducing agent, and then reacted with a linker-cytotoxin conjugate to produce an ADC. The reduction and conjugation reactions were buffered in 20 mM sodium phosphate, 20 mM Borate, and 5 mM EDTA. The experiments were performed with model antibody IGN523 at a concentration of 5 mg/mL. The linker-cytotoxin conjugate used was DBM(C6)-MMAF, synthesized according to Example 5.

TABLE 2
IGN523 DBM-MMAF Conjugation Response
Surface Model DoE
DoE JMP 10.0.0 RSM model N = 64

| | | | | | | | | Linker- | |
|---|---|---|---|---|---|---|---|---|---|
| | | Reduction | Reduction | Reduction | TCEP Molar | Conjugation | Conjugation | Cytotoxin | Conjugation |
| Experiment | Block | pH | Time | Temp (° C.) | eq. | Time (Hr) | Time (min) | Molar eq | Temp (° C.) |
| 1 | 1 | 8.2 | 2.5 | 37 | 6 | 2 | 120.0 | 4.0 | 20.0 |
| 2 | 1 | 8.2 | 1.0 | 28.5 | 10 | 1.475 | 88.5 | 7 | 37.0 |
| 3 | 1 | 8.2 | 1.0 | 20 | 10 | 0.95 | 57.0 | 7 | 20.0 |
| 4 | 1 | 8.2 | 4.0 | 20 | 8 | 0.5 | 30.0 | 5 | 37.0 |
| 5 | 1 | 8.2 | 4.0 | 37 | 7 | 0.5 | 30.0 | 3 | 28.5 |
| 6 | 1 | 8.2 | 1.0 | 37 | 6 | 2 | 120.0 | 7 | 20.0 |
| 7 | 1 | 8.2 | 1.0 | 37 | 6 | 0.5 | 30.0 | 7 | 28.5 |
| 8 | 1 | 8.2 | 2.4 | 37 | 6 | 1.4 | 84.0 | 3 | 20.0 |
| 9 | 1 | 7.8 | 4.0 | 37 | 10 | 0.5 | 30.0 | 5 | 37.0 |
| 10 | 1 | 7.8 | 2.5 | 37 | 8 | 0.5 | 30.0 | 7 | 20.0 |
| 11 | 1 | 7.8 | 2.8 | 28.5 | 8 | 1.25 | 75.0 | 4 | 28.5 |
| 12 | 1 | 7.8 | 2.3 | 37 | 6 | 2 | 120.0 | 3 | 28.5 |
| 13 | 1 | 7.4 | 1.0 | 20 | 10 | 0.5 | 30.0 | 7 | 37.0 |
| 14 | 1 | 7.4 | 2.8 | 28.5 | 10 | 2 | 120.0 | 3 | 20.0 |
| 15 | 1 | 7.4 | 2.8 | 20 | 7 | 2 | 120.0 | 7 | 20.0 |
| 16 | 1 | 7.4 | 4.0 | 37 | 6 | 2 | 120.0 | 5 | 20.0 |
| 17 | 2 | 8.2 | 2.5 | 37 | 6 | 2 | 120.0 | 5.5 | 20 |
| 18 | 2 | 8.2 | 1 | 37 | 10 | 0.5 | 30.0 | 4 | 28.5 |
| 19 | 2 | 8.2 | 4 | 20 | 10 | 0.5 | 30.0 | 4 | 20 |
| 20 | 2 | 8.2 | 4 | 20 | 8 | 2 | 120.0 | 10 | 20 |
| 21 | 2 | 8.2 | 2.8 | 28.5 | 7 | 1.325 | 79.5 | 5.5 | 20 |
| 22 | 2 | 8.2 | 4 | 37 | 6 | 1.325 | 79.5 | 10 | 28.5 |
| 23 | 2 | 8.2 | 2.4 | 20 | 6 | 2 | 120.0 | 5.5 | 37 |
| 24 | 2 | 8.2 | 4 | 28.5 | 6 | 2 | 120.0 | 4 | 37 |
| 25 | 2 | 7.8 | 1 | 20 | 8 | 2 | 120.0 | 4 | 20 |
| 26 | 2 | 7.8 | 2.7 | 37 | 6 | 1.325 | 79.5 | 6.8 | 37 |
| 27 | 2 | 7.8 | 1.5 | 28.5 | 6 | 1.7 | 102.0 | 5.5 | 37 |
| 28 | 2 | 7.8 | 1.5 | 28.5 | 6 | 1.7 | 102.0 | 5.5 | 37 |
| 29 | 2 | 7.4 | 4 | 20 | 10 | 0.725 | 43.5 | 10 | 20 |
| 30 | 2 | 7.4 | 2.7 | 20 | 10 | 0.5 | 30.0 | 4 | 37 |
| 31 | 2 | 7.4 | 1 | 37 | 10 | 1.7 | 102.0 | 4 | 37 |
| 32 | 2 | 7.4 | 4 | 20 | 8 | 2 | 120.0 | 4 | 37 |
| 33 | 3 | 8.2 | 2.5 | 37 | 6 | 2 | 120.0 | 5.5 | 20.0 |
| 34 | 3 | 8.2 | 4 | 37 | 10 | 0.5 | 30.0 | 10 | 20 |
| 35 | 3 | 8.2 | 4 | 37 | 10 | 2 | 120.0 | 5.5 | 20 |
| 36 | 3 | 8.2 | 1 | 37 | 10 | 2 | 120.0 | 4 | 20 |
| 37 | 3 | 8.2 | 2.8 | 37 | 8 | 2 | 120.0 | 10 | 37 |
| 38 | 3 | 8.2 | 2.8 | 37 | 8 | 2 | 120.0 | 10 | 37 |
| 39 | 3 | 8.2 | 4 | 20 | 6 | 1.475 | 88.5 | 4 | 20 |
| 40 | 3 | 7.8 | 3 | 28.5 | 8 | 1.175 | 70.5 | 5.5 | 28.5 |
| 41 | 3 | 7.8 | 4 | 37 | 8 | 1.175 | 70.5 | 4 | 20 |
| 42 | 3 | 7.8 | 4 | 37 | 8 | 1.175 | 70.5 | 4 | 20 |
| 43 | 3 | 7.4 | 1 | 37 | 10 | 1.4 | 84.0 | 6.8 | 20 |
| 44 | 3 | 7.4 | 1 | 37 | 10 | 1.4 | 84.0 | 6.8 | 20 |
| 45 | 3 | 7.4 | 1.6 | 28.5 | 8 | 0.8 | 48.0 | 4 | 20 |
| 46 | 3 | 7.4 | 1.6 | 28.5 | 8 | 0.8 | 48.0 | 4 | 20 |
| 47 | 3 | 7.4 | 4 | 37 | 6 | 0.5 | 30.0 | 5.5 | 20 |
| 48 | 3 | 7.4 | 4 | 37 | 6 | 0.5 | 30.0 | 5.5 | 20 |
| 49 | 4 | 8.2 | 2.5 | 37 | 6 | 2 | 120.0 | 5.5 | 20.0 |
| 50 | 4 | 8.2 | 4 | 20 | 10 | 1.475 | 88.5 | 4 | 37 |
| 51 | 4 | 8.2 | 4 | 20 | 10 | 1.475 | 88.5 | 4 | 37 |
| 52 | 4 | 8.2 | 2.7 | 28.5 | 8 | 1.1 | 66.0 | 5.5 | 28.5 |
| 53 | 4 | 8.2 | 2.7 | 28.5 | 8 | 1.1 | 66.0 | 5.5 | 28.5 |
| 54 | 4 | 8.2 | 2.7 | 20 | 6 | 0.5 | 30.0 | 10 | 20 |
| 55 | 4 | 8.2 | 1 | 20 | 6 | 0.95 | 57.0 | 4 | 37 |
| 56 | 4 | 8.2 | 2.4 | 37 | 6 | 0.5 | 30.0 | 4 | 37 |
| 57 | 4 | 7.8 | 1.8 | 20 | 10 | 2 | 120.0 | 6.8 | 37 |
| 58 | 4 | 7.8 | 1.3 | 20 | 7 | 0.5 | 30.0 | 5.5 | 20 |
| 59 | 4 | 7.8 | 2.7 | 20 | 6 | 1.55 | 93.0 | 10 | 37 |
| 60 | 4 | 7.8 | 3.9 | 28.5 | 6 | 0.5 | 30.0 | 5.5 | 37 |
| 61 | 4 | 7.8 | 3.1 | 20 | 6 | 0.5 | 30.0 | 4 | 37 |
| 62 | 4 | 7.4 | 4 | 28.5 | 10 | 2 | 120.0 | 10 | 37 |
| 63 | 4 | 7.4 | 2.5 | 37 | 7 | 1.025 | 61.5 | 10 | 37 |
| 64 | 4 | 7.4 | 2.5 | 20 | 7 | 1.325 | 79.5 | 4 | 28.5 |

For the experiments in Table 2, reactants were prepared and products were analyzed as follows.

IGN523 was buffer exchanged by 10 mL Zeba column 40 kD cutoff (Thermo Cat No. 87772) into 20 mM Sodium borate, 20 mM Sodium Phosphate, 100 mM NaCl pH 7.4, pH 7.8, or pH 8.2. Concentration after buffer exchange was measured by absorbance at A280 using a Thermo Evolution 220 spectrophotometer. IGN523 pH 7.4, 7.8 or 8.2 solution was diluted to 5 mg/mL in the respective buffer. Aliquots of 100 μL were made into 2.0 mL o-ring cap tubes (Sarstedt) as indicated on the DoE Block chart provided in Appendix A.

TCEP was diluted to the indicated starting concentration such that 5% addition by volume to the reaction tube would result in the desired number of TCEP molatr equivalents in relation to IGN523 molar equivalents. TCEP was diluted in water. After addition of 5 μL of TCEP to the reaction, tubes were briefly mixed by vortex and placed at the indicated temperature for the indicated time according to the DoE Block chart provided in Appendix A.

After reduction, 5 μL was removed from the reduction reaction for an iodoacetamide (IAM) cap control to be run on SDS-PAGE. Cap control was taken to assess the amount of reduction of IGN523 at each of the given conditions. 5 μL was removed and diluted in 45 μL of 30 mM IAM, 100 mM sodium phosphate, 150 mM NaCl, pH 6.8. Capping samples were incubated for at least 30 minutes at room temperature. For SDS-PAGE analysis, 10 μL of diluted IAM cap control was further diluted with 30 μL of Non-Reducing sample buffer and 10 μL was loaded on a 4-12% Tris-Glycine Gel (Life Technologies). Separated proteins were visualized by Sypro Orange stain (Life Technologies) on a Typhoon Trio (GE Lifesciences).

DBM(C6)-MMAF (Stock solution of 10 mM in dimethylacetamide (DMA) solvent) was further diluted in DMA as indicated on the DoE Chart provided in Appendix A. Drug linker was added at 10% volume of the starting volume. 10 μL of diluted DBM was added to each reaction, tubes were briefly mixed by vortex and placed at the indicated temperature for the indicated time according to the DoE Block chart provided in Appendix A.

The conjugation reaction was stopped by buffer exchange into 20 mM histidine, 7% (w/v) sucrose, 20 mM NaCl, pH 6.0 using 0.5 mL Zeba column 7 kD cutoff (Thermo Cat No. 89882). Buffer exchange was done according to the manufacturers instructions. After analysis, remaining samples were stored frozen.

Concentration of the completed reactions was determined by A280 absorbance using a Nanodrop (Thermo). Samples for hydrophobic interaction chromatography (HIC) and size exclusion chromatography (SEC) were first diluted to 1 mg/mL in 100 mM sodium phosphate, 150 mM NaCl, pH 6.8.

HIC was done using a Tosoh Butyl-NPR column (4.6 mm×10 cm) with a gradient over 16 minutes from 100% mobile phase A (1.5M ammonium sulfate, 25 mM sodium phosphate, pH 6.95) to 70% mobile phase B (25 mM sodium phosphate pH 6.96/isopropanol (75%/25%)).

SEC was done using a Waters BEH SEC 200 column with 100 mM sodium phosphate, 150 mM NaCl, pH 6.8 as the mobile phase.

HIC and SEC were run on a Waters Acquity Bio H Class UPLC. HIC and SEC were analyzed by Empower® software (Waters).

Mass Spectrometry was performed on a Waters Acquity Bio H Class UPLC in line with a Waters QTOF.

Parameters were fit to terms in a quadratic equation (y=ax²+bx+c, where y=DAR 4). Specifically, DAR homogeneity (DAR=4) was calculated using the following equation:

DAR 4=257.617012484113+1.59168731396224*((:pH−7.8)/0.399999999999999)+4.67843384284532*((:Reduction Time−2.5)/1.5)+−0.486481222203335*((:Reduction Temp−28.5)/8.5)+5.9329588097101*((:TCEP Molar eq.−7)/3)+−3.69330908000285*((:Conjugation Time−1.25)/0.75)+−18.4106249618604*((:Linker-Cytotoxin Molar Eq.−7.5)/2.5)+2.27828810007445*((:Conjugation Temp−28.5)/8.5)+((:pH−7.8)/0.399999999999999)*(((:pH−7.8)/0.399999999999999)*0.795827857524658)+((:pH−7.8)/0.399999999999999)*(((:Reduction Time−2.5)/1.5)*−6.15368213908698)+((:Reduction Time−2.5)/1.5)*(((: Reduction Time−2.5)/1.5)*4.58908738519519)+((:pH−7.8)/0.399999999999999)*(((:Reduction Temp−28.5)/8.5)*−5.58658326074429)+((:Reduction Time−2.5)/1.5)*(((:Reduction Temp−28.5)/8.5)*−2.72570329357844)+((:Reduction Temp−28.5)/8.5)*(((:Reduction Temp−28.5)/8.5)*−0.954198959391965)+((:pH−7.8)/0.399999999999999)*(((:TCEP Molar eq.−7)/3)*−4.36978946805627)+((:Reduction Time-2.5)/1.5)*(((:TCEP Molar eq.−7)/3)*−1.7535338056395)+((:Reduction Temp−28.5)/8.5)*(((:TCEP Molar eq.−7)/3)*2.49208510179499)+((:TCEP Molar eq.−7)/3)*(((:TCEP Molar eq.−7)/3)*−7.3444899495457)+((:pH−7.8)/0.399999999999999)*(((:Conjugation Time−1.25)/0.75)*1.25909066392585)+((:Reduction Time−2.5)/1.5)*(((:Conjugation Time−1.25)/0.75)*−2.25556085737926)+((:Reduction Temp−28.5)/8.5)*(((:Conjugation Time−1.25)/0.75)*1.3035085290802)+((:TCEP Molar eq.−7)/3)*(((:Conjugation Time−1.25)/0.75)*0.731377001912696)+((:Conjugation Time−1.25)/0.75)*(((:Conjugation Time−1.25)/0.75)1.69126630078065)+((:pH-7.8)/0.399999999999999)*(((:Linker-Cytotoxin Molar Eq.−7.5)/2.5)*−0.972073990540948)+((:Reduction Time−2.5)/1.5)*(((:Linker-Cytotoxin Molar Eq.−7.5)/2.5)*2.84625607268034)+((:Reduction Temp−28.5)/8.5)*(((:Linker-Cytotoxin Molar Eq.−7.5)/2.5)*−0.500454843305044)+((:TCEP Molar eq.−7)/3)*(((:Linker-Cytotoxin Molar Eq.−7.5)/2.5)*1.90988517039372)+((:Conjugation Time−1.25)/0.75)*(((:Linker-Cytotoxin Molar Eq.−7.5)/2.5)*−2.38259827949913)+((:Linker-Cytotoxin Molar Eq.−7.5)/2.5)*(((:Linker-Cytotoxin Molar Eq.−7.5)/2.5)*−25.0200498957236)+((:pH−7.8)/0.399999999999999)*(((:Conjugation Temp−28.5)/8.5)*−7.38948735548731)+((:Reduction Time−2.5)/1.5)*(((:Conjugation Temp−28.5)/8.5)*0.437280546180644)+((:Reduction Temp−28.5)/8.5)*(((:Conjugation Temp−28.5)/8.5)*−1.68417019161346)+((:TCEP Molar eq.−7)/3)*(((:Conjugation Temp−28.5)/8.5)*−3.03856893270271)+((:Conjugation Time−1.25)/0.75)*(((:Conjugation Temp−28.5)/8.5)*1.06169879580724)+((:Linker-Cytotoxin Molar Eq.−7.5)/2.5)*(((:Conjugation Temp−28.5)/8.5)*1.64099404293451)+((:Conjugation Temp−28.5)/8.5)*(((:Conjugation Temp−28.5)/8.5)*−2.30205921744132) Equation 1

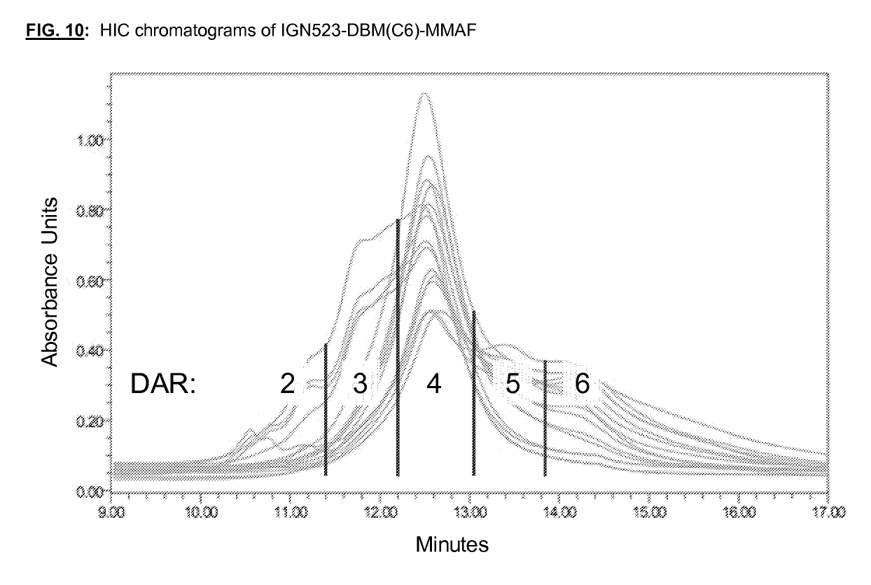

FIG. 10 shows 28 out of the 63 HIC chromatograms generated as part of the DoE experiment. The 28 chromatograms were randomly selected so the DAR heterogeneity could be better visualized. As may be seen in the figure, the full factorial design space yielded heterogeneous drug loading, with some of the chromatograms indicating very low drug loading with a DAR of one, two, or three (DAR=1, 2, or 3); other chromatograms indicating overloading at DAR five or six (DAR=5 or 6); and still other chromatograms indicating a relatively homogeneous DAR four profile (DAR=4; “DAR 4”).

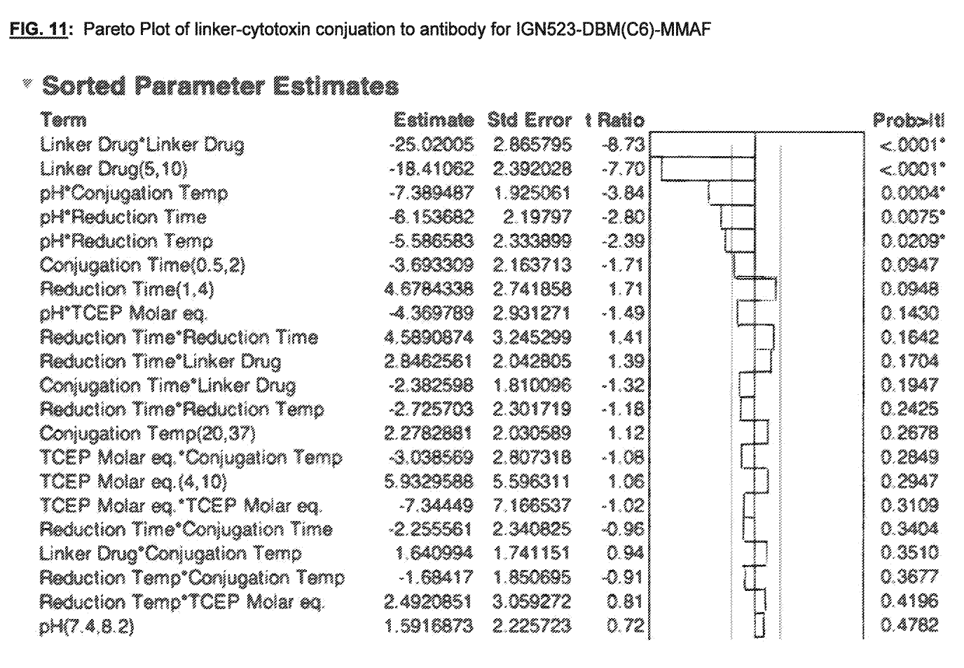

FIG. 11 shows a Pareto Plot of process parameters and process parameter combinations, sorted in order of greatest to least influence on fidelity of conjugation of the antibody to the linker-cytotoxin. As may be seen in the figure, the Linker-Cytotoxin molar equivalents parameter (“Linker-Cytotoxin”) and the combination which is the square of the Linker-Cytotoxin molar equivalents parameter (“Linker-Cytotoxin*Linker-Cytotoxin”) had the greatest influence on the fidelity of conjugation. To the right of the figure is also shown the statistical probability for each parameter or parameter combination Asterixed parameters or parameter combinations were those calculated to be statistically significant, and were ranked in order of significance as Linker-Cytotoxin*Linker-Cytotoxin>Linker-Cytotoxin>pH*Conjugation Temperature>Reduction pH*Reduction Time>Reduction pH*Reduction Temperature.

Additional experiments were conducted varying the number of Linker-Cytotoxin molar equivalents and TCEP molar equivalents.

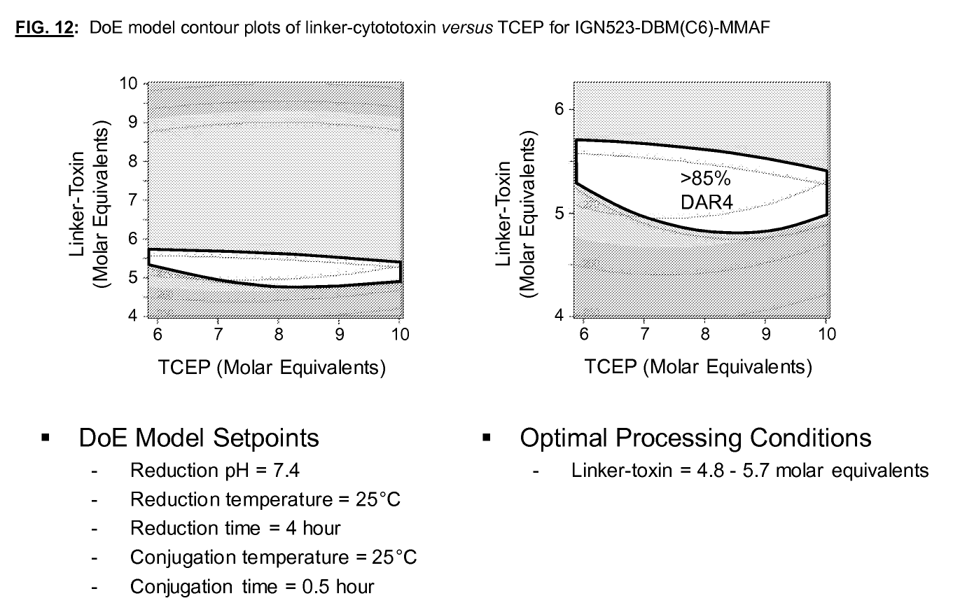

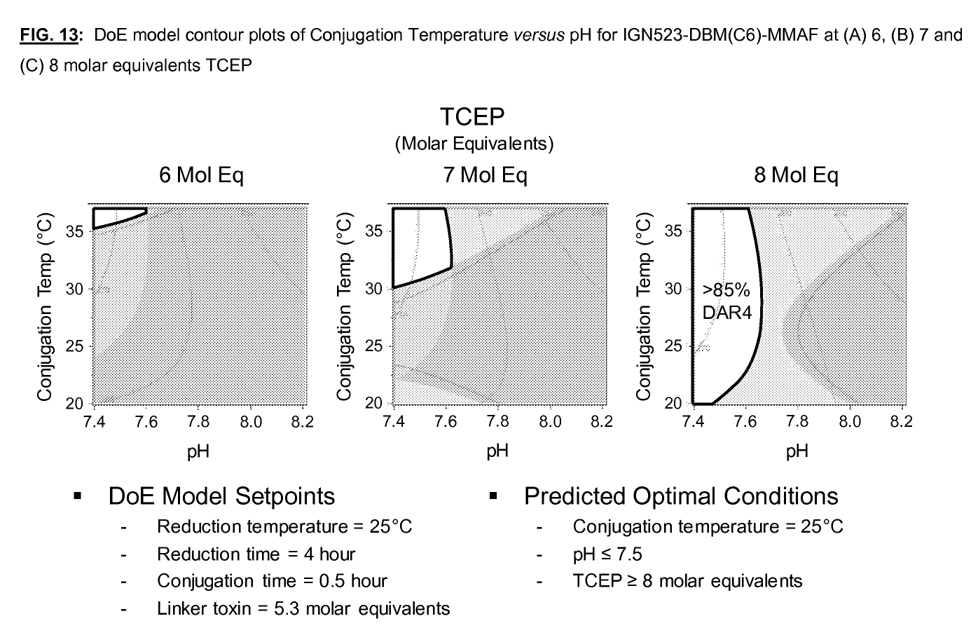

FIG. 12 shows a contour plot of Linker-Cytotoxin molar equivalents versus TCEP molar equivalents, where all other parameters were kept constant (Reduction pH=7.4; Reduction Temperature=25° C.; Conjugation Temperature=25° C.; Reduction Time=4 hours; Conjugation Time=0.5 hours). The white space in the contour plots represents the model's predictions of conditions where DAR 4 exceeds 85%.

As may be seen in the left-hand and right-hand panels of the figure, optimal results (DAR 4) were obtained for Linker-Cytotoxin equivalents between 4.8 to 5.7 molar equivalents and TCEP equivalents between 6 to 10 molar equivalents. The process parameter, Linker-Cytotoxin equivalents, had a tight tolerance of between 4.8 to 5.7 molar equivalents, and was therefore designated a “critical control parameter” or “CCP.”

Additional experiments were conducted varying the conjugation temperature and the pH of the reduction reaction.

FIG. 13 shows a contour plot of Conjugation Temperature versus Reduction pH for three values of TCEP molar equivalents, where all other parameters were kept constant (Reduction Temperature=25° C.; Reduction Time=4 hours; Conjugation Time=0.5 hours; and Linker-Cytotoxin molar equivalents=5.3). The left-hand, middle, and right-hand panels show the effect of increasing molar equivalents of TCEP, for 6, 7 and 8 molar equivalents, respectively. The white space in the contour plots represents the model's predictions of conditions where DAR 4 exceeds 85%.

As may be seen in the left-hand panel of the figure, a relatively high conjugation temperature of 35-37° C. was required to achieve conditions where DAR 4 exceeds 85%, when the reaction occurred at pH 7.4. As may be seen in the middle panel of the figure, an increase in the TCEP molar equivalents from 6 to 7 reduced the required conjugation temperature to 30° C. As may be seen in the right-hand panel of the figure, a further increase in the TCEP molar equivalents from 7 to 8 further reduced the required conjugation temperature to 20° C.

Antibodies are less prone to aggregation and deamidation when processed, for example, at lower temperatures and pH values. This example shows that excess TCEP, relative to Linker-Cytotoxin molar equivalents required to reduce the 4 interchain disulfide bonds in an IgG1 antibody, allows these milder, more optimal, and likely, more universal and robust processing conditions.

In additional experiments, four continuous process parameters were selected and evaluated over a narrow range, as shown in Table 3 below. The four continuous process parameters were: (1) Reduction Time, (2) Reduction pH, (3) TCEP equivalents and (4) Linker-Cytotoxin molar equivalents.

TABLE 3
Evaluating Lower pH and Narrower Linker-Cytotoxin Equivalents

| | Value/Range | | Value Type |
|---|---|---|---|
| Reduction Parameters | | | |
| Antibody Concentration | 5.0 | mg/mL | Fixed |
| Temperature | 25° | C. | Fixed |
| Time | 2-4 | hours | Continuous |
| pH | 7.0-7.8 | | Continuous |
| TCEP | 4-10 molar | | Continuous |
| | equivalents | | |
| Conjugation Parameters | | | |
| Temperature | 25° | C. | Fixed |
| Time | 0.5 | hours | Fixed |

| | | |
|---|---|---|
| Linker-Cytotoxin | 4.5-6.0 molar | Continuous |
| | equivalents | |

For these experiments, a full factorial design was used for the selected four process parameters, which resulted in 24 separate experiments. The reduction and conjugation reactions were buffered in 20 mM sodium phosphate, 20 mM Borate, and 5 mM EDTA. Two model antibodies, IGN523 and trastuzumab, were used at concentrations of 5 mg/mL for each antibody. The Linker-Cytotoxin used was DBM(C6)-MMAF.

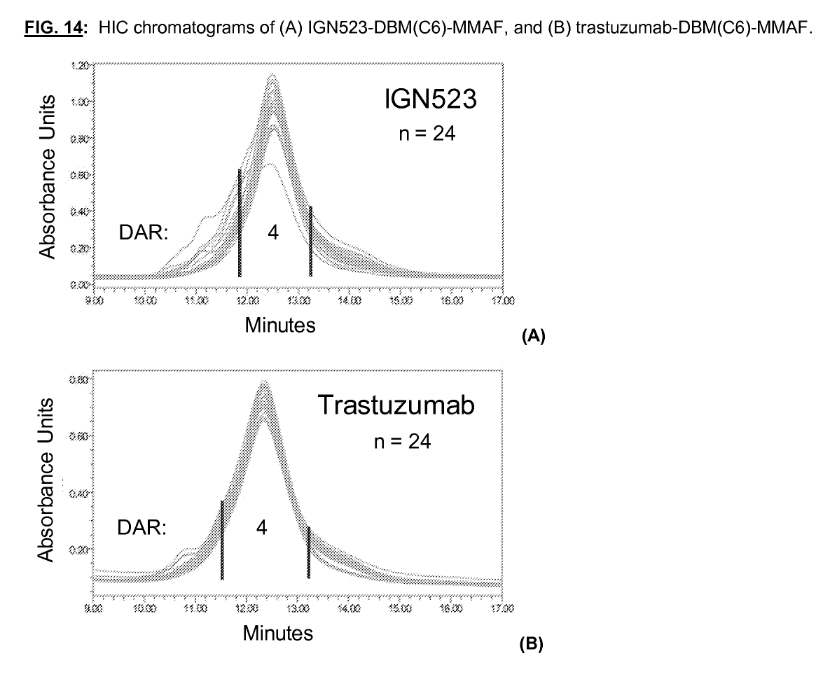

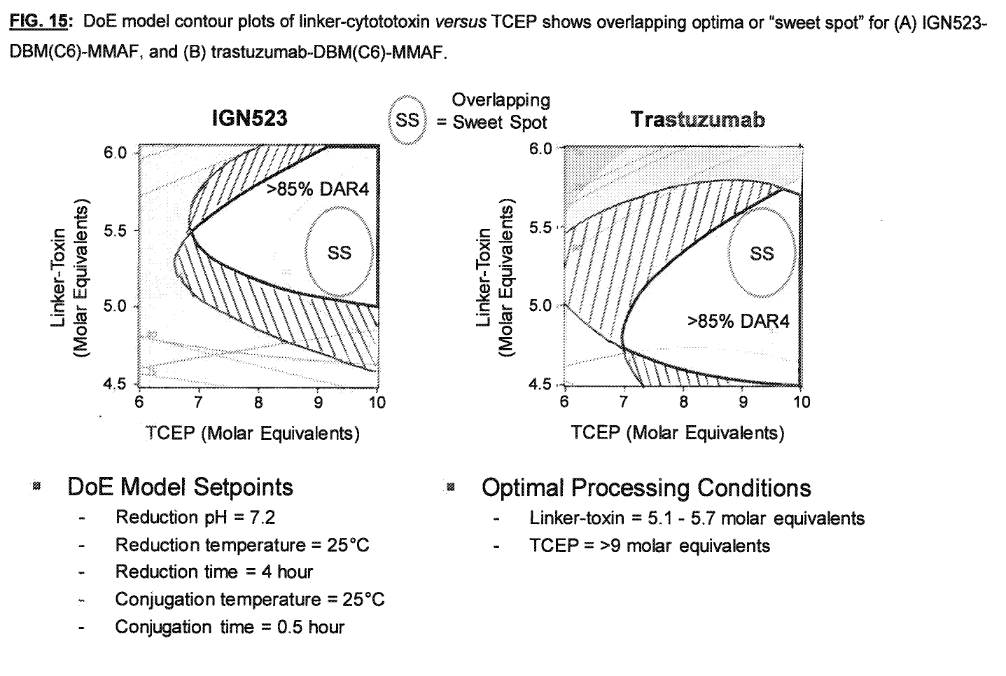

FIG. 14 shows 24 HIC chromatograms for IGN523 (A) and trastuzumab (B). As may be seen in the figure, IGN523 and trastuzumab yielded similar conjugation profiles. For IGN523, some of the 24 experiments resulted in under-loading (e.g., DAR=1, 2, or 3), whereas some of the conditions yielded the target homogeneous DAR 4. For trastuzumab, many of the conditions yielded the target DAR 4, suggesting that trastuzumab is a well behaved antibody.

FIG. 15 shows DoE contour plots of linker-cytototoxin equivalents versus TCEP equivalents for (A) IGN523-DBM(C6)-MMAF, and (B) trastuzumab-DBM(C6)-MMAF. All other parameters were kept constant (Reduction pH=7.2; Reduction Temperature=25° C.; Reduction Time=4 hours; Conjugation Temperature=25° C.; and Conjugation Time=0.5 hours). The white space in the contour plots represents the model's predictions of conditions where DAR 4 exceeds 85% (“optimal subregion” or “sweet spot”). The cross-hatched space (///) indicates over-conjugation (e.g., DAR=5 or 6), and the cross-hatched space (\\\) indicates under-conjugation (e.g., DAR=1, 2, or 3). As may be seen in the figure, IGN523 and trastuzumab had similar DoE contour plots which overlap in their optimal subregions. For example, IGN523 had an optimal range of approximately 5 to 6 Linker-Cytotoxin molar equivalents, while trastuzumab had an optimal range of approximately 4.5 to 5.5, such that there is an overlapping area of roughly 5.1 to 5.7 Linker-Cytotoxin molar equivalents. As may also be seen in the figure, an increase in TCEP molar equivalents correlated with a broader range of Linker-Cytotoxin molar equivalents within the optimal subregion. The Linker-Cytotoxin parameter had a tight tolerance (hence its designation as a critical control parameter or CCP). Therefore, it is highly advantageous to have excess TCEP, for example, >9 TCEP molar equivalents.

One process parameter, Linker-Cytotoxin equivalents, was evaluated at discreet values, as shown in Table 4.

TABLE 4
Evaluating Linker-Cytotoxin Equivalents at Discreet Values

| Reduction Parameters | Value | | Value Type |
|---|---|---|---|
| Antibody Concentration | 5.0 | mg/mL | Fixed |
| Temperature | 25° | C. | Fixed |
| Time | 3.5 | hours | Fixed |
| pH | 7.2 | | Fixed |
| TCEP | 9.5 molar | | Fixed |
| | equivalents | | |
| Conjugation Parameters | Value/Range | | Value Type |
| Temperature | 25° | C. | Fixed |
| Time | 0.5 | hours | Fixed |

| | | |
|---|---|---|
| Linker-Cytotoxin | 5.1-5.8 molar | Discrete |
| | equivalents | |

In these experiments, the reduction and conjugation reactions were buffered in 20 mM sodium phosphate, 20 mM Borate, and 5 mM EDTA. Three model antibodies, IGN523, trastuzumab, and IGN786, were used at concentrations of 5 mg/mL for each antibody. The experiments used three discreet values of Linker-Cytotoxin equivalents: 5.2, 5.5, and 5.8 molar equivalents for IGN523 and trastuzumab; and 5.1, 5.4, and 5.7 molar equivalents for IGN786. The Linker-Cytotoxin used in the experiment was DBM(C6)-MMAF.

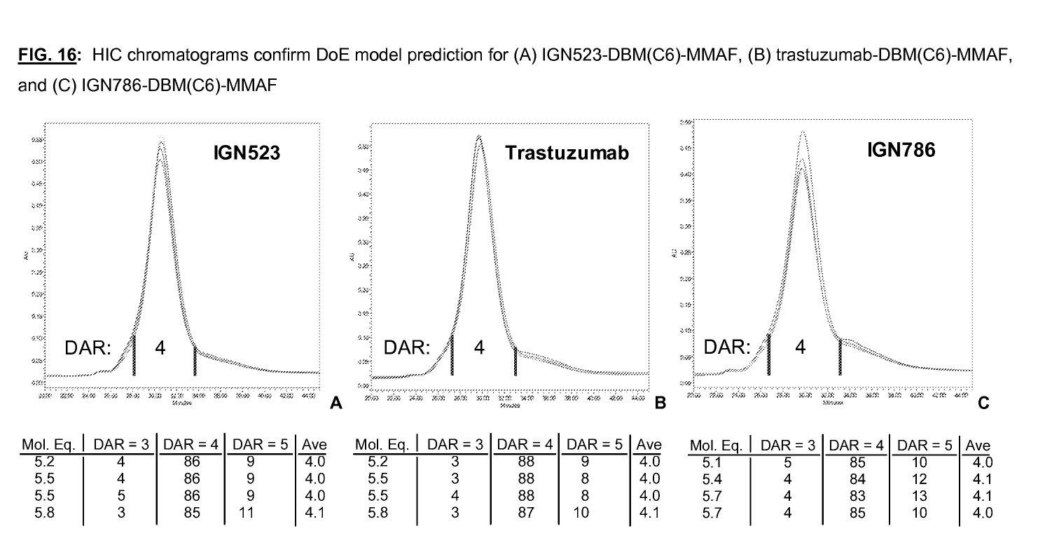

FIG. 16 shows HIC chromatograms confirming DoE model prediction for (A) IGN523-DBM(C6)-MMAF, (B) trastuzumab-DBM(C6)-MMAF, and (C) IGN786-DBM(C6)-MMAF. As may be seen in the figure, for all of the linker-cytotoxin molar equivalents tested (5.2, 5.5, and 5.8 for IGN523 and trastuzumab; and 5.1, 5.4, and 5.7 for IGN786), the selected optimal conditions yielded a DAR 4>80%.

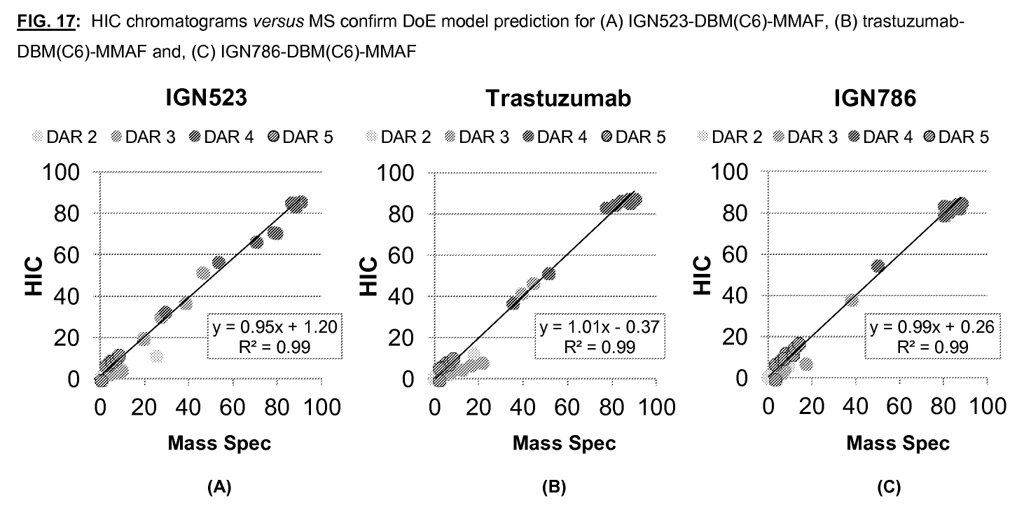

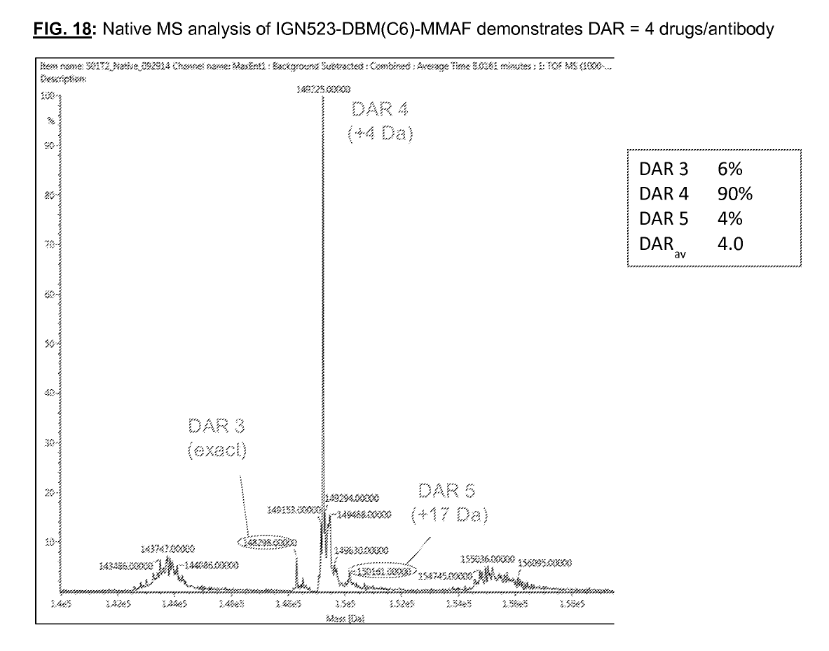

FIG. 17 shows HIC chromatograms versus MS confirming DoE model prediction for (A) IGN523-DBM(C6)-MMAF, (B) trastuzumab-DBM(C6)-MMAF and, (C) IGN786-DBM(C6)-MMAF. As may be seen in this figure, comparison of HIC and MS obtained values for DAR using a linear fit shows excellent agreement (R²=0.99).

FIG. 18 shows native MS analysis of IGN523-DBM(C6)-MMAF conjugated at the optimal 5.5 molar equivalents of DBM-MMAF. DAR 4 equals 90%, with an average DAR of 4.0.

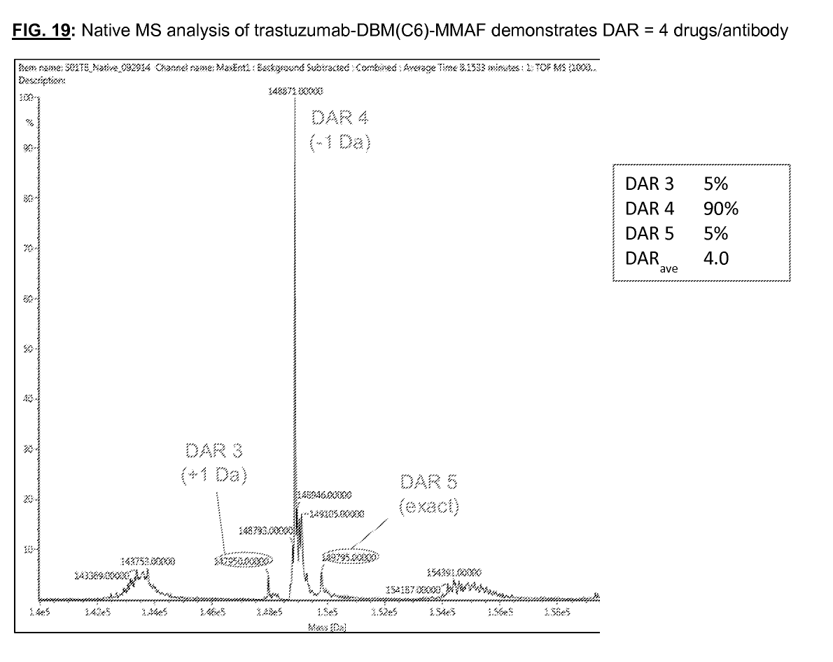

FIG. 19 shows native MS analysis of trastuzumab-DBM(C6)-MMAF conjugated at the optimal 5.5 molar equivalents of DBM-MMAF. DAR 4 equals 90%, with an average DAR of 4.0.

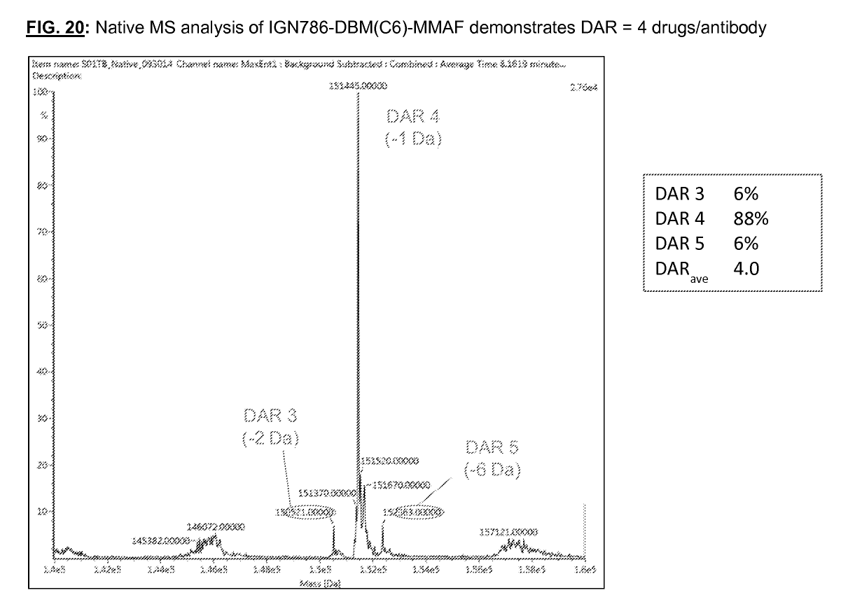

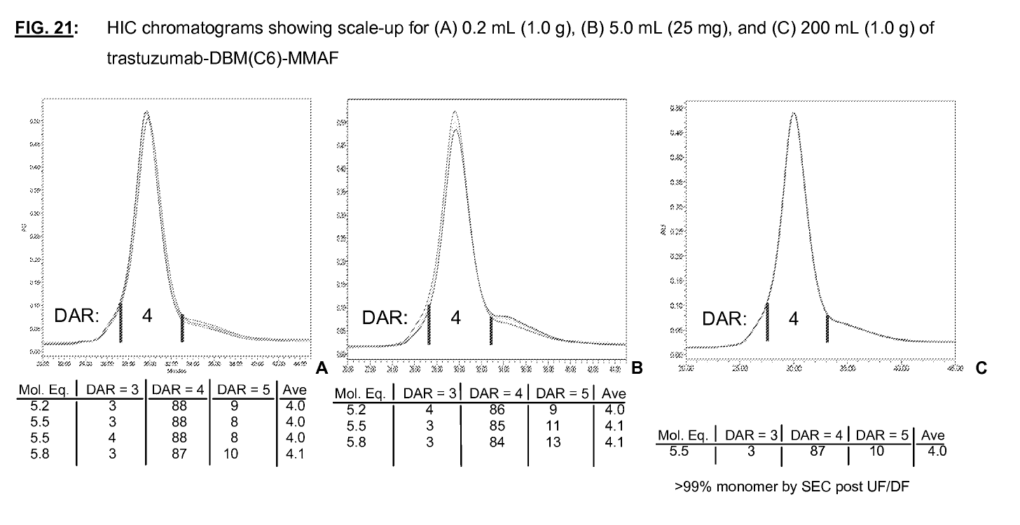

FIG. 20 shows native MS analysis of IGN786-DBM(C6)-MMAF conjugated at the optimal 5.5 molar equivalents of DBM-MMAF. DAR 4 equals 88%, with an average DAR of 4.0.

Additional experiments were conducted to investigate the scalability of methods for making ADCs. As shown above, ADCs with a homogeneous DAR 4 under optimal conditions using different antibodies (e.g., IGN523, trastuzumab, and IGN786) were prepared on a small scale. For the scalability experiments, varying amounts of Linker-Cytotoxin were used with trastuzumab as a model antibody, for example, 5.2, 5.5, and 5.8 molar equivalents of Linker-Cytotoxin, as shown in Table 5.

TABLE 5
Scale-up Experiment

| Reduction Parameters | Value | | Value Type |
|---|---|---|---|
| Temperature | 25° | C. | Fixed |
| Time | 3.5 | hours | Fixed |
| pH | 7.2 | | Fixed |
| TCEP | 9.5 molar | | Fixed |
| | equivalents | | |
| Conjugation Parameters | Value/Range | | Value Type |
| Temperature | 25° | C. | Fixed |
| Time | 0.5 | hours | Fixed |

| | | |
|---|---|---|
| Linker-Cytotoxin | 5.2, 5.5 & 5.8 | Discrete |
| | molar equiv | |

The reduction and conjugation reactions were buffered in 20 mM sodium phosphate, 20 mM Borate, and 5 mM EDTA.

The experiment was performed with trastuzumab in the following increasing amounts: 1.0 mg in 0.2 mL, 25 mg in 5.0 mL, and 1000 mg in 200 mL.

The experiments used three discreet values of Linker-Cytotoxin equivalents: 5.2, 5.5, and 5.8 molar equivalents.

FIG. 21 shows HIC chromatograms for (A) 1.0 mg in 0.2 mL, (B) 25 mg in 5.0 mL, and (C) 1000 mg in 200 mL of trastuzumab-DBM(C6)-MMAF. As may be seen in the figure, the process scales up over the 1000-fold scale range, achieving DAR 4>85% over 5.2, 5.5, and 5.8 molar equivalents Linker-Cytotoxin. In particular, the data generated at the 1000 g scale (C) shows high homogeneity as evidenced by DAR 4 at 87%. Moreover, the percent monomer after the UF/DF buffer exchange step was greater than 99%.

As disclosed herein, an ADC with a homogeneous DAR 4 profile may be made by opening the interchain disulfide bonds of the IgG1 using an appropriate reducing agent, and reacting a linker-cytotoxin (e.g., DBM(C6)-MMAF) with the two cysteines of an opened disulfide bond to give a “stapled” or “snapped” antibody conjugate with one linker-cytotoxin per disulfide connected through two thioether bonds. As shown in FIG. 1, human IgG1 antibodies, such as IGN523, trastuzumab, and IGN78, have 4 interchain disulfide bonds.

As disclosed herein, an ADC with a homogenous DAR 2 or 3 profile may be made by opening the interchain disulfide bonds of a mutated IgG1, wherein one or both of the hinge cysteines have been mutated to another amino acid (e.g., alanine), using an appropriate reducing agent, and reacting a linker-cytotoxin (e.g., DBM(C6)-MMAF) with the two cysteines of an opened disulfide bond to give a “stapled” or “snapped” antibody conjugate with one linker-cytotoxin per disulfide connected through two thioether bonds. ADCs with a homogenous DAR2 or DAR3 profile may also be made as described herein with mutated IgG2, IgG3 or IgG4 antibodies.

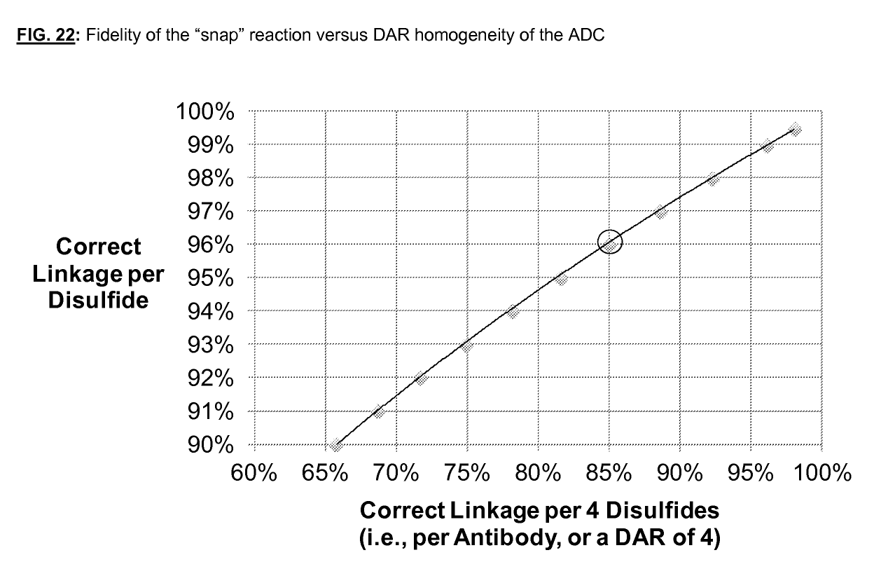

FIG. 22 shows the fidelity of the coupling reaction versus DAR homogeneity of the ADC. As shown in the figure, in order to achieve an ADC with a DAR 4>85%, it is necessary to couple greater than 96% of the disulfides on a per disulfide basis with linker-cytotoxin (e.g., the fidelity of the coupling reaction must be greater than 96% to achieve DAR>85%). The present example demonstrates that at the optimal processing parameters determined by DoE, the fidelity of the coupling reaction of DBM(C6)-MMAF to IgG1 (e.g., IGN523, trastuzumab, and IGN78) is greater than 96% to achieve the observed DAR 4>85%.

### Example 11: Characterization of Homogeneous ADCs

### Example 11A

This example describes characterization of homogeneous ADCs made with the linker-cytotoxin conjugates and antibodies disclosed herein.

A. Trastuzumab and IGN523

Trastuzumab and an exemplary anti-CD98 antibody comprising the VH and VL sequences in Table B (designated herein as “IGN523”) were also prepared and conjugated with linker-cytotoxins according to the methods disclosed herein. Excess reducing agent and a slight excess (5 eq.) of the linker-cytotoxin were used to obtain ADCs with DARs of approximately 4 drugs/antibody. The resulting ADCs were purified via size exclusion chromatagraphy (SEC) to remove excess reagents. The purified ADCs were characterized as described below.

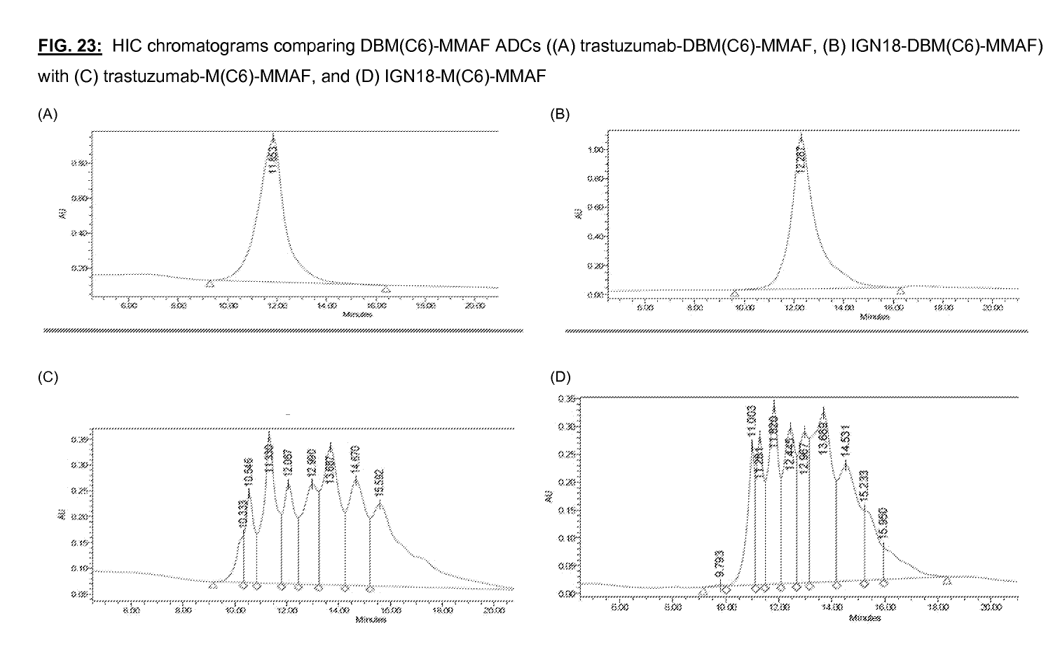

The relative homogeneity and DARs (drugs/antibody ratio) of ADCs were determined using hydrophobic interaction chromatography (HIC) and native LC/MS analysis. HIC analysis enables resolution of ADC fractions containing different DARs due to an increase in hydrophobicity of ADCs with higher DARs. HIC analysis of the ADCs showed that ADCs conjugated with DBM(C6)-MMAF eluted as single homogeneous peaks with retention times consistent with DARs of 4 drugs/antibody (see FIGS. 23 (A) and (B)). In contrast, ADCs containing a an MC (e.g., M(C6) or maleimidocaproyl) linker demonstrated highly heterogeneous HIC profiles with DARs ranging from 0 to 8 drugs/antibody (see FIGS. 23 (C) and (D)).

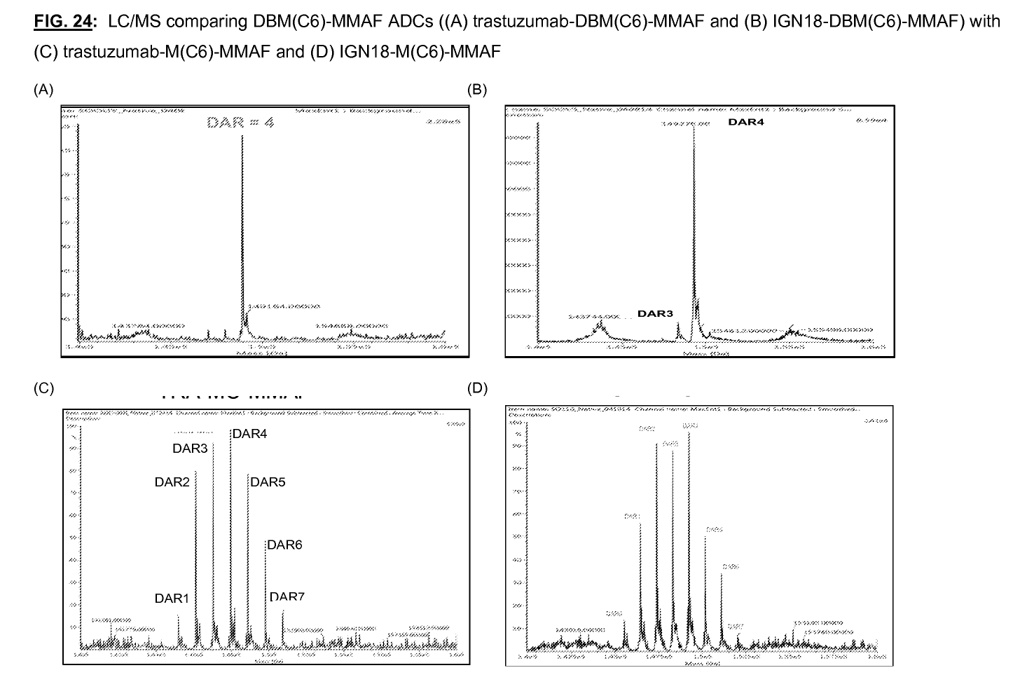

LC/MS analysis of the ADCs confirmed the HIC results and provided accurate molecular weights for the different ADC components. The relative DAR compositions determined by LC/MS are comparable to those determined by HIC and the observed molecular weights are consistent with those calculated based on the predicted structures of the ADCs (see FIGS. 24 (A)-(D)). The LC/MS results indicate that ADCs conjugated with DBM(C6)-MMAF are >95% homogeneous with DARs of 4 drugs/antibody.

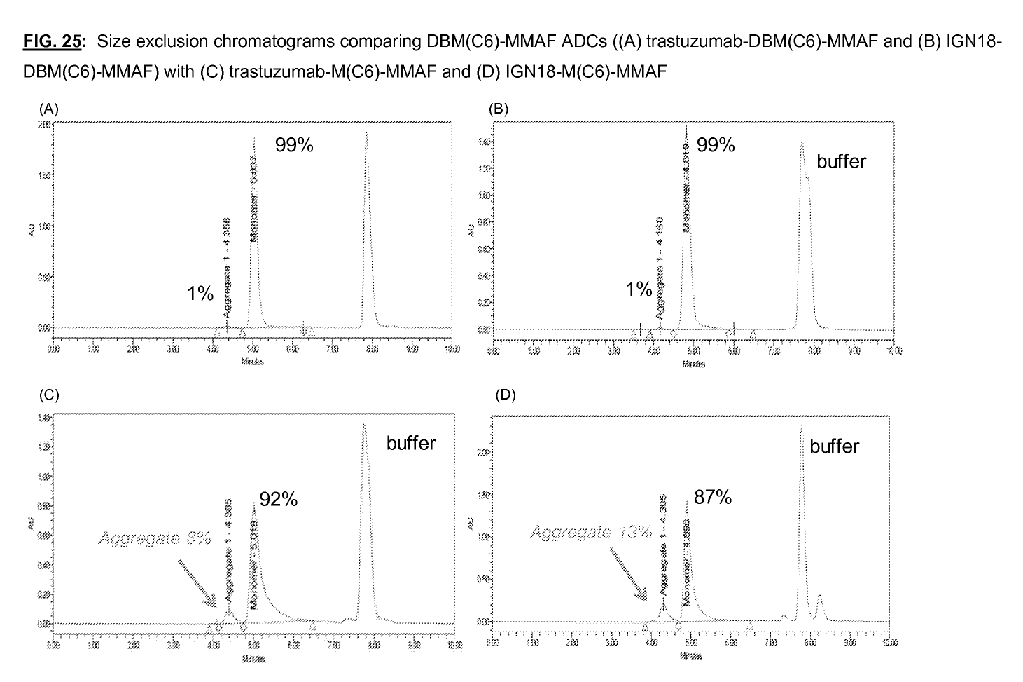

The monomeric purity of the ADCs was evaluated by size exclusion chromatography (SEC). The SEC traces shown in FIG. 25 (A)-(D) indicate that ADCs conjugated with DBM(C6)-MMAF contain less than 2% high molecular weight aggregates and have comparable purity to the unconjugated parent antibodies (data not shown). In contrast, ADCs containing an MC linker contained up to 13% aggregated protein (see FIGS. 25 (C) and (D)).

B. Trastuzumab, Bevacizumab, Rituximab, and Cetuximab

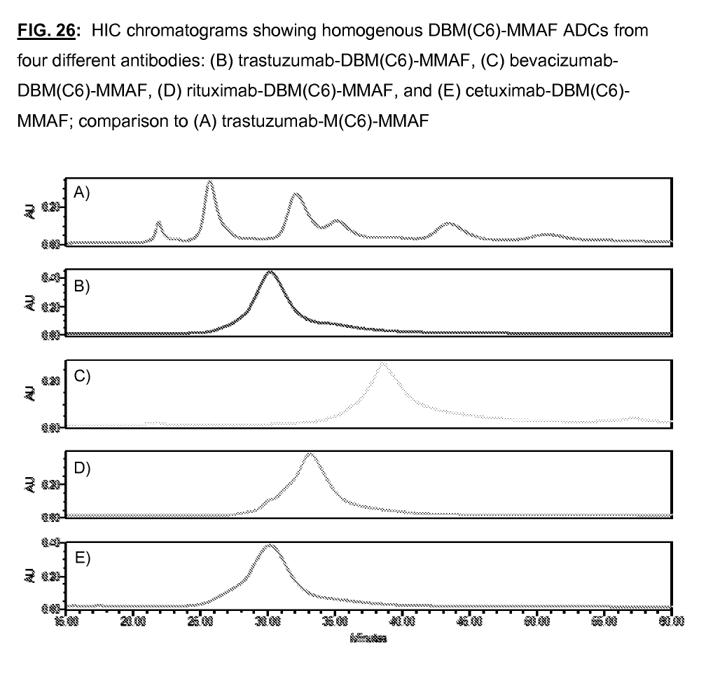

Trastuzumab, bevacizumab, rituximab, and cetuximab, were also prepared and conjugated with linker-cytotoxins according to the methods of Part A above. HIC analysis of the ADCs showed that ADCs conjugated with DBM(C6)-MMAF eluted as single homogeneous peaks with retention times consistent with DARs of 4 drugs/antibody (see FIGS. 26 (B)-(E)). In contrast, an ADC containing an MC linker demonstrated highly heterogeneous HIC profiles with DARs ranging from 0 to 8 drugs/antibody (see FIG. 26 (A) for trastuzumab-M(C6)-MMAF).

C. Other Exemplary Monoclonal Antibodies

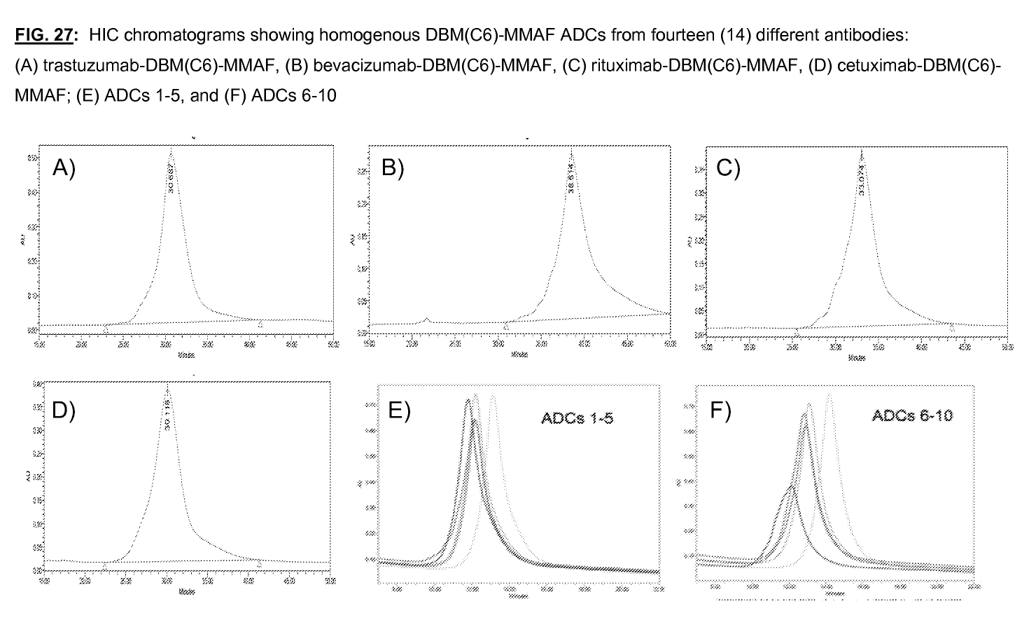

Ten other exemplary monoclonal antibodies with different antigen specificities were also prepared and conjugated with linker-cytotoxins according to the methods of Part A above. HIC analysis of the resulting ADCs conjugated with DBM(C6)-MMAF afforded single homogeneous peaks with retention times consistent with DARs of 4 drugs/antibody (see FIGS. 27 (E) and (F)).

### Example 11B

This example describes characterization of homogeneous ADCs made with the linker-cytotoxin conjugates and antibodies disclosed herein.

A. Trastuzumab and IGN523

Trastuzumab and an exemplary anti-CD98 antibody comprising the VH and VL sequences in Table B (designated herein as “IGN523”) were also prepared and conjugated with linker-cytotoxins according to the methods disclosed herein. Excess reducing agent and a slight excess (5 eq.) of the linker-cytotoxin were used to obtain ADCs with DARs of approximately 4 drugs/antibody. The resulting ADCs were purified via size exclusion chromatagraphy (SEC) to remove excess reagents. The purified ADCs were characterized as described below.

The relative homogeneity and DARs (drugs/antibody ratio) of ADCs were determined using hydrophobic interaction chromatography (HIC) and native LC/MS analysis. HIC analysis enables resolution of ADC fractions containing different DARs due to an increase in hydrophobicity of ADCs with higher DARs. HIC analysis of the ADCs showed that ADCs conjugated with CPM(C6)-MMAF eluted as single homogeneous peaks with retention times consistent with DARs of 4 drugs/antibody (see FIG. 6).

LC/MS analysis of the ADCs confirmed the HIC results and provided accurate molecular weights for the different ADC components. The relative DAR compositions determined by LC/MS are comparable to those determined by HIC and the observed molecular weights are consistent with those calculated based on the predicted structures of the ADCs (see FIG. 7, FIG. 8, and FIG. 9). The LC/MS results indicate that ADCs conjugated with CPM(C6)-MMAF are >85% homogeneous with DARs of 4 drugs/antibody.

The monomeric purity of the ADCs was evaluated by size exclusion chromatography (SEC). The SEC traces shown in FIG. 5 indicate that ADCs conjugated with CPM(C6)-MMAF contain less than 3% high molecular weight aggregates and have comparable purity to the unconjugated parent antibodies (data not shown).

### Example 12: Activity of ADCs

### Example 12A

A. In Vitro Cytotoxicity of ADCs

(1) Anti-HER2 Antibodies:

An exemplary anti-HER2 antibody, trastuzumab (Herceptin®), was purchased and conjugated with linker-cytotoxins for use in primary ADC assays. Antibody drug conjugates for the primary ADC assays were prepared with trastuzumab (Herceptin®) as described herein (see, e.g., Example 9) using linker-cytotoxin conjugate prepared as described herein (see, e.g., Examples 5 and 6).

For the primary ADC assays, carcinoma cell lines were routinely passaged in RPMI media (LifeTech) supplemented with 10-20% fetal calf serum (LifeTech). To assay toxicity, cells were plated in 384-well plates (Greiner), for example, at 3,000 cells (or 5,000 cells) per well in 30 μL (or 40 μL) of media.

For the primary ADC assays with anti-HER2 antibodies, the ovarian carcinoma cell line SKOV-3 is used (obtained from ATCC as HTB-77). For these assays, DBM(C6)-MMAF-conjugated trastuzumab (Herceptin®) antibodies are serially-diluted, for example, from 10 nM or 100 nM, in RPMI and added to appropriate wells in duplicate using an iPipette liquid handler (Apricot Designs). Cell plates are then incubated for three days, followed by lysis in Cell-Titer Glo assay reagent (Promega). For these assays, luminescence is quantified on a Synergy HT plate reader (BioTek) and graphed. IC₅₀s are calculated by fitting to a four-parameter sigmoidal fit (GraphPad).

When tested in these assays, trastuzumab-DBM(C6)-MMAF had an IC₅₀ (nM) of 0.115.

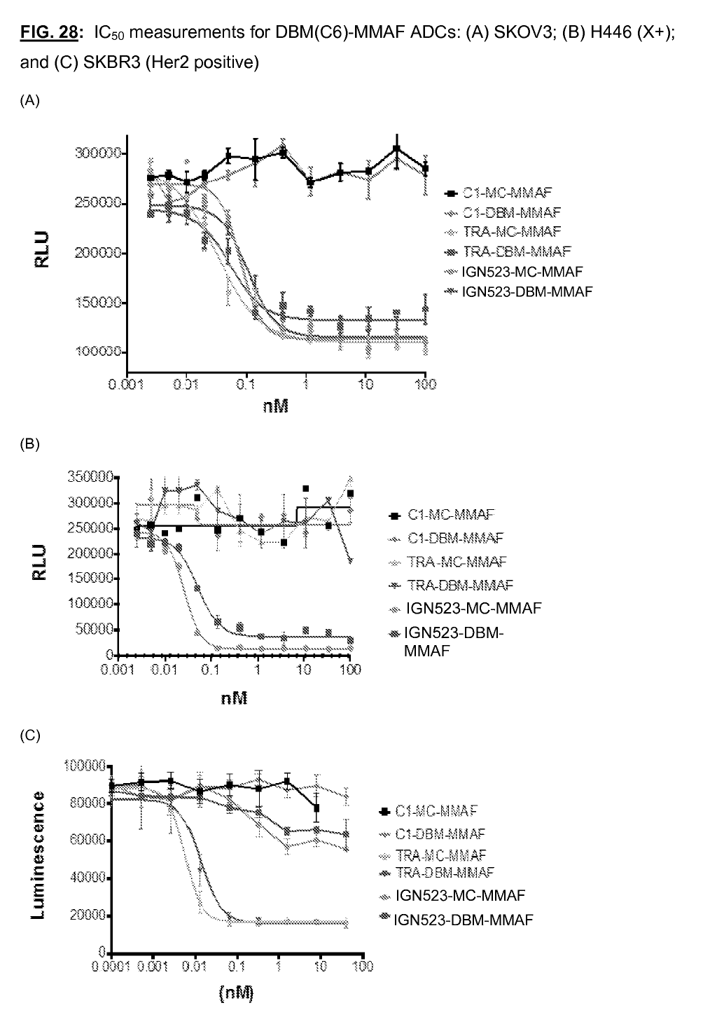

Additional assays with SKOV3 (Her²⁺ & CD98⁺) cells, H446 cells (CD98⁺) and SKBR3 (ErbB2⁺) cells, were performed as described above. The first cell line (SKOV3) expresses both ErbB2 and CD98 antigens. The second cell line (H446) expresses CD98 but not ErbB2. The third cell line (SKBR3) expresses high levels of ErbB2 but does not express CD98. DBM(C6)-MMAF-conjugated trastuzumab inhibited growth of SKOV3 and SKBR3 cells at sub nanomolar concentrations, but did not inhibit growth of H446 cells lacking the Her2 or ErbB2 antigen (see FIG. 28 and Table 6). The lack of inhibitory activity observed for H446 cells suggests minimal non-specific cell killing occurs.

TABLE 6
ADC Assays-IC₅₀ Data (nM)

| ADC | SKBr3 | H446 | SKOV3 |
|---|---|---|---|
| trastuzumab-MC-MMAF | 0.006 | >100 | 0.03 |
| trastuzumab-DBM(C6)-MMAF | 0.01 | >100 | 0.05 |
| IGN523-MC-MMAF | >100 | 0.03 | 0.08 |
| IGN523-DBM(C6)-MMAF | >100 | 0.05 | 0.1 |

The affinity of DBM(C6)-MMAF-conjugated trastuzumab for its purified antigen, ErbB2, was also determined using surface plasmon resonance (SPR) on a Biacore instrument.

Each antibody or ADC was diluted to a concentration of 100 nM and captured onto a Goat anti-human Fc surface (Invitrogen) on a BioRad ProteOn XPR 36 system. The running buffer included 10 mM HEPES pH 7.4, 150 mM NaCl, 0.005% tween-20 and 0.1 mg/ml BSA. All data were collected at 25 PC. Data were processed and fit in Scrubber-Pro6 (Biological Software Pty Ltd). Responses were referenced using the reference channel as well as the buffer blank injection. Data were fit to a 1:1 interaction model.

The results indicate that trastuzumab-DBM(C6)-MMAF had a K_{D} of 0.24 nM (see Table 7), consistent with the binding affinities measured for unconjugated tratuzumab and trastuzumab-MC-MMAF.

TABLE 7
Antigen Binding Affinity Determined via SPR (Biacore)

| ADC | Antigen | K_{D} (nM) |
|---|---|---|
| trastuzumab | Her2 | 0.23 |
| trastuzumab-MC-MMAF | Her2 | 0.25 |
| trastuzumab-DBM(C6)-MMAF | Her2 | 0.24 |
| IGN523 | CD98 | 0.14 |
| IGN523-MC-MMAF | CD98 | 0.16 |
| IGN523-DBM(C6)-MMAF | CD98 | 0.18 |

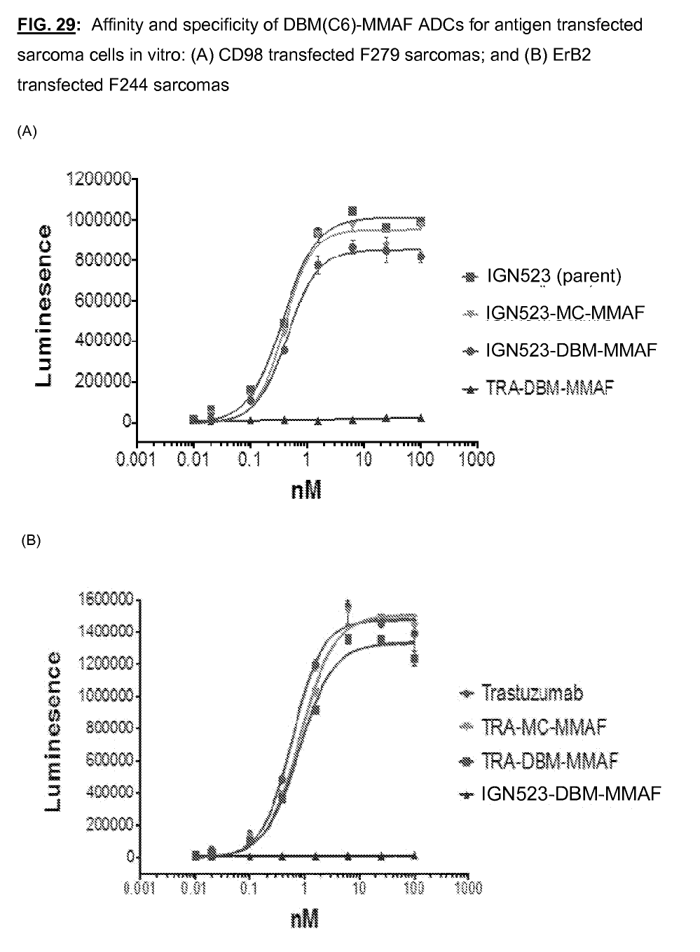

In addition, primary ADC assays using ErB2 transfected F244 sarcoma cells were performed and the results are shown in FIG. 29 (A).

(2) Anti-CD98 Antibodies:

An exemplary anti-CD98 antibody comprising the VH and VL sequences in Table B (designated herein as “IGN523”), was prepared and conjugated with linker-cytotoxins for use in primary ADC assays. Antibody drug conjugates for the primary ADC assays were prepared and tested as described in part (1) above with SKOV-3 cells.

When tested in these assays, IGN523-DBM(C6)-MMAF had an IC₅₀ (nM) of 0.112.

Additional assays with SKOV3 (Her²⁺ & CD98⁺) cells, H446 cells (CD98⁺) and SKBR3 (ErbB2⁺) cells, were performed as described above. MC-MMAF-conjugated IGN523 and DBM(C6)-MMAF-conjugated IGN523 inhibited growth of SKOV3 and H446 cells at sub nanomolar concentrations, but did not inhibit growth of SKBR3 cells lacking the CD98 antigen (see FIG. 28 and Table 6). The lack of inhibitory activity observed for SKBR3 cells suggests minimal non-specific cell killing Occurs.

The affinity of DBM(C6)-MMAF-conjugated IGN523 for its purified antigen, CD98, was also determined using surface plasmon resonance (SPR) on a Biacore instrument, as described above. The results indicate that IGN523-DBM(C6)-MMAF had a K_{D} of 0.18 nM (see Table 7), consistent with the binding affinities measured for unconjugated IGN523 and IGN523-MC-MMAF.

In addition, primary ADC assays using C98 transfected F279 sarcoma cells were performed using a cell-based ELISA protocol and are shown in FIG. 29 (B).

B. Pharmacokinetics of ADCs

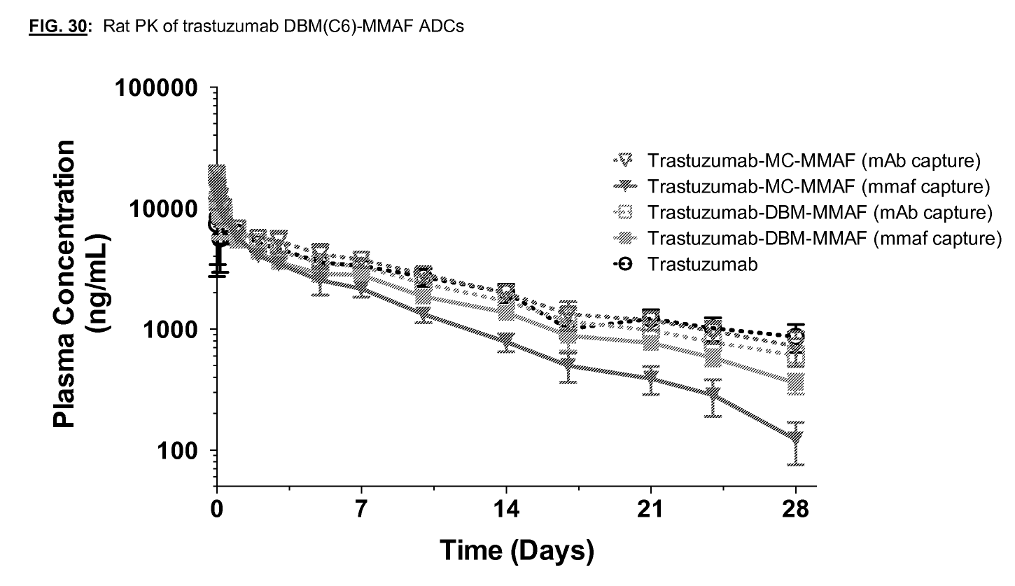

Pharmacokinetic studies were conducted in rats with antibodies and ADCs. For these experiments, trastuzumab was used as a model antibody. Results are shown in Table 8 and FIG. 30. In FIG. 30, trastuzumab-mc-MMAF is represented by the upside-down triangles, and trastuzumab-DBM-MMAF is represented by the squares. Naked trastuzumab was used as a control and is represented by the circles. For these experiments, the rats received one 1 mg/kg dose on day 0. In FIG. 30, the filled-in symbols represent trastuzumab-mc-MMAF and trastuzumab-DBM-MMAF where the ADC was captured on the PK ELISA via MMAF; therefore, the filled in symbols represent intact antibody drug-conjugate. In the Table 8, the calculated half-life and clearance values are shown. The trastuzumab-mc-MMAF captured via MMAF (i.e., intact ADC) had a half-life of 5±1 day, whereas the trastuzumab-mc-MMAF captured via the mAb (i.e., total mAb) had a half-life of 8. This is in contrast to the trastuzumab-DBM-MMAF, where the intact ADC had a half-life of 8±1 and the total mAb had a half-life of 9±1.

TABLE 8
Pharmacokinetics of Trastuzumab ADCs

| | TrzmAb | TrzmAb-DBM-MMAF | | TrzmAb-mc-MMAF | |
|---|---|---|---|---|---|
| | Assay capture: | | | | |
| | TrzmAb | TrzmAb | MMAF | TrzmAb | MMAF |
| T_{1/2} (day) | 10 ± 1 | 9 ± 1 | 8 ± 1 | 8 | 5 ± 1 |
| CL | 13 ± 2 | 14 ± 1 | 18 ± 1 | 13 | 23 ± 3 |
| (mL/day/kg) | | | | | |

C. In Vivo Cytotoxicity of ADCs

Antibodies and ADCs were tested for their anti-tumor activity in animal-tumor models (e.g., murine xenograft models). Exemplary studies were conducted with anti-HER2 antibodies (e.g., trastuzumab), anti-CD98 antibodies (e.g., IGN523), and anti-C16orf54 antibodies (e.g., IGN786), and antibody-drug conjugates (ADCs) of these antibodies. For these studies, various tumor cell lines were used, as obtained from ATCC (Manassis, Va.), the German Collection of Microorganisms and Cell Cultures (DSMZ, Braunschweig, Germany), or the Japanese Collection of Research Bioresources Cell Bank (JCRB, Osaka, Japan) and cultured according to the suppliers' protocols. Animals were obtained from Taconic (Hudson, N.Y.).

(1) Anti-HER2 Antibodies:

An exemplary anti-HER2 antibody, trastuzumab (Herceptin®), was purchased and conjugated with linker-cytotoxins for use in in vivo xenograft animal studies. Antibody drug conjugates for the animal studies were prepared with trastuzumab (Herceptin®) as described herein (see, e.g., Example 9) using linker-cytotoxin conjugate prepared as described herein (see, e.g., Examples 5 and 6).

For in vivo studies conducted with trastuzumab and ADCs of trastuzumab, the ovarian carcinoma cell line SKOV-3 was used, and was obtained from ATCC (HTB-77).

4-6 week-old immunodeficient NOG female mice were used. For the SKOV-3 tumor model, mice were subcutaneously injected on the right flank with 2.6×10⁶ viable cells (SKOV-3) in a mixture of PBS (without magnesium or calcium) and BD Matrigel™ (BD Biosciences). Once the tumor reached a size between 65-200 mm³ mice were randomized. Antibodies or ADCs were administered weekly, and bodyweights and tumors were measured once and twice weekly, respectively. Tumor volume was calculated as described (van der Horst et al. (2009) Neoplasia 11: 355-364). Experiments were performed on groups of at least eight animals per experimental point.

Statistical significance between treatment and control groups was calculated using the Graphpad Prism® software package and applying Student's two-tailed t-test. A p-value of less than 0.05 was considered significant.

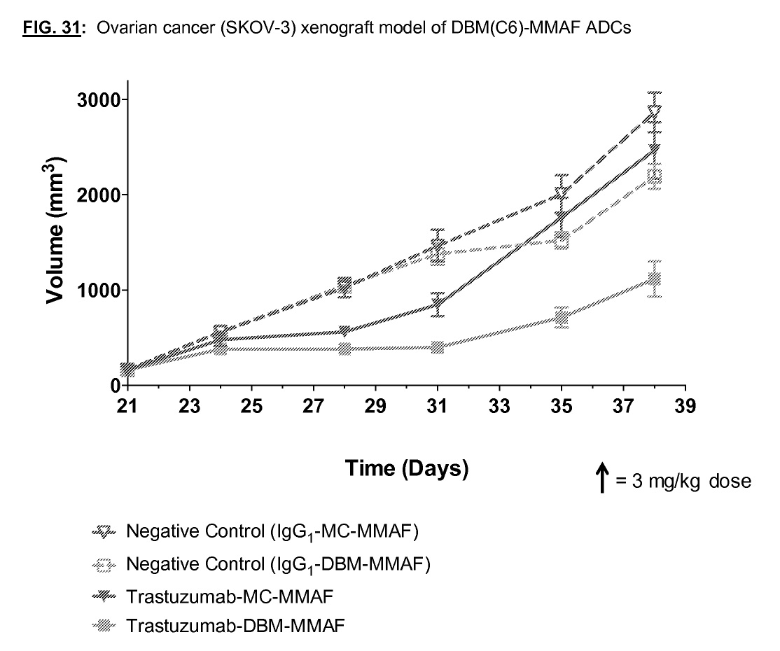

Results are shown in Table 9 and FIG. 31. Trastuzumab-DBM-MMAF, when dosed on day 21 and day 28 at 3 mg/kg, had a statistically significant tumor growth inhibition (TGI) of 65% on day 38, whereas trastuzumab-mc-MMAF did not show statistically significant tumor growth inhibition.

TABLE 9
Ovarian Cancer (SKOV-3) Xenograft Model

| | Volume ± SD | Day 38 | |
|---|---|---|---|
| | (mm³) | TGI (%) | p-Value |
| Negative Control | 2865 ± 209 | — | |
| (IgG₁-MC-MMAF) | | | |
| Negative Control | 2193 ± 130 | | |
| (IgG₁₋DBM-MMAF) | | | |
| trastuzumab-MC-MMAF | 2465 ± 296 | −15 | 0.3 |
| trastuzumab-DBM-MMAF | 1116 ± 186 | −65 | 0.0004 |

(2) Anti-CD98 Antibodies:

An exemplary anti-CD98 antibody comprising the VH and VL sequences in Table B (designated herein as “IGN523”), was prepared and conjugated with linker-cytotoxins for use in in vivo xenograft animal studies. Antibody drug conjugates for the animal studies were prepared with IGN523 as described herein (see, e.g., Example 9) using linker-cytotoxin conjugate prepared as described herein (see, e.g., Examples 5 and 6).

For studies conducted with IGN523 and ADCs of IGN523, the small cell lung cancer cell line H446 was used, and was obtained from ATCC (HTB-171).

4-6 week-old immunodeficient NOD-SCID female mice were used. For the H446 tumor model, mice were subcutaneously injected on the right flank with 2×10⁶ viable cells (H446) in a mixture of PBS (without magnesium or calcium) and BD Matrigel™ (BD Biosciences). Once the tumor reached a size between 65-200 mm³ mice were randomized. Antibodies and ADCs were administered and results analyzed as described above for trastuzumab.

Results are shown in Table 10.

TABLE 10
Lung Cancer (H446) Xenograft Model

| | End of Study | | |
|---|---|---|---|
| | [Starting vol: 152 ± 27] | | |
| | Volume | TGI | wrt. |
| | [mm3] | [%, d 72] | C1.18.4 |
| Negative Control | 3272 ± 134 | 0.4 | 0.9573 |
| (IgG₁-MC-MMAF) | | | |
| Negative Control | 3261 ± 178 | 0.7 | 0.9263 |
| (IgG₁₋DBM-MMAF) | | | |
| IGN523-MC-MMAF | 0 ± 0 | −105 | 2E−7 |
| IGN523-DBM-C6-MMAF | 0 ± 0 | −105 | 2E−7 |

(3) Anti-C16orf54 Antibodies:

An exemplary anti-C16orf54 antibody comprising the VH and VL sequences in Table C (designated herein as “IGN786”), was prepared and conjugated with linker-cytotoxins for use in in vivo xenograft animal studies. Antibody drug conjugates for the animal studies were prepared with IGN786 as described herein (see, e.g., Example 9) using linker-cytotoxin conjugate prepared as described herein (see, e.g., Examples 5 and 6).

For studies conducted with IGN786 and ADCs of IGN786, several cell lines were used. In some experiments, PL21 cells, acute myeloid leukemia cells obtained from DSMZ (ACC 536) were used at a concentration of 3.7×10⁶ cells. In some experiments, IGN-LYMPH-003 cells, from a patient-derived mantle cell lymphoma, were used at a concentration 5×10⁶ cells. In some experiments, THP-1 cells, acute myeloid leukemia cells obtained from the ATCC (TIB202), were used at a concentration of 4.5×10⁶ cells. In some experiments, OCI-AML-3 cells, acute myeloid leukemia cells obtained from DSMZ (ACC 582), were used at a concentration of 4.3×10⁶ cells, 4.2×10⁶ cells, and 4.2×10⁶ cells.

For experiments with OCI-AML-3 cells, 4-6 week-old immunodeficient CB17.SCID female mice were used.

Results are shown in Table 11.

TABLE 11
Acute Myeloid Leukemia Cancer (OCI-
AML-3 cells) Xenograft Model

| | Volume* | Std Dev | TGI | |
|---|---|---|---|---|
| Treatments | [mm³] | [mm³] | [%] | p-value |
| Negative Control | 3024 | 1337 | — | — |
| (IgG₁₋DBM-MMAF) | | | | |
| IGN786-DBM-C6-MMAF | 188 | 80 | −100 | 0.000150 |

### Example 12B

A. In Vitro Cytotoxicity of ADCs

(1) Anti-HER2 Antibodies:

An exemplary anti-HER2 antibody, trastuzumab (Herceptin®), was purchased and conjugated with linker-cytotoxins for use in primary ADC assays. Antibody drug conjugates for the primary ADC assays were prepared with trastuzumab (Herceptin®) as described herein (see, e.g., Example 9) using linker-cytotoxin conjugate prepared as described herein (see, e.g., Examples 5 and 6).

For the primary ADC assays, carcinoma cell lines were routinely passaged in RPMI media (LifeTech) supplemented with 10-20% fetal calf serum (LifeTech). To assay toxicity, cells were plated in 384-well plates (Greiner), for example, at 3,000 cells (or 5,000 cells) per well in 30 μL (or 40 μL) of media.

For the primary ADC assays with anti-HER2 antibodies, the ovarian carcinoma cell line SKOV-3 is used (obtained from ATCC as HTB-77). For these assays, CPM(C6)-MMAF-conjugated trastuzumab (Herceptin®) antibodies are serially-diluted, for example, from 10 nM or 100 nM, in RPMI and added to appropriate wells in duplicate using an iPipette liquid handler (Apricot Designs). Cell plates are then incubated for three days, followed by lysis in Cell-Titer Glo assay reagent (Promega). For these assays, luminescence is quantified on a Synergy HT plate reader (BioTek) and graphed. IC₅₀s are calculated by fitting to a four-parameter sigmoidal fit (GraphPad).

When tested in these assays, trastuzumab-CPM(C6)-MMAF had an IC₅₀ (nM) of 0.043.

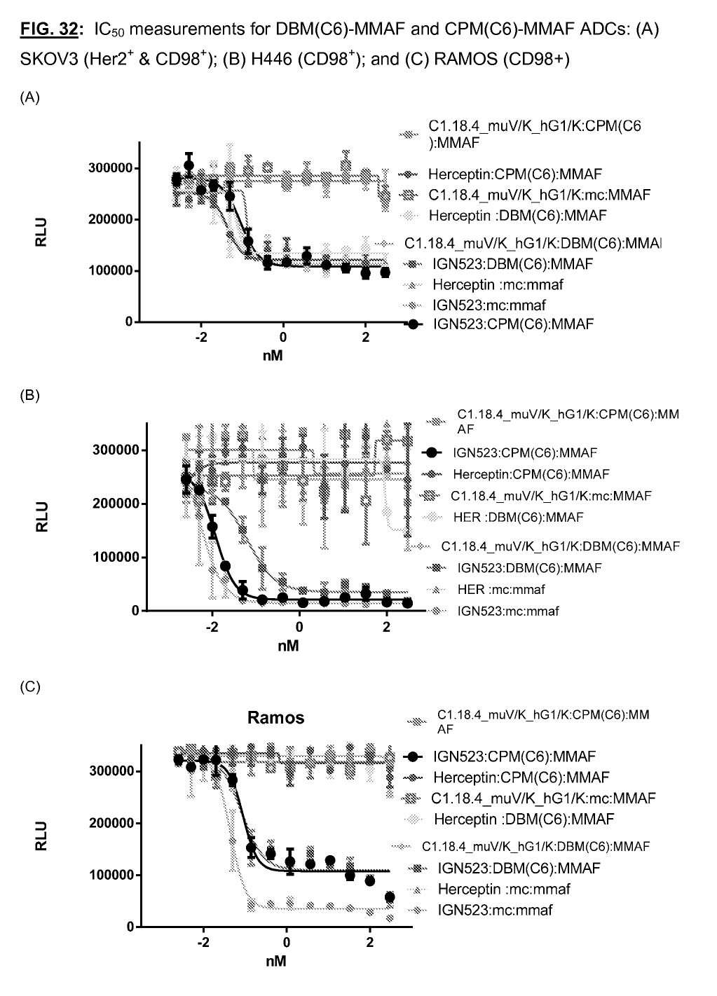

Additional assays with SKOV3 (Her²⁺ & CD98⁺) cells, H446 cells (CD98⁺) and RAMOS (CD98⁺) cells, were performed as described above. The first cell line (SKOV3) expresses both ErbB2 and CD98 antigens. The other two cell lines (H446 and RAMOS) express CD98 but not ErbB2. CPM(C6)-MMAF-conjugated trastuzumab inhibited growth of SKOV3 cells at sub nanomolar concentrations, but did not inhibit growth of H446 or RAMOS cells lacking the Her2 or ErbB2 antigen (see FIG. 32 and Table 12). The lack of inhibitory activity observed for H446 cells suggests minimal non-specific cell killing occurs.

TABLE 12
ADC Assays - IC₅₀ Data (nM)

| ADC | RAMOS | H446 | SKOV3 |
|---|---|---|---|
| trastuzumab-MC-MMAF | >50 | >50 | 0.03 |
| trastuzumab-CPM(C6)-MMAF | >50 | >50 | 0.04 |
| IGN523-MC-MMAF | 0.04 | 0.005 | 0.08 |
| IGN523-CPM(C6)-MMAF | 0.09 | 0.01 | 0.09 |

The affinity of CPM(C6)-MMAF-conjugated trastuzumab for its purified antigen, ErbB2, was also determined using surface plasmon resonance (SPR) on a Biacore instrument.

Each antibody or ADC was diluted to a concentration of 100 nM and captured onto a Goat anti-human Fc surface (Invitrogen) on a BioRad ProteOn XPR 36 system. The running buffer included 10 mM HEPES pH 7.4, 150 mM NaCl, 0.005% tween-20 and 0.1 mg/ml BSA. All data were collected at 25° C. Data were processed and fit in Scrubber-Pro6 (Biological Software Pty Ltd). Responses were referenced using the reference channel as well as the buffer blank injection. Data were fit to a 1:1 interaction model.

The results indicate that trastuzumab-CPM(C6)-MMAF had a K_{D} of 0.22 nM (see Table 13), consistent with the binding affinities measured for unconjugated tratuzumab and trastuzumab-MC-MMAF.

TABLE 13
Antigen Binding Affinity Determined via SPR (Biacore)

| ADC | Antigen | K_{D} (nM) |
|---|---|---|
| trastuzumab | Her2 | 0.24 |
| trastuzumab-MC-MMAF | Her2 | 0.24 |
| trastuzumab-CPM(C6)-MMAF | Her2 | 0.22 |

B. Pharmacokinetics of ADCs

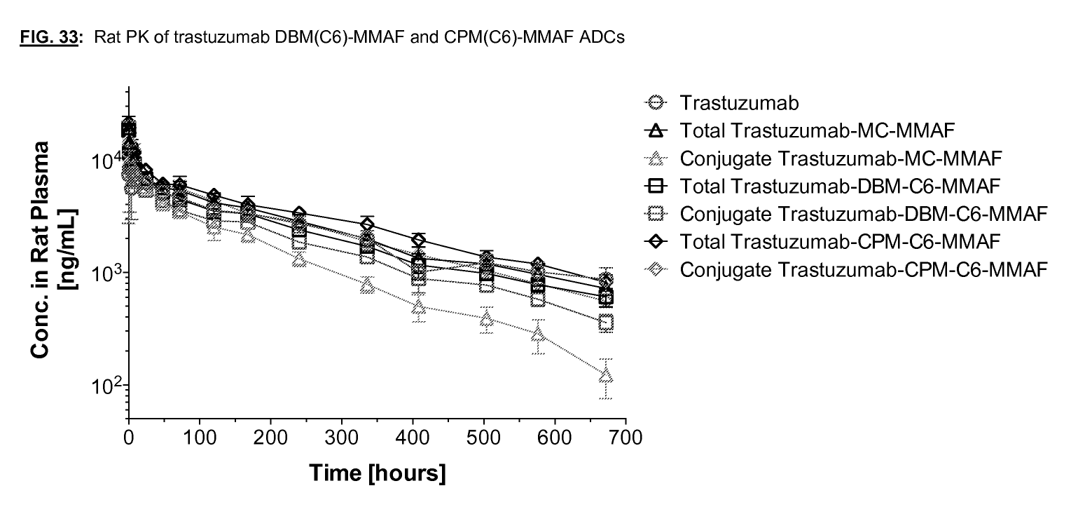

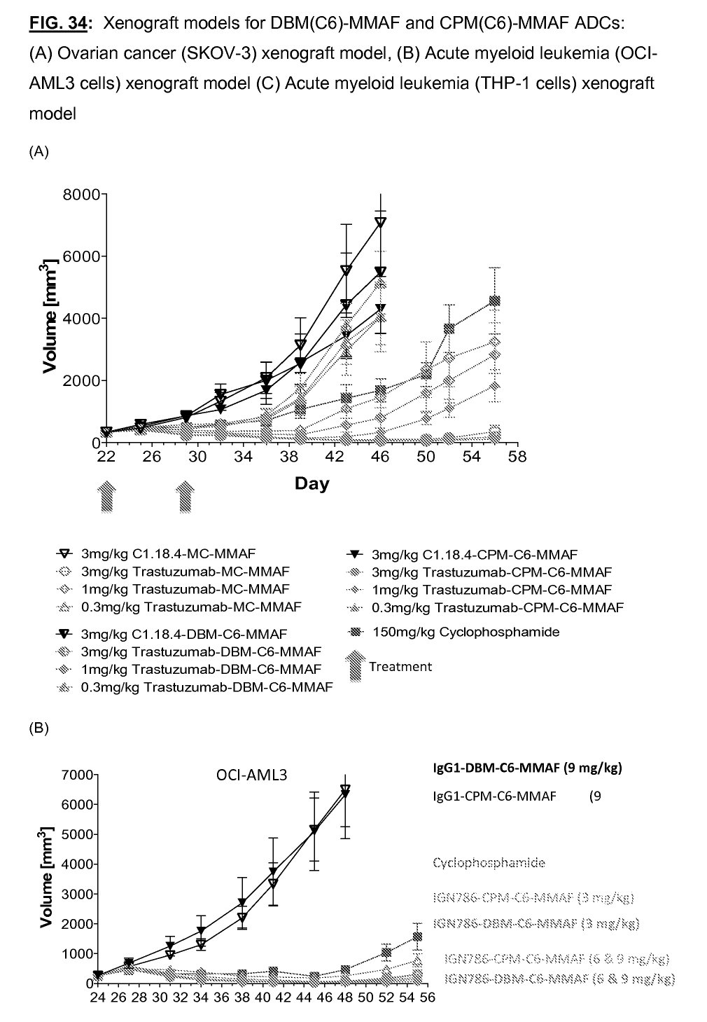

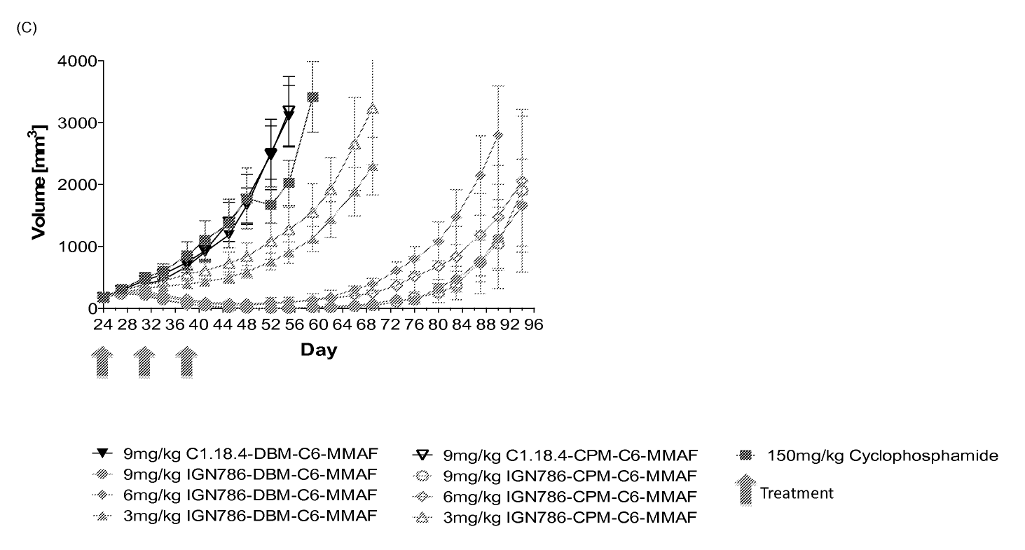

Pharmacokinetic studies were conducted in rats with antibodies and ADCs. For these experiments, trastuzumab was used as a model antibody. Results are shown in Table 14 and FIG. 33. Naked trastuzumab was used as a control and is represented by the circles. For these experiments, the rats received one 1 mg/kg dose on day 0. In Table 14, the calculated half-life and clearance values are shown. The trastuzumab-mc-MMAF captured via MMAF (i.e., intact ADC) had a half-life of 5±1 day, whereas the trastuzumab-mc-MMAF captured via the mAb (i.e., total mAb) had a half-life of 8. This is in contrast to the trastuzumab-CPM-MMAF, where the intact ADC had a half-life of 8±1 and the total mAb had a half-life of 9±1.

TABLE 14
Pharmacokinetics of Trastuzumab ADCs

| | TrzmAb | TrzmAb-CPM-MMAF | | TrzmAb-mc-MMAF | |
|---|---|---|---|---|---|
| | Assay capture: | | | | |
| | TrzmAb | TrzmAb | MMAF | TrzmAb | MMAF |
| T_{1/2} (day) | 10 ± 1 | 9 ± 1 | 8 ± 1 | 8 | 5 ± 1 |
| CL | 13 ± 2 | 10 ± 1 | 12 ± 1 | 13 | 23 ± 3 |
| (mL/day/kg) | | | | | |

C. In Vivo Cytotoxicity of ADCs

Antibodies and ADCs were tested for their anti-tumor activity in animal-tumor models (e.g., murine xenograft models). Exemplary studies were conducted with anti-HER2 antibodies (e.g., trastuzumab), anti-CD98 antibodies (e.g., IGN523), and anti-C16orf54 antibodies (e.g., IGN786), and antibody-drug conjugates (ADCs) of these antibodies. For these studies, various tumor cell lines were used, as obtained from ATCC (Manassis, Va.), the German Collection of Microorganisms and Cell Cultures (DSMZ, Braunschweig, Germany), or the Japanese Collection of Research Bioresources Cell Bank (JCRB, Osaka, Japan) and cultured according to the suppliers' protocols. Animals were obtained from Taconic (Hudson, N.Y.).

(1) Anti-HER2 Antibodies:

An exemplary anti-HER2 antibody, trastuzumab (Herceptin®), was purchased and conjugated with linker-cytotoxins for use in in vivo xenograft animal studies. Antibody drug conjugates for the animal studies were prepared with trastuzumab (Herceptin®) as described herein (see, e.g., Example 9) using linker-cytotoxin conjugate prepared as described herein (see, e.g., Examples 5 and 6).

For in vivo studies conducted with trastuzumab and ADCs of trastuzumab, the ovarian carcinoma cell line SKOV-3 was used, and was obtained from ATCC (HTB-77).

4-6 week-old immunodeficient NOG female mice were used. For the SKOV-3 tumor model, mice were subcutaneously injected on the right flank with 2.6×10⁶ viable cells (SKOV-3) in a mixture of PBS (without magnesium or calcium) and BD Matrigel™ (BD Biosciences). Once the tumor reached a size between 65-200 mm³ mice were randomized. Antibodies or ADCs were administered weekly, and bodyweights and tumors were measured once and twice weekly, respectively. Tumor volume was calculated as described (van der Horst et al. (2009) Neoplasia 11: 355-364). Experiments were performed on groups of at least eight animals per experimental point.

Statistical significance between treatment and control groups was calculated using the Graphpad Prism® software package and applying Student's two-tailed t-test. A p-value of less than 0.05 was considered significant.

Results are shown in Table 15 and FIG. 34 (A). Trastuzumab-CPM-MMAF, when dosed on day 21 and day 28 at 3 mg/kg, had a statistically significant tumor growth inhibition (TGI) of 107% on day 46.

TABLE 15
Ovarian Cancer (SKOV-3) Xenograft Model

| | Volume ± SD | Day 46 | |
|---|---|---|---|
| | (mm³) | TGI (%) | p-Value |
| Negative Control | 5485 ± 1969 | — | — |
| (IgG₁-DBM-MMAF) | | | |
| Negative Control | 4295 ± 792 | — | — |
| (IgG₁₋CPM-MMAF) | | | |
| trastuzumab-DBM-MMAF | 56 ± 11 | −105 | 0.0330 |
| trastuzumab-CPM-MMAF | 52 ± 6 | −107 | 0.0033 |

(3) Anti-C16orf54 Antibodies:

An exemplary anti-C16orf54 antibody comprising the VH and VL sequences in Table C (designated herein as “IGN786”), was prepared and conjugated with linker-cytotoxins for use in in vivo xenograft animal studies. Antibody drug conjugates for the animal studies were prepared with IGN786 as described herein (see, e.g., Example 9) using linker-cytotoxin conjugate prepared as described herein (see, e.g., Examples 5 and 6).

For studies conducted with IGN786 and ADCs of IGN786, several cell lines were used. In some experiments, PL21 cells, acute myeloid leukemia cells obtained from DSMZ (ACC 536) were used at a concentration of 3.7×10⁶ cells. In some experiments, IGN-LYMPH-003 cells, from a patient-derived mantle cell lymphoma, were used at a concentration 5×10⁶ cells. In some experiments, THP-1 cells, acute myeloid leukemia cells obtained from the ATCC (TIB202), were used at a concentration of 4.5×10⁶ cells. In some experiments, OCI-AML-3 cells, acute myeloid leukemia cells obtained from DSMZ (ACC 582), were used at a concentration of 4.3×10⁶ cells, 4.2×10⁶ cells, and 4.2×10⁶ cells.

For experiments with OCI-AML-3 cells, 4-6 week-old immunodeficient CB17.SCID female mice were used. Results are shown in Table 16 and FIG. 34 (B) for OCI-AML3 cells, and in Table 17 and FIG. 34 (C) for THP-1 cells.

TABLE 16
Acute Myeloid Leukemia Cancer (OCI-AML-3
cells) Xenograft Model (Day 48)

| | Volume* | Std Dev | TGI | |
|---|---|---|---|---|
| Treatments | [mm³] | [mm³] | [%] | p-value |
| Negative Control | 6336 | 1477 | — | — |
| (IgG₁₋DBM-C6-MMAF) | | | | |
| IGN786-DBM-C6-MMAF | 70 | 14 | −103 | 0.0132 |
| Negative Control | 6499 | 1249 | — | — |
| (IgG₁₋CPM-C6-MMAF) | | | | |
| IGN786-CPM-C6-MMAF | 74 | 24 | −103 | 0.0068 |

TABLE 17
Acute Myeloid Leukemia Cancer (THP-1
cells) Xenograft Model (Day 55)

| | Volume* | Std Dev | TGI | |
|---|---|---|---|---|
| Treatments | [mm³] | [mm³] | [%] | p-value |
| Negative Control | 3106 | 499 | — | — |
| (IgG₁₋DBM-C6-MMAF) | | | | |
| IGN786-DBM-C6-MMAF | 11 | 22 | −106 | 0.0004 |
| Negative Control | 3329 | 475 | — | — |
| (IgG₁₋CPM-C6-MMAF) | | | | |
| IGN786-CPM-C6-MMAF | 3 | 3 | −106 | 0.0002 |

### Example 13: Additional Methods for Making ADCs

### Example 13A

This example provides additional methods for making ADCs using the linker-cytotoxin conjugates and antibody hinge mutants disclosed herein.

An optional DAR (drugs-antibody ratio) is desirable for ADCs, including, for example, a DAR of 2, 3, or 4. For example, the following schemes illustrates general schemes for preparation of homogenous ADCs with DAR=2, 3, or 4, as disclosed herein, which may be made by the methods disclosed herein.

For example, for ADCs with IgG1 antibodies, one or both of the hinge cysteines may be mutated to another amino acid (e.g., alanine) to prepare ADC with a DAR of 3 or 2, respectively.

For example, for ADCs with IgG2 antibodies, two, three or four of the hinge cysteines may be mutated to another amino acid (e.g., alanine) to prepare ADC with a DAR of 4, 3 or 2, respectively.

For example, for ADCs with IgG3 antibodies, nine, ten or eleven of the hinge cysteines may be mutated to another amino acid (e.g., alanine) to prepare ADC with a DAR of 4, 3 or 2, respectively.

For another example, for ADCs with IgG4 antibodies, one or both of the hinge cysteines may be mutated to another amino acid (e.g., alanine) for prepare ADC with a DAR of 3 or 2, respectively.

Illustrative General Schemes for Preparation of ADCs with DAR=2 or 3:

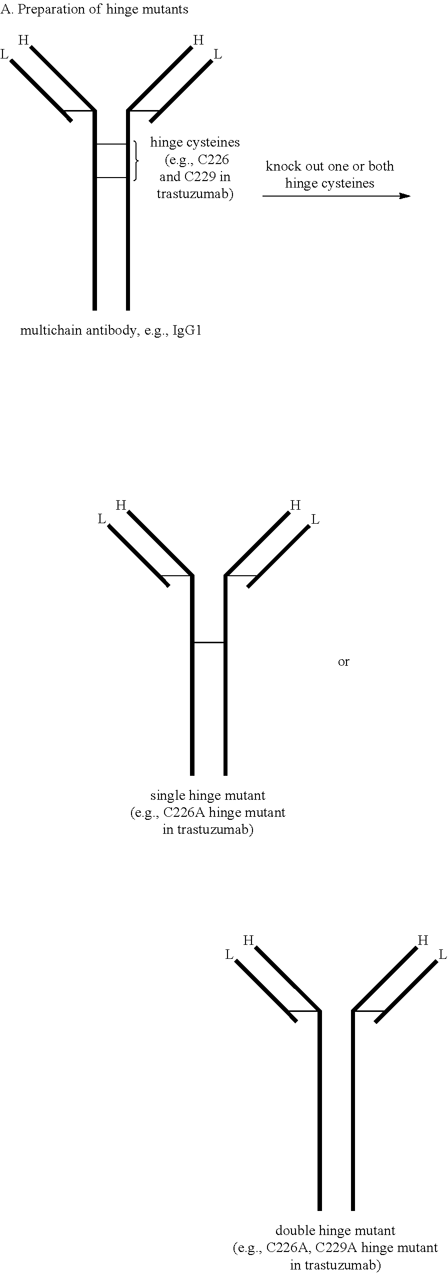

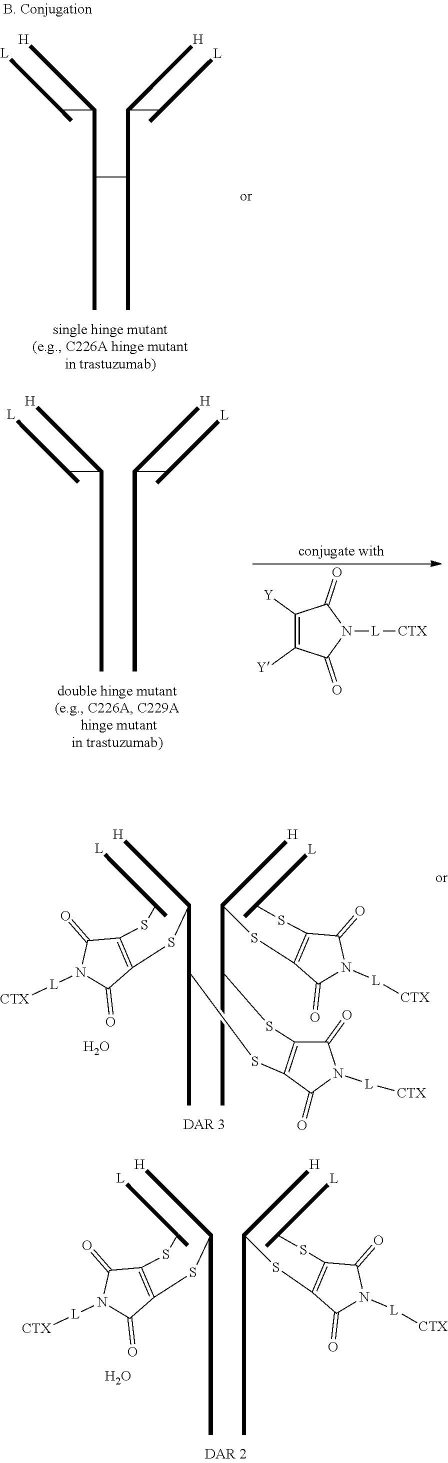

### A. Preparation of Hinge Mutants

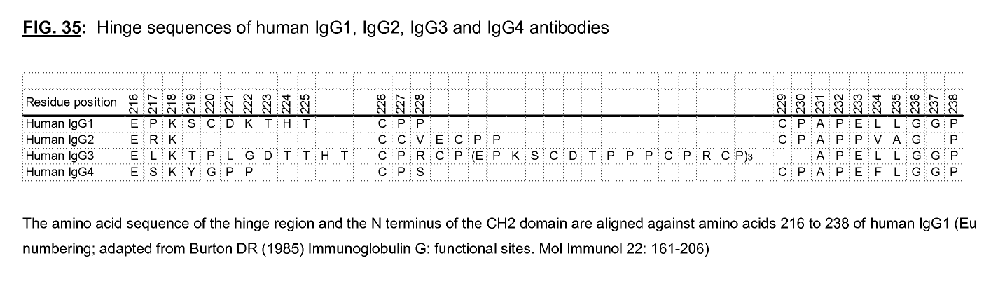

The hinge regions of the human IgG1 and IgG4 heavy chains contain two cysteine residues, whereas the hinge region of the human IgG3 heavy chains contains eleven cysteine residues, and the hinge region of the human IgG4 heavy chains contains four cysteine residues (see FIG. 35). IgG hinge mutants were generated by mutating one or both cysteines in the hinge region to structurally related amino acids, for example, alanines. Hinge residues are numbered using human IgG1 Eu numbering (Burton D R (1985) Immunoglobulin G: functional sites. Mol Immunol 22: 161-206). Tables 18-21 provide the correspondence between the IMGT unique numbering for C-DOMAIN, the IMGT exon numbering, the Eu and Kabat numberings for human IgG1 (Table 18), IgG2 (Table 19), IgG3 (Table 20) and IgG4 (Table 21).

TABLE 18
Human IgG1

| | IGHG1 | | | |
|---|---|---|---|---|
| | amino acid | | Eu | Kabat |
| IMGT numbering | translation | IMGT exon | numbering | numbering |
| for the hinge | J00228 | numbering | [1](4) | [2](4) |
| 1 | (E) | 1 | 216 | 226 |
| 2 | P | 2 | 217 | 227 |
| 3 | K | 3 | 218 | 228 |
| 4 | S | 4 | 219 | 232 |
| 5 | C | 5 | 220 | 233 |
| 6 | D | 6 | 221 | 234 |
| 7 | K | 7 | 222 | 235 |
| 8 | T | 8 | 223 | 236 |
| 9 | H | 9 | 224 | 237 |
| 10 | T | 10 | 225 | 238 |
| 11 | C | 11 | 226 | 239 |
| 12 | P | 12 | 227 | 240 |
| 13 | P | 13 | 228 | 241 |
| 14 | C | 14 | 229 | 242 |
| 15 | P | 15 | 230 | 243 |

TABLE 19
Human IgG2

| | IGHG2 | | | |
|---|---|---|---|---|
| | amino acid | | Eu | Kabat |
| IMGT numbering | translation | IMGT exon | numbering | numbering |
| for the hinge | J00230 | numbering | [1](4) | [2](4) |
| 1 | (E) | 1 | 216 | 226 |
| 2 | R | 2 | 217 | 227 |
| 3 | K | 3 | 218 | 228 |
| 4 | C | 4 | 219 | 232 |
| 5 | C | 5 | 220 | 233 |
| 6 | V | 6 | 222 | 235 |
| 7 | E | 7 | 224 | 237 |
| 8 | C | 8 | 226 | 239 |
| 9 | P | 9 | 227 | 240 |
| 10 | P | 10 | 228 | 241 |
| 11 | C | 11 | 229 | 242 |
| 12 | P | 12 | 230 | 243 |

TABLE 20
Human IgG3

| | H1 | | | | | | | |
|---|---|---|---|---|---|---|---|---|
| | IGHG3 | | | | | IGHG3 | | |
| IMGT | amino | | | | IMGT | amino | H2 | |
| numbering | acid | IMGT | Eu | Kabat | numbering | acid | IMGT | Eu |
| for the | translation | exon | numbering | numbering | for the | translation | exon | numbering |
| hinge | X03604 | numbering | [1](4) | [2](4) | hinge | X03604 | numbering | [1] |
| 1 | (E) | 1 | 216 | 226 | 1 | (E) | 1 | — |
| 2 | L | 2 | 217 | 227 | 2 | P | 2 | — |
| 3 | K | 3 | 218 | 228 | 3 | K | 3 | — |
| 4 | T | 4 | — | 229 | 4 | S | 4 | — |
| 5 | P | 5 | — | 230 | 5 | C | 5 | — |
| 6 | L | 6 | 219 | 232 | 6 | D | 6 | — |
| 7 | G | 7 | 220 | 233 | 7 | T | 7 | — |
| 8 | D | 8 | 221 | 234 | 8 | P | 8 | — |
| 9 | T | 9 | 222 | 235 | 9 | P | 9 | — |
| 10 | T | 10 | 223 | 236 | 10 | P | 10 | — |
| 11 | H | 11 | 224 | 237 | 11 | C | 11 | — |
| 12 | T | 12 | 225 | 238 | 12 | P | 12 | — |
| 13 | C | 13 | 226 | 239 | 13 | R | 13 | — |
| 14 | P | 14 | 227 | 240 | 14 | C | 14 | — |
| 15 | R | 15 | 228 | 241 | 15 | P | 15 | — |
| 16 | C | 16 | — | 241A | 16 | — | — | — |
| 17 | P | 17 | — | 241B | 17 | — | — | — |
| | IMGT | H2 | H3 | | | H4 | | |
| | numbering | Kabat | IMGT | Eu | Kabat | IMGT | Eu | Kabat |
| | for the | numbering | exon | numbering | numbering | exon | numbering | numbering |
| | hinge | [2] | numbering | [1] | [2] | numbering | [1] | [2] |
| | 1 | 241C | 1 | — | 241R | 1 | — | 241GG |
| | 2 | 241D | 2 | — | 241S | 2 | — | 241HH |
| | 3 | 241E | 3 | — | 241T | 3 | — | 241II |
| | 4 | 241F | 4 | — | 241U | 4 | — | 241JJ |
| | 5 | 241G | 5 | — | 241V | 5 | — | 241KK |
| | 6 | 241H | 6 | — | 241W | 6 | — | 241LL |
| | 7 | 241I | 7 | — | 241X | 7 | — | 241MM |
| | 8 | 241J | 8 | — | 241Y | 8 | — | 241NN |
| | 9 | 241K | 9 | — | 241Z | 9 | — | 241OO |
| | 10 | 241L | 10 | — | 241AA | 10 | — | 241PP |
| | 11 | 241M | 11 | — | 241BB | 11 | — | 241QQ |
| | 12 | 241N | 12 | — | 241CC | 12 | — | 241RR |
| | 13 | 241O | 13 | — | 241DD | 13 | — | 241SS |
| | 14 | 241P | 14 | — | 241EE | 14 | 229 | 242 |
| | 15 | 241Q | 15 | — | 241FF | 15 | 230 | 243 |
| | 16 | — | — | — | — | — | — | — |
| | 17 | — | — | — | — | — | — | — |

TABLE 21
Human IgG4

| | IGHG4 | | | |
|---|---|---|---|---|
| | amino acid | | Eu | Kabat |
| IMGT numbering | translation | IMGT exon | numbering | numbering |
| for the hinge | K01316 | numbering | [1] | [2] |
| 1 | (E) | 1 | 216 | 226 |
| 2 | S | 2 | 217 | 227 |
| 3 | K | 3 | 218 | 228 |
| 4 | Y | 4 | — | 229 |
| 5 | G | 5 | — | 230 |
| 6 | P | 6 | 224 | 237 |
| 7 | P | 7 | 225 | 238 |
| 8 | C | 8 | 226 | 239 |
| 9 | P | 9 | 227 | 240 |
| 10 | S | 10 | 228 | 241 |
| 11 | C | 11 | 229 | 242 |
| 12 | P | 12 | 230 | 243 |

(1) J00228 corresponds to the IGHG1*01 allele (Alignment of alleles: Human IGHG1) and to a G1m1, 17 chain (G1m allotypes). The Eu gamma1 chain is encoded by the IGHG1*03 allele (CH1 K120 > R, CH3 D12 > E and L14 > M) and is a G1m3 chain (G1m allotypes).
(2) The IGHG1, IGHG3 and IGHG4 CH2 exons encode 110 amino acids. The IGHG2 CH2 exon encodes 109 amino acids, due to a 3 nt deletion corresponding to codon 3 (position 1.4 in the IMGT unique numbering for C-DOMAINs).
(3) The last two amino acids of the IGHG CH3 exons belong to the CHS which encodes the heavy chain C-terminus found in the secreted immunoglobulins.
(4) In Kabat [2], Eu index from 219 to 221 should have been aligned with the Eu protein (pp. 671). As a consequence, Kabat positions 232, 233 and 234 correspond to Eu index positions 219, 220 and 221, respectively (pp. 670-678).
(5) MGT labels (concepts of description) are written in capital letters.
References:
[1] Edelman, G. M. et al., Proc. Natl. Acad. USA, 63, 78-85 (1969). PMID: 5257969
[2] Kabat, E. A. et al., Sequences of proteins of immunological interest. 5th Edition - US Department of Health and Human Services, NIH publication no 91-3242, pp 662, 680, 689 (1991).

The sequences for wild-type and mutant IGN523, IGN786 and trastuzumab antibodies are listed below.

The amino acid sequence for the IGN523 wild-type heavy chain (VH is shown as amino acids 1-116) is shown in Table D.

TABLE D
QVQLVQSGAEVKKPGSSVKVSCKASGNAFTNYLIEWVRQAPGQGLEWMGV
INPGSGITNYNEKFKGKATITADKSTSTAYMELSSLRSEDTAVYYCSGSA
NWFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG (the
hinge cysteines are at position 225 and 228)
(SEQ ID NO: 7)

The amino acid sequence for the IGN523 single C229A heavy chain mutant (VH is shown as amino acids 1-116) is shown in Table E.

TABLE E
QVQLVQSGAEVKKPGSSVKVSCKASGNAFTNYLIEWVRQAPGQGLEWMGV
INPGSGITNYNEKFKGKATITADKSTSTAYMELSSLRSEDTAVYYCSGSA
NWFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTAPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG (the
mutated residue is underlined) (SEQ ID NO: 8)

The amino acid sequence for the IGN523 single C229A heavy chain mutant (VH is shown as amino acids 1-116) is shown in Table F.

TABLE F
QVQLVQSGAEVKKPGSSVKVSCKASGNAFTNYLIEWVRQAPGQGLEWMGV
INPGSGITNYNEKFKGKATITADKSTSTAYMELSSLRSEDTAVYYCSGSA
NWFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPAPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG (the
mutated residue is underlined) (SEQ ID NO: 9)

The amino acid sequence for the IGN523 double C226A C229A heavy chain (VH is shown as amino acids 1-116) mutant is shown in Table G.

TABLE G
QVQLVQSGAEVKKPGSSVKVSCKASGNAFTNYLIEWVRQAPGQGLEWMGV
INPGSGITNYNEKFKGKATITADKSTSTAYMELSSLRSEDTAVYYCSGSA
NWFAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTAPPAPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG (the
mutated residues are underlined) (SEQ ID NO: 10)

The amino acid sequence for the IGN523 wild-type light chain with signal sequence is shown in Table H.

TABLE H
MSVPTQVLGLLLLWLTDARCDIVMTQSPDSLAVSLGERATINCKSSQSLL
YSSNQKNYLAWYQQKPGQPPKLLIYWASTRDSGVPDRFTGSGSGTDFTLT
ISSLQAEDVAVYYCQRYYGYPWTFGGGTKVEIKRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGEC
(SEQ ID NO: 11)

The amino acid sequence for the trastuzumab wild-type heavy chain (VH is shown as amino acids 1-120) is shown in Table I.

TABLE I
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(thehinge cysteines are at position 229 and 232)
(SEQ ID NO: 12)

The amino acid sequence for the trastuzumab single C226A heavy chain mutant (VH is shown as amino acids 1-120) is shown in Table J.

TABLE J
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTAPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 13)

The amino acid sequence for the trastuzumab single C229A heavy chain mutant (VH is shown as amino acids 1-120) is shown in Table K.

TABLE K
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 14)

The amino acid sequence for the trastuzumab double C226A C229A heavy chain mutant (VH is shown as amino acids 1-120) is shown in Table L.

TABLE L
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTAPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residues are underlined) (SEQ ID
NO: 15)

The amino acid sequence for the trastuzumab wild-type light chain variable region (VL) is shown in Table M.

TABLE M
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIK (SEQ ID NO: 16)

The amino acid sequence for the IGN786 wild-type heavy chain (VH is shown as amino acids 1-121) is shown in Table N.

TABLE N
QVQLQESGPGLVKPSDTLSLTCAVSGYSITSDYAWNWIRQPPGKGLEWMG
YISYSGSIRYNPSLKSRITISRDTSKNQFSLKLSSVTAVDTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(thehinge cysteines are at position 229 and 232)
(SEQ ID NO: 17)

The amino acid sequence for the IGN786 single C226A heavy chain (VH is shown as amino acids 1-121) mutant is shown in Table O.

TABLE O
QVQLQESGPGLVKPSDTLSLTCAVSGYSITSDYAWNWIRQPPGKGLEWMG
YISYSGSIRYNPSLKSRITISRDTSKNQFSLKLSSVTAVDTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTAPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 18)

The amino acid sequence for the IGN786 single C229A heavy chain mutant (VH is shown as amino acids 1-121) is shown in Table P.

TABLE P
QVQLQESGPGLVKPSDTLSLTCAVSGYSITSDYAWNWIRQPPGKGLEWMG
YISYSGSIRYNPSLKSRITISRDTSKNQFSLKLSSVTAVDTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTCPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 19)

The amino acid sequence for the IGN786 double C226A C229A heavy chain mutant (VH is shown as amino acids 1-121) is shown in Table Q.

TABLE Q
QVQLQESGPGLVKPSDTLSLTCAVSGYSITSDYAWNWIRQPPGKGLEWMG
YISYSGSIRYNPSLKSRITISRDTSKNQFSLKLSSVTAVDTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTAPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residues are underlined) (SEQ ID
NO: 20)

The amino acid sequence for the IGN786 wild-type light chain variable region (VL) is shown in Table R.

TABLE R
DIVMTQSPDSLAVSLGERVTLNCKSSQNLLYSTNQKNYLAWYQQKPGQPP
KLLIYWASTRESGVPDRFSGSGSGTDFTLTISSVQAEDLAVYYCQQYYSY
RTFGQGTKLEIK (SEQ ID NO: 21)

The amino acid sequence for the IGN786-B wild-type heavy chain (VH is shown as amino acids 1-121) is shown in Table S.

TABLE S
QVQLQESGPGLVKPSQTLSLTCTVSGYSITSDYAWNWIRQPPGKGLEWMG
YISYSGSIRYNPSLKSRITISRDTSKNQFSLKLSSVTAADTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(thehinge cysteines are at position 229 and 232)
(SEQ ID NO: 22)

The amino acid sequence for the IGN786-B single C226A heavy chain mutant (VH is shown as amino acids 1-121) is shown in Table T.

TABLE T
QVQLQESGPGLVKPSQTLSLTCTVSGYSITSDYAWNWIRQPPGKGLEWMG
YISYSGSIRYNPSLKSRITISRDTSKNQFSLKLSSVTAADTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTAPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 23)

The amino acid sequence for the IGN786-B single C229A heavy chain mutant (VH is shown as amino acids 1-121) is shown in Table U.

TABLE U
QVQLQESGPGLVKPSQTLSLTCTVSGYSITSDYAWNWIRQPPGKGLEWMG
YISYSGSIRYNPSLKSRITISRDTSKNQFSLKLSSVTAADTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTCPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residue is underlined) (SEQ ID NO: 24)

The amino acid sequence for the IGN786-B double C226A C229A heavy chain mutant (VH is shown as amino acids 1-121) is shown in Table V.

TABLE V
QVQLQESGPGLVKPSQTLSLTCTVSGYSITSDYAWNWIRQPPGKGLEWMG
YISYSGSIRYNPSLKSRITISRDTSKNQFSLKLSSVTAADTAVYYCAREK
YDNYYYAMDYWGQGTLVTVSSASTKGPSVFPLAPSSKSTSGGTAALGCLV
KDYFPEPVTVSWNSGALTSGVHTFPAVLQSSGYSLSSVVTVPSSSLGTQT
YICNVNHKPSNTKVDKRVEPKSCDKTHTAPPAPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
(themutated residues are underlined) (SEQ ID
NO: 25)

The amino acid sequence for the IGN786-B wild-type light chain variable region (VL) is shown in Table W.

TABLE W
DIVMTQSPDSLAVSLGERVTLNCKSSQNLLYSTNQKNYLAWYQQKPGQPP
KLLIYWASTRESGVPDRFSGSGSGTDFTLTISSVQAEDLAVYYCQQYYSY
RTFGQGTKLEIK (SEQ ID NO: 26)

B. Conjugation

Prior to conjugation each antibody was buffer exchanged into PBS+5 mM EDTA, pH 7.4 and diluted to a concentration of 5 mg/mL. Each ADC was prepared using the general protocol of TCEP reduction for 2 h at 37° C. followed by addition of link-cytotoxin conjugate from a 10 mM DMA or DMSO stock solution. After 0.5 h at room temperature the ADCs were purified by buffer exchange on sephadex PD-10 spin columns to afford pure ADCs.

For DBM-C6-MMAF conjugates, the following equivalents of TCEP and DBM(C6)MMAF were used: trastuzumab wildtype—8×TCEP, 5.5×DBM(C6)MMAF; trastuzumab(C226A)—8×TCEP, 4.25×DBM(C6)MMAF; trastuzumab(C226AC229A)—6×TCEP, 2.75×DBM(C6)MMAF.

For DBM-VAP-MMAE conjugates, the following equivalents were used: trastuzumab wildtype—8×TCEP, 5.75×DBM(C6)-VAP-MMAE; trastuzumab(C226A)—6×TCEP, 4×DBM(C6)-VAP-MMAE; trastuzumab(C226AC229A)—4×TCEP, 3×DBM(C6)-VAP-MMAE.

For the DBM-VAP-MMAE conjugations additional DMSO was added to the conjugation reaction such that the final concentration of organic solvent was 10%.

### Example 13B

This example provides additional methods for making ADCs using the linker-cytotoxin conjugates and antibody hinge mutants disclosed herein.

A. Preparation of Hinge Mutants

Hinge mutants were made as in Example 13A.

B. General Conjugation Procedure for DAR 2 CPM(C6)-Val-Ala-PBD ADCs

Prior to conjugation the antibody was buffer exchanged into PBS+5 mM EDTA, pH 7.4 and adjusted to a concentration of 10 mg/mL. The antibody was then reduced by addition of 8 equivalents (relative to antibody concentration) of TCEP from a freshly prepared TCEP stock solution. After incubation at 37° C. for 2h the antibody was buffer exchanged into PBD+5 mM EDTA, pH 7.4 to remove any residual TCEP. Three equivalents (relative to antibody concentration) of CPM(C6)-Val-Ala-PBD (from 10 mM DMSO stock solution) were prepared in a volume of propylene glycol equal to half the volume of the antibody solution. The antibody was diluted with propylene glycol such that the concentration of propylene glycol was 33%. The CPM(C6)-Val-Ala-PBD solution was then added to the antibody such that the final concentration of propylene glycol was 50%. For example, if the antibody solution was 1 mL, 500 μL of propylene glycol was added to antibody and the CPM(C6)-Val-Ala-PBD was prepared in an additional 500 μL propylene glycol. After addition of the linker-toxin the total volume of propylene glycol added was 1 mL, for a final concentration of 50%. After 1 h reaction at room temperature the ADCs were purified twice by buffer exchange on sephadex PD-10 spin columns to afford pure ADCs. The following scheme depicts the general conjugation procedure described above.

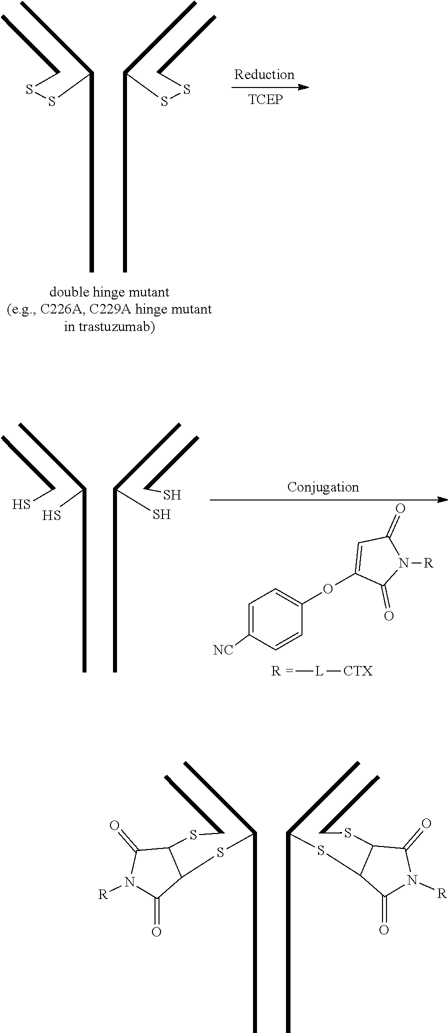

Following the above procedure, the following ADCs were made:

(A) trastuzumab(C226AC229A)-CPM(C6)-Val-Ala-PBD,

(B) IGN523(C226AC229A)-CPM(C6)-Val-Ala-PBD, and

(C) IGN786(C226AC229A)-CPM(C6)-Val-Ala-PBD.

### Example 14: Further Characterization of Homogeneous ADCs

This example describes characterization of homogeneous ADCs made with the linker-cytotoxin conjugates and antibody hinge mutants disclosed herein.

### Example 14A

The relative homogeneity and DARs (drugs/antibody ratio) of ADCs prepared according to Example 13A were determined using hydrophobic interaction chromatography (HIC) and native LC/MS analysis.

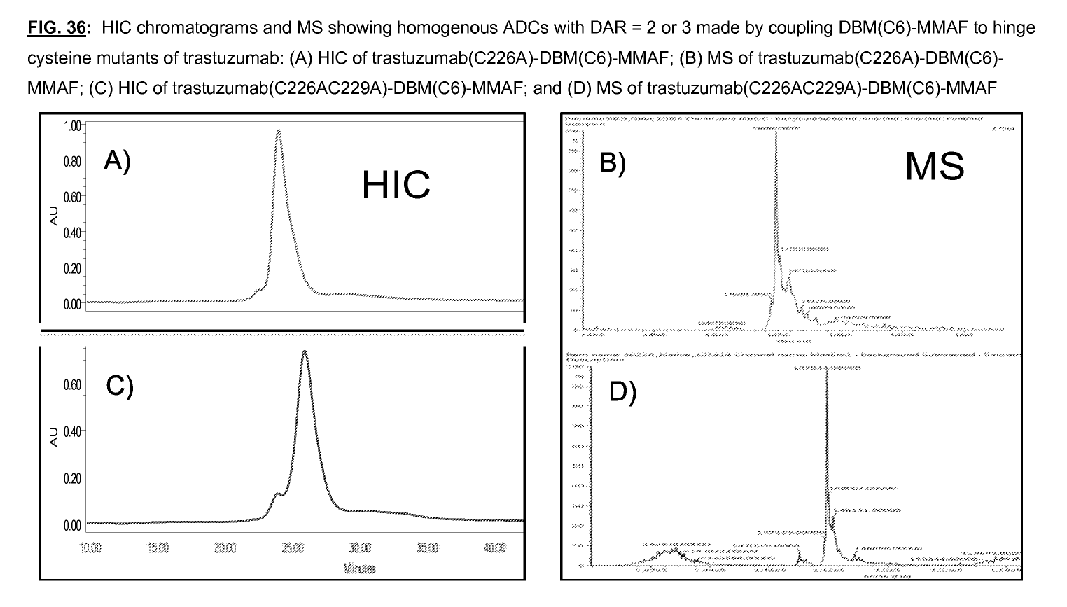

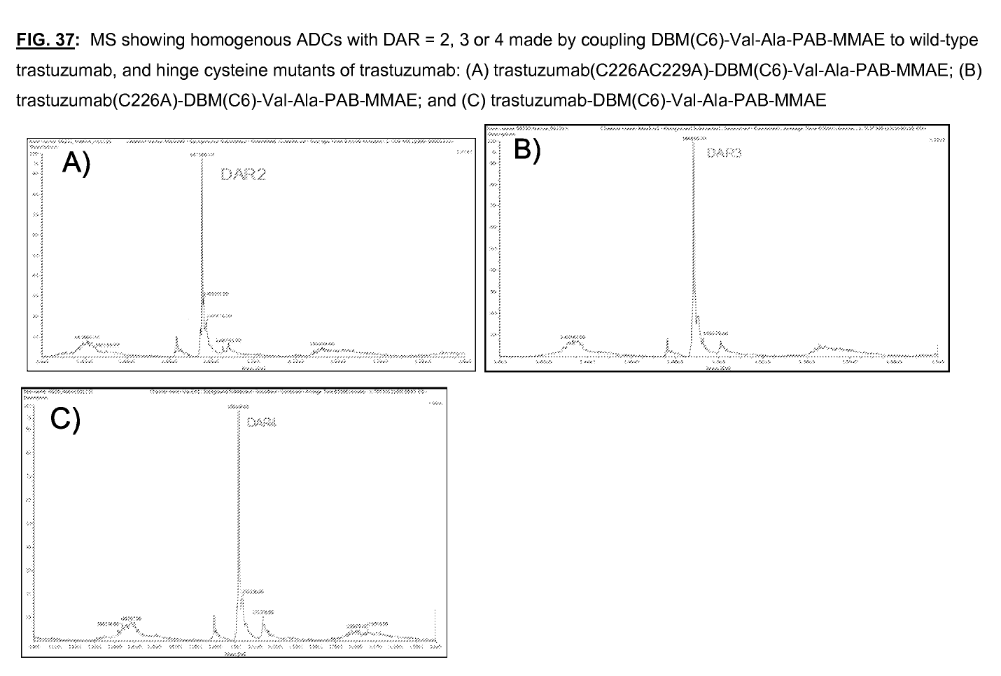

HIC analysis showed that trastuzumab(C226A)-DBM(C6)-MMAF eluted as a single homogeneous peak with a retention time consistent with a DAR of 3 drugs/antibody (see FIG. 36 (A)); whereas trastuzumab(C226AC229A)-DBM(C6)-MMAF eluted as a single homogeneous peak with a retention time consistent with a DAR of 2 drugs/antibody (see FIG. 36 (C)). The relative DAR compositions determined by LC/MS are comparable to those determined by HIC and the observed molecular weights are consistent with DARs of 2 and 3 (see FIGS. 36 (B) and (D)).

HIC analysis showed that trastuzumab(C226A)-DBM(C6)-VAP-MMAE eluted as a single homogeneous peak with a retention time consistent with a DAR of 3 drugs/antibody; whereas trastuzumab(C226AC229A)-DBM(C6)-VAP-MMAE eluted as a single homogeneous peak with a retention time consistent with a DAR of 2 drugs/antibody (data not shown). The relative DAR compositions determined by LC/MS are comparable to those determined by HIC and the observed molecular weights are consistent with DARs of 2 and 3 (see FIGS. 37 (A) and (B)).

### Example 14B

The relative homogeneity and DARs (drugs/antibody ratio) of ADCs prepared according to Example 13B were determined using hydrophobic interaction chromatography (HIC) and native LC/MS analysis.

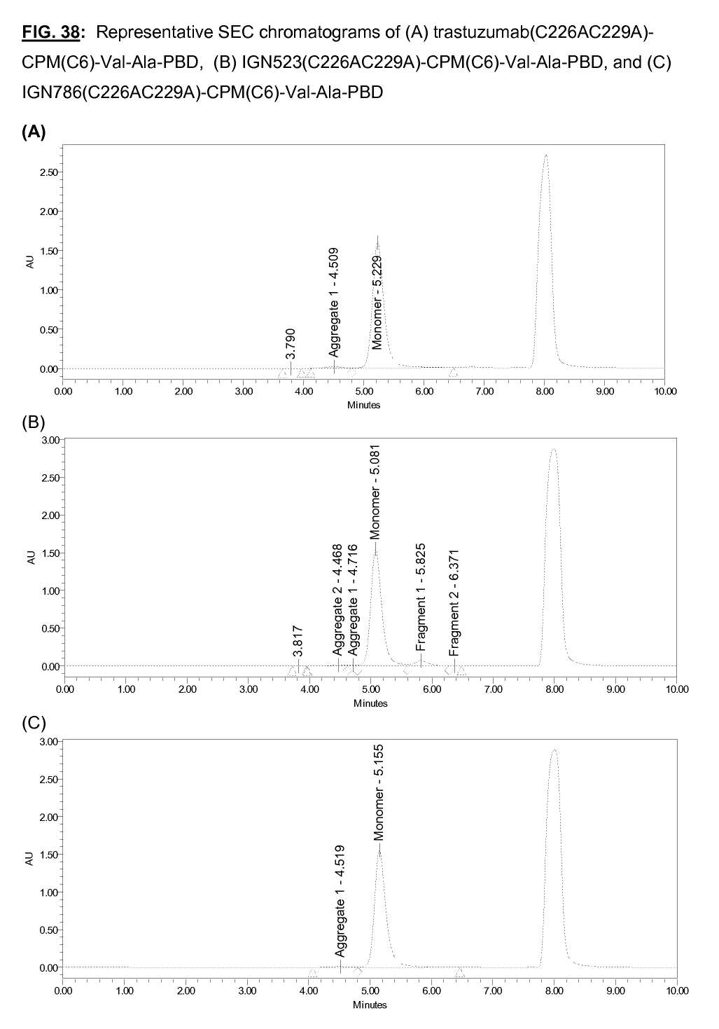

FIG. 38 shows representative SEC chromatograms of (A) trastuzumab(C226AC229A)-CPM(C6)-Val-Ala-PBD, (B) IGN523(C226AC229A)-CPM(C6)-Val-Ala-PBD, and (C) IGN786(C226AC229A)-CPM(C6)-Val-Ala-PBD.

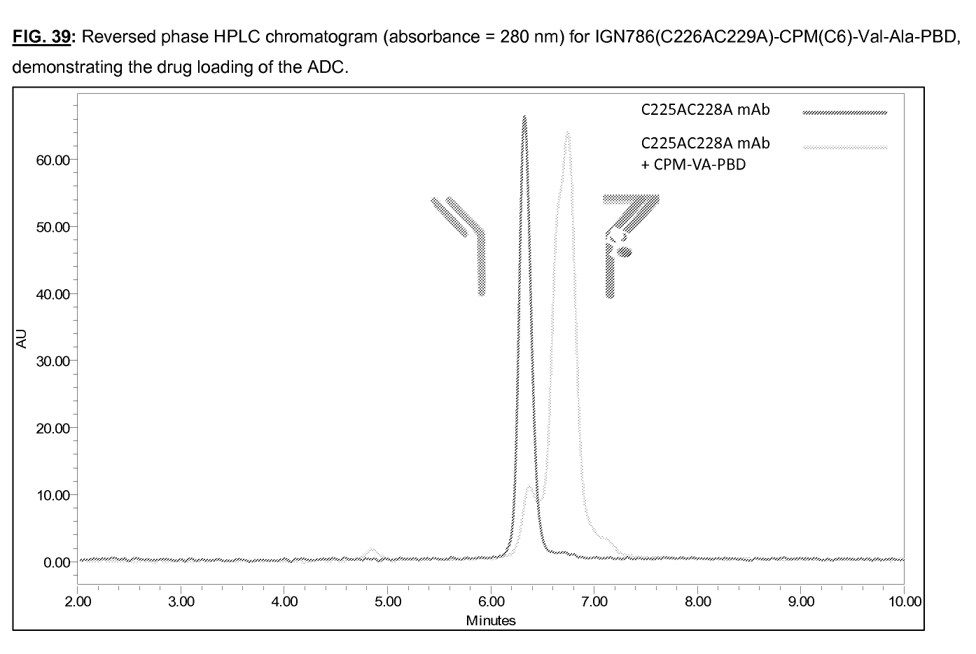

FIG. 39 shows an example of a reversed phase HPLC chromatogram for IGN786(C226AC229A)-CPM(C6)-Val-Ala-PBD, demonstrating the drug loading of the ADC.

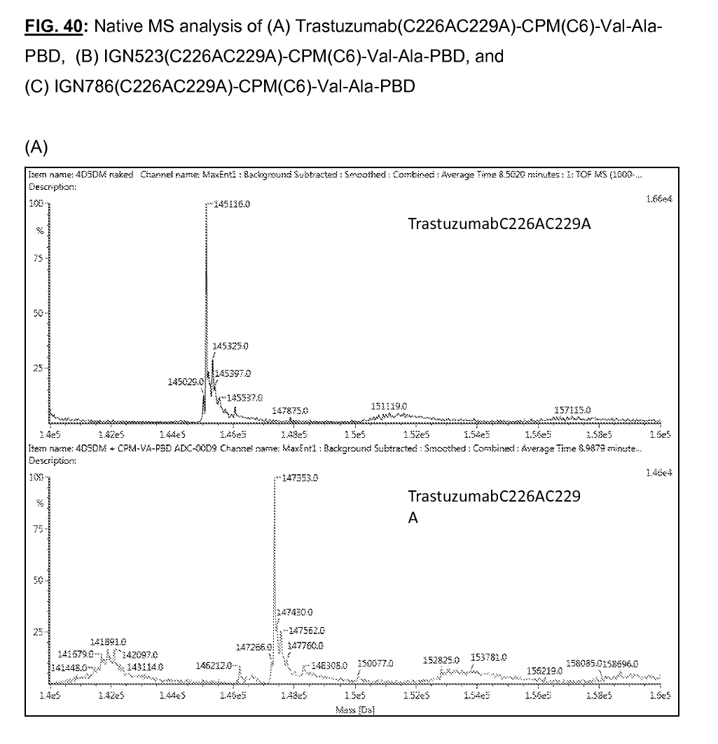

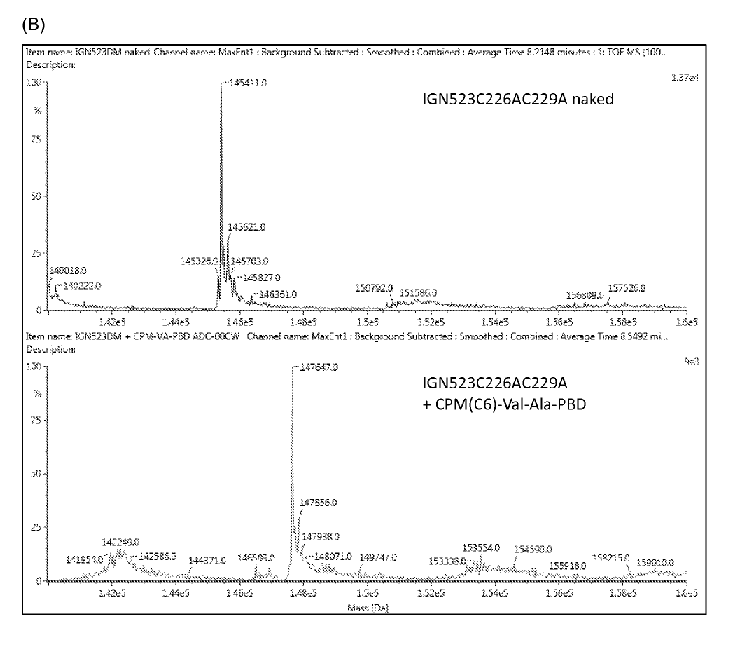

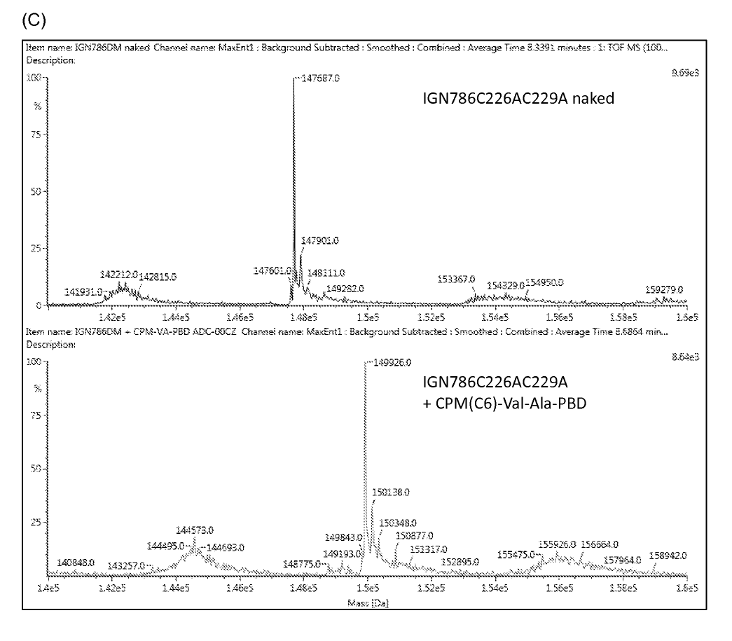

FIG. 40 shows native MS analysis of (A) trastuzumab(C226AC229A)-CPM(C6)-Val-Ala-PBD, (B) IGN523(C226AC229A)-CPM(C6)-Val-Ala-PBD, and (C) IGN786(C226AC229A)-CPM(C6)-Val-Ala-PBD, demonstrating highly homogeneous loading of linker-toxin.

In vitro cytotoxicity studies of ADCs prepared according to Example 13B were also determined.

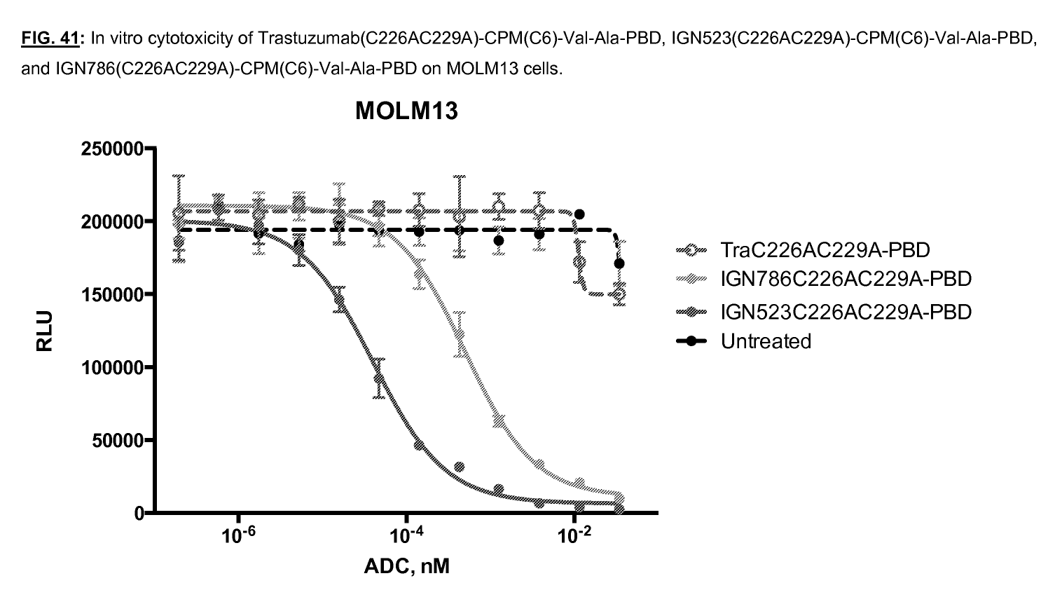

FIG. 41 shows in vitro cytotoxicity study on MOLM13 cells (CD98⁺, HER2⁻, SAIL⁺) using IGN523(C226AC229A)-CPM(C6)-Val-Ala-PBD, IGN786(C226AC229A)-CPM(C6)-Val-Ala-PBD, and trastuzumab(C226AC229A)-CPM(C6)-Val-Ala-PBD as a non-binding control, thus demonstrating potent efficacy and antigen specificity. The IC₅₀ values for this experiment are summarized in Table 22.

TABLE 22
IC₅₀ values for PBD ADCs

| | MOLM13 |
|---|---|
| ADC | IC₅₀ (nM) |
| trastuzumab(C226AC229A)-CPM(C6)-Val-Ala-PBD | >0.01 |
| IGN523(C226AC229A)-CPM(C6)-Val-Ala-PBD | 0.00004 |
| IGN786(C226AC229A)-CPM(C6)-Val-Ala-PBD | 0.0005 |

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

### APPENDIX A: IGN523 CONJUGATION DOE CALCULATIONS

(1) Master Calculation Sheet Experiment 13: DBM(C8)-MMAF (6 pages)
(2) Antibody Master Stock Concentration and Sub-Master Stocks Preparation (2 pages)
(3) TCEP Sub Master Stock Solutions Calculation Sheet (1 page)
(4) Drug Linker Master and Sub-Master Stock Solutions Calculation Sheet (2 pages)

### (1) Master Calculation Sheet Experiment 13: DBM(C6)-MMAF

PARAMETERS Continued.
MASTER CALCULATION SHEET EXPERIMENT 13: DBM(C8)-MMAF PARAMETERS

| Exp # | Block | Lot Code | Sample ID | Reduction pH | Reduction Time | Reduction Temp |
|---|---|---|---|---|---|---|
| 1 | 1 | GEN-002N | S01LN | 8.2 | 2.5 | 37 |
| 2 | 1 | GEN-002N | S01JG | 8.2 | 1.0 | 28.5 |
| 3 | 1 | GEN-002N | S01JH | 8.2 | 1.0 | 20 |
| 4 | 1 | GEN-002N | S01JI | 8.2 | 4.0 | 20 |
| 5 | 1 | GEN-002N | S01JJ | 8.2 | 4.0 | 37 |
| 6 | 1 | GEN-002N | S01JK | 8.2 | 1.0 | 37 |
| 7 | 1 | GEN-002N | S01JL | 8.2 | 1.0 | 37 |
| 8 | 1 | GFN-002N | S01JM | 8.2 | 2.4 | 37 |
| 9 | 1 | GEN-002N | S01JN | 7.8 | 4.0 | 37 |
| 10 | 1 | GEN 002N | S01JO | 7.8 | 2.5 | 37 |
| 11 | 1 | GEN-002N | S01JP | 7.8 | 2.8 | 28.5 |
| 12 | 1 | GEN-002N | S01JQ | 7.8 | 2.3 | 37 |
| 13 | 3, | GEN-002N | S01JR | 7.4 | 1.0 | 20 |
| 14 | 1 | GEN-002N | S01JS | 7.4 | 2.8 | 28.5 |
| 15 | 1 | GEN-002N | S01JT | 7.4 | 2.8 | 20 |
| 16 | 1 | GEN-002N | S01JU | 7.4 | 4.0 | 37 |
| 17 | 2. | GEN-002M | S01LR | 8.2 | 2.5 | 37 |
| 18 | 2 | GEN-002M | S01JV | 8.2 | 1 | 37 |
| 19 | 2 | GEN-002M | S01JW | 8.2 | 4 | 20 |
| 20 | 2 | GEN-002M | S01JX | 8.2 | 4 | 20 |
| 21 | 2 | GEN-002M | S01JY | 8.2 | 2.8 | 28.5 |
| 22 | 2 | GEN-002M | S01JZ | 8.2 | 4 | 37 |
| 23 | 2 | GEN-002M | S01K0 | 8.2 | 2.4 | 20 |
| 24 | 2 | GEN-002M | S01K1 | 8.2 | 4 | 28.5 |
| 25 | 2 | GEN-002M | S01K2 | 7.8 | 1 | 2.0 |
| 26 | 2 | GEN-002M | S01K3 | 7.8 | 2.7 | 37 |
| 27 | 2 | GEN-002M | S01K4 | 7.8 | 1 5 | 28.5 |
| 28 | 2 | GEN-002M | S01K5 | 7.8 | 1.5 | 28.5 |
| 29 | 2 | GEN-002M | S01K6 | 7.4 | 4 | 20 |
| 30 | 2 | GEN-002M | S01K7 | 7.4 | 2.7 | 20 |
| 31 | 2 | GEN-002M | S01K8 | 7.4 | 1 | 37 |
| 32 | 2 | GEN-002M | S01K9 | 7.4 | 4 | 2.0 |
| 33 | 2-3 | GEN-002L | S01LQ | 8.2 | 2.5 | 37 |
| 34 | 2-3 | GEN-002L | S01KA | 8.2 | 4 | 37 |
| 35 | 2-3 | GEN-002L | S01KB | 8.2 | 4 | 37 |
| 36 | 2-3 | GEN-002L | S01KC | 8.2 | 1 | 37 |
| 37 | 2-3 | GEN-002L | S01KD | 8.2 | 2.8 | 37 |
| 38 | 2-3 | GEN-002L | S01KE | 8.2 | 2.8 | 37 |
| 39 | 2-3 | GEN-002L | S01KF | 8.2 | 4 | 20 |
| 40 | 2-3 | GEN-002L | S01KG | 7.8 | 3 | 28.5 |
| 41 | 2-3 | GEN-002L | S01KH | 7.8 | 4 | 37 |
| 42 | 3-4 | GEN-002L | S01KI | 7.8 | 4 | 37 |
| 43 | 3-4 | GEN-002L | S01KJ | 7.4 | i | 37 |
| 44 | 3-4 | GEN-002L | S01KK | 7.4 | i | 37 |
| 45 | 3-4 | GEN-002L | S01KL | 7.4 | 1.6 | 28.5 |
| 46 | 3-4 | GEN-002L | S01KM | 7.4 | 1.6 | 28.5 |
| 47 | 3-4 | GEN-002L | S01KN | 7.4 | 4 | 37 |
| 48 | 3-4 | GEN-002L | S01KO | 7.4 | 4 | 37 |
| 49 | 4 | GEN-002K | S01LP | 8.2 | 2.5 | 37 |
| 50 | 4 | GEN-002K | S01KP | 8.2 | 4 | 20 |
| 51 | 4 | GEN-002K | S01KQ | 8.2 | 4 | 20 |
| 52 | 4 | GEN-002K | S01KR | 8.2 | 2.7 | 28.5 |
| 53 | 4 | GEN-002K | S01KS | 8.2 | 2.7 | 28.5 |
| 54 | 4 | GEN-002K | S01KT | 8.2 | 2.7 | 20 |
| 55 | 4 | GEN-002K | S01KU | 8.2 | 1 | 20 |
| 56 | 4 | GEN-002K | S01KV | 8.2 | 2.4 | 37 |
| 57 | 4 | GEN-002K | S01KW | 7.8 | 1.8 | 20 |
| 58 | 4 | GEN-002K | S01KX | 7.8 | 1.3 | 20 |
| 59 | 4 | GEN-002K | S01KY | 7.8 | 2.7 | 20 |
| 60 | 4 | GEN-002K | S01KZ | 7.8 | 3.9 | 28.5 |
| 61 | 4 | GEN-002K | S01L0 | 7.8 | 3.1 | 20 |
| 62 | 4 | GEN-002K | S01L1 | 7.4 | 4 | 28.5 |
| 63 | 4 | GEN-002K | S01L2 | 7.4 | 2.5 | 37 |
| 64 | 4 | GEN-002K | S01L3 | 7.4 | 2.5 | 20 |

| | | | | | | Starting |
|---|---|---|---|---|---|---|
| | | | | | | Rxn |
| | TCEP | Conjugation | Conjugation | Linker-Toxin | | Volume |
| | Molar | Time | Time | Molar | Conjugation | per tube |
| Exp # | eq. | (Hr) | (min) | eq | Temp | (mL) |
| 1 | 6 | 2 | 120.0 | 4.0 | 20.0 | 0.10 |
| 2 | 10 | 1.475 | 88.5 | 7 | 37.0 | 0.10 |
| 3 | 10 | 0.95 | 57.0 | 7 | 20.0 | 0.10 |
| 4 | 8 | 0.5 | 30.0 | 5 | 37.0 | 0.10 |
| 5 | 7 | 0.5 | 30.0 | 3 | 28.5 | 0.10 |
| 6 | 6 | 2 | 120.0 | 7 | 20.0 | 0.10 |
| 7 | 6 | 0.5 | 30.0 | 7 | 28.5 | 0.10 |
| 8 | 6 | 1.4 | 84.0 | 3 | 20.0 | 0.10 |
| 9 | 10 | 0.5 | 30.0 | 5 | 37.0 | 0.10 |
| 10 | 8 | 0.5 | 30.0 | 7 | 20.0 | 0.10 |
| 11 | 8 | 1.25 | 75.0 | 4 | 28.5 | 0.10 |
| 12 | 6 | 2 | 120.0 | 3 | 28.5 | 0.10 |
| 13 | 10 | 0.5 | 30.0 | 7 | 37.0 | 0.10 |
| 14 | 10 | 2 | 120.0 | 3 | 20.0 | 0.10 |
| 15 | 7 | 2 | 120.0 | 7 | 7.0,0 | 0.10 |
| 16 | 6 | 2 | 120.0 | 5 | 20.0 | 0.10 |
| 17 | 6 | 2 | 120.0 | 5.5 | 20 | 0.10 |
| 18 | 10 | 0.5 | 30.0 | 4 | 28.5 | 0.10 |
| 19 | 10 | 0.5 | 30.0 | 4 | 20 | 0.10 |
| 20 | 8 | 2 | 120.0 | 10 | 20 | 0.10 |
| 21 | 7 | 1.325 | 79.5 | 5.5 | 20 | 0.10 |
| 22 | 6 | 1.325 | 79.5 | 10 | 28.5 | 0.10 |
| 23 | 6 | 2 | 120.0 | 5.5 | 37 | 0.10 |
| 24 | 6 | 2 | 120.0 | 4 | 37 | 0.10 |
| 25 | S | 2 | 120.0 | 4 | 20 | 0.10 |
| 26 | 6 | 1.325 | 79.5 | 6.8 | 37 | 0.10 |
| 27 | 6 | 1.7 | 102.0 | 5.5 | 37 | 0.10 |
| 28 | 6 | 1.7 | 102.0 | 5.5 | 37 | 0.10 |
| 29 | 10 | 0.725 | 43.5 | 10 | 20 | 0.10 |
| 30 | 10 | 0.5 | 30.0 | 4 | 37 | 0.10 |
| 31 | 10 | 1.7 | 102.0 | 4 | 37 | 0.10 |
| 32 | 8 | 2 | 120.0 | 4 | 37 | 0.10 |
| 33 | 6 | 2 | 120.0 | 5.5 | 20.0 | 0.10 |
| 34 | 10 | 0.5 | 30.0 | 10 | 20 | 0.10 |
| 35 | 10 | 2 | 120.0 | 5.5 | 20 | 0.10 |
| 36 | 10 | 2 | 120.0 | 4 | 20 | 0.10 |
| 37 | 8 | 2 | 120.0 | 10 | 37 | 0.10 |
| 38 | 8 | 2 | 120.0 | 10 | 37 | 0.10 |
| 39 | 6 | 1.475 | 88.5 | 4 | 20 | 0.10 |
| 40 | 8 | 1.175 | 70.5 | 5.5 | 28.5 | 0.10 |
| 41 | 8 | 1.175 | 70.5 | 4 | 20 | 0.10 |
| 42 | 8 | 1.175 | 70.5 | 4 | 20 | 0.10 |
| 43 | 10 | 1.4 | 84.0 | 6.8 | 20 | 0.10 |
| 44 | 10 | 1.4 | 84.0 | 6.8 | 20 | 0.10 |
| 45 | 8 | 0.8 | 48.0 | 4 | 20 | 0.10 |
| 46 | 8 | 0.8 | 48.0 | 4 | 20 | 0.10 |
| 47 | 6 | 0.5 | 30.0 | 5.5 | 20 | 0.10 |
| 48 | 6 | 0.5 | 30.0 | 5.5 | 20 | 0.10 |
| 49 | 6 | 2 | 120.0 | 5.5 | 20.0 | 0.10 |
| 50 | 10 | 1.475 | 88.5 | 4 | 37 | 0.10 |
| 51 | 10 | 1.475 | 88.5 | 4 | 37 | 0.10 |
| 52 | 8 | 1.1 | 66.0 | 5.5 | 28.5 | 0.10 |
| 53 | 8 | 1.1 | 66.0 | 5.5 | 28.5 | 0.10 |
| 54 | 6 | 0.5 | 30.0 | 10 | 20 | 0.10 |
| 55 | 6 | 0.95 | 57.0 | 4 | 37 | 0.10 |
| 56 | 6 | 0.5 | 30.0 | 4 | 37 | 0.10 |
| 57 | 10 | 2 | 120.0 | 6.8 | 37 | 0.10 |
| 58 | 1 | 0.5 | 30.0 | 5.5 | 20 | 0.10 |
| 59 | 6 | 1.55 | 93.0 | 10 | 37 | 0.10 |
| 60 | 6 | 0.5 | 30.0 | 5.5 | 37 | 0.10 |
| 61 | 6 | 0.5 | 30.0 | 4 | 37 | 0.10 |
| 62 | 10 | 2 | 120.0 | 10 | 37 | 0.10 |
| 63 | 7 | 1.025 | 61.5 | 10 | 37 | 0.10 |
| 64 | 7 | 1.325 | 79.5 | 4 | 28.5 | 0.10 |

REDUCTION STEP

| | Reduction Step | | | | | Total Sub- | |
|---|---|---|---|---|---|---|---|
| | Min. Rxn | Rxn Volume | Protein | Total | Sub-aliquoting | aliquoting Vol. | Total |
| | Volume by pH | Overage | [c] | Per Rxn Set | Volume per | per TCEP Set | per Set |
| Exp # | (mL) | (mL) | (mg/mL) | (mg) | TCEP Set | (mL) | (mg) |
| 1. | | | | | 0.10 | 0.10 | 0.50 |
| 2. | | | | | 0.10 | 0.10 | 0.50 |
| 3. | | | | | 0.10 | 0.10 | 0.50 |
| 4. | | | | | 0.10 | 0.10 | 0.50 |
| 5. | | | | | 0.10 | 0.10 | 0.50 |
| 6. | 0.8 | 0.70 | 5.00 | 3.50 | 0.10 | 0.10 | 0.50 |
| 7. | | | | | 0.10 | 0.10 | 0.50 |
| 8. | | | | | 0.10 | 0.10 | 0.50 |
| 9. | | | | | 0.10 | 0.10 | 0.50 |
| 10. | | | | | 0.10 | 0.10 | 0.50 |
| 11. | 0.5 | 0.60 | 5.00 | 3.00 | 0.10 | 0.10 | 0.50 |
| 12. | | | | | 0.10 | 0.10 | 0.50 |
| 13. | | | | | 0.10 | 0.10 | 6.50 |
| 14. | | | | | 0.10 | 0.10 | 0.50 |
| 15. | | | | | 0.10 | 0.10 | 0.50 |
| 16. | 0.5 | 0.60 | 5.00 | 3.00 | 0.10 | 0.10 | 0.50 |
| 17. | | | | | 0.10 | 0.10 | 6.50 |
| 18. | | | | | 0.10 | 0.10 | 0.50 |
| 19. | | | | | 0.10 | 0.10 | 0.50 |
| 20. | | | | | 0.10 | 0.10 | 0.50 |
| 21. | | | | | 0.10 | 0.10 | 0.50 |
| 22. | | | | | 0.10 | 0.10 | 0.50 |
| 23. | 0.6 | 0.70 | 5.00 | 3.50 | 0.10 | 0.10 | 0.50 |
| 24. | | | | | 0.10 | 0.10 | 0.50 |
| 25. | | | | | 0.10 | 0.10 | 0.50 |
| 26. | 0.3 | 0.40 | 5.00 | 2.00 | 0.10 | 0.10 | 6.50 |
| 27. | | | | | 0.10 | 0.10 | 0.50 |
| 28. | | | | | 0.10 | 0.1o | 0.50 |
| 29. | | | | | 0.10 | 0.10 | 0.50 |
| 30. | | | | | 0.10 | 0.10 | 0.50 |
| 31. | | | | | 0.10 | 0.10 | 6.50 |
| 32. | 0.6 | 0.70 | 5.00 | 3.50 | 0.10 | 0.10 | 0.50 |
| 33. | 0.6 | 1.00 | 5.00 | 5.00 | 0.10 | 0.10 | 0.50 |
| 34. | | | | | 0.10 | 0.10 | 0.50 |
| 35. | | | | | 0.10 | 0.10 | 0.50 |
| 36. | | | | | 0.10 | 0.10 | 0.50 |
| 37. | | | | | 0.10 | 0.10 | 0.50 |
| 38. | | | | | 0.10 | 0.10 | 0.50 |
| 39. | 0.6 | 1.00 | 5.00 | 5.00 | 0.10 | 0.10 | 0.50 |
| 40. | | | | | 0.10 | 0.10 | 0.50 |
| 41. | | | | | 0.10 | 0.10 | 0.50 |
| 42. | | | | | 0.10 | 0.10 | 0.50 |
| 43. | | | | | 0.10 | 0.10 | 0.50 |
| 44. | | | | | 0.10 | 0.10 | 0.50 |
| 45. | 0.6 | 0.70 | 5.00 | 3.50 | 0.10 | 0.10 | 0.50 |
| 46. | | | | | 0.10 | 0.10 | 0.50 |
| 47. | | | | | 0.10 | 0.10 | 0.50 |
| 48. | 0.3 | 0.40 | 5.00 | 2.00 | 0.10 | 0.10 | 0.50 |
| 49. | | | | | 0.10 | 0.10 | 0.50 |
| 50. | | | | | 0.10 | 0.10 | 0.50 |
| 51. | | | | | 0.10 | 0.10 | 0.50 |
| 52. | | | | | 0.10 | 0.10 | 0.50 |
| 53. | | | | | 0.10 | 0.10 | 0.50 |
| 54. | 0.5 | 0.60 | 5.00 | 3.00 | 0.10 | 0.10 | 0.50 |
| 55. | | | | | 0.10 | 0.10 | 0.50 |
| 56. | | | | | 0.10 | 0.10 | 0.50 |
| 57. | | | | | 0.10 | 0.10 | 0.50 |
| 58. | | | | | 0.10 | 0.10 | 0.50 |
| 59. | | | | | 0.10 | 0.10 | 0.50 |
| 60. | 0.6 | 0.70 | 5.00 | 3.50 | 0.10 | 0.10 | 0.50 |
| 61. | | | | | 0.10 | 0.10 | 0.50 |
| 62. | | | | | 0.10 | 0.10 | 0.50 |
| 63. | | | | | 0.10 | 0.10 | 0.50 |
| 64. | 0.4 | 0.50 | 5.00 | 2.50 | 0.10 | 0.10 | 0.50 |

| | | | TCEP | Stock [C] | Volume | | |
|---|---|---|---|---|---|---|---|
| | 1gG1 | TCEP | volume to | of TCEP | Subtraction | New Total | New |
| | Protein | moles | add per rxn | required | for IAM | Rxn | Protein |
| | moles | to add | tube/set | for Rxn | capping | volume | Conc. |
| Exp # | per set | to rxn | (uL) | (mM) | (mL) | (mL) | (mg/mL) |
| 1. | 3.38E−09 | 2.03E−08 | 5.00 | 4.05 | 0.005 | 0.10 | 4.76 |
| 2. | 3.38E−09 | 3.38E−08 | 5.00 | 6.76 | 0.005 | 0.10 | 4.76 |
| 3. | 3.38E−09 | 3.38E−08 | 5.00 | 6.76 | 0.005 | 0.10 | 4.76 |
| 4. | 3.38E−09 | 2.70E−08 | 5.00 | 5.41 | 0.005 | 0.10 | 4.76 |
| 5. | 3.38E−09 | 2.36E−08 | 5.00 | 4.73 | 0.005 | 0.10 | 4.76 |
| 6. | 3.38E−09 | 2.03E−08 | 5.00 | 4.05 | 0.005 | 0.10 | 4.76 |
| 7. | 3.38E−09 | 2.03E−08 | 5.00 | 4.05 | 0.005 | 0.10 | 4.76 |
| 8. | 3.38E−09 | 2.03E−08 | 5.00 | 4.05 | 0.005 | 0.10 | 4.76 |
| 9. | 3.38E−09 | 3.38E−08 | 5.00 | 6.76 | 0.005 | 0.10 | 4.76 |
| 10. | 3.38E−09 | 2.70E−08 | 5.00 | 5.41 | 0.005 | 0.10 | 4.76 |
| 11. | 3.38E−09 | 2.70E−08 | 5.00 | 5.41 | 0.005 | 0.10 | 4.76 |
| 12. | 3.38E−09 | 2.03E−08 | 5.00 | 4.05 | 0.005 | 0.10 | 4.76 |
| 13. | 3.38E−09 | 3.38E−08 | 5.00 | 6.76 | 0.005 | 0.10 | 4.76 |
| 14. | 3.38E−09 | 3.38E−08 | 5.00 | 6.76 | 0.005 | 0.10 | 4.76 |
| 15. | 3.38E−09 | 2.38E−08 | 5.00 | 4.73 | 0.005 | 0.10 | 4.76 |
| 16. | 3.38E−09 | 2.03E−08 | 5.00 | 4.05 | 0.005 | 0.10 | 4.76 |
| 17. | 3.38E−09 | 2.03E−08 | 5.00 | 4.05 | 0.005 | 0.10 | 4.76 |
| 18. | 3.38E−09 | 3.38E−08 | 5.00 | 6.76 | 0.005 | 0.10 | 4.76 |
| 19. | 3.38E−09 | 3.38E−08 | 5.00 | 6.76 | 0.005 | 0.10 | 4.76 |
| 20. | 3.38E−09 | 2.70E−08 | 5.00 | 5.41 | 0.005 | 0.10 | 4.76 |
| 21. | 3.38E−09 | 2.36E−08 | 5.00 | 4.73 | 0.005 | 0.10 | 4.76 |
| 22. | 3.38E−09 | 2.03E−08 | 5.00 | 4.05 | 0.005 | 0.10 | 4.76 |
| 23. | 3.38E−09 | 2.03E−08 | 5.00 | 4.05 | 0.005 | 0.10 | 4.76 |
| 24. | 3.38E−09 | 2.03E−08 | 5.00 | 4.05 | 0.005 | 0.10 | 4.76 |
| 25. | 3.38E−09 | 2.70E−08 | 5.00 | 5.41 | 0.005 | 0.10 | 4.76 |
| 26. | 3.38E−09 | 2.03E−08 | 5.00 | 4.05 | 0.005 | 0.10 | 4.76 |
| 27. | 3.38E−09 | 2.03E−08 | 5.00 | 4.05 | 0.005 | 0.10 | 4.76 |
| 28. | 3.38E−09 | 2.03E−08 | 5.00 | 4.05 | 0.005 | 0.10 | 4.76 |
| 29. | 3.38E−09 | 3.38E−08 | 5.00 | 6.76 | 0.005 | 0.10 | 4.76 |
| 30. | 3.38E−09 | 3.38E−08 | 5.00 | 6.76 | 0.005 | 0.10 | 4.76 |
| 31. | 3.38E−09 | 3.38E−08 | 5.00 | 6.76 | 0.005 | 0.10 | 4.76 |
| 32. | 3.38E−09 | 2.70E−08 | 5.00 | 5.41 | 0.005 | 0.10 | 4.76 |
| 33. | 3.38E−09 | 3.38E−G8 | 5.00 | 6.76 | 0.005 | 0.10 | 4.76 |
| 34. | 3.38E−09 | 3.38E−G8 | 5.00 | 6.76 | 0.005 | 0.10 | 4.76 |
| 35. | 3.38E−09 | 3.38E−08 | 5.00 | 6.75 | 0.005 | 0.10 | 4.76 |
| 36. | 3.38E−09 | 2.70E−08 | 5.00 | 5.41 | 0.005 | 0.10 | 4.76 |
| 37. | 3.38E−09 | 2.70E−08 | 5.00 | 5.41 | 0.005 | 0.10 | 4.76 |
| 38. | 3.38E−09 | 2.03E−08 | 5.00 | 4.05 | 0.005 | 0.10 | 4.76 |
| 39. | 3.38E−09 | 3.38E−G8 | 5.00 | 6.76 | 0.005 | 0.10 | 4.76 |
| 40. | 3.38E−09 | 2.70E−08 | 5.00 | 5.41 | 0.005 | 0.10 | 4.76 |
| 41. | 3.38E−09 | 2.70E−08 | 5.00 | 5.41 | 0.005 | 0.10 | 4.76 |
| 42. | 3.38E−09 | 2.70E−08 | 5.00 | 5.41 | 0.005 | 0.10 | 4.76 |
| 43. | 3.38E−09 | 3.38E−08 | 5.00 | 6.76 | 0.005 | 0.10 | 4.76 |
| 44. | 3.38E−09 | 3.38E−08 | 5.00 | 6.75 | 0.005 | 0.10 | 4.76 |
| 45. | 3.38E−09 | 2.70E−08 | 5.00 | 5.41 | 0.005 | 0.10 | 4.76 |
| 46. | 3.38E−09 | 2.70E−08 | 5.00 | 5.41 | 0.005 | 0.10 | 4.76 |
| 47. | 3.38E−09 | 2.03E−08 | 5.00 | 4.05 | 0.005 | 0.10 | 4.76 |
| 48. | 3.38E−09 | 2.03E−08 | 5.00 | 4.05 | 0.005 | 0.10 | 4.76 |
| 49. | 3.38E−09 | 2.03E−08 | 5.00 | 4.05 | 0.005 | 0.10 | 4.76 |
| 50. | 3.38E−09 | 3.38E−08 | 5.00 | 6.76 | 0.005 | 0.10 | 4.76 |
| 51. | 3.38E−09 | 3.38E−08 | 5.00 | 6.76 | 0.005 | 0.10 | 4.76 |
| 52. | 3.38E−09 | 2.70E−08 | 5.00 | 5.41 | 0.005 | 0.10 | 4.76 |
| 53. | 3.38E−09 | 2.70E−08 | 5.00 | 5.41 | 0.005 | 0.10 | 4.76 |
| 54. | 3.38E−09 | 2.03E−G8 | 5.00 | 4.05 | 0.005 | 0.10 | 4.76 |
| 55. | 3.38E−09 | 2.03E−08 | 5.00 | 4.05 | 0.005 | 0.10 | 4.76 |
| 56. | 3.38E−09 | 2.03E−08 | 5.00 | 4.05 | 0.005 | 0.10 | 4.76 |
| 57. | 3.38E−09 | 3.38E−08 | 5.00 | 6.76 | 0.005 | 0.10 | 4.76 |
| 58. | 3.38E−09 | 2.36E−08 | 5.00 | 4.73 | 0.005 | 0.10 | 4.76 |
| 59. | 3.38E−09 | 2.03E−08 | 5.00 | 4.05 | 0.005 | 0.10 | 4.76 |
| 60. | 3.38E−09 | 2.03E−08 | 5.00 | 4.05 | 0.005 | 0.10 | 4.76 |
| 61. | 3.38E−09 | 2.03E−08 | 5.00 | 4.05 | 0.005 | 0.10 | 4.76 |
| 62. | 3.38E−09 | 3.38E−08 | 5.00 | 6.76 | 0.005 | 0.10 | 4.76 |
| 63. | 3.38E−09 | 2.36E−08 | 5.00 | 4.73 | 0.005 | 0.10 | 4.76 |
| 64. | 3.38E−09 | 2.36E−08 | 5.00 | 4.73 | 0.005 | 0.10 | 4.76 |

CONJUGATION STEP

| | Conjugation Step | | | | | |
|---|---|---|---|---|---|---|
| | | Conjugation | | | | Stock [C] of (x)- |
| | Rxn Volume per | Starting | New | | Drug-Linker | MMAF linker |
| | tube (mL) aliquote | Amount | JGN523 | Drug-Linker | volume to add | required for |
| | from Start Cond. | per tube | Protein | moles to | per rxn tube | each rxn tube |
| Exp# | After TCEP | (mg) | moles | add to rxn | (uL) | (mM) |
| 1 | 0.1 | 0.48 | 3.2175E−09 | 1.287E−08 | 10.0 | 1.29 |
| 2. | 0.1 | 0.48 | 3.2175E−09 | 2.25225E−08 | 10.0 | 2.25 |
| 3. | 0.1 | 0.48 | 3.2175E−09 | 2.25225E−08 | 10.0 | 2.25 |
| 4. | 0.1 | 0.48 | 3.2175E−09 | 1.60875E−08 | 10.0 | 1.61 |
| 5. | 0.1 | 0.48 | 3.2175E−09 | 9.65251E−09 | 10.0 | 0.97 |
| 6. | 0.1 | 0.48 | 3.2175E−09 | 2.25225E−08 | 10.0 | 2.25 |
| 7. | 0.1 | 0.48 | 3.2175E−09 | 2.25225E−08 | 10.0 | 2.25 |
| 8. | 0.1 | 0.48 | 3.2175E−09 | 9.65251E−09 | 10.0 | 0.97 |
| 9. | 0.1 | 0.48 | 3.2175E−09 | 1.60875E−08 | 10.0 | 1.61 |
| 10. | 0.1 | 0.48 | 3.2175E−09 | 2.25225E−08 | 10.0 | 2.25 |
| 11. | 0.1 | 0.48 | 3.2175E−09 | 1.287E−08 | 10.0 | 1.29 |
| 12. | 0.1 | 0.48 | 3.2175E−09 | 9.65251E−09 | 10.0 | 0.97 |
| 13. | 0.1 | 0.48 | 3.2175E−09 | 2.25225E−03 | 10.0 | 2.25 |
| 14. | 0.1 | 0.48 | 3.2175E−09 | 9.65251E−09 | 10.0 | 0.97 |
| 15. | 0.1 | 0.48 | 3.2175E−09 | 2.25225E−05 | 10.0 | 2.25 |
| 16. | 0.1 | 0.48 | 3.2175E−09 | 1.60575E−08 | 10.0 | 1.61 |
| 17. | 0.1 | 0.48 | 3.2175E−09 | 1.76963E−03 | 10.0 | 1.77 |
| 18. | 0.1 | 0.48 | 3.2175E−09 | 1.287E−05 | 10.0 | 1.29 |
| 19. | 0.1 | 0.48 | 3.2175E−09 | 1.287E−08 | 10.0 | 1.29 |
| 20. | 0.1 | 0.48 | 3.2175E−09 | 3.2175E−08 | 10.0 | 3.22 |
| 21. | 0.1 | 0.48 | 3.2175E−09 | 1.76963E−08 | 10.0 | 1.77 |
| 22. | 0.1 | 0.48 | 3.2175E−09 | 3.2175E−08 | 10.0 | 3.22 |
| 23. | 0.1 | 0.48 | 3.2175E−09 | 1.76963E−08 | 10.0 | 1.77 |
| 24. | 0.1 | 0.48 | 3.2175E−09 | 1.287E−08 | 10.0 | 1.29 |
| 25. | 0.1 | 0.48 | 3.2175E−09 | 1.287E−08 | 10.0 | 1.29 |
| 26. | 0.1 | 0.48 | 3.2175E−09 | 2.1879E−08 | 10.0 | 2.19 |
| 27. | 0.1 | 0.48 | 3.2175E−09 | 1.76963E−08 | 10.0 | 1.77 |
| 28. | 0.1 | 0.48 | 3.2175E−09 | 1.76963E−08 | 10.0 | 1.77 |
| 29. | 0.1 | 0.48 | 3.2175E−09 | 3.2175E−08 | 10.0 | 3.22 |
| 30. | 0.1 | 0.48 | 3.2175E−09 | 1.287E−08 | 10.0 | 1.29 |
| 31. | 0.1 | 0.48 | 3.2175E−09 | 1.287E−08 | 10.0 | 1.29 |
| 32. | 0.1 | 0.48 | 3.2175E−09 | 1.287E−08 | 10.0 | 1.29 |
| 33. | 0.1 | 0.48 | 3.2175E−09 | 1.76963E−08 | 10.0 | 1.77 |
| 34. | 0.1 | 0.48 | 3.2175E−09 | 3.2175E−08 | 10.0 | 3.22 |
| 35. | 0.1 | 0.48 | 3.2175E−09 | 1.76963E−88 | 10.0 | 1.77 |
| 36. | 0.1 | 0.48 | 3.2175E−09 | 1.287E−08 | 10.0 | 1.29 |
| 37. | 0.1 | 0.48 | 3.2175E−09 | 3.2175E−05 | 10.0 | 3.22 |
| 38. | 0.1 | 0.48 | 3.2175E−09 | 3.2175E−08 | 10.0 | 3.22 |
| 39. | 0.1 | 0.48 | 3.2175E−09 | 1.287E−08 | 10.0 | 1.29 |
| 40. | 0.1 | 0.48 | 3.2175E−09 | 1.76963E−08 | 10.0 | 1.77 |
| 41. | 0.1 | 0.48 | 3.2175E−09 | 1.287E−08 | 10.0 | 1.29 |
| 42. | 0.1 | 0.48 | 3.2175E−09 | 1.287E−08 | 10.0 | 1.29 |
| 43. | 0.1 | 0.48 | 3.2175E−09 | 2.1879E−08 | 10.0 | 2.19 |
| 44. | 0.1 | 0.48 | 3.2175E−09 | 2.1879E−08 | 10.0 | 2.19 |
| 45. | 0.1 | 0.48 | 3.2175E−09 | 1.287E−08 | 10.0 | 1.29 |
| 46. | 0.1 | 0.48 | 3.2175E−09 | 1.287E−08 | 10.0 | 1.29 |
| 47. | 0.1 | 0.48 | 3.2175E−09 | 1.76963E−08 | 10.0 | 1.77 |
| 48. | 0.1 | 0.48 | 3.2175E−09 | 1.75963E−08 | 10.0 | 1.77 |
| 49. | 0.1 | 0.48 | 3.2175E−09 | 1.76963E−08 | 10.0 | 1.77 |
| 50. | 0.1 | 0.48 | 3.2175E−09 | 1.287E−08 | 10.0 | 1.29 |
| 51. | 0.1 | 0.48 | 3.2175E−09 | 1.287E−08 | 10.0 | 1.29 |
| 52. | 0.1 | 0.48 | 3.2175E−09 | 1.76963E−08 | 10.0 | 1.77 |
| 53. | 0.1 | 0.48 | 3.2175E−09 | 1.75963E−08 | 10.0 | 1.77 |
| 54. | 0.1 | 0.48 | 3.2175E−09 | 3.2175E−08 | 10.0 | 3.22 |
| 55. | 0.1 | 0.48 | 3.2175E−09 | 1.287E−08 | 10.0 | 1.29 |
| 56. | 0.1 | 0.48 | 3.2175E−09 | 1.287E−08 | 10.0 | 1.29 |
| 57. | 0.1 | 0.48 | 3.2175E−09 | 2.1879E−08 | 10.0 | 2.19 |
| 58. | 0.1 | 0.48 | 3.2175E−09 | 1.76963E−08 | 10.0 | 1.77 |
| 59. | 0.1 | 0.48 | 3.2175E−09 | 3.2175E−08 | 10.0 | 3.22 |
| 60. | 0.1 | 0.48 | 3.2175E−09 | 1.76963E−08 | 10.0 | 1.77 |
| 61. | 0.1 | 0.48 | 3.2175E−09 | 1.287E−08 | 10.0 | 1.29 |
| 62. | 0.1 | 0.48 | 3.2175E−09 | 3.2175E−08 | 10.0 | 3.22 |
| 63. | 0.1 | 0.48 | 3.2175E−09 | 3.2175E−08 | 10.0 | 3.22 |
| 64. | 0.1 | 0.48 | 3.2175E−09 | 1.287E−08 | 10.0 | 1.29 |

### (2) Antibody Master Stock Concentration and Sub-Master Stocks Preparation

Antibody Master Stock Concentration A(280)-A(320)

| Buffer | | Dilution | Dilution | | | | Correction | Concentration |
|---|---|---|---|---|---|---|---|---|
| # | Buffer Composition | 1 (1:10) | 2 (1:10) | average | Stan Dev | % CV | for dilution | mg/mL |
| 1 | IGN523 20 mM Sodium phosphate, 20 mM | 1.476 | 1.435 | 1.4555 | 0.028991 | 1.99% | 14.555 | 9.451298701 |
| | Sodium Borate, 100 mM NaCl, | | | | | | | |
| | 5 mM EDTA, pH 8.2 | | | | | | | |
| 2 | IGN523 20 mM Sodium phosphate, 20 mM | 1.51 | 1.475 | 1.4925 | 0.024749 | 1.66% | 14.925 | 9.691558442 |
| | Sodium Borate, 100 mM NaCi, | | | | | | | |
| | 5 mM EDTA, pH 7.8 | | | | | | | |
| 3 | 1GN523 20 mM Sodium phosphate, 20 mM | 1.548 | 1.567 | 1.5575 | 0.013435 | 0.86% | 15.575 | 10.11363636 |
| | Sodium Borate, 100 mM NaCl, | | | | | | | |
| | 5 mM EDTA, pH 7.4 | | | | | | | |

Sub-Master Stocks Preparation

| | Total Amount | | Sub-Master | | | |
|---|---|---|---|---|---|---|
| | Required per | | Stock Target | Master | Diluent | |
| | Buffer Type | Target conc. | Volume | Stock Vol | Buffer | Sub-Master |
| Antibody | (mg) | (mg/mL) | (ml) | (mL) | (mL) | Stock Name |
| 1 | 20.00 | 5.00 | 4.00 | 2.12 | 1.88 | IGN pH 8.2 |
| 2 | 20.00 | 5.00 | 4.00 | 2.06 | 1.94 | IGN pH 7.8 |
| 3 | 11.00 | 5.00 | 2.20 | 1.09 | 1.11 | IGN pH 7.4 |

| | BSA | Dilution 1 | Dilution 2 | | | | Correction for | Concentration | |
|---|---|---|---|---|---|---|---|---|---|
| Buffer # | Control | (1:4) | (1:4) | average | Stan Dev | % CV | dilution | mg/mL | % Recovery |
| 1 | | 0.341 | 0.332 | 0.3365 | 0.006364 | 1.89% | 1.346 | 2.064417 | 103% |
| 2 | | 0.335 | 0.324 | 0.3295 | 0.007778 | 2.36% | 1.318 | 2.021472 | 101% |
| 3 | | 10.334 | 0.335 | 0.3345 | 0.00707 | 0.21% | 1.338 | 2.052147 | 103% |

### (3) TCEP Sub Master Stock Solutions Calculation Sheet

TCEP Sub Master Stock Solutions Calculation Sheet
TCEP Master Stock Solution 50 mM

| Target Master Stock [c] | 50 | mM | | |
|---|---|---|---|---|
| Target conc. | 10 | mM | | |
| Target Vol | 0.7 | mL | | |
| Stock vol req'd | 0.14 | mL | | |
| DMSO diluent | 0.84 | mL | | |
| FW | 286.65 | g/mol | | |
| Target [c] | 0.05 | Molar | | |
| Target weight | 10.000 | grams | | |
| weight out | 0.14333 | grams | 143.325 | mg |

| | | |
|---|---|---|
| Final weight | 10 | grams |
| actual weight | 0.1421 | grams |
| Molarity Actual | 0.04957265 | M |
| mMoiar Actual | 49.57264957 | mM |
| Final QSd weight | 10.000 | grams |

Note:
Master stock sol'n frozen at −80 C. (Thaw fresh vial before use . . . discard after 24 hours).

TCEP Sub-Master Stock Preparation

| | | TCEP Target | | Total Volume | | |
|---|---|---|---|---|---|---|
| | TCEP Target | Volume by | | Re'd with | TCEP Master | Diluent |
| | Conc. | Buffer Type | Volume | Excess | Stock Volume | Buffer |
| Buffer | (mM) | (uL) | Excess factor | (mL) | (uL) | (uL) |
| 1 | 6.76 | 80 | 1.2 | 0.10 | 13.0 | 83.0 |
| 1 | 4.05 | 100 | 1.2 | 0.12 | 9.7 | 110.3 |
| 1 | 4.73 | 50 | 1.2 | 0.06 | 5.7 | 54.3 |
| 1 | 5.41 | 70 | 1.5 | 0.11 | 11.4 | 93.6 |

### (4) Drug Linker Master and Sub-Master Stock Solutions Calculation Sheet

Drug linker Master arid Sub-Master Stock Solutions Calculation Sheet
DBM-mmaF acid Master Stock In DMA 10 mM

| | |
|---|---|
| Block 1 8 Aug. 2014 | Use DBM Lot SB 154-70 20140807 Lot SB 154-70 20140807 |
| Block 2 Aug. 15, 2014 | Use DBM Lot SB 154-74 80814 |
| Block 3-4 Aug. 18, 2014 | Use DBM Lot SB 154-74 8081.4 |

Sub-Master Stock Preparation

| | |
|---|---|
| DBM-C6-mmaf 4 eq | DBM-C6-mmaf 5.5 eq |

| | | | | | |
|---|---|---|---|---|---|
| Master Stock[c] | 10 | mM | Master Stock[c] | 10 | mM |
| Target [c] | 1.29 | mM | Target [c] | 1.77 | mM |
| Target Vol | 0.115 | mL | Target Vol | 0.0805 | mL |
| Stock vol req'd | 14.8 | uL | Stock vol req'd | 14.2 | uL |
| DMA diluent | 100.2 | uL | DMA diluent | 66.3 | uL |

| | |
|---|---|
| DBM-C6-mmaf 6.8 eq | DBM-C6-mmaf 10 eq |

| | | | | | |
|---|---|---|---|---|---|
| Master Stock[c] | 10 | mM | Master Stock[c] | 10 | mM |
| Target [c] | 2.19 | mM | Target [c] | 3.22 | mM |
| Target Vol | 0.013 | mL | Target Vol | 0.072 | mL |
| Stock vol req'd | 2.8 | uL | Stock vol req'd | 23.2 | uL |
| DMA diluent | 10.2 | uL | DMA diluent | 48.8 | uL |

Calculations

| | Drug-Linker | Stock [C] of (X)- | | |
|---|---|---|---|---|
| | volume to add | mmaf linke required | Min. Volume by sub- | Total Sub-Master |
| | per rxn tube | for each rxn tube | Master Type | Volume with Excess |
| | (uL) | (mM) | (uL) | (uL) |
| DBM-C6-mmaf 10 eq | 60 | 3.22 | 60 | 78 |
| DBM-C6-mmaf 6.8 eq | 10 | 2.19 | 10 | 13 |
| DBM-C6-mmaf 5.5 eq | 70 | 1.77 | 70 | 80.5 |
| DBM-C6-mmaf 4 eq | 100 | 1.29 | 100 | 115 |

| DBM-mmaF | |
|---|---|
| Formula Weight | Mass needed |
| (g/mol) | (mg) |
| 1000 | 0.570465 |

1. An antibody-drug conjugate of the following formula (I): 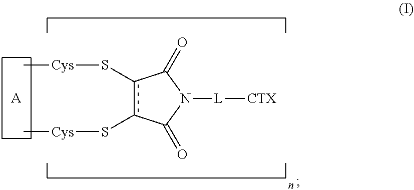
or a pharmaceutically acceptable salt thereof,
wherein:
A is an antibody;
the two depicted cysteine residues are from an opened cysteine-cysteine disulfide bond in A;
L is a cleavable or a noncleavable linker;
CTX is an auristatin, a pyrrolobenzodiazepine, calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond;
the [symbol] bond represents a single or a double bond; and
n is an integer of 1 to 4.

2. The antibody-drug conjugate of claim 1, which has the following formula (Ia) or formula (Ib):

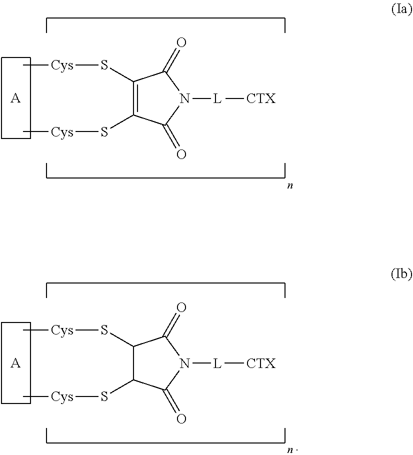

3. (canceled)

4. The antibody-drug conjugate of claim 1, wherein CTX is an auristatin bonded to L by an amide bond or a carbamate bond.

5. The antibody-drug conjugate of claim 4, wherein CTX is monomethylauristatin F.

6. The antibody-drug conjugate of claim 4, wherein CTX is monomethylauristatin E.

7.-8. (canceled)

9. The antibody-drug conjugate of claim 8, wherein L is —(CH₂)ₘC(O)—, wherein m is an integer of 5 to 11.

10.-11. (canceled)

12. The antibody-drug conjugate of claim 1, wherein A is a monoclonal antibody, and optionally wherein A comprises two heavy chains and two light chains wherein one or more cysteines in the hinge region of the heavy chains of A have been replaced by another amino acid.

13. (canceled)

14. The antibody-drug conjugate of claim 1, wherein A is an antibody that is specific to a cancer antigen, and optionally wherein the cancer antigen is CD33 (Siglec3), CD30 (TNFRSF8), HER2 (ERbB-2), EGFR, VEGF-A, CD22 (Siglec2), CD79b, CD22 (Siglec2), GPNMB, CD19 (B4), CD56 (NCAM), CD138 (SDC1), PSMA (FOLH1), CD74 (DHLAG), PSMA (FOLH1), CEACAM5 (CD66e), EGP1 (TROP2), FOLR1, CD37, Muc-16, Endothelial receptor (ETB), STEAP1, CD19, CD20, CD70 (TNFSF7), SLC44A4, Nectin-4, AGS-16, Guanylyl cyclase C, Muc-1, CD70 (TNFSF7), Her3 (ErbB-3), mesothelin, NaPi2b, LIV1, SLITRK6, ENPP3, TF, 5T4, BCMA, SCLC, Integrin, CD70 (TNFSF7), CA9 (MN), CFC1B (Cripto), CD98, C10orf54, or C16orf54.

15.-17. (canceled)

18. The antibody-drug conjugate of claim 1, wherein the opened cysteine-cysteine disulfide bond in A is an interchain disulfide bond.

19. (canceled)

20. The antibody-drug conjugate of claim 1 or 18, wherein L is —(CH₂)₅C(O)— and n is 4.

21. (canceled)

22. The antibody-drug conjugate of claim 1, which is of one the following formulas: 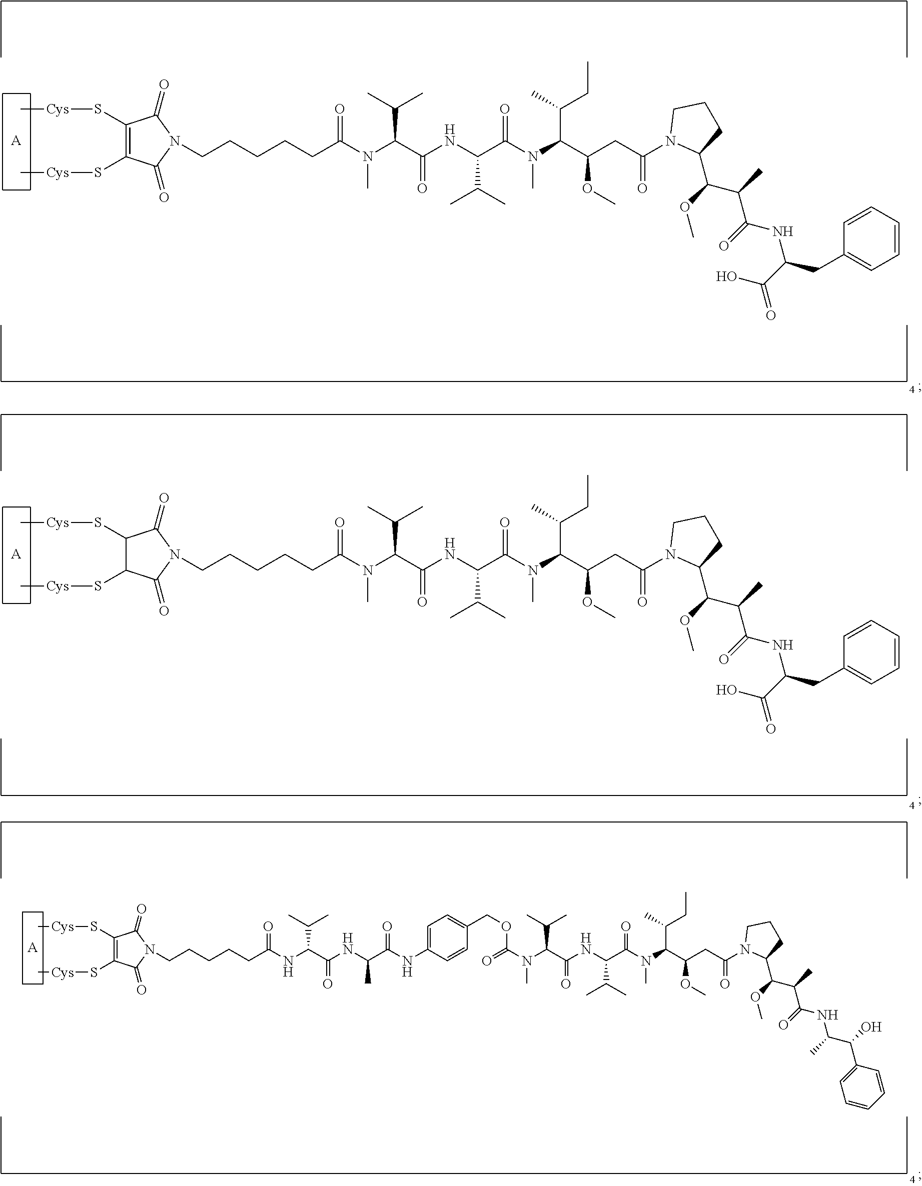 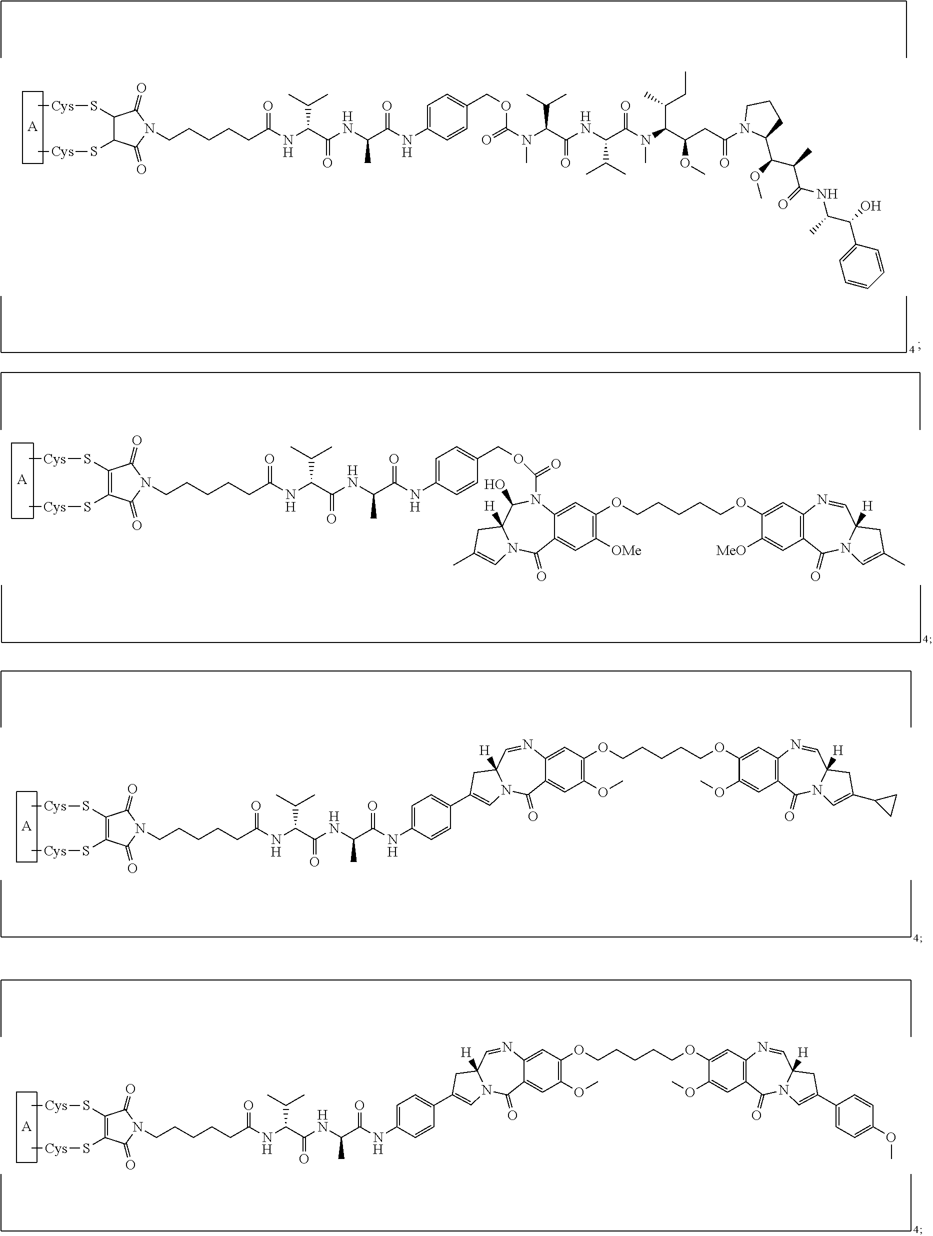 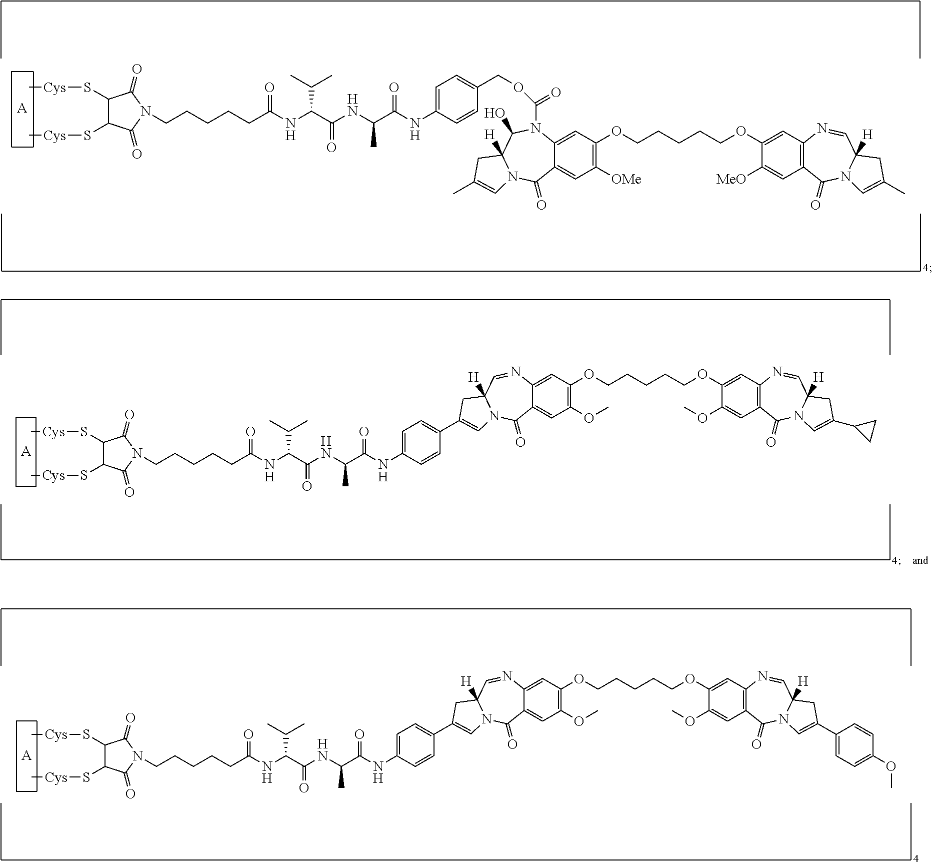
or a pharmaceutically acceptable salt thereof.

23.-29. (canceled)

30. The antibody-drug conjugate of claim 1, wherein A is trastuzumab, bevacizumab, rituximab, cetuximab, IGN523, or IGN786.

31. The antibody-drug conjugate of claim 1, wherein A comprises:
a VH sequence that comprises SEQ ID NO: 1 and a VL sequence that comprises SEQ ID NO: 2; a VH sequence that comprises SEQ ID NO: 3 and a VL sequence that comprises SEQ ID NO: 4;
a VH sequence that comprises SEQ ID NO: 5 and a VL sequence that comprises SEQ ID NO: 6;
a heavy chain sequence that comprises SEQ ID NO: 7 and a light chain sequence that comprises SEQ ID NO: 11;
a heavy chain sequence that comprises SEQ ID NO: 8 and a light chain sequence that comprises SEQ ID NO: 11;
a heavy chain sequence that comprises SEQ ID NO: 9 and a light chain sequence that comprises SEQ ID NO: 11;
a heavy chain sequence that comprises SEQ ID NO: 10 and a light chain sequence that comprises SEQ ID NO: 11;
a heavy chain sequence that comprises SEQ ID NO: 12 and a light chain sequence that comprises SEQ ID NO: 16;
a heavy chain sequence that comprises SEQ ID NO: 13 and a light chain sequence that comprises SEQ ID NO: 16;
a heavy chain sequence that comprises SEQ ID NO: 14 and a light chain sequence that comprises SEQ ID NO: 16; or
a heavy chain sequence that comprises SEQ ID NO: 15 and a light chain sequence that comprises SEQ ID NO: 16;
a heavy chain sequence that comprises SEQ ID NO: 17 and a light chain sequence that comprises SEQ ID NO: 21;
a heavy chain sequence that comprises SEQ ID NO: 18 and a light chain sequence that comprises SEQ ID NO: 21;
a heavy chain sequence that comprises SEQ ID NO: 19 and a light chain sequence that comprises SEQ ID NO: 21;
a heavy chain sequence that comprises SEQ ID NO: 20 and a light chain sequence that comprises SEQ ID NO: 21;
a heavy chain sequence that comprises SEQ ID NO: 22 and a light chain sequence that comprises SEQ ID NO: 26;
a heavy chain sequence that comprises SEQ ID NO: 23 and a light chain sequence that comprises SEQ ID NO: 26;
a heavy chain sequence that comprises SEQ ID NO: 24 and a light chain sequence that comprises SEQ ID NO: 26; or
a heavy chain sequence that comprises SEQ ID NO: 25 and a light chain sequence that comprises SEQ ID NO: 26.

32.-35. (canceled)

36. A linker-cytotoxin conjugate of one of the following formulas (IIa), (IIb), and (IIc): 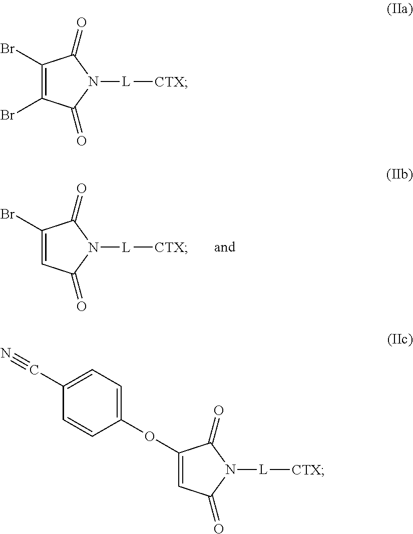
or an enantiomer, diasteriomer, or mixtures thereof;
wherein:
L is a cleavable or noncleavable linker; and
CTX is an auristatin, a pyrrolobenzodiazepine, calicheamicin, doxorubicin, camptothecin, duocarmycin, DM1, DM4, a maytansinoid, or a tubulysin, wherein CTX is bonded to L by an amide bond, a carbamate bond, a disulfide bond, an ether bond, a thioether bond, or an ester bond.

37. The linker-cytotoxin conjugate of claim 36, wherein CTX is an auristatin bonded to L by an amide bond or a carbamate bond.

38.-41. (canceled)

42. The linker-cytotoxin conjugate of claim 36, wherein L is —(CH₂)ₘC(O)—, wherein m is an integer of 5 to 11.

43.-44. (canceled)

45. The linker-cytotoxin conjugate of claim 36, which has one of the following structures:

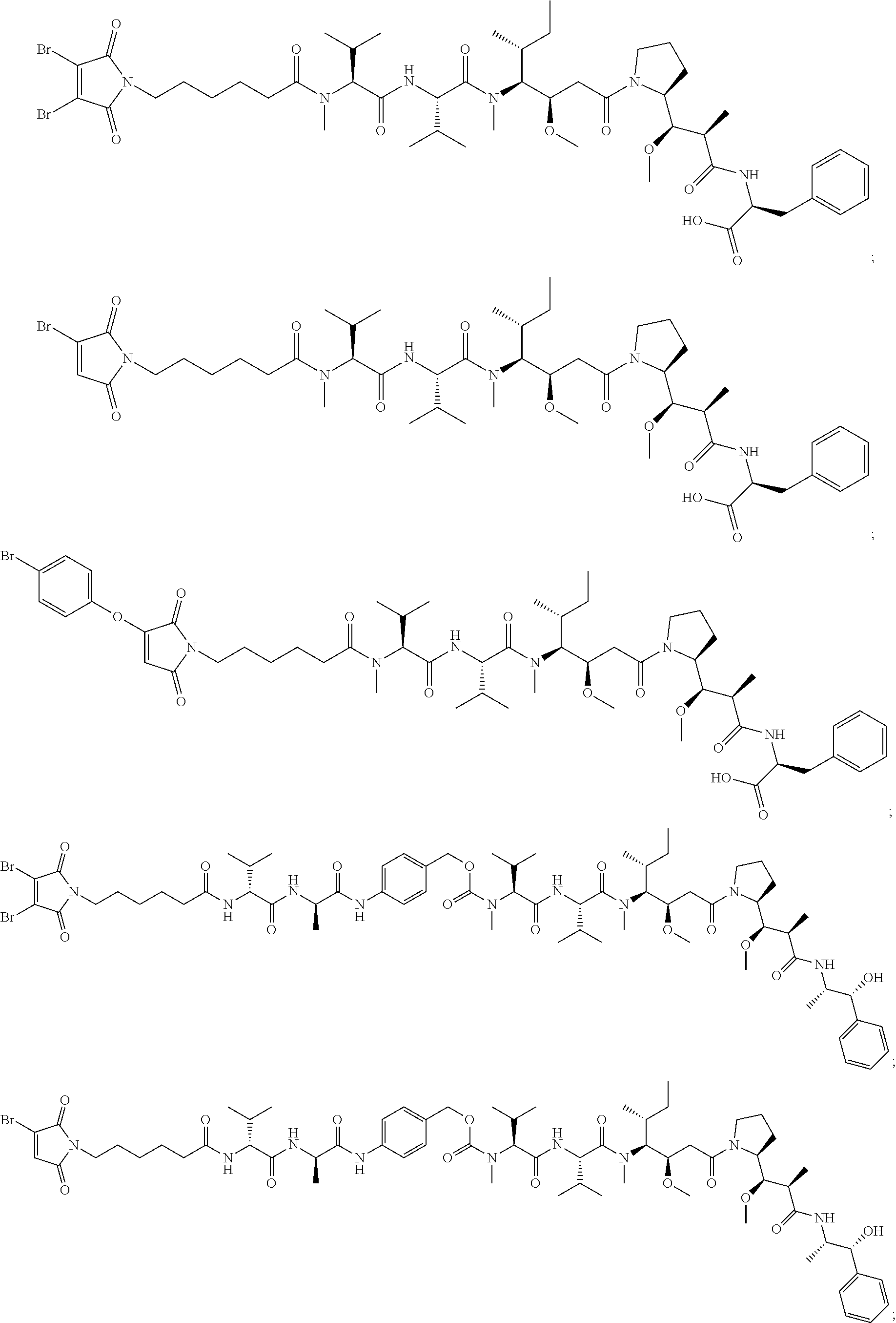

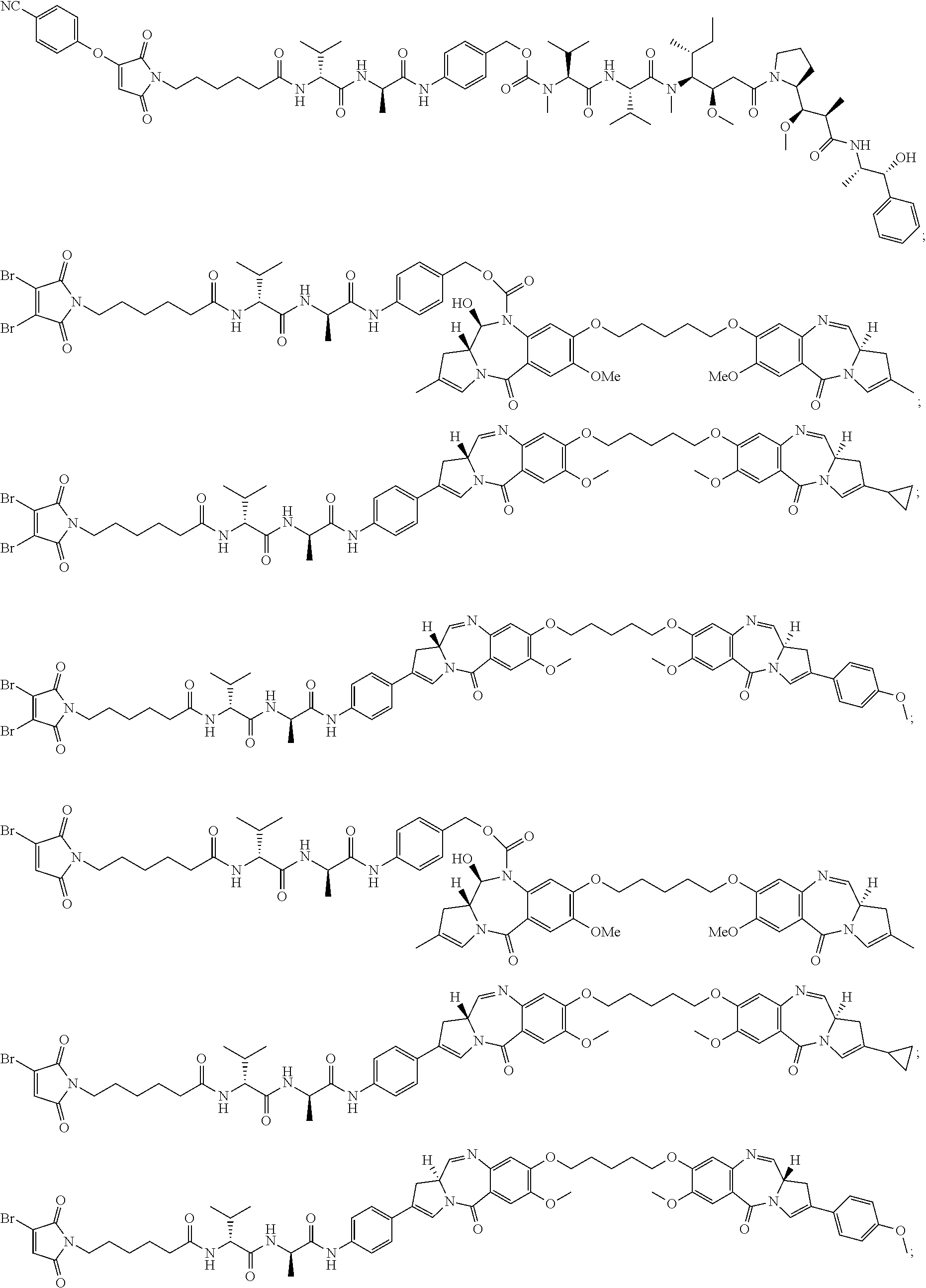

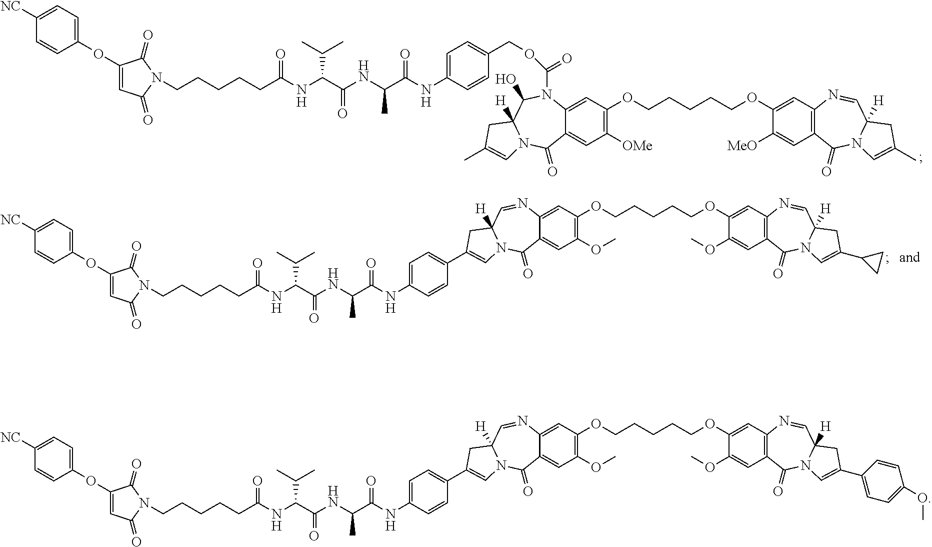

46.-53. (canceled)

54. A pharmaceutical composition comprising the antibody-drug conjugate of claim 1 or a pharmaceutically acceptable salt thereof, and a pharmaceutically acceptable diluent, carrier or excipient.

55. A method of treating a cancer by administering to a human suffering therefrom an effective amount of the antibody-drug conjugate of claim 1 or a pharmaceutically acceptable thereof.

56.-106. (canceled)

107. An antibody-drug conjugate comprising an antibody comprising:
a VH sequence that comprises SEQ ID NO: 1 and a VL sequence that comprises SEQ ID NO: 2; a VH sequence that comprises SEQ ID NO: 3 and a VL sequence that comprises SEQ ID NO: 4;
a VH sequence that comprises SEQ ID NO: 5 and a VL sequence that comprises SEQ ID NO: 6;
a heavy chain sequence that comprises SEQ ID NO: 7 and a light chain sequence that comprises SEQ ID NO: 11;
a heavy chain sequence that comprises SEQ ID NO: 8 and a light chain sequence that comprises SEQ ID NO: 11;
a heavy chain sequence that comprises SEQ ID NO: 9 and a light chain sequence that comprises SEQ ID NO: 11;
a heavy chain sequence that comprises SEQ ID NO: 10 and a light chain sequence that comprises SEQ ID NO: 11;
a heavy chain sequence that comprises SEQ ID NO: 12 and a light chain sequence that comprises SEQ ID NO: 16;
a heavy chain sequence that comprises SEQ ID NO: 13 and a light chain sequence that comprises SEQ ID NO: 16;
a heavy chain sequence that comprises SEQ ID NO: 14 and a light chain sequence that comprises SEQ ID NO: 16; or
a heavy chain sequence that comprises SEQ ID NO: 15 and a light chain sequence that comprises SEQ ID NO: 16;
a heavy chain sequence that comprises SEQ ID NO: 17 and a light chain sequence that comprises SEQ ID NO: 21;
a heavy chain sequence that comprises SEQ ID NO: 18 and a light chain sequence that comprises SEQ ID NO: 21;
a heavy chain sequence that comprises SEQ ID NO: 19 and a light chain sequence that comprises SEQ ID NO: 21;
a heavy chain sequence that comprises SEQ ID NO: 20 and a light chain sequence that comprises SEQ ID NO: 21;
a heavy chain sequence that comprises SEQ ID NO: 22 and a light chain sequence that comprises SEQ ID NO: 26;
a heavy chain sequence that comprises SEQ ID NO: 23 and a light chain sequence that comprises SEQ ID NO: 26;
a heavy chain sequence that comprises SEQ ID NO: 24 and a light chain sequence that comprises SEQ ID NO: 26; or
a heavy chain sequence that comprises SEQ ID NO: 25 and a light chain sequence that comprises SEQ ID NO: 26.

108.-149. (canceled)


SEQUENCE LISTING (30 sequences)

SEQ ID NO: 1 - protein, 120 aa, Artificial Sequence [sequence omitted]

SEQ ID NO: 2 - protein, 107 aa, Artificial Sequence [sequence omitted]

SEQ ID NO: 3 - protein, 135 aa, Artificial Sequence
  heavy chain variable region sequence of an exemplary anti-CD98 antibody designated as IGN523
MEWSWVFLFFLSVTTGVHSQVQLVQSGAEVKKPGSSVKVSCKASGNAFTNYLIEWVRQAPGQGLEWMGVINPGSGITNYNEKFKGKATITADKSTSTAYMELSSLRSEDTAVYYCSGSANWFAYWGQGTLVTVSS

SEQ ID NO: 4 - protein, 133 aa, Artificial Sequence
  light chain variable region sequence of an exemplary anti-CD98 antibody designated as IGN523
MSVPTQVLGLLLLWLTDARCDIVMTQSPDSLAVSLGERATINCKSSQSLLYSSNQKNYLAWYQQKPGQPPKLLIYWASTRDSGVPDRFTGSGSGTDFTLTISSLQAEDVAVYYCQRYYGYPWTFGGGTKVEIK

SEQ ID NO: 5 - protein, 121 aa, Artificial Sequence
  heavy chain variable region sequence of an exemplary anti-C16orf54 antibody designated as IGN786
QVQLQESGPGLVKPSDTLSLTCAVSGYSITSDYAWNWIRQPPGKGLEWMGYISYSGSIRYNPSLKSRITISRDTSKNQFSLKLSSVTAVDTAVYYCAREKYDNYYYAMDYWGQGTLVTVSS

SEQ ID NO: 6 - protein, 112 aa, Artificial Sequence
  light chain variable region sequence of an exemplary anti-C16orf54 antibody designated as IGN786
DIVMTQSPDSLAVSLGERVTLNCKSSQNLLYSTNQKNYLAWYQQKPGQPPKLLIYWASTRESGVPDRFSGSGSGTDFTLTISSVQAEDLAVYYCQQYYSYRTFGQGTKLEIK

SEQ ID NO: 7 - protein, 445 aa, Homo sapiens
  amino acid sequence for the IGN523 wild-type heavy chain
QVQLVQSGAEVKKPGSSVKVSCKASGNAFTNYLIEWVRQAPGQGLEWMGVINPGSGITNYNEKFKGKATITADKSTSTAYMELSSLRSEDTAVYYCSGSANWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 445 aa, Artificial Sequence
  amino acid sequence for the IGN523 single C226A heavy chain mutant
QVQLVQSGAEVKKPGSSVKVSCKASGNAFTNYLIEWVRQAPGQGLEWMGVINPGSGITNYNEKFKGKATITADKSTSTAYMELSSLRSEDTAVYYCSGSANWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 445 aa, Artificial Sequence
  amino acid sequence for the IGN523 single C229A heavy chain mutant
QVQLVQSGAEVKKPGSSVKVSCKASGNAFTNYLIEWVRQAPGQGLEWMGVINPGSGITNYNEKFKGKATITADKSTSTAYMELSSLRSEDTAVYYCSGSANWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 445 aa, Artificial Sequence
  amino acid sequence for the IGN523 double C226A-C229A heavy chain mutant
QVQLVQSGAEVKKPGSSVKVSCKASGNAFTNYLIEWVRQAPGQGLEWMGVINPGSGITNYNEKFKGKATITADKSTSTAYMELSSLRSEDTAVYYCSGSANWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 240 aa, Homo sapiens
  amino acid sequence of the IGN523 wild-type light chain
MSVPTQVLGLLLLWLTDARCDIVMTQSPDSLAVSLGERATINCKSSQSLLYSSNQKNYLAWYQQKPGQPPKLLIYWASTRDSGVPDRFTGSGSGTDFTLTISSLQAEDVAVYYCQRYYGYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 449 aa, Homo sapiens
  amino acid sequence of the trastuzumab wild-type heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 449 aa, Artificial Sequence
  amino acid sequence for the trastuzumab single C226A heavy chain mutant
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 449 aa, Artificial Sequence
  amino acid sequence for the trastuzumab single C229A heavy chain mutant
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 449 aa, Artificial Sequence
  amino acid sequence for the trastuzumab double C226A C229A heavy chain mutant
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 107 aa, Homo sapiens [sequence omitted]

SEQ ID NO: 17 - protein, 449 aa, Homo sapiens
  amino acid sequence for the IGN786 wild-type heavy chain
QVQLQESGPGLVKPSDTLSLTCAVSGYSITSDYAWNWIRQPPGKGLEWMGYISYSGSIRYNPSLKSRITISRDTSKNQFSLKLSSVTAVDTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 449 aa, Artificial Sequence
  amino acid sequence for the IGN786 single C226A heavy chain mutant
QVQLQESGPGLVKPSDTLSLTCAVSGYSITSDYAWNWIRQPPGKGLEWMGYISYSGSIRYNPSLKSRITISRDTSKNQFSLKLSSVTAVDTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 449 aa, Artificial Sequence
  amino acid sequence for the IGN786 single C229A heavy chain mutant
QVQLQESGPGLVKPSDTLSLTCAVSGYSITSDYAWNWIRQPPGKGLEWMGYISYSGSIRYNPSLKSRITISRDTSKNQFSLKLSSVTAVDTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 449 aa, Artificial Sequence
  amino acid sequence for the IGN786 double C226A-C229A heavy chain mutant
QVQLQESGPGLVKPSDTLSLTCAVSGYSITSDYAWNWIRQPPGKGLEWMGYISYSGSIRYNPSLKSRITISRDTSKNQFSLKLSSVTAVDTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 112 aa, Homo sapiens; identical to SEQ ID NO: 6
  amino acid sequence for the IGN786 wild-type light chain

SEQ ID NO: 22 - protein, 449 aa, Homo sapiens
  amino acid sequence for the IGN786-B wild-type heavy chain
QVQLQESGPGLVKPSQTLSLTCTVSGYSITSDYAWNWIRQPPGKGLEWMGYISYSGSIRYNPSLKSRITISRDTSKNQFSLKLSSVTAADTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 449 aa, Artificial Sequence
  amino acid sequence for the IGN786-B single C226A heavy chain mutant
QVQLQESGPGLVKPSQTLSLTCTVSGYSITSDYAWNWIRQPPGKGLEWMGYISYSGSIRYNPSLKSRITISRDTSKNQFSLKLSSVTAADTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTAPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 449 aa, Artificial Sequence
  amino acid sequence for the IGN786-B single C229A heavy chain mutant
QVQLQESGPGLVKPSQTLSLTCTVSGYSITSDYAWNWIRQPPGKGLEWMGYISYSGSIRYNPSLKSRITISRDTSKNQFSLKLSSVTAADTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTCPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 449 aa, Artificial Sequence
  amino acid sequence for the IGN786-B double C226A-C229A heavy chain mutant
QVQLQESGPGLVKPSQTLSLTCTVSGYSITSDYAWNWIRQPPGKGLEWMGYISYSGSIRYNPSLKSRITISRDTSKNQFSLKLSSVTAADTAVYYCAREKYDNYYYAMDYWGQGTLVTVSSASTKGPSVFPLAPSSKSTSGGTAALGCLVKDYFPEPVTVSWNSGALTSGVHTFPAVLQSSGYSLSSVVTVPSSSLGTQTYICNVNHKPSNTKVDKRVEPKSCDKTHTAPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 112 aa, Homo sapiens; identical to SEQ ID NO: 6
  amino acid sequence for the IGN786-B wild-type light chain

SEQ ID NO: 27 - protein, 23 aa, Homo sapiens
  hinge sequence of Human IgG1 antibody
EPKSCDKTHTCPPCPAPELLGGP

SEQ ID NO: 28 - protein, 19 aa, Homo sapiens
  hinge sequence of Human IgG2 antibody
ERKCCVECPPCPAPPVAGP

SEQ ID NO: 29 - protein, 70 aa, Homo sapiens
  hinge sequence of Human IgG3 antibody
ELKTPLGDTTHTCPRCPEPKSCDTPPPCPRCPEPKSCDTPPPCPRCPEPKSCDTPPPCPRCPAPELLGGP

SEQ ID NO: 30 - protein, 20 aa, Homo sapiens
  hinge sequence of Human IgG4 antibody
ESKYGPPCPSCPAPEFLGGP